(19) United States
(12) Patent Application Publication
(10) Pub. No.: US 2020/0149070 A1
(43) Pub. Date: May 14, 2020

(54) METHODS AND COMPOSITIONS FOR GENOME EDITING

(71) Applicant: Ligandal, Inc., San Francisco, CA (US)

(72) Inventors: Andre Ronald Watson, San Francisco, CA (US); Christian Foster, Oakland, CA (US); Shuailiang Lin, San Francisco, CA (US)

(21) Appl. No.: 16/393,886

(22) Filed: Apr. 24, 2019

Related U.S. Application Data
(60) Provisional application No. 62/661,992, filed on Apr. 24, 2018.
(60) Provisional application No. 62/685,240, filed on Jun. 14, 2018.

(51) Int. Cl. C12N 15/90; C12N 9/22; C12N 15/113; C12N 15/52; C07K 14/705

(52) U.S. Cl. CPC C12N 15/90; C12N 9/22; C12N 15/113; C12N 15/52; B82Y 5/00; C12N 2310/20; C07K 2317/622; C07K 2317/55; C07K 14/70596

(57) ABSTRACT

Provided are methods and compositions for genome editing using a delivery vehicle with multiple payloads. In some embodiments the delivery vehicle includes a payload that includes (a) one or more sequence specific nucleases that cleave the cell's genome or one or more nucleic acids encoding same, (b) a first donor DNA, which includes a nucleotide sequence that is inserted into the cell's genome, where insertion of said nucleotide sequence produces, in the cell's genome at the site of insertion, a target sequence (e.g., an attP site) for a site-specific recombinase; (c) the site-specific recombinase (or a nucleic acid encoding same) (e.g., ϕC31, ϕC31 RDF, Cre, FLP), where the site-specific recombinase recognizes said target sequence; and (d) a second donor DNA, which includes a nucleotide sequence that is inserted into the cell's genome as a result of recognition of said target sequence by the site-specific recombinase.

### CROSS-REFERENCE

This application claims the benefit of U.S. Provisional Patent Application No. 62/661,992, filed Apr. 24, 2018, and of U.S. Provisional Patent Application No. 62/685,240, filed Jun. 14, 2018, both of which applications are incorporated herein by reference in their entirety.

### SEQUENCE LISTING

This application contains a Sequence Listing, which was submitted in ASCII format via EFS-Web, and is hereby incorporated by reference in its entirety. The ASCII copy, created on Feb. 3, 2020, is named 2020-02-03_Ligandal-8005U502-Sequence-Listing and is 146 KB in size.

### INTRODUCTION

Genome editing remains an inefficient process in most circumstances. While many techniques exist for performing site specific gene editing, clinical translation is hampered by inadequate delivery technologies—especially when considering in vivo delivery. As such, compositions and methods for efficient genome editing remain an important unmet need.

### SUMMARY

Provided are compositions and methods for genome editing using a delivery vehicle with multiple payloads. In some embodiments, subject methods include introducing a delivery vehicle into a cell, where the delivery vehicle includes a payload that includes (a) one or more sequence specific nucleases that cleave the cell's genome (e.g., a meganuclease, a homing endonuclease, a zinc finger nuclease (ZFN), a TALEN, a type I or type III CRISPR/Cas cleavage complex, a class 2 CRISPR/Cas effector protein—an RNA-guided CRISPR/Cas polypeptide—such as Cas9, CasX, CasY, Cpf1 (Cas12a), Cas13, MAD7, and the like) or one or more nucleic acids that encode the one or more sequence specific nucleases [(a) is referred to herein as a nuclease composition]; (b) a first donor DNA, which includes a nucleotide sequence that is inserted into the cell's genome, where insertion of said nucleotide sequence produces, in the cell's genome at the site of insertion, a target sequence (e.g., an attP site) for a site-specific recombinase [(b) is referred to herein as a target donor composition]; (c) the site-specific recombinase (or a nucleic acid encoding same) (e.g., ϕC31, ϕC31 RDF, Cre, FLP), where the site-specific recombinase recognizes said target sequence [(c) is referred to herein as a recombinase composition]; and (d) a second donor DNA, which includes a nucleotide sequence that is inserted into the cell's genome as a result of recognition of said target sequence by the site-specific recombinase [(d) is referred to herein as an insert donor composition]. A delivery vehicle that includes (a), (b), (c), and (d) facilitates insertion of large sequences into the genome by insertion of one or more target sites for a site-specific recombinase followed by recombinase-mediated insertion of a nucleotide sequence of interest. In some cases, the inserted nucleotide sequence of interest (from the insert donor composition) is 10 kilobase pairs (kbp) or more (e.g., from 15 kbp to 100 kbp, from 30 kbp to 100 kbp, or from 50 kbp to 100 kbp).

In some cases insertion of the nucleotide sequence of the first donor DNA produces a first target sequence for the site-specific recombinase at a first location in the cell's genome and a second target sequence for the site-specific recombinase at a second location in the cell's genome (e.g., insertion of two attP sites). In some cases the nuclease composition cleaves the cell's genome at two locations, and the target donor composition includes two of the first donor DNAs, each of which includes a nucleotide sequence that is inserted into the cell's genome, thereby producing a first target sequence for the site-specific recombinase at a first location in the cell's genome and a second target sequence for the site-specific recombinase at a second location in the cell's genome (e.g., insertion of two attP sites). In some cases the second donor DNA includes two target sequences (e.g., attB sites) for the site-specific recombinase, where the two target sequences flank the nucleotide sequence that is inserted into the cell's genome.

The delivery vehicle can be introduced into a cell/delivered to a cell in vitro, ex vivo, or in vivo. One advantage of delivering multiple payloads as part of the same delivery vehicle (e.g., nanoparticle) is that the efficiency of each payload is not diluted. As an illustrative example, if payload A and payload B are delivered in two separate packages/vehicles (package A and package B, respectively), then the efficiencies are multiplicative, e.g., if package A and package B each have a 1% transfection efficiency, the chance of delivering payload A and payload B to the same cell is 0.01% (1%×1%). However, if payload A and payload B are both delivered as part of the same delivery vehicle, then the chance of delivering payload A and payload B to the same cell is 1%, a 100-fold improvement over 0.01%.

Delivery vehicles can include, but are not limited to, non-viral vehicles, viral vehicles, nanoparticles (e.g., a nanoparticle that includes a targeting ligand and/or a core comprising an anionic polymer composition, a cationic polymer composition, and a cationic polypeptide composition), liposomes, micelles, water-oil-water emulsion particles, oil-water emulsion micellar particles, multilamellar water-oil-water emulsion particles, a targeting ligand (e.g., peptide targeting ligand) conjugated to a charged polymer polypeptide domain (wherein the targeting ligand provides for targeted binding to a cell surface protein, and the charged polymer polypeptide domain is condensed with a nucleic acid payload and/or is interacting electrostatically with a protein payload), a targeting ligand (e.g., peptide targeting ligand) conjugated to payload (where the targeting ligand provides for targeted binding to a cell surface protein), etc. In some cases payloads are introduced into the cell as deoxyribonucleoprotein complex(s) and/or a ribo-deoxyribonucleoprotein complex(s).

The provided compositions and methods can be used for genome editing at any locus in any cell type (e.g., to engineer T-cells, e.g., in vivo). For example, a CD8+ T-cell population or mixture of CD8+ and CD4+ T-cells can be programmed to transiently or permanently express an appropriate TCRα/TCRβ pair of CDR1, CDR2, and/or CDR3 domains for antigen recognition.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 depicts a schematic representation of one example of a subject method. In this particular example: two target sequences (e.g., attP sites) are inserted into the genome (using the donor DNA of the target donor composition) after cleavage of the genome with a sequence specific nuclease; two target sequences (e.g., attB sites) are present on the donor DNA of the insert donor composition; and two residual sites (e.g., attR sites) remain present in the genome after insertion of sequence from the donor DNA (of the insert donor composition) using a site-specific recombinase (e.g., PhiC31 ϕC31)).

FIG. 2 depicts a schematic representation of an example embodiment of a delivery vehicle (in the depicted case, one type of nanoparticle).

FIG. 3 depicts a schematic representation of an example embodiment of a delivery vehicle (in the depicted case, one type of nanoparticle). In this case, the depicted nanoparticle is multi-layered, having a core (which includes a first payload) surrounded by a first sheddable layer, which is surrounded by an intermediate layer (which includes an additional payload), which is surrounded by a second sheddable layer, which is surface coated (i.e., includes an outer shell).

FIG. 4 (panels A-B) depicts schematic representations of example configurations of a targeting ligand of a surface coat of a subject nanoparticle. The delivery molecules depicted include a targeting ligand conjugated to an anchoring domain that is interacting electrostatically with a sheddable layer of a nanoparticle. Note that the targeting ligand can be conjugated at the N- or C-terminus (left of each panel), but can also be conjugated at an internal position (right of each panel). The molecules in panel A include a linker while those in panel B do not.

FIG. 5A provides a schematic drawing of an example embodiment of a donor vehicle (in the depicted case, example configurations of a subject delivery molecule). The targeting ligand can be conjugated at the N- or C-terminus (left of the figure), but can also be conjugated at an internal position (right of the figure). This figure shows delivery molecules including a linker as well as a targeting ligand conjugated to a payload.

FIG. 5B provides a schematic drawing of an example embodiment of a donor vehicle (in the depicted case, example configurations of a subject delivery molecule). The targeting ligand can be conjugated at the N- or C-terminus (left of the figure), but can also be conjugated at an internal position (right of the figure). This figure shows delivery molecules including do not have a linker but do have a targeting ligand conjugated to a payload.

FIG. 5C provides a schematic drawing of an example embodiment of a donor vehicle (in the depicted case, example configurations of a subject delivery molecule). The targeting ligand can be conjugated at the N- or C-terminus (left of the figure), but can also be conjugated at an internal position (right of the figure). This figure shows delivery molecules including a linker and a targeting ligand conjugated to a charged polymer polypeptide domain that is condensed with a nucleic acid payload (and/or interacting, e.g. electrostatically, with a protein payload).

FIG. 5D provides a schematic drawing of an example embodiment of a donor vehicle (in the depicted case, example configurations of a subject delivery molecule). The targeting ligand can be conjugated at the N- or C-terminus (left of the figure), but can also be conjugated at an internal position (right of the figure). This figure shows delivery molecules that do not have a linker but do have a targeting ligand conjugated to a charged polymer polypeptide domain that is condensed with a nucleic acid payload (and/or interacting, e.g. electrostatically, with a protein payload).

FIG. 6 provides non-limiting examples of nuclear localization signals (NLSs) that can be used (e.g., as part of a nanoparticle, e.g., as an NLS-containing peptide; as part of/conjugated to an NLS-containing peptide, an anionic polymer, a cationic polymer, and/or a cationic polypeptide; and the like). The figure is adapted from Kosugi et al., J Biol Chem. 2009 Jan. 2; 284(1):478-85. (Class 1, top to bottom (SEQ ID NOs: 201-221); Class 2, top to bottom (SEQ ID NOs: 222-224); Class 4, top to bottom (SEQ ID NOs: 225-230); Class 3, top to bottom (SEQ ID NOs: 231-245); Class 5, top to bottom (SEQ ID NOs: 246-264)].

FIG. 7 depicts schematic representations of the mouse hematopoietic cell lineage, and markers that have been identified for various cells within the lineage.

FIG. 8 depicts schematic representations of the human hematopoietic cell lineage, and markers that have been identified for various cells within the lineage.

FIG. 9 depicts schematic representations of miRNA factors that can be used to influence cell differentiation and/or proliferation.

FIG. 10 depicts schematic representations of protein factors that can be used to influence cell differentiation and/or proliferation.

FIG. 11 depicts the average particle size of nanoparticles with the compositions of Study A.

FIG. 12 depicts zeta potential of nanoparticles with the compositions of Study A.

FIG. 13 depicts the polydispersity index of nanoparticles with the compositions of Study A.

FIG. 14 depicts a chart describing the polydispersity index and stability of nanoparticles with the compositions of Study B.

FIG. 15 depicts the average particle size of nanoparticles with the compositions of Study B.

FIG. 16 depicts zeta potential of nanoparticles with the compositions of Study B.

FIG. 17 depicts the polydispersity index of nanoparticles with the compositions of Study B.

FIG. 18 depicts a chart describing the polydispersity index and stability of nanoparticles with the compositions of Study B.

FIG. 19 depicts the particle size distribution of nanoparticles with compositions of Study C.

FIG. 20 depicts the zeta potential of nanoparticles with the compositions of Study C.

FIG. 21 depicts the polydispersity index of nanoparticles with the compositions of Study C.

FIG. 22 depicts the polydispersity index and stability of nanoparticles with the compositions of Study C.

FIG. 23 depicts the fluorescence values obtained with the SYBR GOLD Inclusion assay of nanoparticles with the compositions of Study C.

FIG. 24 depicts the characterization of RNP-H2B (mini core) particles.

FIG. 25 depicts the characterization of RNP-H2B-PLE20:PDE20 (core) particles.

FIG. 26 depicts the serum stability of RNP-H2B-PLE20:PDE20 (core) particles.

FIG. 27 depicts the polydispersity evaluation of ligand-modified particles after one day.

FIG. 28 depicts the polydispersity evaluation of ligand-modified particles after 72 hours or seven days.

FIG. 29 depicts cells, nuclei, and nanoparticles as measured by automated pipeline sampling.

FIG. 30 depicts changes in cellular particle colocalization over 14.5 hours after various treatments.

FIG. 31 depicts the percentage of Cas9-eGFP cells over 14.5 hours after various treatments.

FIG. 32 depicts nuclear particle integration over 14.5 hours after various treatments.

FIG. 33 depicts the percentage of Cas9-eGFP cells over 14.5 hours after various treatments.

FIG. 34A depicts T-cell and PBMC targeting in untreated samples.

FIG. 34B depicts the human primary pan T-cell data corresponding to FIG. 34A.

FIG. 34C depicts the human primary PBMCs data corresponding to FIG. 34A.

FIG. 35A depicts T-cell and PBMC targeting in core particles.

FIG. 35B depicts the human primary pan T-cell data corresponding to FIG. 35A.

FIG. 35C depicts the human primary PBMCs data corresponding to FIG. 35A.

FIG. 36A depicts T-cell and PBMC targeting in samples treated with ligand poly(L-arginine).

FIG. 36B depicts the human primary pan T-cell data corresponding to FIG. 36A.

FIG. 36C depicts the human primary PBMCs data corresponding to FIG. 36A.

FIG. 37A depicts T-cell and PBMC targeting in samples with ligand CD3_CD3e_(4GS)2_9R_N_1.

FIG. 37B depicts the human primary pan T-cell data corresponding to FIG. 37A.

FIG. 37C depicts the human primary PBMCs data corresponding to FIG. 37A.

FIG. 38A depicts T-cell and PBMC targeting in samples with ligand CD8_(4GS)2_9R_N.

FIG. 38B depicts the human primary pan T-cell data corresponding to FIG. 38A.

FIG. 38C depicts the human primary PBMCs data corresponding to FIG. 38A.

FIG. 39A depicts T-cell and PBMC targeting in samples with ligand CD28_mCD80_(4GS)2_9R_N.

FIG. 39B depicts the human primary pan T-cell data corresponding to FIG. 39A.

FIG. 39C depicts the human primary PBMCs data corresponding to FIG. 39A.

FIG. 40A depicts T-cell and PBMC targeting in samples with ligands CD28_mCD86_(4GS)2_9R_N.

FIG. 40B depicts the human primary pan T-cell data corresponding to FIG. 40A.

FIG. 40C depicts the human primary PBMCs data corresponding to FIG. 40A.

FIG. 41A depicts T-cell and PBMC targeting in samples with ligands IL2R_mIL2_4 GS_2_9R_N_1.

FIG. 41B depicts the human primary pan T-cell data corresponding to FIG. 41A.

FIG. 41C depicts the human primary PBMCs data corresponding to FIG. 41A.

FIG. 42A depicts T-cell and PBMC targeting in samples with ligands CD3_CD3e_(4GS)2_9R_N_1 and CD8_(4GS)2_9R_N.

FIG. 42B depicts the human primary pan T-cell data corresponding to FIG. 42A.

FIG. 42C depicts the human primary PBMCs data corresponding to FIG. 42A.

FIG. 43A depicts T-cell and PBMC targeting in samples with ligands CD3_CD3e_(4GS)2_9R_N_1 and CD28_mCD80_(4GS)2_9R_N.

FIG. 43B depicts the human primary pan T-cell data corresponding to FIG. 43A.

FIG. 43C depicts the human primary PBMCs data corresponding to FIG. 43A.

FIG. 44A depicts T-cell and PBMC targeting in samples with ligands CD3_CD3e_(4GS)2_9R_N_1 and CD28_mCD86_(4GS)2_9R_N.

FIG. 44B depicts the human primary pan T-cell data corresponding to FIG. 44A.

FIG. 44C depicts the human primary PBMCs data corresponding to FIG. 44A.

FIG. 45A depicts T-cell and PBMC targeting in samples with ligands CD3_CD3e_(4GS)2_9R_N_1 and IL2R_mIL2_4 GS_2_9R_N_1.

FIG. 45B depicts the human primary pan T-cell data corresponding to FIG. 45A.

FIG. 45C depicts the human primary PBMCs data corresponding to FIG. 45A.

FIG. 46A depicts T-cell and PBMC targeting in samples with ligands CD3_CD3e_(4GS)2_9R_N_1 and poly(L-arginine)n=10.

FIG. 46B depicts the human primary pan T-cell data corresponding to FIG. 46A.

FIG. 46C depicts the human primary PBMCs data corresponding to FIG. 46A.

FIG. 47A depicts T-cell and PBMC targeting in samples with ligands CD2B_mCD80_(4GS)2_9R_N and CD28_mCD86_(4GS)2_9R_N.

FIG. 47B depicts the human primary pan T-cell data corresponding to FIG. 47A.

FIG. 47C depicts the human primary PBMCs data corresponding to FIG. 47A.

FIG. 48A depicts T-cell and PBMC targeting in samples with ligands CD3_CD3e_(4GS)2_9R_N_1, CD2B_mCD86_(4GS)2_9R_N, and CD8_4 GS_2_9R_N_1.

FIG. 48B depicts the human primary pan T-cell data corresponding to FIG. 48A.

FIG. 48C depicts the human primary PBMCs data corresponding to FIG. 48A.

FIG. 49A depicts T-cell and PBMC targeting in samples with ligands CD3_CD3e_(4GS)2_9R_N_1, CD8_4 GS_2_9R_N_1, and IL2R_mIL2_4 GS_2_9R_N_1.

FIG. 49B depicts the human primary pan T-cell data corresponding to FIG. 49A.

FIG. 49C depicts the human primary PBMCs data corresponding to FIG. 49A.

FIG. 50A depicts T-cell and PBMC targeting in samples with ligands CD3_CD3e_(4GS)2_9R_N_1, CD2B_mCD80_(4GS)2_9R_N, and CD8_4 GS_2_9R_N_1.

FIG. 50B depicts the human primary pan T-cell data corresponding to FIG. 50A.

FIG. 50C depicts the human primary PBMCs data corresponding to FIG. 50A.

FIG. 51A depicts T-cell and PBMC targeting in samples with CD3_CD3e_(4GS)2_9R_N_1, CD2B_mCD86_(4GS)2_9R_N, and CD28_mCD80_(4GS_)2_9R_N_1.

FIG. 51B depicts the human primary pan T-cell data corresponding to FIG. 51A.

FIG. 51C depicts the human primary PBMCs data corresponding to FIG. 51A.

FIG. 52A depicts T-cell and PBMC targeting in samples with ligands CD8_4 GS_2_9R_N_1, CD28_mCD80_(4GS)2_9R_N, and CD28_mCD86_(4GS)2_9R_N.

FIG. 52B depicts the human primary pan T-cell data corresponding to FIG. 52A.

FIG. 52C depicts the human primary PBMCs data corresponding to FIG. 52A.

FIG. 53A depicts T-cell and PBMC targeting in samples with ligands CD8_4 GS_2_9R_N_1, CD28_mCD80_(4GS)2_9R_N, and IL2R_mIL2_4 GS_2_9R_N_1.

FIG. 53B depicts the human primary pan T-cell data corresponding to FIG. 53A.

FIG. 53C depicts the human primary PBMCs data corresponding to FIG. 53A.

FIG. 54A depicts T-cell and PBMC targeting in samples with ligands CD8_4 GS_2_9R_N_1, CD28_mCD86_(4GS)2_9R_N, IL2R_mIL2_4 GS_2_9R_N_1.

FIG. 54B depicts the human primary pan T-cell data corresponding to FIG. 54A.

FIG. 54C depicts the human primary PBMCs data corresponding to FIG. 54A.

FIG. 55 depicts the image analysis, Pan-T cell flow analysis, and PBMC flow analysis of samples with different ligands.

FIG. 56 depicts the image analysis, Pan-T cell flow analysis, and PBMC flow analysis of samples with ligand sets that are different from those of FIG. 55.

FIG. 57 depicts a general schematic of nanoparticle synthesis. This involves addition of payloads, cationic/anionic peptides, and ligands, demonstrating varying orders of addition and degrees of freedom. Peptide, payload, and ligand examples are given and include different mer lengths and D/L isomers.

FIG. 58A depicts volumes of PLR50, buffer, and RNP used in the formulation of each nanoparticle added stepwise.

FIG. 58B depicts volumes of PLR50, buffer, and RNP used in the formulation of each nanoparticle added stepwise continued.

FIG. 58C depicts volumes of DNA, PLR50, and buffer used in the formulation of each nanoparticle added stepwise.

FIG. 58D depicts volumes of DNA, PLR50 and buffer used in the formulation of each nanoparticle added stepwise (continued).

FIG. 58E depicts the nanoparticle well ID (location in 96-well plate where it was synthesized and measured for size, zeta potential, and SYBR fluorescence) and its conversion to Nanoparticle ID (reference to the 96-well cell transfection plate) in FIG. 61I.

FIG. 59A depicts particle sizes of nanoparticles synthesized in 2C.1.1.1. Particle sizes were measured in triplicate via a Wyatt Mobius Zeta Potential and DLS Detector. Sizes are reported as average hydrodynamic diameter (nm)±standard deviation in a heatmap which correlates to the nanoparticle 96-well ID.

FIG. 59B depicts zeta potentials of nanoparticles synthesized in 2C.1.1.1. Particle zeta potentials were measured in triplicate via a Wyatt Mobius Zeta Potential and DLS Detector. Zeta potentials are reported as average zeta potentials (mV)±standard deviation in a heatmap which correlates to the nanoparticle 96-well ID.

FIG. 59C depicts the average (Overnight) condensation index of each particle in 2C.1.1.1 using SYBR fluorescence assay. The condensation index is calculated as [(Well of Interest Fluorescence−Free DNA Fluorescence)/Free DNA Fluorescence]*100 and is reported as average condensation index±standard deviation in a heatmap which correlates to the nanoparticle 96-well ID. The more condensed nanoparticles will have higher shielding, less fluorescence, and thus a more negative condensation index.

FIG. 60A depicts the plate layout for 20.1.1.1 transfection of HEK293-GFP cells. NPs were dosed at 20 uL and 10 uL on duplicate plates. Row B (gold highlight) shows Layer 1 Charge Ratios, Row C (orange highlight) shows Outer Layer Charge Ratios, green highlight shows CRISPRMAX transfection controls, grey highlight shows Lipofectamine3000 transfection controls. The DNA Mix includes the ssODN and PhiC31 expression and donor plasmids.

FIG. 60B depicts 20.1.1.1 Flow results for Day 5 post transfection, monitoring GFP and Alexa647 (NP) fluorescence. Live cells were gated based on FSC/SSC scatter and % GFP− and % NP+ are shown for live gate. No significant GFP KD is observed for NP wells, although lipofection controls give robust results. The 20 uL dose of NP has significant NP signal at Day 5, while 10 uL dose does not. RFP expression was observed only in the lipofection control with Tag-RFP plasmid: 20% RFP+ of Live, well C11 (data not shown). Flow analysis on Day 10 and Day 14 post transfection showed no more NP Alexa-647 signal, no GFP knock-down, and no RFP expression for NP groups. RFP expression decreased over time in plasmid lipofection controls (7% RFP+ Day 10, 2% RFP+ Day 14), while GFP knock-down in CRISPRMAX controls (B10, 010) remained constant, consistent with heritable gene editing.

FIG. 60C depicts results of 20.1.1.1 Day 5 post-transfection genomic analysis for the RNP+ DNA Mix lipofection control (well 010), showing editing of the GFP locus (25% KD) and HDR (knock-in) of the attP ssODN (6%) using Synthego's ICE-KI analysis of Sanger sequencing data. The guide target sequence is set forth in SEQ ID NO: 278. The sequences in the alignment are set forth from top to bottom in SEQ ID NOs: 304-319.

FIG. 61A depicts volumes of PLR50, buffer, and RNP used in the formulation of each nanoparticle added stepwise.

FIG. 61B depicts volumes of PLR50, buffer, and RNP used in the formulation of each nanoparticle added stepwise (continued).

FIG. 61C depicts volumes of DNA, Ligand Mix, ALEXA-647 nanoparticle label, and buffer used in the formulation of each nanoparticle added stepwise.

FIG. 61D depicts volumes of PLR50, buffer, and RNP used in the formulation of each nanoparticle added stepwise (continued).

FIG. 61E depicts the nanoparticle well ID (location in 96-well plate where it was synthesized and measured for size, zeta potential, and SYBR fluorescence) and its conversion to Nanoparticle ID (reference to the 96-well cell transfection plate) in FIG. 61I.

FIG. 61F depicts particle sizes of nanoparticles synthesized in 2C.2.1.1. Particle sizes were measured in triplicate via a Wyatt Mobius Zeta Potential and DLS Detector. Sizes are reported as average hydrodynamic diameter (nm)±standard deviation in a heatmap which correlates to the nanoparticle 96-well ID.

FIG. 61G depicts zeta potentials of nanoparticles synthesized in 2C.2.1.1. Particle zeta potentials were measured in triplicate via a Wyatt Mobius Zeta Potential and DLS Detector. Zeta potentials are reported as average zeta potentials (mV)±standard deviation in a heatmap which correlates to the nanoparticle 96-well ID.

FIG. 61H depicts the average (Overnight) condensation index of each particle in 2C.2.1.1 using SYBR fluorescence assay. The condensation index is calculated as [(Well of Interest Fluorescence−Free DNA Fluorescence)/Free DNA Fluorescence]*100 and is reported as average condensation index±standard deviation in a heatmap which correlates to the nanoparticle 96-well ID. The more condensed nanoparticles will have higher shielding, less fluorescence, and thus a more negative condensation index.

FIG. 61I depicts the plate layout for 2C.2.1.1 transfection of stimulated PBMCs. NPs were dosed at 10 uL per well of 60,000 stimulated PBMCs. Row B (gold highlight) shows Layer 1 Charge Ratios, Row C (orange highlight) shows Outer Layer Charge Ratios. Green highlighted wells (B12-G12 and G11) are nucleofection controls. NP18, NP39, and NP25 were the highest performing nanoparticle groups in terms of gene editing efficiency (TCR k/d; 6%, 5% and 5% Sanger sequencing efficiency, respectively). Bolded values indicate TCR locus cutting values >1% for the highlighted samples as determined by Sanger sequencing.

FIG. 61J depicts cell viability (% Live) of transfected cells from experiment 2C.2.1.1 as measured via flow cytometry. Following PBS wash of nanoparticles from overnight transfection, stimulated PBMCs were gathered for Day 1 flow analysis. Cell Viability was assayed by Annexin V staining. Well E6 had an error.

FIG. 61K depicts cell viability (% Dead/Apoptotic) of transfected cells from experiment 2C.2.1.1 as measured via flow cytometry. Following PBS wash of nanoparticles from overnight transfection, stimulated PBMCs were gathered for Day 1 flow analysis. Cell Viability was assayed by Annexin V staining. Well E6 had an error. poptotic and dead cells. Well E6 had an error.

FIG. 61L depicts cellular uptake (AF647-NP+ and EGFP-Cas9+) of various nanoparticle formulations from 2C.2.1.1. Following PBS wash of nanoparticles from overnight transfection, stimulated PBMCs were gathered for Day 1 flow analysis. Shown is GFP (Cas9-GFP) and Alexa647 (NP) fluorescence and % GFP+ and % NP+ are shown for live gate (Annexin V negative). Very little NP signal (Alexa647) is observed in T cells compared to HEK-293 cells, suggesting rapid degradation of the AF647-bound endosomal escape peptide results in the most efficient subcellular Cas9 RNP delivery. Almost all trace NP+ levels (1-2%) are coincident with the highest-value Cas9-GFP signals. CD3 staining showed that >95% of cells were CD3+ (not shown), indicating that CD3/CD28 stimulation resulted in selective proliferation of T Cells.

FIG. 61M depicts flow analysis of TCR expression on Day 4 post-transfection in 2C.2.1.1. Nucleofection wells with RNP+/−ssODN have robust TCR KD, while nucleofection of all components (F12) and NP wells do not. No NP signal (Alexa647 or Cas9-GFP) is observed, and only the RFP plasmid nucleofection control well (G12) shows RFP expression at 10% of live cells (data not shown).

FIG. 61N depicts flow analysis of TCR expression on Day 14 post-transfection in 2C.2.1.1. No NP signal (Alexa647 or Cas9-GFP) or RFP expression is observed, even from the plasmid nucleofection control.

FIG. 61O depicts ICE scores of Day 14 Sanger sequencing data for TRAC locus. High ICE scores show robust editing in nucleofection samples, similar to TCR KD by flow. Three NPs (boxed) showed modest TCR editing by Synthego's ICE platform. No samples showed HDR (knock-in) with the attP ssODN.

FIG. 61P depicts Sanger sequencing results of 2C.2.1.1, a study performed prior to 2C.1.2.1 whereby cores were further optimized to result in higher nanoparticle uptake efficiencies and subcellular release kinetics. 2C.1.2.1 further included variable ratios of PLE to PDE, along with variable ratios of histone fragments, PLR10, PLR50, NLS-modified histones, and/or endosomal escape—NLS peptide. Prior to decoration in targeting ligands, various nanoparticle cores were assessed for their biological performance (cellular uptake, cellular persistence over time, viability, and gene editing) in an initial set of screens intended to optimize nanoparticle cores for rapid subcellular vs. extended subcellular release, as is further detailed in 2C.1.2.1.

FIG. 61Q depicts Sanger sequencing results of 2C.2.1.1 on Day 14 post transfection, a study performed prior to 2C.1.2.1 whereby cores were further optimized to result in higher nanoparticle uptake efficiencies and subcellular release kinetics. Prior to decoration in targeting ligands, various nanoparticle cores were assessed for their biological performance (cellular uptake, cellular persistence over time, viability, and gene editing) in an initial set of screens intended to optimize nanoparticle cores for rapid subcellular vs. extended subcellular release. The sequence of the guide target is set forth in SEQ ID NO: 171.

FIG. 62A depicts volumes of cationic peptides and buffer used in the formulation of each nanoparticle added as step one.

FIG. 62B depicts volumes of RNP, DNA, or DNA+PLE/PDE used in the formulation of each nanoparticle added as step two. Nanoparticles were incubated for 10 minutes at this stage.

FIG. 62C depicts volumes of RNP, DNA, or DNA+PLE/PDE used in the formulation of each nanoparticle added as step 3. Nanoparticles were incubated for another 10 minutes at this stage.

FIG. 62D depicts volumes of cationic peptide, nanoparticle ALEXA-647 label, and buffer used in the formulation of each nanoparticle added as step 3. Nanoparticles were incubated for another 10 minutes at this stage.

FIG. 62E depicts the nanoparticle well ID (location in 96-well plate where it was synthesized and measured for size, zeta potential, and SYBR fluorescence) and its conversion to Nanoparticle ID (reference to the 96-well cell transfection plate) in FIG. 61I.

FIG. 62F depicts example calculations of the required cationic polypeptide volume from a 0.1% stock solution to achieve a charge ratio of 10.

FIG. 62G depicts example calculations of the required cationic polypeptide volume from a 0.1% stock solution to achieve a charge ratio of 4.

FIG. 62H depicts particle sizes of nanoparticles synthesized in 2C.1.2.1. Particle sizes were measured in triplicate via a Wyatt Mobius Zeta Potential and DLS Detector. Sizes are reported as average hydrodynamic diameter (nm)±standard deviation in a heatmap which correlates to the nanoparticle 96-well ID.

FIG. 62I depicts zeta potentials of nanoparticles synthesized in 2C.1.2.1. Particle zeta potentials were measured in triplicate via a Wyatt Mobius Zeta Potential and DLS Detector. Zeta potentials are reported as average zeta potentials (mV)±standard deviation in a heatmap which correlates to the nanoparticle 96-well ID.

FIG. 62J depicts the average (Overnight) condensation index of each particle in 2C.1.2.1 using SYBR fluorescence assay. The condensation index is calculated as [(Well of Interest Fluorescence−Free DNA Fluorescence)/Free DNA Fluorescence]*100 and is reported as average condensation index±standard deviation in a heatmap which correlates to the nanoparticle 96-well ID. The more condensed nanoparticles will have higher shielding, less fluorescence, and thus a more negative condensation index.

FIG. 62K depicts the nanoparticle transfection layout for 2C.1.2.1 in HEK293-GFP cells. Groups highlighted yellow are a single (10 ul) dose of the corresponding nanoparticle indicated in FIGS. 62A-62E, while groups highlighted in blue are a double (20 ul) dose of the corresponding nanoparticle indicated in FIGS. 62A-62E. Green highlight shows CRISPRMAX transfection controls, grey highlight shows Lipofectamine3000 transfection controls. The DNA Mix includes the ssODN and PhiC31 expression and donor plasmids.

FIG. 62L depicts 2C.1.2.1 Flow results for Day 3 post transfection, monitoring Alexa647 fluorescence (NP signal). Live cells were gated based on FSC/SSC scatter and % NP+ is shown for live gate.

FIG. 62M depicts 2C.1.2.1 flow results for Day 3 post transfection, monitoring GFP fluorescence. Live cells were gated based on FSC/SSC scatter and % GFP− is shown for live gate. RFP expression was observed only in the lipofection control with Tag-RFP plasmid: 51% RFP+ of Live, well B6 (data not shown).

FIG. 62N depicts Day 3 flow plots of GFP and Alexa647 (NP) for selected NP-transfected samples from 2C.1.2.1 (HEK293-GFP) that show GFP KD in FIG. 62F. Live cell gate (based on FSC/SSC) is shown. Decrease in GFP expression is seen +/−NP signal, suggesting fast degradation of NP peptide shell and release of RNP payload.

FIG. 62O depicts 2C.1.2.1 Flow results for Day 3 post transfection, monitoring GFP fluorescence Alexa647 fluorescence (NP signal). Live cells were gated based on FSC/SSC scatter and percentages are shown for live gate. Green highlighted wells are top hits for % GFP− (shown in 62F) and pink highlighted wells are top hits for % NP+ (shown in 62E).

FIG. 62P depicts contrast-enhanced images (top-left, bottom-left; see ImageJ Script) and associated threshold maps (top-right, bottom-right) applied to AF647-labeled nanoparticles transfected into HEK293-GFP cells and corresponding to well E5 of 2C.1.2.1. Bright green areas represent GFP− areas with high degrees of NP-induced fluorescence, whereas red areas indicate GFP+ areas absent of NP-induced fluorescence. NP fluorescence was acquired by a BioTek Cytation 5 Texas Red Filter Cube (Part Number: 1225102), whereby colocalization studies and comparison to flow cytometry results with AF647 (Cy5 channel) demonstrated that NP+ pixels were indistinguishable from RFP+ pixels. These threshold maps were used to generate Pearson coefficients, M1 & M2 coefficients, and overlap coefficients for each well position.

FIG. 62Q depicts a Costes' threshold map applied to AF647-labeled nanoparticles transfected into HEK293-GFP cells and corresponding to well E5 of 2C.1.2.1 generated on Day 6 post-transfection and corresponding to FIG. 62P. Bright green areas represent GFP− areas with high degrees of NP-induced fluorescence, whereas red areas indicate GFP+ areas absent of NP-induced fluorescence.

FIG. 62R depicts representative data corresponding to segmentation and Castes' threshold maps for well F5 of 2C.1.2.1 generated on Day 6 post-transfection, and demonstrates automatic thresholding via an imaging script. These threshold maps were used to generate Pearson coefficients, M1 & M2 coefficients, and overlap coefficients for each well position.

FIG. 62S depicts representative data corresponding to automated generation of threshold maps for well B6 of 2C.1.2.1 (RFP plasmid-only positive control), and demonstrates automatic thresholding via an imaging script. These threshold maps were used to generate Pearson coefficients, M1 & M2 coefficients, and overlap coefficients for each well position in other wells. Confirmation of Texas Red channel (RFP+) in the absence of Cy5 channel (NP−) and visibility of RFP+ as a NP+ indicator were used for further thresholding in this experiment when comparing other wells.

FIG. 62T depicts RFP and DAPI overlap images. Well B5 displays no Texas Red channel due to the absence of RFP insertions (as seen in B6 RFP plasmid Lipofectamine control). Wells E8, E9 and F9 (nanoparticle groups) display high visibly high degrees of nanoparticle uptake, where certain wells (e.g. E5) display Manders' Coefficients of M1=0.01 (fraction of GFP+ overlapping NP+) vs. M2=0.907 (fraction of NP+ overlapping GFP+). This indicates a strong relationship between particle uptake and the absence of GFP expression.

FIG. 62U depicts day 3 NP uptake and GFP knockdown of 2C.1.2.1, whereby samples are bimodally sorted according to % GFP− (descending values from B4 to E4 above), and % NP+ (ascending values from E7 to F5 above). Remarkably, NP+ live cell proportions remain similar between days 3 and 6 for the best-performing nanoparticle-uptake groups. This suggests that various components of the particles are efficiently entering the cell, but not efficiently releasing their payloads or reaching the appropriate compartment(s), and that these nanoparticles may have delayed release kinetics. Top-performing GFP knockdown particles on day 3 decreased in relative knockdown efficiency by day 6. Particle degradation is modeled by Δ(Day3NP+ %−Day6NP+ %). Gene editing efficiency as accounts for toxicity of NP+ edited cells is modeled by Δ(Day6GFP− %−Day3GFP− %). Comparison of these two ratios allows for establishing an optimal “core nanoparticle” for subsequent coating in targeting ligands, whereby a balance in % NP+ cells at day 3 and % GFP− cells at day 6 is sought. According to these selection criteria, NP13, NP15, NP06, and NP14 (black rectangle) are top nanoparticle candidates for further ligand-targeted layering and optimization of cellular targeting vs. subcellular release efficiencies. In FIG. 35A, “core particles” (by the same definition, e.g. comprising only anionic and/or cationic polypeptides without ligands) are shown to achieve comparable uptake efficiencies to the lowest-performing groups in 2C.1.2.1 and 2C.2.1.1, whereby decoration in various targeting ligands increases cellular uptake and CRISPR-Cas9 RNP delivery by more than 10× efficiency (FIGS. 30-56).

FIG. 62V depicts day 6 NP uptake and GFP knockdown of 2C.1.2.1, whereby samples are bimodally sorted according to % GFP− (descending values from B4 to E4 above), and % NP+ (ascending values from E7 to F5 above). Remarkably, NP+ live cell proportions remain similar between days 3 and 6 for the best-performing nanoparticle-uptake groups. This suggests that various components of the particles are efficiently entering the cell, but not efficiently releasing their payloads or reaching the appropriate compartment(s), and that these nanoparticles may have delayed release kinetics. Top-performing GFP knockdown particles on day 3 decreased in relative knockdown efficiency by day 6. Particle degradation is modeled by Δ(Day3NP+ %−Day6NP+ %). Gene editing efficiency as accounts for toxicity of NP+ edited cells is modeled by Δ(Day6GFP− %−Day3GFP− %). Comparison of these two ratios allows for establishing an optimal “core nanoparticle” for subsequent coating in targeting ligands, whereby a balance in % NP+ cells at day 3 and % GFP− cells at day 6 is sought. According to these selection criteria, NP13, NP15, NP06, and NP14 (black rectangle) are top nanoparticle candidates for further ligand-targeted layering and optimization of cellular targeting vs. subcellular release efficiencies. In FIG. 35A, “core particles” (by the same definition, e.g. comprising only anionic and/or cationic polypeptides without ligands) are shown to achieve comparable uptake efficiencies to the lowest-performing groups in 2C.1.2.1 and 2C.2.1.1, whereby decoration in various targeting ligands increases cellular uptake and CRISPR-Cas9 RNP delivery by more than 10× efficiency (FIGS. 30-56).

FIG. 62W depicts top-performing day 3 GFP knockdown particles to top-performing day 6 uptake particles. Remarkably, NP+ live cell proportions remain similar between days 3 and 6 for the best-performing nanoparticle-uptake groups and GFP− cells seemingly rely on rapid NP metabolism, while NP that exhibit low toxicities and high uptake percentages exhibit low payload activity. This suggests that various components of the particles are efficiently entering the cell, but not efficiently releasing their payloads or reaching the appropriate compartment(s), and that these nanoparticles may have delayed release kinetics with limited cellular toxicity. Certain orders of addition and formulations may also disrupt gRNA-Cas9 activity due to strong electrostatic interactions. Top-performing GFP knockdown particles on day 3 decreased in relative knockdown efficiency by day 6. Samples E7, E4, F7 and E5 (black rectangle) increased in gene editing efficiency between days 3 and 6, and had the highest ratio of edited cells to NP+ cells of all formulations studied in 2C.1.2.1.

FIG. 62X depicts comparative Day 3 vs. Day 6 live NP+ cells (% of total live cells that contain nanoparticles). Shown are various formulations in terms of orders of addition, inclusion of PDE/PLE, PLR10, PLR50, and/or histone-derived fragments, and their associated transfection efficiencies of 4/5-component nanoparticles. NP17 is observed to achieve higher transfection efficiencies in E8, at 50% the dose of NP17 in G8, suggesting optimized subcellular trafficking may increase the NP+ cell numbers. Further optimization of condensation indices, zeta potentials and ligand coatings will yield improvements to cell-specificity and subcellular release efficiencies, as evidenced by formulations with approximately 10% lower-than-global-maximum condensation indices performing best in terms of ratio of NP+ live cells vs. GFP− edited live cells (FIGS. 62B-62C) and additional formulation studies on RNP-only delivery systems (FIGS. 11-56). In FIG. 35A, “core particles” (e.g. comprising only anionic and/or cationic polypeptides without ligands) are shown to achieve comparable uptake efficiencies to the lowest-performing groups in 2C.1.2.1 and 2C.2.1.1, whereby decoration in various targeting ligands increases cellular uptake and CRISPR-Cas9 RNP delivery by more than 10× efficiency (FIGS. 30-56). Creating a nanoparticle with a sufficiently below-global-maximum condensation index, optionally comprising ratios of histone-derived sequences and PLE and/or PDE, allows for optimized rational selection of targeting ligands and nanoparticle surface chemistries balanced with subcellular trafficking and release capabilities.

FIG. 62Y depicts comparative Day 3 vs. Day 6 live NP+ cells (% of total live cells that contain nanoparticles or are GFP−, or both) as defined by black rectangles in FIGS. 62M-62O. These particles had an increase in GFP− live cells present following transfection when the two time-points were compared, suggesting delayed or extended release of nanoparticle payloads. Additionally, GFP− live cell frequencies are similar to transfection efficiencies, suggesting efficient subcellular release, degradation of nanoparticles+AF647 fluorophore, and subsequent payload activity. These nanoparticle cores are ideal templates for further layering with targeting ligands as depicted in FIGS. 30-56.

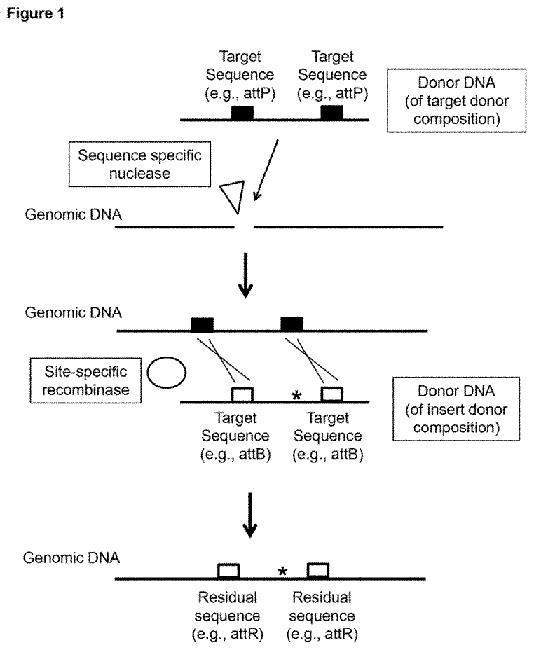

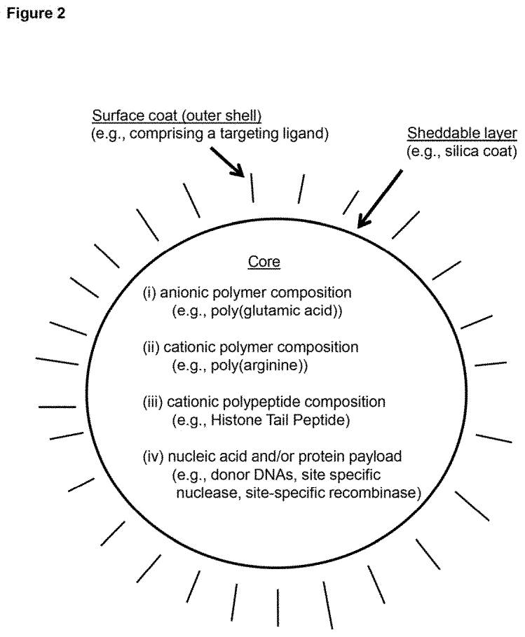

### DETAILED DESCRIPTION

As summarized above, provided are compositions and methods for genome editing using a delivery vehicle with multiple payloads. In some embodiments, subject methods include introducing a delivery vehicle into a cell, where the delivery vehicle includes a payload that includes (a) one or more sequence specific nucleases that cleave the cell's genome (e.g., a meganuclease, a homing endonuclease, a zinc finger nuclease (ZFN), a TALEN, a type I or type III CRISPR/Cas cleavage complex, a class 2 CRISPR/Cas effector protein—an RNA-guided CRISPR/Cas polypeptide—such as Cas9, CasX, CasY, Cpf1 (Cas12a), Cas13, MAD7, and the like) or one or more nucleic acids that encode the one or more sequence specific nucleases [(a) is referred to herein as a nuclease composition]; (b) a first donor DNA, which includes a nucleotide sequence that is inserted into the cell's genome, where insertion of said nucleotide sequence produces, in the cell's genome at the site of insertion, a target sequence (e.g., an attP site) for a site-specific recombinase [(b) is referred to herein as a target donor composition]; (c) the site-specific recombinase (or a nucleic acid encoding same) (e.g., ϕC31, ϕC31 RDF, Cre, FLP), where the site-specific recombinase recognizes said target sequence [(c) is referred to herein as a recombinase composition]; and (d) a second donor DNA, which includes a nucleotide sequence that is inserted into the cell's genome as a result of recognition of said target sequence by the site-specific recombinase [(d) is referred to herein as an insert donor composition].

Before the present methods and compositions are described, it is to be understood that this invention is not limited to the particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the endonuclease” includes reference to one or more endonucleases and equivalents thereof, known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### Methods and Compositions

Provided are methods and compositions for efficient genome editing. In some embodiments, a subject method includes introducing into a cell, a delivery vehicle with a payload that includes (a) [referred to herein as a nuclease composition] one or more sequence specific nucleases that cleave the cell's genome (or one or more nucleic acids that encode the one or more sequence specific nucleases), (b) [referred to herein as a target donor composition] a first donor DNA that includes a nucleotide sequence that is inserted into the cell's genome, where insertion of said nucleotide sequence produces, in the cell's genome at the site of insertion, a target sequence (target site, e.g., an attP site) for a site-specific recombinase; (c) [referred to herein as a recombinase composition] the site-specific recombinase (or a nucleic acid encoding same), where the site-specific recombinase recognizes said target sequence; and (d) [referred to herein as an insert donor composition] a second donor DNA, which includes a nucleotide sequence of interest that is inserted into the cell's genome as a result of recognition of said target sequence by the site-specific recombinase.

A nucleic acid encoding (a) a site specific nuclease (also referred to herein as a sequence specific nuclease) or (b) a site-specific recombinase, can be any nucleic acid of interest, e.g., as a nucleic acid payload of a delivery vehicle it can be linear or circular, and can be a plasmid, a viral genome, an RNA, etc. The term “nucleic acid” encompasses modified nucleic acids. In some cases, the nucleic acid molecule can be a mimetic, can include a modified sugar backbone, one or more modified internucleoside linkages (e.g., one or more phosphorothioate and/or heteroatom internucleoside linkages), one or more modified bases, and the like (as long as the nucleic acid can be transcribed and/or translated into the protein).

### I. Nuclease Composition (Sequence Specific Nuclease)

A subject nuclease composition includes one or more sequence specific nucleases (also referred to herein as site-specific nucleases), or one or more nucleic acids encoding the one or more sequence specific nucleases. A subject site specific nuclease is one that can introduce a cut (double stranded or single stranded) in genomic DNA in a sequence specific manner. Some site specific nucleases are engineered proteins (e.g., zinc finger nucleases (ZFNs) and transcription activator-like effector nucleases (TALENs)) and in some cases such proteins are used to generate nicks (single strand breaks) or as protein pairs to generate blunt or staggered ends. In some cases a site specific nuclease is one that generates a blunt double stranded cut (e.g., a class 2 CRISPR/Cas effector protein such as Cas9, CasX, CasY, Cpf1, Cas13, and the like). In some cases a site specific nuclease is one that naturally generates a blunt single strand cut (e.g., a class 2 CRISPR/Cas effector protein such as Cas9). but has been mutated such that the protein is a nickase (cuts only one strand of DNA). Nickase proteins such as a mutated nickase Cas9 can be used to generate single strand breaks or to generate staggered ends by using two guide RNAs that target opposite strands of the target DNA. Thus, in some cases a subject method includes using a sequence specific nickase (e.g., a nickase class 2 CRISPR/Cas effector protein such as a nickase Cas9) with two guide RNAs to generate a staggered cut at (at least) one of two genomic locations. In some cases a subject method includes using a sequence specific nickase (e.g., a nickase class 2 CRISPR/Cas effector protein such as a nickase Cas9) with four guide RNAs to generate two staggered cuts at two genomic locations.

Any convenient site specific nuclease (e.g., gene editing protein such as any convenient programmable gene editing protein) can be used. Examples of suitable programmable gene editing proteins include but are not limited to transcription activator-like effector nucleases (TALENs), zinc-finger nucleases (ZFNs), type I or type III CRISPR/Cas effector proteins, and Class 2 CRISPR/Cas RNA-guided polypeptides (effectors) such as Cas9, CasX, CasY, Cpf1, Cas13, MAD7, and the like). Examples of site specific nuclease that can be used include but are not limited to transcription activator-like effector nucleases (TALENs); zinc-finger nucleases (ZFNs); type I or type III CRISPR/Cas nucleases; CRISPR/Cas RNA-guided polypeptides (effector proteins) such as Cas9, CasX, CasY, Cpf1, Cas13, MAD7, and the like); meganucleases (e.g., I-SceI, I-CeuI, I-CreI, I-DmoI, I-ChuI, I-DirI, I-FlmuI, I-FlmuII, I-AniI, I-SceIV, I-CsmI, I-PanI, I-PanII, I-PanMI, I-ScelI, I-PpoI, I-SceIII, I-LtrI, I-GpiI, I-GZeI, I-OnuI, I-HjeMI, I-MsoI, I-TevI, I-TevII, I-TevIII, PI-MleI, PI-MtuI, PI-PspI, PI-Tli I, PI-Tli II, PI-SceV, and the like); and homing endonucleases. A site specific nuclease can be delivered (as part of a delivery vehicle) to a cell as protein or as a nucleic acid (RNA or DNA) encoding the protein.

In some cases a delivery vehicle is used to deliver a nucleic acid encoding a gene editing tool (i.e., a component of a gene editing system, e.g., a site specific cleaving system such as a programmable gene editing system). For example, a nucleic acid payload can include one or more of: (i) a CRISPR/Cas guide RNA, (ii) a DNA encoding a CRISPR/Cas guide RNA, (iii) a DNA and/or RNA encoding a programmable gene editing protein such as a zinc finger protein (ZFP) (e.g., a zinc finger nuclease—ZFN), a transcription activator-like effector (TALE) protein (e.g., fused to a nuclease—TALEN), a type I or type III CRISPR/Cas nuclease, and/or a CRISPR/Cas RNA-guided polypeptide (e.g., Cas9, CasX, CasY, Cpf1, Cas13, MAD7, and the like); (iv) a DNA and/or RNA encoding a meganuclease; (v) a DNA and/or RNA encoding a homing endonuclease; and (iv) a Donor DNA molecule (first and/or second subject donor DNAs).

In some cases a subject delivery vehicle is used to deliver a protein payload, e.g., a protein such as a ZFN, a TALEN, a type I or type III CRISPR/Cas nuclease, and/or a CRISPR/Cas RNA-guided polypeptide (e.g., Cas9, CasX, CasY, Cpf1, Cas13, MAD7, and the like), a meganuclease, and a homing endonuclease. Cas13, MAD7,

Depending on the nature of the system and the desired outcome, a gene editing system (e.g. a site specific gene editing system such as a programmable gene editing system) can include a single component (e.g., a ZFP, a ZFN, a TALE, a TALEN, a meganuclease, and the like) or can include multiple components. In some cases a gene editing system includes at least two components. For example, in some cases a gene editing system (e.g. a programmable gene editing system) includes (i) a donor DNA molecule nucleic acid; and (ii) a gene editing protein (e.g., a programmable gene editing protein such as a ZFP, a ZFN, a TALE, a TALEN, a DNA-guided polypeptide such as Natronobacterium gregoryi Argonaute (NgAgo), a type I or type III CRISPR/Cas nuclease, and/or a CRISPR/Cas RNA-guided polypeptide (e.g., Cas9, CasX, CasY, Cpf1, Cas13, MAD7, and the like), or a nucleic acid molecule encoding the gene editing protein (e.g., DNA or RNA such as a plasmid or mRNA). As another example, in some cases a gene editing system (e.g. a programmable gene editing system) includes (i) a CRISPR/Cas guide RNA, or a DNA encoding the CRISPR/Cas guide RNA; and (ii) a CRISPR/CAS RNA-guided polypeptide (e.g., Cas9, CasX, CasY, Cpf1, Cas13, MAD7, and the like), or a nucleic acid molecule encoding the RNA-guided polypeptide (e.g., DNA or RNA such as a plasmid or mRNA). As another example, in some cases a gene editing system (e.g. a programmable gene editing system) includes (i) an NgAgo-like guide DNA; and (ii) a DNA-guided polypeptide (e.g., NgAgo), or a nucleic acid molecule encoding the DNA-guided polypeptide (e.g., DNA or RNA such as a plasmid or mRNA). In some cases a gene editing system (e.g. a programmable gene editing system) includes at least three components: (i) a donor DNA molecule; (ii) a CRISPR/Cas guide RNA, or a DNA encoding the CRISPR/Cas guide RNA; and (iii) a CRISPR/Cas RNA-guided polypeptide (e.g., Cas9, CasX, CasY, or Cpf1), or a nucleic acid molecule encoding the RNA-guided polypeptide (e.g., DNA or RNA such as a plasmid or mRNA). In some cases a gene editing system (e.g. a programmable gene editing system) includes at least three components: (i) a donor DNA molecule; (ii) an NgAgo-like guide DNA, or a DNA encoding the NgAgo-like guide DNA; and (iii) a DNA-guided polypeptide (e.g., NgAgo), or a nucleic acid molecule encoding the DNA-guided polypeptide (e.g., DNA or RNA such as a plasmid or mRNA).

In some embodiments, a payload of a delivery vehicle includes one or more gene editing tools. The term “gene editing tool” is used herein to refer to one or more components of a gene editing system. Thus, in some cases the payload includes a gene editing system and in some cases the payload includes one or more components of a gene editing system (i.e., one or more gene editing tools). For example, a target cell might already include one of the components of a gene editing system and the user need only add the remaining components. In such a case the payload of a subject nanoparticle does not necessarily include all of the components of a given gene editing system. As such, in some cases a payload includes one or more gene editing tools.

As an illustrative example, a target cell might already include a gene editing protein (e.g., a ZFP, a TALE, a DNA-guided polypeptide (e.g., NgAgo), a type I or type III CRISPR/Cas nuclease, and/or a CRISPR/Cas RNA-guided polypeptide (e.g., Cas9, CasX, CasY, Cpf1, Cas13, MAD7, and the like, and/or a DNA or RNA encoding the protein, and therefore the payload can include one or more of: (i) a donor DNA molecule; and (ii) a CRISPR/Cas guide RNA, or a DNA encoding the CRISPR/Cas guide RNA; or an NgAgo-like guide DNA. Likewise, the target cell may already include a CRISPR/Cas guide RNA and/or a DNA encoding the guide RNA or an NgAgo-like guide DNA, and the payload can include one or more of: (i) a donor DNA molecule; and (ii) a CRISPR/Cas RNA-guided polypeptide (e.g., Cas9, CasX, CasY, Cpf1, Cas13, MAD7, and the like), or a nucleic acid molecule encoding the RNA-guided polypeptide (e.g., DNA or RNA such as a plasmid or mRNA); or a DNA-guided polypeptide (e.g., NgAgo), or a nucleic acid molecule encoding the DNA-guided polypeptide.

For additional information related to programmable gene editing tools (e.g., CRISPR/Cas RNA-guided proteins such as Cas9, CasX, CasY, Cpf1 (Cas12a), and Cas13, Zinc finger proteins such as Zinc finger nucleases, TALE proteins such as TALENs, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 July 34(7):768-73; and Shmakov et al., Nat Rev Microbiol. 2017 March; 15(3):169-182; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

### II. Target Donor Composition (First Donor DNA)

A subject target donor composition includes a first donor DNA. The first donor DNA can be linear or circular and can be in any convenient format (e.g., plasmid, minicircle, linear, etc.). The donor DNA of the target donor composition can include one or more target sequences (target sites) that are inserted into the genome and are then recognized (and utilized) by the site-specific recombinase. In some cases the donor DNA of the target donor composition does not include a target site, but insertion of the donor DNA results in such a target site being present in the genome (e.g., a target site can be generated at the junction of an insert sequence of the donor and the genome, e.g., in some cases where the donor and the genome have sticky ends).

The donor DNA (the first donor DNA) of the target donor composition can be single stranded or double stranded.

In some cases, the second donor DNA (the donor DNA of the insert donor composition) has a length of (has a total of) 10 or more base pairs (bp) (e.g., 20 or more, 30 or more, 50 or more, 100 or more, 200 or more, 500 or more, 1,000 or more, 5,000 or more, 10,000 or more, 50,000 or more, or 75,000 or more bp). In other words, in some cases a subject donor DNA has 10 or more bp (e.g., 20 or more, 30 or more, 50 or more, 100 or more, 200 or more, 500 or more, 1,000 or more, 5,000 or more, 10,000 or more, 50,000 or more, or 75,000 or more bp).

In some cases a subject second donor DNA (the donor DNA of the insert donor composition) has a total of from (has a length of from) 10 base pairs (bp) to 150 kilobase pairs (kbp) [in nucleotides (nt) instead of ‘bp’ if single stranded] (e.g., from 10 bp to 100 kbp, 70 kbp, 10 bp to 50 kbp, 10 bp to 40 kbp, 10 bp to 25 kbp, 10 bp to 15 kbp, 10 bp to 10 kbp, 10 bp to 1 kbp, 10 bp to 750 bp, 10 bp to 500 bp, 10 bp to 250 bp, 10 bp to 150 bp, 10 bp to 100 bp, 10 bp to 50 bp, 18 bp to 150 kbp, 18 bp to 100 kbp, 18 bp to 70 kbp, 18 bp to 50 kbp, 18 bp to 40 kbp, 18 bp to 25 kbp, 18 bp to 15 kbp, 18 bp to 10 kbp, 18 bp to 1 kbp, 18 bp to 750 bp, 18 bp to 500 bp, 18 bp to 250 bp, 18 bp to 150 bp, 25 bp to 150 kbp, 25 bp to 100 kbp, 25 bp to 70 kbp, 25 bp to 50 kbp, 25 bp to 40 kbp, 25 bp to 25 kbp, 25 bp to 15 kbp, 25 bp to 10 kbp, 25 bp to 1 kbp, 25 bp to 750 bp, 25 bp to 500 bp, 25 bp to 250 bp, 25 bp to 150 bp, 50 bp to 150 kbp, 50 bp to 100 kbp, 50 bp to 70 kbp, 50 bp to 50 kbp, 50 bp to 40 kbp, 50 bp to 25 kbp, 50 bp to 15 kbp, 50 bp to 10 kbp, 50 bp to 1 kbp, 50 bp to 750 bp, 50 bp to 500 bp, 50 bp to 250 bp, 50 bp to 150 bp, 100 bp to 150 kbp, 100 bp to 100 kbp, 100 bp to 70 kbp, 100 bp to 50 kbp, 100 bp to 40 kbp, 100 bp to 25 kbp, 100 bp to 15 kbp, 100 bp to 10 kbp, 100 bp to 1 kbp, 100 bp to 750 bp, 100 bp to 500 bp, 100 bp to 250 bp, 200 bp to 150 kbp, 200 bp to 100 kbp, 200 bp to 70 kbp, 200 bp to 50 kbp, 200 bp to 40 kbp, 200 bp to 25 kbp, 200 bp to 15 kbp, 200 bp to 10 kbp, 200 bp to 1 kbp, 200 bp to 750 bp, 200 bp to 500 bp, 10 kbp to 150 kbp, 10 kbp to 100 kbp, 10 kbp to 70 kbp, 10 kbp to 50 kbp, 10 kbp to 40 kbp, 10 kbp to 25 kbp, 50 kbp to 150 kbp, 50 kbp to 100 kbp, 50 kbp to 70 kbp, 70 kbp to 150 kbp, or 70 kbp to 100 kbp). In some cases a subject first donor DNA (the donor DNA of the target donor composition) has a total of from 10 bp to 50 kbp. In some cases a subject first donor DNA (the donor DNA of the target donor composition) has a total of from 10 bp to 10 kbp. In some cases a subject first donor DNA (the donor DNA of the target donor composition) has a total of from 50 kbp to 100 kbp. In some cases a subject first donor DNA (the donor DNA of the target donor composition) has a total of from 10 bp to 10 kbp. In some cases a subject first donor DNA (the donor DNA of the target donor composition) has a total of from 10 bp to 1 kbp. In some cases a subject first donor DNA (the donor DNA of the target donor composition) has a total of from 20 bp to 50 kbp. In some cases a subject first donor DNA (the donor DNA of the target donor composition) has a total of from 20 bp to 10 kbp. In some cases a subject first donor DNA (the donor DNA of the target donor composition) has a total of from 20 bp to 1 kbp.

In some cases the donor DNA includes one or more of: a mimetic, a modified sugar backbone, a non-natural internucleoside linkages (e.g., one or more phosphorothioate and/or heteroatom internucleoside linkages), a modified base, and the like.

The nucleotide sequence of the donor DNA (the first donor DNA) of the target donor composition that is inserted into the cell's genome can have any convenient length. For example, in some cases the sequence has a length of (has a total of) 10 or more base pairs (bp) (e.g., 20 or more, 30 or more, 50 or more, 100 or more, 200 or more, 500 or more, 1,000 or more, 5,000 or more, 10,000 or more, 50,000 or more, or 75,000 or more bp). In other words, in some cases the nucleotide sequence of the donor DNA (the first donor DNA) of the target donor composition that is inserted into the cell's genome has 10 or more bp (e.g., 20 or more, 30 or more, 50 or more, 100 or more, 200 or more, 500 or more, 1,000 or more, 5,000 or more, 10,000 or more, 50,000 or more, or 75,000 or more bp).

In some cases the nucleotide sequence of the donor DNA (the first donor DNA) of the target donor composition that is inserted into the cell's genome has a total of from (has a length of from) 10 base pairs (bp) to 150 kilobase pairs (kbp) [in nucleotides (nt) instead of ‘bp’ if single stranded] (e.g., from 10 bp to 100 kbp, 70 kbp, 10 bp to 50 kbp, 10 bp to 40 kbp, 10 bp to 25 kbp, 10 bp to 15 kbp, 10 bp to 10 kbp, 10 bp to 1 kbp, 10 bp to 750 bp, 10 bp to 500 bp, 10 bp to 250 bp, 10 bp to 150 bp, 10 bp to 100 bp, 10 bp to 50 bp, 18 bp to 150 kbp, 18 bp to 100 kbp, 18 bp to 70 kbp, 18 bp to 50 kbp, 18 bp to 40 kbp, 18 bp to 25 kbp, 18 bp to 15 kbp, 18 bp to 10 kbp, 18 bp to 1 kbp, 18 bp to 750 bp, 18 bp to 500 bp, 18 bp to 250 bp, 18 bp to 150 bp, 25 bp to 150 kbp, 25 bp to 100 kbp, 25 bp to 70 kbp, 25 bp to 50 kbp, 25 bp to 40 kbp, 25 bp to 25 kbp, 25 bp to 15 kbp, 25 bp to 10 kbp, 25 bp to 1 kbp, 25 bp to 750 bp, 25 bp to 500 bp, 25 bp to 250 bp, 25 bp to 150 bp, 50 bp to 150 kbp, 50 bp to 100 kbp, 50 bp to 70 kbp, 50 bp to 50 kbp, 50 bp to 40 kbp, 50 bp to 25 kbp, 50 bp to 15 kbp, 50 bp to 10 kbp, 50 bp to 1 kbp, 50 bp to 750 bp, 50 bp to 500 bp, 50 bp to 250 bp, 50 bp to 150 bp, 100 bp to 150 kbp, 100 bp to 100 kbp, 100 bp to 70 kbp, 100 bp to 50 kbp, 100 bp to 40 kbp, 100 bp to 25 kbp, 100 bp to 15 kbp, 100 bp to 10 kbp, 100 bp to 1 kbp, 100 bp to 750 bp, 100 bp to 500 bp, 100 bp to 250 bp, 200 bp to 150 kbp, 200 bp to 100 kbp, 200 bp to 70 kbp, 200 bp to 50 kbp, 200 bp to 40 kbp, 200 bp to 25 kbp, 200 bp to 15 kbp, 200 bp to 10 kbp, 200 bp to 1 kbp, 200 bp to 750 bp, 200 bp to 500 bp, 10 kbp to 150 kbp, 10 kbp to 100 kbp, 10 kbp to 70 kbp, 10 kbp to 50 kbp, 10 kbp to 40 kbp, 10 kbp to 25 kbp, 50 kbp to 150 kbp, 50 kbp to 100 kbp, 50 kbp to 70 kbp, 70 kbp to 150 kbp, or 70 kbp to 100 kbp). In some cases the nucleotide sequence of the donor DNA (the first donor DNA) of the target donor composition that is inserted into the cell's genome has a total of from 10 bp to 50 kbp. In some cases the nucleotide sequence of the donor DNA (the first donor DNA) of the target donor composition that is inserted into the cell's genome has a total of from 10 bp to 10 kbp. In some cases the nucleotide sequence of the donor DNA (the first donor DNA) of the target donor composition that is inserted into the cell's genome has a total of from 50 kbp to 100 kbp. In some cases the nucleotide sequence of the donor DNA (the first donor DNA) of the target donor composition that is inserted into the cell's genome has a total of from 10 bp to 10 kbp. In some cases the nucleotide sequence of the donor DNA (the first donor DNA) of the target donor composition that is inserted into the cell's genome has a total of from 10 bp to 1 kbp. In some cases the nucleotide sequence of the donor DNA (the first donor DNA) of the target donor composition that is inserted into the cell's genome has a total of from 20 bp to 50 kbp. In some cases the nucleotide sequence of the donor DNA (the first donor DNA) of the target donor composition that is inserted into the cell's genome has a total of from 20 bp to 10 kbp. In some cases the nucleotide sequence of the donor DNA (the first donor DNA) of the target donor composition that is inserted into the cell's genome has a total of from 20 bp to 1 kbp.

In some embodiments, two target sites are inserted into the genome (in order to accommodate insertion of a single nucleotide sequence of interest from a second donor DNA—an insert donor composition—described in more detail below). In other words, in some embodiments insertion of the nucleotide sequence of the first donor DNA of the target donor composition produces a first target sequence for a site-specific recombinase at a first location in the cell's genome and a second target sequence for a site-specific recombinase at a second location in the cell's genome, This can be accomplished using any convenient approach. For example, in some cases, the two target sites can be present on the same first donor DNA. In some cases, each of the two target sites is inserted into the genome on a separate first donor DNA, and thus the payload of a subject deliver vehicle can in some cases include two different first donor DNAs (e.g., in some cases this would require cleavage of the genome in two different locations, e.g., using two different site-specific nucleases or a single CRISPR/Cas effector protein with at least two different guide RNAs, which could be included as part of the nuclease composition).

Once two target sites are produced/inserted into the genome (e.g., either using one or two first donor DNAs, and one or multiple site specific nucleases—or one or multiple guide RNAs with a CRISPR/Cas effector protein), the two target sites can be separated by 1,000,000 base pairs (bp) or less (e.g., 500,000 bp or less, 100,000 bp or less, 50,000 bp or less, 10,000 bp or less, 1,000 bp or less, 750 bp or less, or 500 bp or less). In some cases the two target sites are separated by 100,000 bp or less. In some cases the two locations are separated by 50,000 bp or less. In some embodiments, the two target sites are separated by a range of from 5 to 1,000,000 base pairs (bp) (e.g., from 5 to 500,000, 5 to 100,000, 5 to 50,000, 5 to 10,000, 5 to 5,000, 5 to 1,000, 5 to 500, 10 to 1,000,000, 10 to 500,000, 10 to 100,000, 10 to 50,000, 10 to 10,000, 10 to 5,000, 10 to 1,000, 10 to 500, 50 to 1,000,000, 50 to 500,000, 50 to 100,000, 50 to 50,000, 50 to 10,000, 50 to 5,000, 50 to 1,000, 50 to 500, 100 to 1,000,000, 100 to 500,000, 100 to 100,000, 100 to 50,000, 100 to 10,000, 100 to 5,000, 100 to 1,000, 100 to 500, 300 to 1,000,000, 300 to 500,000, 300 to 100,000, 300 to 50,000, 300 to 10,000, 300 to 5,000, 300 to 1,000, 300 to 500, 500 to 1,000,000, 500 to 500,000, 500 to 100,000, 500 to 50,000, 500 to 10,000, 500 to 5,000, 500 to 1,000, 1,000 to 1,000,000, 1,000 to 500,000, 1,000 to 100,000, 1,000 to 50,000, 1,000 to 10,000, or 1,000 to 5,000 bp).

In some cases the two target sites are separated by a range of from 20 to 1,000,000 bp. In some cases the two target sites are separated by a range of from 20 to 500,000 bp. In some cases the two target sites are separated by a range of from 20 to 150,000 bp. In some cases the two target sites are separated by a range of from 20 to 50,000 bp. In some cases the two target sites are separated by a range of from 20 to 20,000 bp. In some cases the two target sites are separated by a range of from 20 to 15,000 bp. In some cases the two target sites are separated by a range of from 20 to 10,000 bp.

In some cases the two target sites are separated by a range of from 500 to 1,000,000 bp. In some cases the two target sites are separated by a range of from 500 to 500,000 bp. In some cases the two target sites are separated by a range of from 500 to 150,000 bp. In some cases the two target sites are separated by a range of from 500 to 50,000 bp. In some cases the two target sites are separated by a range of from 500 to 20,000 bp. In some cases the two target sites are separated by a range of from 500 to 15,000 bp. In some cases the two target sites are separated by a range of from 500 to 10,000 bp.

In some cases the two target sites are separated by a range of from 1,000 to 1,000,000 bp. In some cases the two target sites are separated by a range of from 1,000 to 500,000 bp. In some cases the two target sites are separated by a range of from 1,000 to 150,000 bp. In some cases the two target sites are separated by a range of from 1,000 to 50,000 bp. In some cases the two target sites are separated by a range of from 1,000 to 20,000 bp. In some cases the two target sites are separated by a range of from 1,000 to 15,000 bp. In some cases the two target sites are separated by a range of from 1,000 to 10,000 bp.

In some cases the two target sites are separated by a range of from 5,000 to 1,000,000 bp. In some cases the two target sites are separated by a range of from 5,000 to 500,000 bp. In some cases the two target sites are separated by a range of from 5,000 to 150,000 bp. In some cases the two target sites are separated by a range of from 5,000 to 50,000 bp. In some cases the two target sites are separated by a range of from 5,000 to 20,000 bp. In some cases the two target sites are separated by a range of from 5,000 to 15,000 bp. In some cases the two target sites are separated by a range of from 5,000 to 10,000 bp.

If the first donor DNA is double stranded, each end of the donor DNA, independently, can have a 5′ or 3′ single stranded overhang, or can be a blunt end. For example, in some cases both ends of the donor DNA have a 5′ overhang. In some cases both ends of the donor DNA have a 3′ overhang. In some cases one end of the donor DNA has a 5′ overhang while the other end has a 3′ overhang. Each overhang can be any convenient length. In some cases, the length of each overhang can be, independently, 2-200 nucleotides (nt) long (see, e.g., 2-150, 2-100, 2-50, 2-25, 2-20, 2-15, 2-12, 2-10, 2-8, 2-7, 2-6, 2-5, 3-150, 3-100, 3-50, 3-25, 3-20, 3-15, 3-12, 3-10, 3-8, 3-7, 3-6, 3-5, 4-150, 4-100, 4-50, 4-25, 4-20, 4-15, 4-12, 4-10, 4-8, 4-7, 4-6, 5-150, 5-100, 5-50, 5-25, 5-20, 5-15, 5-12, 5-10, 5-8, or 5-7 nt). In some cases the length of each overhang can be, independently, 2-20 nt long. In some cases the length of each overhang can be, independently, 2-15 nt long. In some cases the length of each overhang can be, independently, 2-10 nt long. In some cases the length of each overhang can be, independently, 2-7 nt long.

In some embodiments the donor DNA has at least one adenylated 3′ end.

Any convenient target site can be used (e.g., can be included in the nucleotide sequence of the first donor DNA that is inserted into the genome). In some cases the target site is 15 or more bp (or nt) long (e.g., 18 or more, 20 or more, 25 or more, or 30 or more bp). In some cases the target site has a length of from 15 to 50 bp (or nt) (e.g., 15 to 45, 15 to 40, 15 to 35, 18 to 50, 18 to 45, 18 to 40, 18 to 35, 20 to 50, 20 to 45, 20 to 40, 20 to 35, 25 to 50, 25 to 45, 25 to 40, 25 to 35, 30 to 50, 30 to 45, 30 to 40, 30 to 35). Examples of target sites include, but are not limited to: LoxP (recognized by Cre), LoxP2722 (recognized by Cre), att [e.g., attB ϕC31), attP ϕC31), attL ϕC31 RDF), attR ϕC31 RDF), RS (recognized by R), gix (recognized by Gin)], see, e.g., U.S. Pat. Nos. 9,902,970; 9,783,822; 9,233,174; 8,546,135; 8,399,254; 8,058,506; 7,670,823; and 5,888,732; all of which are hereby incorporated by reference for their teachings related to target sites and/or the corresponding site-specific recombinases).

### III. Recombinase Composition (Site-Specific Recombinase)

A subject recombinase composition includes one or more sequence specific recombinases (also referred to herein as site-specific recombinases), or one or more nucleic acids encoding the one or more sequence specific recombinases. A subject site specific recombinase is one that can recognize one or more target sites (see above) of the genome (after insertion of sequence from the first donor DNA) and the one or more target sites of the second donor DNA (the donor DNA of the insert donor composition)—and catalyze the insertion of a sequence of interest from the second donor DNA into the genome. Several site specific recombinases are known in the art and any convenient site specific recombinase can be used. Examples of site specific recombinases include, but are not limited to: ϕC31, ϕC31 RDF, Cre, and FLP. In addition, representative site specific recombinases can include, but are not limited to: the integrases of ϕC31 (PhiC31), R4, TP901-1, ϕBT1 (PhiBT1), Bxb1, RV-1, AA118, U153, and PFC1 (PhiFC1).

### IV. Insert Donor Composition (Second Donor DNA)

A subject insert donor composition includes a second donor DNA. The second donor DNA can be linear or circular and can be in any convenient format (e.g., plasmid, minicircle, linear, etc.). The donor DNA of the insert donor composition includes a nucleotide sequence of interest that is inserted into the genome by the site-specific recombinase. The donor DNA of the insert donor composition can be single stranded or double stranded, as long as the site-specific recombinase can catalyze insertion of the sequence using the target site that was produced during insertion of the sequence of the first donor DNA (of the target donor composition). As such, the second donor DNA will usually include one or more target sties (see, e.g., the target sites discussed above in regard to first donor DNA) that are recognized by the site specific recombinase in order to facilitate insertion of the sequence of interest into the genome. As would be known to one of ordinary skill in the art, in some cases insertion of the second donor DNA can in some cases result in residual sequence left in the genome. For example, if attB and attP target sites are used, attL and/or attR sequence(s) can be left in the genome after insertion is complete.

In some cases, the second donor DNA (the donor DNA of the insert donor composition) includes one target site. In some cases the second donor DNA (the donor DNA of the insert donor composition) includes one or more target sites (e.g., two or more target sites). In some cases the second donor DNA (the donor DNA of the insert donor composition) includes two target sties. In some cases the nucleotide sequence of interest (the sequence to be inserted into the genome is flanked by two target sites (and in some cases the two target sites are the same, i.e., the nucleotide sequence of interest can in some cases be flanked by two copies of the same target site).

In some cases, the second donor DNA (the donor DNA of the insert donor composition) has a length of (has a total of) 10 or more base pairs (bp) (e.g., 20 or more, 30 or more, 50 or more, 100 or more, 200 or more, 500 or more, 1,000 or more, 5,000 or more, 10,000 or more, 50,000 or more, or 75,000 or more bp). In other words, in some cases a subject donor DNA has 10 or more bp (e.g., 20 or more, 30 or more, 50 or more, 100 or more, 200 or more, 500 or more, 1,000 or more, 5,000 or more, 10,000 or more, 50,000 or more, or 75,000 or more bp).

In some cases a subject second donor DNA (the donor DNA of the insert donor composition) has a total of from (has a length of from) 10 base pairs (bp) to 150 kilobase pairs (kbp) [in nucleotides (nt) instead of ‘bp’ if single stranded] (e.g., from 10 bp to 100 kbp, 70 kbp, 10 bp to 50 kbp, 10 bp to 40 kbp, 10 bp to 25 kbp, 10 bp to 15 kbp, 10 bp to 10 kbp, 10 bp to 1 kbp, 10 bp to 750 bp, 10 bp to 500 bp, 10 bp to 250 bp, 10 bp to 150 bp, 10 bp to 100 bp, 10 bp to 50 bp, 18 bp to 150 kbp, 18 bp to 100 kbp, 18 bp to 70 kbp, 18 bp to 50 kbp, 18 bp to 40 kbp, 18 bp to 25 kbp, 18 bp to 15 kbp, 18 bp to 10 kbp, 18 bp to 1 kbp, 18 bp to 750 bp, 18 bp to 500 bp, 18 bp to 250 bp, 18 bp to 150 bp, 25 bp to 150 kbp, 25 bp to 100 kbp, 25 bp to 70 kbp, 25 bp to 50 kbp, 25 bp to 40 kbp, 25 bp to 25 kbp, 25 bp to 15 kbp, 25 bp to 10 kbp, 25 bp to 1 kbp, 25 bp to 750 bp, 25 bp to 500 bp, 25 bp to 250 bp, 25 bp to 150 bp, 50 bp to 150 kbp, 50 bp to 100 kbp, 50 bp to 70 kbp, 50 bp to 50 kbp, 50 bp to 40 kbp, 50 bp to 25 kbp, 50 bp to 15 kbp, 50 bp to 10 kbp, 50 bp to 1 kbp, 50 bp to 750 bp, 50 bp to 500 bp, 50 bp to 250 bp, 50 bp to 150 bp, 100 bp to 150 kbp, 100 bp to 100 kbp, 100 bp to 70 kbp, 100 bp to 50 kbp, 100 bp to 40 kbp, 100 bp to 25 kbp, 100 bp to 15 kbp, 100 bp to 10 kbp, 100 bp to 1 kbp, 100 bp to 750 bp, 100 bp to 500 bp, 100 bp to 250 bp, 200 bp to 150 kbp, 200 bp to 100 kbp, 200 bp to 70 kbp, 200 bp to 50 kbp, 200 bp to 40 kbp, 200 bp to 25 kbp, 200 bp to 15 kbp, 200 bp to 10 kbp, 200 bp to 1 kbp, 200 bp to 750 bp, 200 bp to 500 bp, 10 kbp to 150 kbp, 10 kbp to 100 kbp, 10 kbp to 70 kbp, 10 kbp to 50 kbp, 10 kbp to 40 kbp, 10 kbp to 25 kbp, 50 kbp to 150 kbp, 50 kbp to 100 kbp, 50 kbp to 70 kbp, 70 kbp to 150 kbp, or 70 kbp to 100 kbp). In some cases a subject second donor DNA (the donor DNA of the insert donor composition) has a total of from 10 bp to 50 kbp. In some cases a subject second donor DNA (the donor DNA of the insert donor composition) has a total of from 10 bp to 10 kbp. In some cases a subject second donor DNA (the donor DNA of the insert donor composition) has a total of from 50 kbp to 100 kbp. In some cases a subject second donor DNA (the donor DNA of the insert donor composition) has a total of from 10 bp to 10 kbp. In some cases a subject second donor DNA (the donor DNA of the insert donor composition) has a total of from 10 bp to 1 kbp. In some cases a subject second donor DNA (the donor DNA of the insert donor composition) has a total of from 20 bp to 50 kbp. In some cases a subject second donor DNA (the donor DNA of the insert donor composition) has a total of from 20 bp to 10 kbp. In some cases a subject second donor DNA (the donor DNA of the insert donor composition) has a total of from 20 bp to 1 kbp.

In some cases the donor DNA includes one or more of: a mimetic, a modified sugar backbone, a non-natural internucleoside linkages (e.g., one or more phosphorothioate and/or heteroatom internucleoside linkages), a modified base, and the like.

The nucleotide sequence of the donor DNA (the second donor DNA) of the insert donor composition that is inserted into the cell's genome can have any convenient length. For example, in some cases the sequence has a length of (has a total of) 10 or more base pairs (bp) (e.g., 20 or more, 30 or more, 50 or more, 100 or more, 200 or more, 500 or more, 1,000 or more, 5,000 or more, 10,000 or more, 50,000 or more, or 75,000 or more bp). In other words, in some cases the nucleotide sequence of the donor DNA (the second donor DNA) of the insert donor composition that is inserted into the cell's genome has 10 or more bp (e.g., 20 or more, 30 or more, 50 or more, 100 or more, 200 or more, 500 or more, 1,000 or more, 5,000 or more, 10,000 or more, 50,000 or more, or 75,000 or more bp).

In some cases the nucleotide sequence of the donor DNA (the second donor DNA) of the insert donor composition that is inserted into the cell's genome has a total of from (has a length of from) 10 base pairs (bp) to 150 kilobase pairs (kbp) [in nucleotides (nt) instead of ‘bp’ if single stranded] (e.g., from 10 bp to 100 kbp, 70 kbp, 10 bp to 50 kbp, 10 bp to 40 kbp, 10 bp to 25 kbp, 10 bp to 15 kbp, 10 bp to 10 kbp, 10 bp to 1 kbp, 10 bp to 750 bp, 10 bp to 500 bp, 10 bp to 250 bp, 10 bp to 150 bp, 10 bp to 100 bp, 10 bp to 50 bp, 18 bp to 150 kbp, 18 bp to 100 kbp, 18 bp to 70 kbp, 18 bp to 50 kbp, 18 bp to 40 kbp, 18 bp to 25 kbp, 18 bp to 15 kbp, 18 bp to 10 kbp, 18 bp to 1 kbp, 18 bp to 750 bp, 18 bp to 500 bp, 18 bp to 250 bp, 18 bp to 150 bp, 25 bp to 150 kbp, 25 bp to 100 kbp, 25 bp to 70 kbp, 25 bp to 50 kbp, 25 bp to 40 kbp, 25 bp to 25 kbp, 25 bp to 15 kbp, 25 bp to 10 kbp, 25 bp to 1 kbp, 25 bp to 750 bp, 25 bp to 500 bp, 25 bp to 250 bp, 25 bp to 150 bp, 50 bp to 150 kbp, 50 bp to 100 kbp, 50 bp to 70 kbp, 50 bp to 50 kbp, 50 bp to 40 kbp, 50 bp to 25 kbp, 50 bp to 15 kbp, 50 bp to 10 kbp, 50 bp to 1 kbp, 50 bp to 750 bp, 50 bp to 500 bp, 50 bp to 250 bp, 50 bp to 150 bp, 100 bp to 150 kbp, 100 bp to 100 kbp, 100 bp to 70 kbp, 100 bp to 50 kbp, 100 bp to 40 kbp, 100 bp to 25 kbp, 100 bp to 15 kbp, 100 bp to 10 kbp, 100 bp to 1 kbp, 100 bp to 750 bp, 100 bp to 500 bp, 100 bp to 250 bp, 200 bp to 150 kbp, 200 bp to 100 kbp, 200 bp to 70 kbp, 200 bp to 50 kbp, 200 bp to 40 kbp, 200 bp to 25 kbp, 200 bp to 15 kbp, 200 bp to 10 kbp, 200 bp to 1 kbp, 200 bp to 750 bp, 200 bp to 500 bp, 10 kbp to 150 kbp, 10 kbp to 100 kbp, 10 kbp to 70 kbp, 10 kbp to 50 kbp, 10 kbp to 40 kbp, 10 kbp to 25 kbp, 50 kbp to 150 kbp, 50 kbp to 100 kbp, 50 kbp to 70 kbp, 70 kbp to 150 kbp, or 70 kbp to 100 kbp). In some cases the nucleotide sequence of the donor DNA (the second donor DNA) of the insert donor composition that is inserted into the cell's genome has a total of from 10 bp to 50 kbp. In some cases the nucleotide sequence of the donor DNA (the second donor DNA) of the insert donor composition that is inserted into the cell's genome has a total of from 10 bp to 10 kbp. In some cases the nucleotide sequence of the donor DNA (the second donor DNA) of the insert donor composition that is inserted into the cell's genome has a total of from 50 kbp to 100 kbp. In some cases the nucleotide sequence of the donor DNA (the second donor DNA) of the insert donor composition that is inserted into the cell's genome has a total of from 10 bp to 10 kbp. In some cases the nucleotide sequence of the donor DNA (the second donor DNA) of the insert donor composition that is inserted into the cell's genome has a total of from 10 bp to 1 kbp. In some cases the nucleotide sequence of the donor DNA (the second donor DNA) of the insert donor composition that is inserted into the cell's genome has a total of from 20 bp to 50 kbp. In some cases the nucleotide sequence of the donor DNA (the second donor DNA) of the insert donor composition that is inserted into the cell's genome has a total of from 20 bp to 10 kbp. In some cases the nucleotide sequence of the donor DNA (the second donor DNA) of the insert donor composition that is inserted into the cell's genome has a total of from 20 bp to 1 kbp.

In some embodiments the donor DNA has at least one adenylated 3′ end.

In some embodiments, insertion of the nucleotide sequence of the second donor DNA into the cell's genome results in operable linkage of the inserted sequence with an endogenous promoter (e.g., (i) a T-cell specific promoter; (ii) a CD3 promoter; (iii) a CD28 promoter; (iv) a stem cell specific promoter; (v) a somatic cell specific promoter; and (vi) a T cell receptor (TCR) Alpha, Beta, Gamma or Delta promoter). In some cases the nucleotide sequence, of the insert donor composition, that is inserted includes a protein-coding sequence that is operably linked to a promoter (e.g., (i) a T-cell specific promoter; (ii) a CD3 promoter; (iii) a CD28 promoter; (iv) a stem cell specific promoter; (v) a somatic cell specific promoter; and (vi) a T cell receptor (TCR) Alpha, Beta, Gamma or Delta promoter).

In some embodiments the nucleotide sequence (of the second donor DNA) that is inserted into the cell's genome encodes a protein. Any convenient protein can be encoded—examples include but are not limited to: a T cell receptor (TCR) protein; a CDR1, CDR2, or CDR3 region of a T cell receptor (TCR) protein; a chimeric antigen receptor (CAR); a cell-specific targeting ligand that is membrane bound and presented extracellularly; a reporter protein (e.g., a fluorescent protein such as GFP, RFP, CFP, YFP, and fluorescent proteins that fluoresce in far red, in near infrared, etc.). In some embodiments the nucleotide sequence (of the second donor DNA) that is inserted into the cell's genome encodes a multivalent (e.g., heteromultivalent) surface receptor (e.g., in some cases where a T-cell is the target cell). Any convenient multivalent receptor could be used and non-limiting examples include: bispecific or trispecific CARs and/or TCRs, or other affinity tags on immune cells. Such an insertion would cause the targeted cell to express the receptors. In some cases multivalence is achieved by inserting separate receptors whereby the inserted receptors function as an OR gate (one or the other triggers activation), or as an AND gate (receptor signaling is co-stimulatory and homovalent binding won't activate/stimulate cell, e.g., a targeted T-cell). A protein encoded by the inserted DNA (e.g., a CAR, a TCR, a multivalent surface receptor) can be selected such that it binds to (e.g., functions to target the cell, e.g., T-cell to) one or more targets selected from: CD3, CD28, CD90, CD45f, CD34, CD80, CD86, CD19, CD20, CD22, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL-2, IL-7, IL-10, IL-12, IL-15, IL-18, TNFα, IFNγ, TGF-β, and α5β1.

In some cases the inserted nucleotide sequence encodes a receptor whereby the target that is targeted (bound) by the receptor is specific to an individual's disease (e.g., cancer/tumor). In some cases the inserted nucleotide sequence encodes a heteromultivalent receptor, whereby the combination of targets that are targeted by the heteromultivalent receptor are specific to an individual's disease (e.g., cancer/tumor). As one illustrative example, an individual's cancer (e.g., tumor, e.g., via biopsy) can be sequenced (nucleic acid sequence, proteomics, metabolomics etc.) to identify antigens of diseased cells that can be targets (such as antigens that are overexpressed by or are unique to a tumor relative to control cells of the individual), and a nucleotide sequence encoding a receptor (e.g., heteromultivalent receptor) that binds to one or more of those targets (e.g., 2 or more, 3 or more, 5 or more, 10 or more, 15 or more, or about 20 of those targets) can be inserted into an immune cell (e.g., an NK cell, a B-Cell, a T-Cell, e.g., using a CAR or TCR) so that the immune cell specifically targets the individual's disease cells (e.g., tumor cells). As such, the inserted nucleotide sequence (of the second donor DNA) can be designed to be diagnostically responsive—in the sense that the encoded receptor(s) (e.g., heteromultivalent receptor(s)) can be designed after receiving unique insights related to a patient's proteomics, genomics or metabolomics (e.g., through sequencing etc.)—thus generating an avid and specific immune system response. In this way, immune cells (such as NK cells, B cell, T cells, and the like) can be genome edited to express receptors such as CAR and/or TCR proteins (e.g., heteromultivalent versions) that are designed to be effective against an individual's own disease (e.g., cancer). In some cases, regulatory T cells can be given similar avidity for tissues affected by autoimmunity following diagnostically-responsive medicine.

In some cases the nucleotide sequence, of the second donor DNA that is inserted into the cell's genome includes a protein-coding nucleotide sequence that does not have introns. In some cases the nucleotide sequence that does not have introns encodes all or a portion of a TCR protein.

In some embodiments more than one delivery vehicle is introduced into a target cell. For example, in some cases a subject method includes introducing a first and a second of said delivery vehicles into the cell, where the nucleotide sequence of the second donor DNA of the first delivery vehicle, that is inserted into the cell's genome, encodes a T cell receptor (TCR) Alpha or Delta subunit, and the nucleotide sequence of the second donor DNA of the second delivery vehicle, that is inserted into the cell's genome, encodes a TCR Beta or Gamma subunit. In some cases a subject method includes introducing a first and a second of said delivery vehicles into the cell, where the nucleotide sequence of the second donor DNA of the first delivery vehicle, that is inserted into the cell's genome, encodes a T cell receptor (TCR) Alpha or Delta subunit constant region, and the nucleotide sequence of the second donor DNA of the second delivery vehicle, that is inserted into the cell's genome, encodes a TCR Beta or Gamma subunit constant region.

In some cases a subject method includes introducing a first and a second of said delivery vehicles into the cell, wherein the nucleotide sequence of the second donor DNA of the first delivery vehicle is inserted within a nucleotide sequence that functions as a T cell receptor (TCR) Alpha or Delta subunit promoter, and the nucleotide sequence of the second donor DNA of the second delivery vehicle is inserted within a nucleotide sequence that functions as a TCR Beta or Gamma subunit promoter. For more information related to TCR proteins and CDRs, see, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21.

In some cases a subject method includes introducing a first and a second of said delivery vehicles into the cell, where the nucleotide sequence of the second donor DNA of the first delivery vehicle, that is inserted into the cell's genome, encodes a T cell receptor (TCR) Alpha or Gamma subunit, and the nucleotide sequence of the second donor DNA of the second delivery vehicle, that is inserted into the cell's genome, encodes a TCR Beta or Delta subunit. In some cases a subject method includes introducing a first and a second of said delivery vehicles into the cell, where the nucleotide sequence of the second donor DNA of the first delivery vehicle, that is inserted into the cell's genome, encodes a T cell receptor (TCR) Alpha or Delta subunit constant region, and the nucleotide sequence of the second donor DNA of the second delivery vehicle, that is inserted into the cell's genome, encodes a TCR Beta or Gamma subunit constant region. In some cases a subject method includes introducing a first and a second of said delivery vehicles into the cell, wherein the nucleotide sequence of the second donor DNA of the first delivery vehicle is inserted within a nucleotide sequence that functions as a T cell receptor (TCR) Alpha or Gamma subunit promoter, and the nucleotide sequence of the second donor DNA of the second delivery vehicle is inserted within a nucleotide sequence that functions as a TCR Beta or Delta subunit promoter. For more information related to TCR proteins and CDRs, see, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21.

### Delivery Vehicles/Payloads

In some embodiments, subject compositions (nuclease composition, target donor composition, recombinase composition, and/or insert donor composition) are delivered to a cell as payloads of the same delivery vehicle). For example, in some cases, (i) a subject first donor DNA; (ii) one or more sequence specific nucleases (such as a meganuclease, a Homing Endonuclease, a Zinc Finger Nuclease, a TALEN, a CRISPR/Cas effector protein) (or more nucleic acids encoding one or more sequence specific nucleases); (iii) a site specific recombinase; and (iv) a second donor DNA, are payloads of the same delivery vehicle. In some such cases the payloads bind together and form one or more: ribonucleoprotein complexes (e.g., a complex that includes a protein and an RNA, e.g., a CRISPR/Cas effector protein and a guide RNA), deoxyribonucleoprotein complexes (e.g., a complex that includes the DNA and protein), and/or ribo-deoxyribonucleoprotein complexes (e.g., a complex that includes protein, DNA, and RNA).

Delivery vehicles can include, but are not limited to, non-viral vehicles, viral vehicles, nanoparticles (e.g., a nanoparticle that includes a targeting ligand and/or a core comprising an anionic polymer composition, a cationic polymer composition, and a cationic polypeptide composition), liposomes, micelles, water-oil-water emulsion particles, oil-water emulsion micellar particles, multilamellar water-oil-water emulsion particles, a targeting ligand (e.g., peptide targeting ligand) conjugated to a charged polymer polypeptide domain (wherein the targeting ligand provides for targeted binding to a cell surface protein, and the charged polymer polypeptide domain is condensed with a nucleic acid payload and/or is interacting electrostatically with a protein payload), a targeting ligand (e.g., peptide targeting ligand) conjugated to payload (where the targeting ligand provides for targeted binding to a cell surface protein).

In some cases, a delivery vehicle is a water-oil-water emulsion particle. In some cases, a delivery vehicle is an oil-water emulsion micellar particle. In some cases, a delivery vehicle is a multilamellar water-oil-water emulsion particle. In some cases, a delivery vehicle is a multilayered particle. In some cases, a delivery vehicle is a DNA origami nanobot. For any of the above a payload (nucleic acid and/or protein) can be inside of the particle, either covalently, bound as nucleic acid complementary pairs, or within a water phase of a particle. In some cases a delivery vehicle includes a targeting ligand, e.g., in some cases a targeting ligand (described in more detail elsewhere herein) coated upon a water-oil-water emulsion particle, upon an oil-water emulsion micellar particle, upon a multilamellar water-oil-water emulsion particle, upon a multilayered particle, or upon a DNA origami nanobot. In some cases a delivery vehicle has a metal particle core, and the payload (e.g., donor DNA and/or site specific nuclease—or nucleic acid encoding same) can be conjugated to (covalently bound to) the metal core.

### Nanoparticles

Nanoparticles of the disclosure include a payload, which can be made of nucleic acid and/or protein. For example, in some cases a subject nanoparticle is used to deliver a nucleic acid payload (e.g., a DNA and/or RNA). In some cases the core of the nanoparticle includes the payload(s). In some such cases a nanoparticle core can also include an anionic polymer composition, a cationic polymer composition, and a cationic polypeptide composition. In some cases the nanoparticle has a metallic core and the payload associates with (in some cases is conjugated to, e.g., the outside of) the core. In some embodiments, the payload is part of the nanoparticle core. Thus the core of a subject nanoparticle can include nucleic acid, DNA, RNA, and/or protein. Thus, in some cases a subject nanoparticle includes nucleic acid (DNA and/or RNA) and protein. In some cases a subject nanoparticle core includes a ribonucleoprotein (RNA and protein) complex. In some cases a subject nanoparticle core includes a deoxyribonucleoprotein (DNA and protein, e.g., donor DNA and ZFN, TALEN, or CRISPR/Cas effector protein) complex. In some cases a subject nanoparticle core includes a ribo-deoxyribonucleoprotein (RNA and DNA and protein, e.g., a guide RNA, a donor DNA and a CRISPR/Cas effector protein) complex. In some cases a subject nanoparticle core includes PNAs. In some cases a subject core includes PNAs and DNAs.

A subject nucleic acid payload can include a morpholino backbone structure. In some case, a subject nucleic acid payload (e.g., a donor DNA and/or a nucleic acid encoding a sequence specific nuclease and/or a nucleic acid encoding a recombinase) can have one or more locked nucleic acids (LNAs). Suitable sugar substituent groups include methoxy (—O—CH₃), aminopropoxy (—OCH₂CH₂CH₂NH₂), allyl (—CH₂—CH═CH₂), —O-allyl (—O—H₂—CH═CH₂) and fluoro (F). 2′-sugar substituent groups may be in the arabino (up) position or ribo (down) position. Suitable base modifications include synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

In some cases, a nucleic acid payload can include a conjugate moiety (e.g., one that enhances the activity, stability, cellular distribution or cellular uptake of the nucleic acid payload). These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Any convenient polynucleotide can be used as a subject nucleic acid payload that is not the donor DNA (e.g., for delivering a site specific nuclease and/or a site specific recombinase). Examples include but are not limited to: species of RNA and DNA including mRNA, m1A modified mRNA (monomethylation at position 1 of Adenosine), morpholino RNA, peptoid and peptide nucleic acids, cDNA, DNA origami, DNA and RNA with synthetic nucleotides, DNA and RNA with predefined secondary structures, and multimers and oligomers of the aforementioned.

As noted above, more than one payload is delivered as part of the same package (delivery vehicle) (e.g., nanoparticle), e.g., in some cases different payloads are part of different cores. One advantage of delivering multiple payloads as part of the same delivery vehicle (e.g., nanoparticle) is that the efficiency of each payload is not diluted. As an illustrative example, if payload A and payload B are delivered in two separate packages/vehicles (package A and package B, respectively), then the efficiencies are multiplicative, e.g., if package A and package B each have a 1% transfection efficiency, the chance of delivering payload A and payload B to the same cell is 0.01% (1%×1%). However, if payload A and payload B are both delivered as part of the same delivery vehicle, then the chance of delivering payload A and payload B to the same cell is 1%, a 100-fold improvement over 0.01%.

Likewise, in a scenario where package A and package B each have a 0.1% transfection efficiency, the chance of delivering payload A and payload B to the same cell is 0.0001% (0.1%×0.1%). However, if payload A and payload B are both delivered as part of the same package (e.g., part of the same nanoparticle—package A) in this scenario, then the chance of delivering payload A and payload B to the same cell is 0.1%, a 1000-fold improvement over 0.0001%.

As such, in some embodiments, one or more gene editing tools (e.g., as described above) and a donor DNA are delivered in combination with (e.g., as part of the same nanoparticle) a protein (and/or a DNA or mRNA encoding same) and/or a non-coding RNA that increases genomic editing efficiency. In some cases, one or more gene editing tools (e.g., as described above) and a donor DNA are delivered in combination with (e.g., as part of the same nanoparticle) a protein (and/or a DNA or mRNA encoding same) and/or a non-coding RNA that controls cell division and/or differentiation.

As non-limiting examples of the above, in some embodiments one or more gene editing tools and a donor DNA can be delivered in combination with one or more of: SCF (and/or a DNA or mRNA encoding SCF), HoxB4 (and/or a DNA or mRNA encoding HoxB4), BCL-XL (and/or a DNA or mRNA encoding BCL-XL), SIRT6 (and/or a DNA or mRNA encoding SIRT6), a nucleic acid molecule (e.g., an siRNA and/or an LNA) that suppresses miR-155, a nucleic acid molecule (e.g., an siRNA, an shRNA, a microRNA) that reduces ku70 expression, and a nucleic acid molecule (e.g., an siRNA, an shRNA, a microRNA) that reduces ku80 expression.

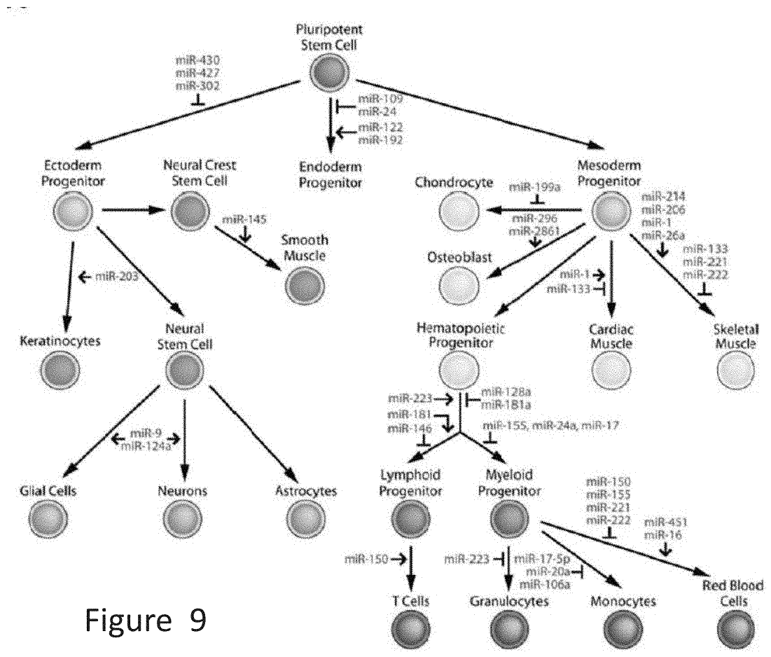

For examples of microRNAs that can be delivered in combination with a gene editing tool (e.g., a site specific nuclease) and a donor DNA, see FIG. 9. For example, the following microRNAs can be used for the following purposes: for blocking differentiation of a pluripotent stem cell toward ectoderm lineage: miR-430/427/302 (see, e.g., MiR Base accession: MI0000738, MI0000772, MI0000773, MI0000774, MI0006417, MI0006418, MI0000402, MI0003716, MI0003717, and MI0003718); for blocking differentiation of a pluripotent stem cell toward endoderm lineage: miR-109 and/or miR-24 (see, e.g., MiR Base accession: MI0000080, MI0000081, MI0000231, and MI0000572); for driving differentiation of a pluripotent stem cell toward endoderm lineage: miR-122 (see, e.g., MiR Base accession: MI0000442 and MI0000256) and/or miR-192 (see, e.g., MiR Base accession: MI0000234 and MI0000551); for driving differentiation of an ectoderm progenitor cell toward a keratinocyte fate: miR-203 (see, e.g., MiR Base accession: MI0000283, MI0017343, and MI0000246); for driving differentiation of a neural crest stem cell toward a smooth muscle fate: miR-145 (see, e.g., MiR Base accession: MI0000461, MI0000169, and MI0021890); for driving differentiation of a neural stem cell toward a glial cell fate and/or toward a neuron fate: miR-9 (see, e.g., MiR Base accession: MI0000466, MI0000467, MI0000468, MI0000157, MI0000720, and MI0000721) and/or miR-124a (see, e.g., MiR Base accession: MI0000443, MI0000444, MI0000445, MI0000150, MI0000716, and MI0000717); for blocking differentiation of a mesoderm progenitor cell toward a chondrocyte fate: miR-199a (see, e.g., MiR Base accession: MI0000242, MI0000281, MI0000241, and MI0000713); for driving differentiation of a mesoderm progenitor cell toward an osteoblast fate: miR-296 (see, e.g., MiR Base accession: MI0000747 and MI0000394) and/or miR-2861 (see, e.g., MiR Base accession: MI0013006 and MI0013007); for driving differentiation of a mesoderm progenitor cell toward a cardiac muscle fate: miR-1 (see, e.g., MiR Base accession: MI0000437, MI0000651, MI0000139, MI0000652, MI0006283); for blocking differentiation of a mesoderm progenitor cell toward a cardiac muscle fate: miR-133 (see, e.g., MiR Base accession: MI0000450, MI0000451, MI0000822, MI0000159, MI0000820, MI0000821, and MI0021863); for driving differentiation of a mesoderm progenitor cell toward a skeletal muscle fate: miR-214 (see, e.g., MiR Base accession: MI0000290 and MI0000698), miR-206 (see, e.g., MiR Base accession: MI0000490 and MI0000249), miR-1 and/or miR-26a (see, e.g., MiR Base accession: MI0000083, MI0000750, MI0000573, and MI0000706); for blocking differentiation of a mesoderm progenitor cell toward a skeletal muscle fate: miR-133 (see, e.g., MiR Base accession: MI0000450, MI0000451, MI0000822, MI0000159, MI0000820, MI0000821, and MI0021863), miR-221 (see, e.g., MiR Base accession: MI0000298 and MI0000709), and/or miR-222 (see, e.g., MiR Base accession: MI0000299 and MI0000710); for driving differentiation of a hematopoietic progenitor cell toward differentiation: miR-223 (see, e.g., MiR Base accession: MI0000300 and MI0000703); for blocking differentiation of a hematopoietic progenitor cell toward differentiation: miR-128a (see, e.g., MiR Base accession: MI0000447 and MI0000155) and/or miR-181a (see, e.g., MiR Base accession: MI0000269, MI0000289, MI0000223, and MI0000697); for driving differentiation of a hematopoietic progenitor cell toward a lymphoid progenitor cell: miR-181 (see, e.g., MiR Base accession: MI0000269, MI0000270, MI0000271, MI0000289, MI0000683, MI0003139, MI0000223, MI0000723, MI0000697, MI0000724, MI0000823, and MI0005450); for blocking differentiation of a hematopoietic progenitor cell toward a lymphoid progenitor cell: miR-146 (see, e.g., MiR Base accession: MI0000477, MI0003129, MI0003782, MI0000170, and MI0004665); for blocking differentiation of a hematopoietic progenitor cell toward a myeloid progenitor cell: miR-155, miR-24a, and/or miR-17 (see, e.g., MiR Base accession: MI0000071 and MI0000687); for driving differentiation of a lymphoid progenitor cell toward a T cell fate: miR-150 (see, e.g., MiR Base accession: MI0000479 and MI0000172); for blocking differentiation of a myeloid progenitor cell toward a granulocyte fate: miR-223 (see, e.g., MiR Base accession: MI0000300 and MI0000703); for blocking differentiation of a myeloid progenitor cell toward a monocyte fate: miR-17-5p (see, e.g., MiR Base accession: MIMAT0000070 and MIMAT0000649), miR-20a (see, e.g., MiR Base accession: MI0000076 and MI0000568), and/or miR-106a (see, e.g., MiR Base accession: MI0000113 and MI0000406); for blocking differentiation of a myeloid progenitor cell toward a red blood cell fate: miR-150 (see, e.g., MiR Base accession: MI0000479 and MI0000172), miR-155, miR-221 (see, e.g., MiR Base accession: MI0000298 and MI0000709), and/or miR-222 (see, e.g., MiR Base accession: MI0000299 and MI0000710); and for driving differentiation of a myeloid progenitor cell toward a red blood cell fate: miR-451 (see, e.g., MiR Base accession: MI0001729, MI0017360, MI0001730, and MI0021960) and/or miR-16 (see, e.g., MiR Base accession: MI0000070, MI0000115, MI0000565, and MI0000566).

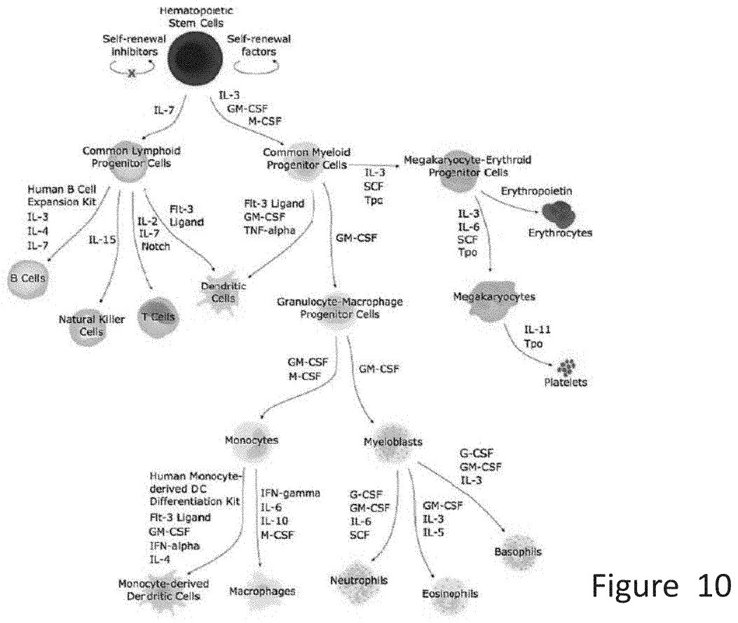

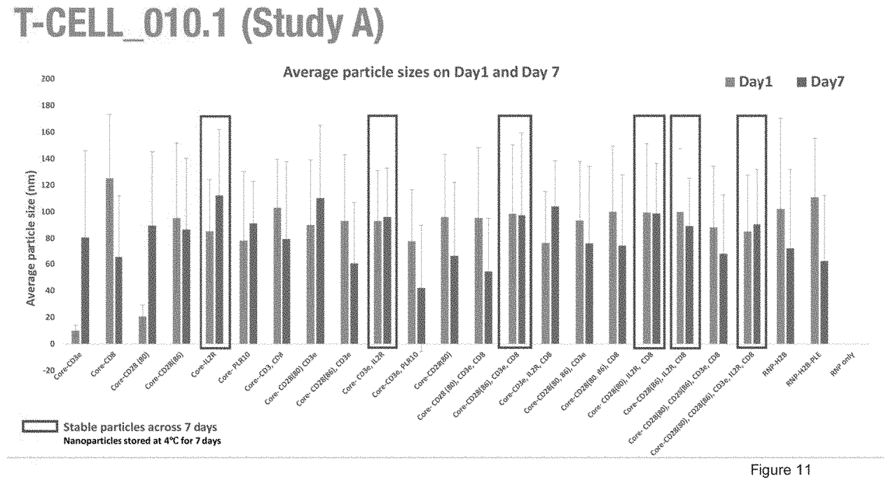

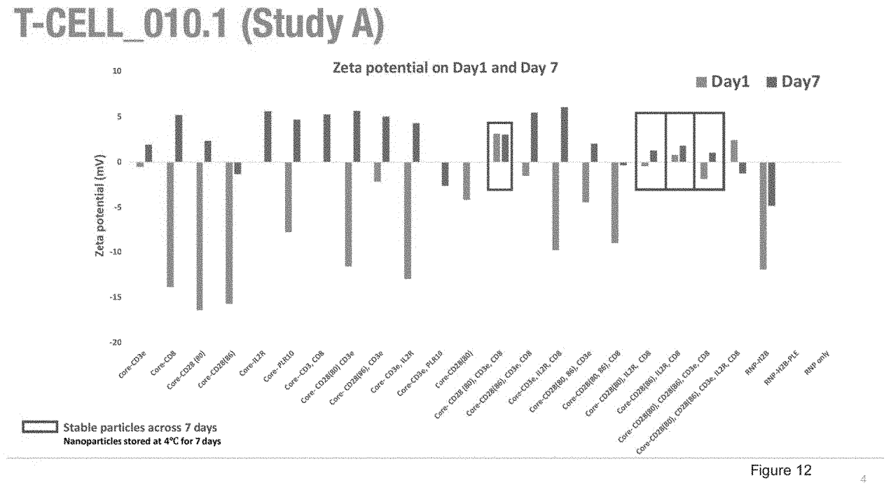

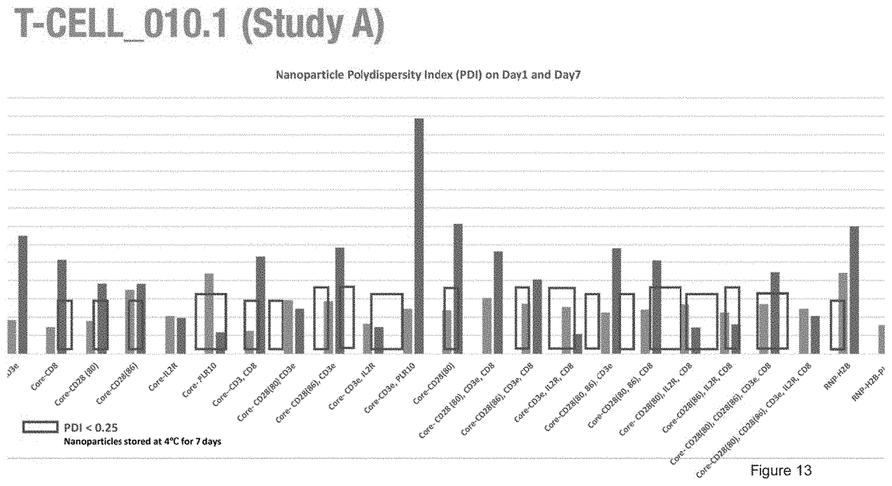

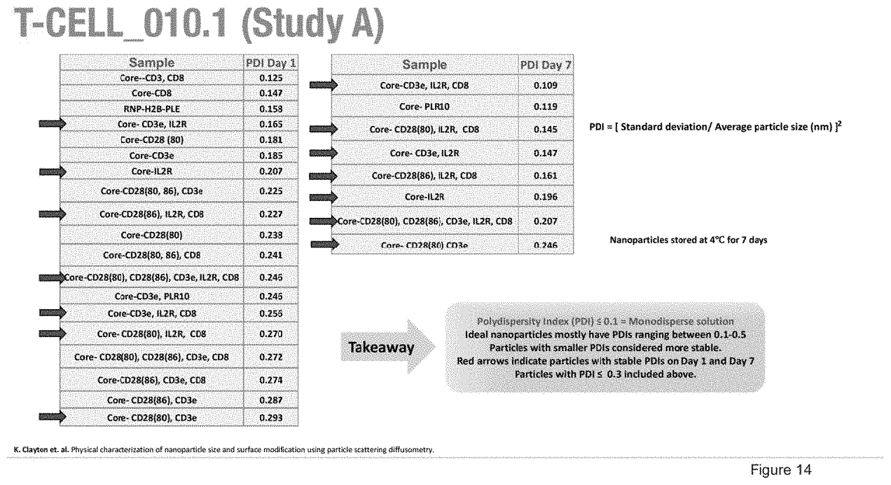

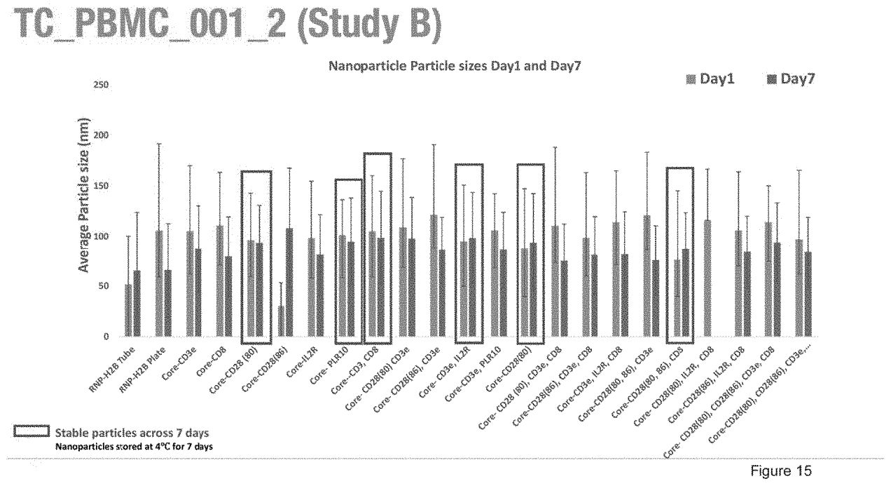

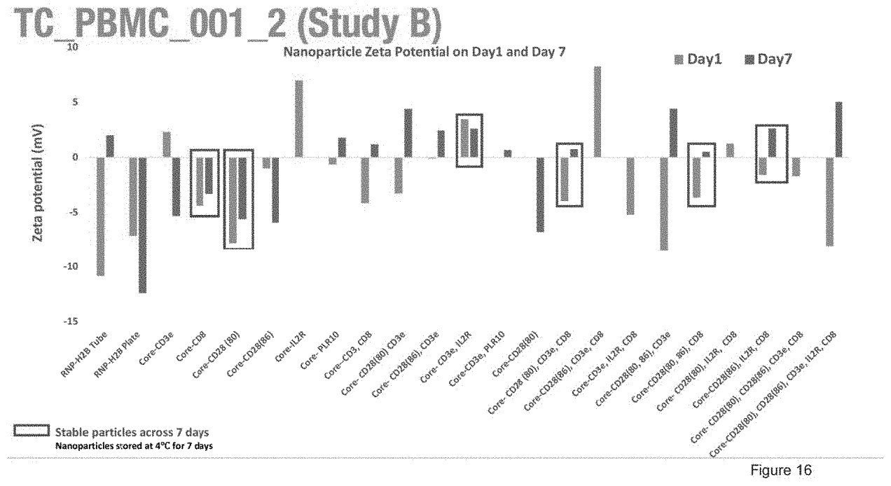

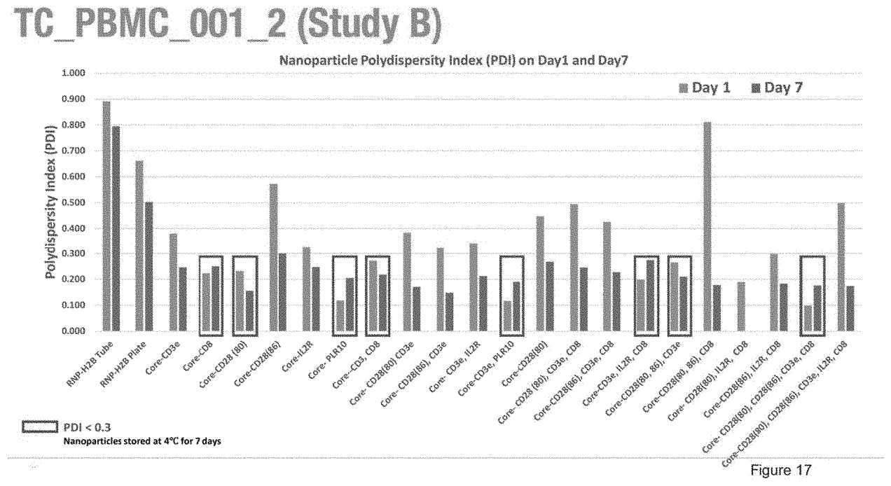

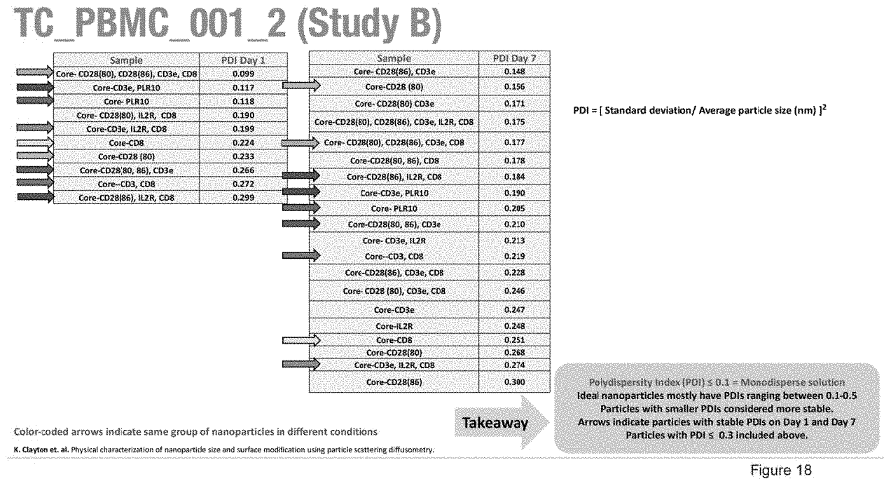

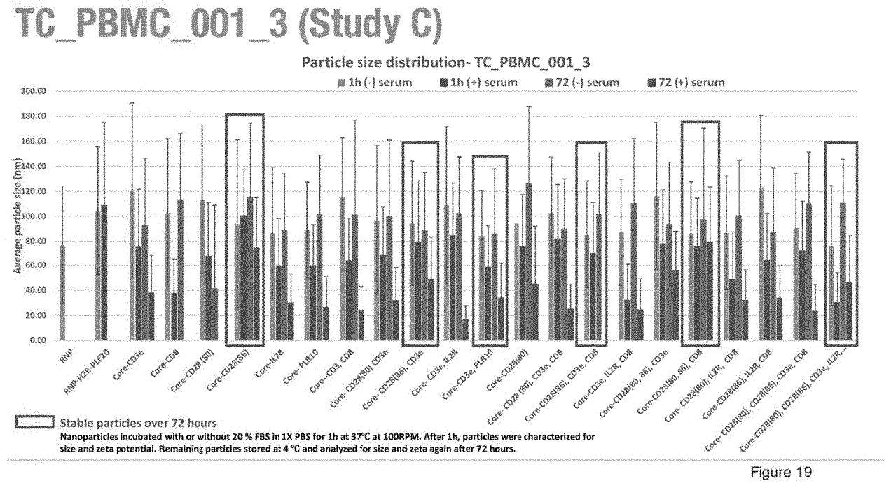

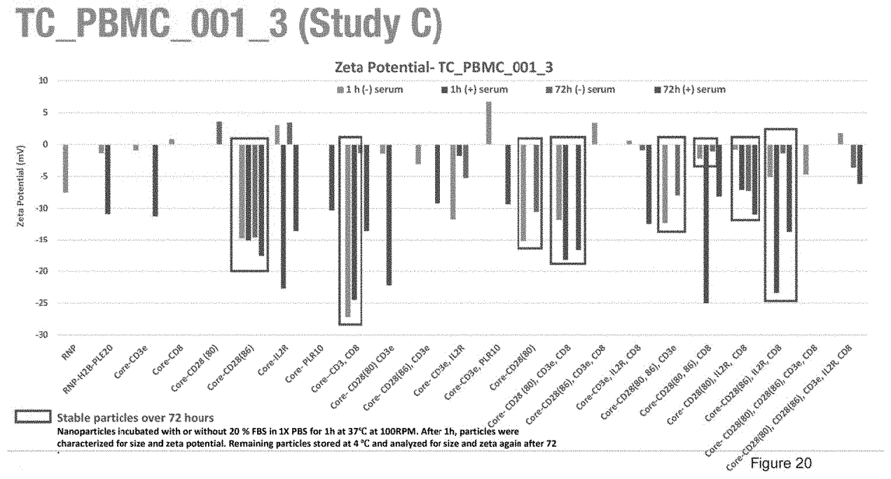

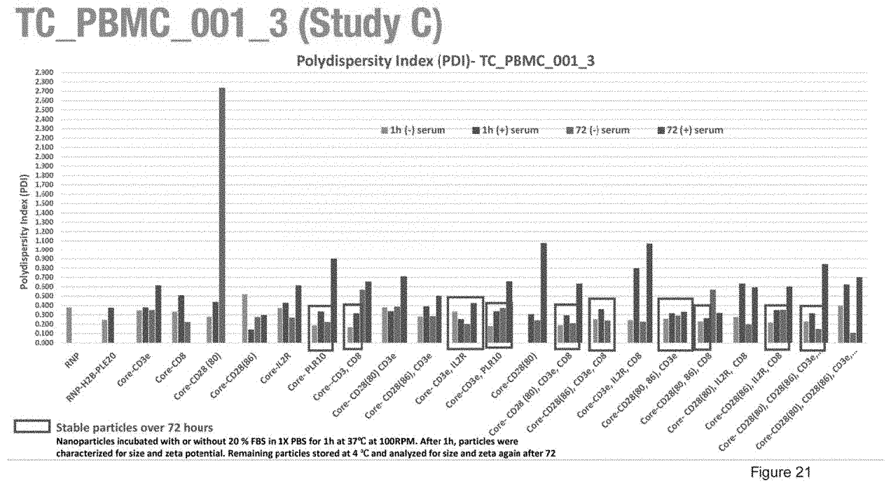

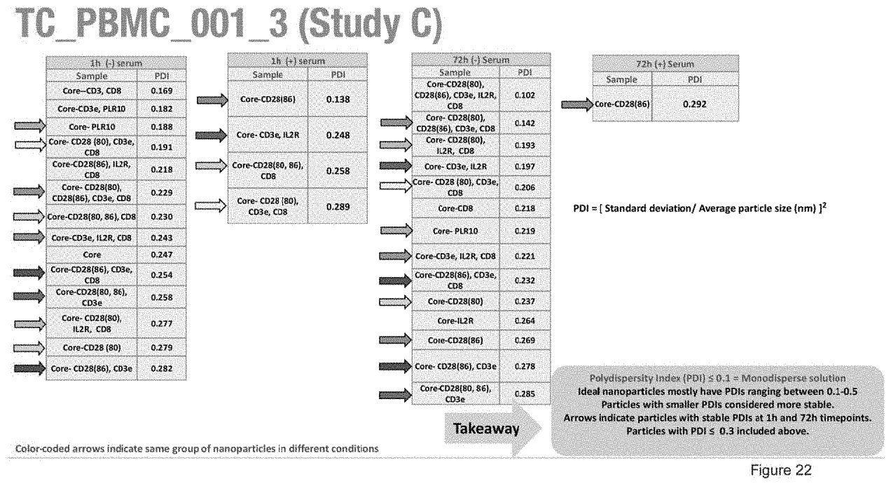

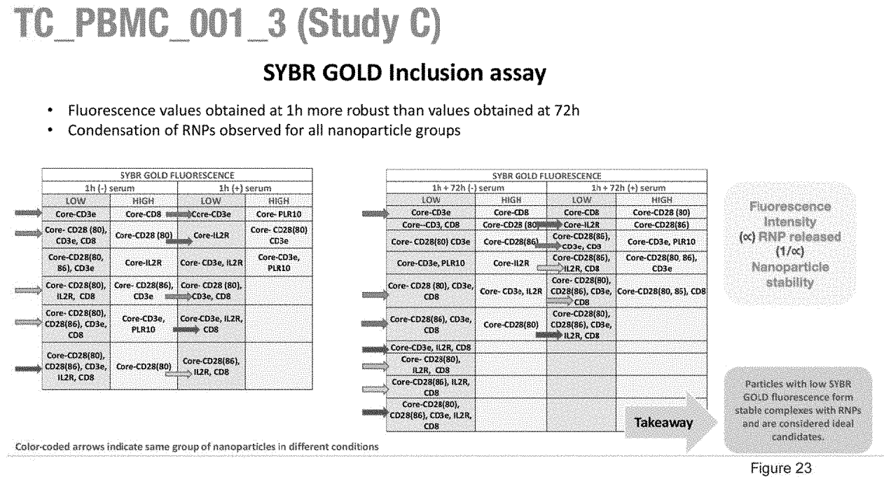

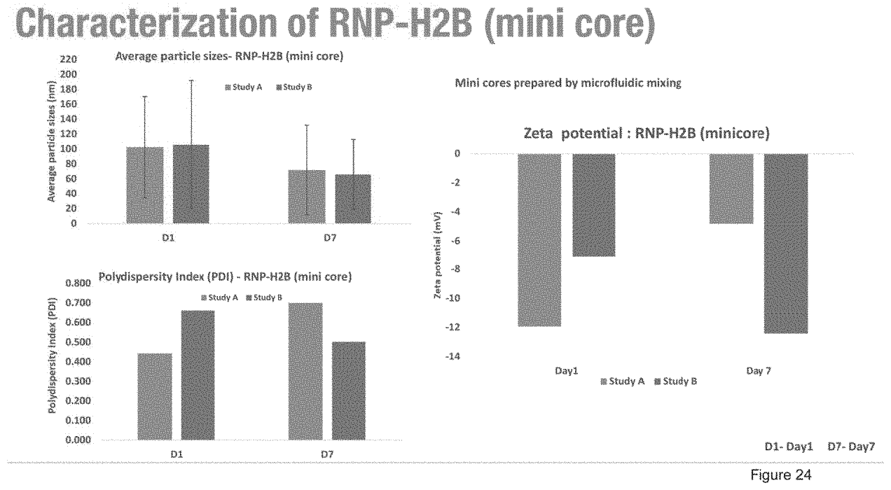

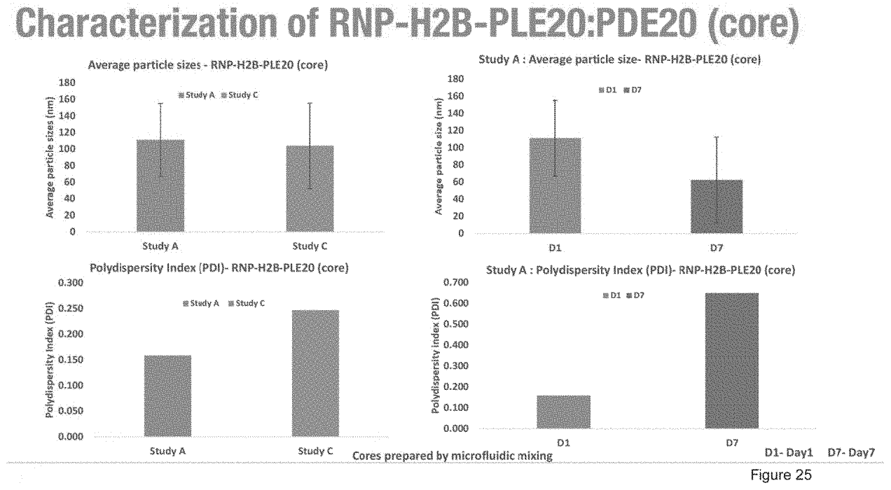

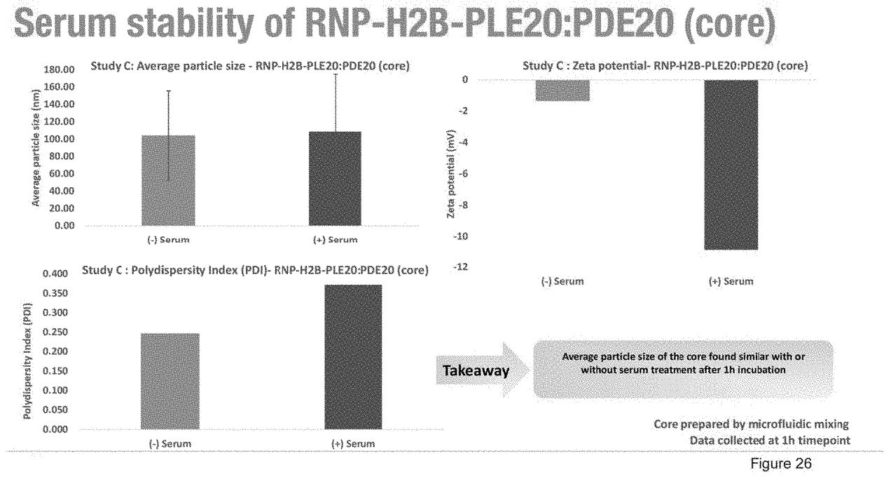

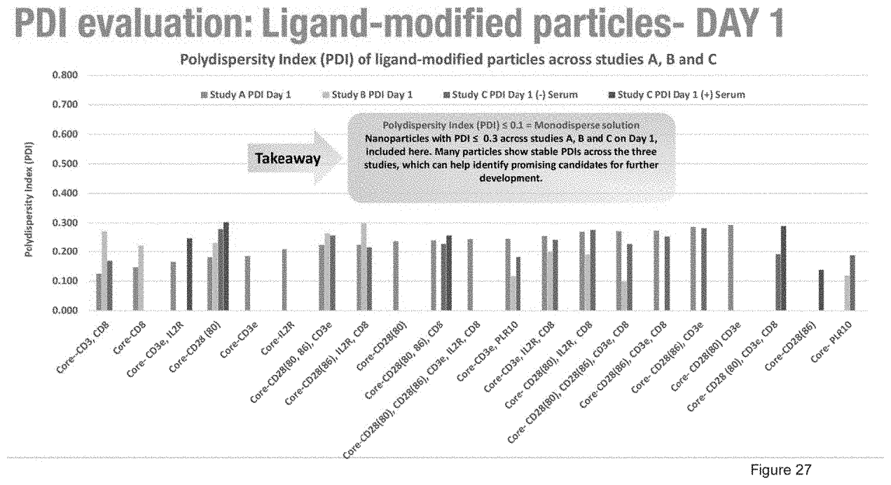

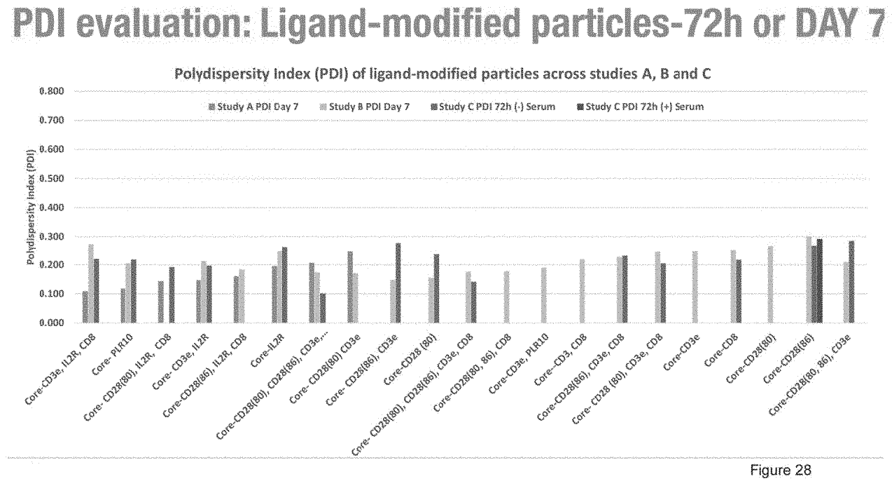

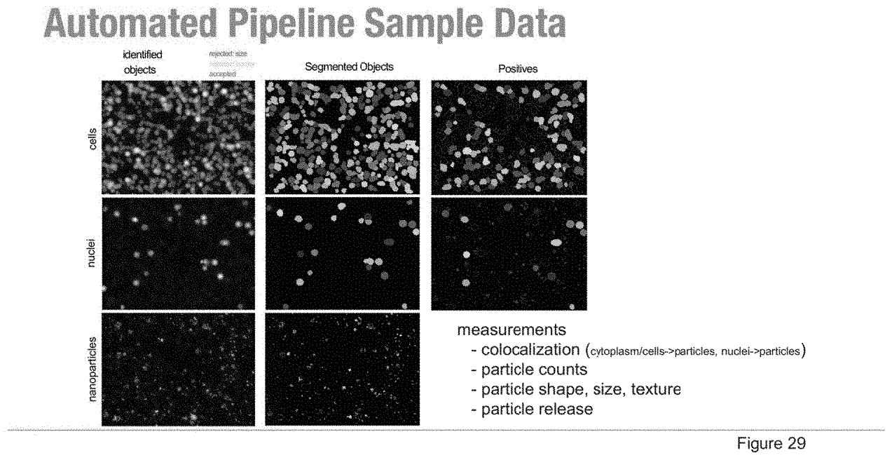

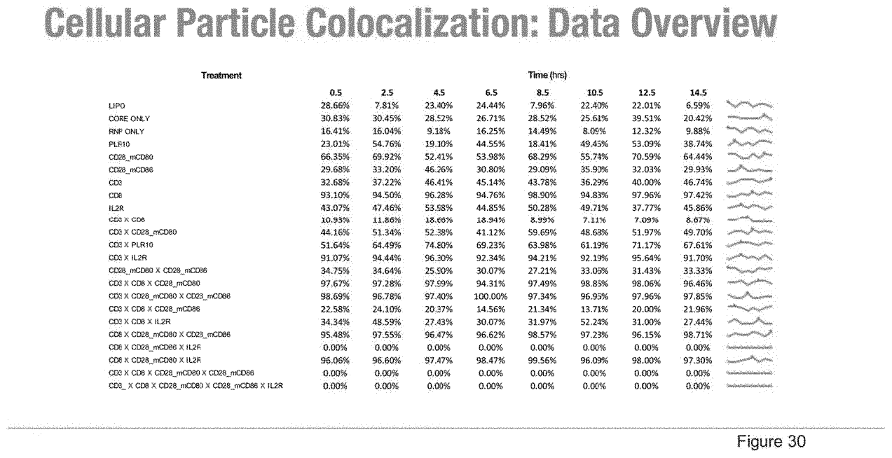

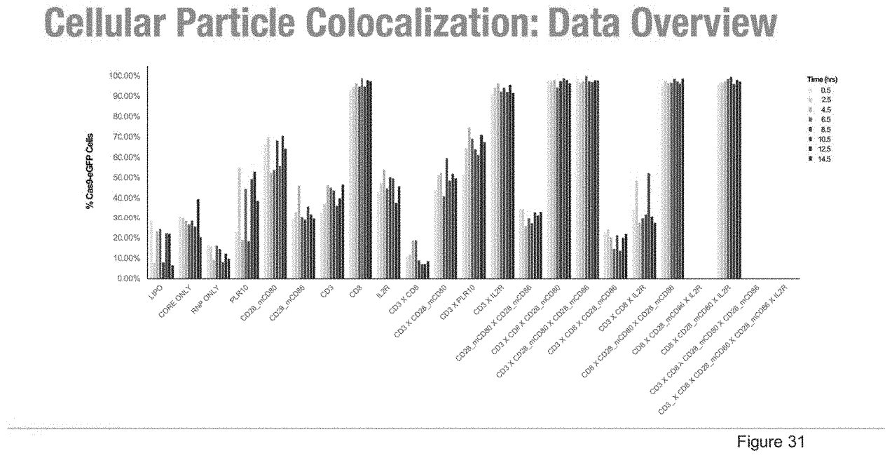

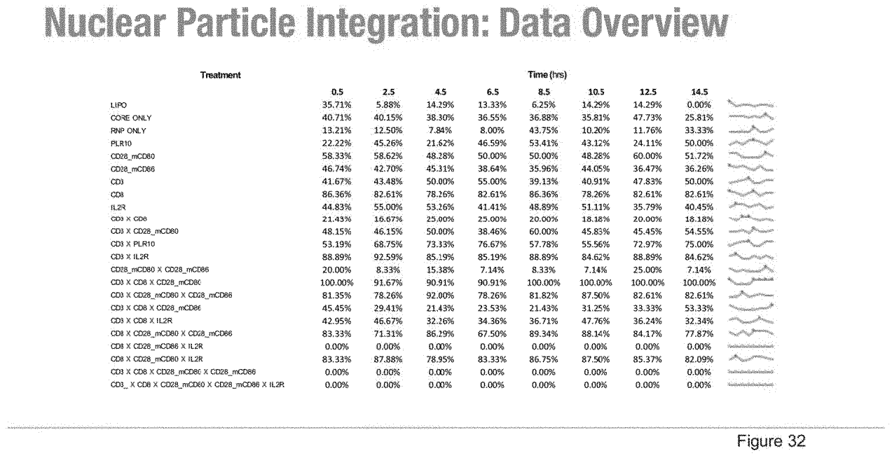

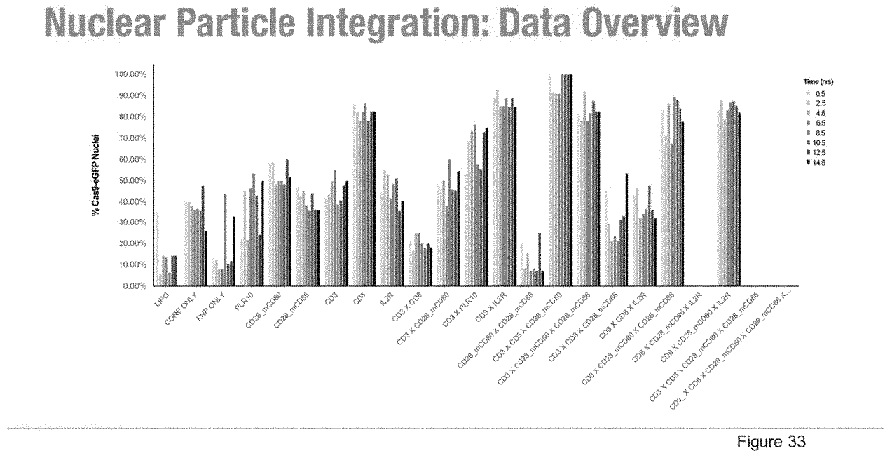

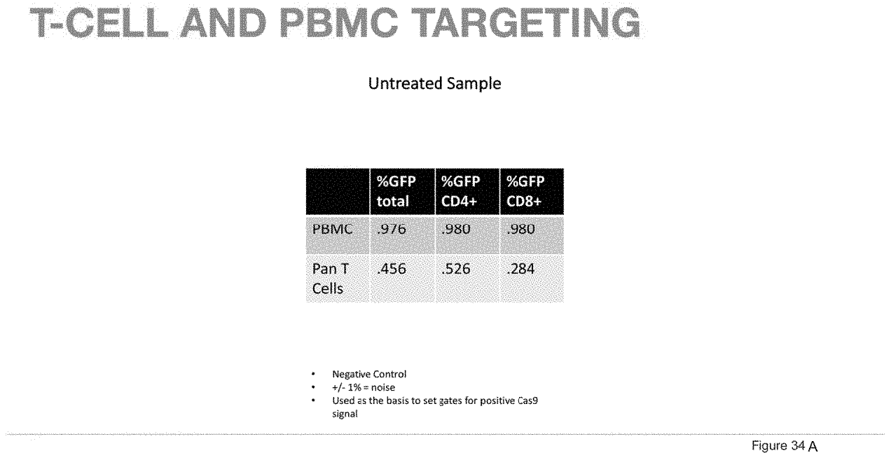

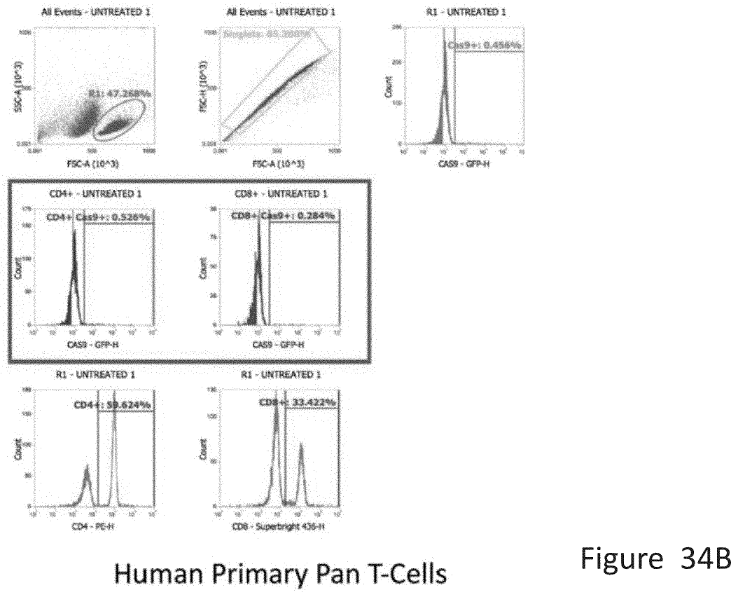

For examples of signaling proteins (e.g., extracellular signaling proteins) that can be delivered (e.g., as protein or as DNA or RNA encoding the protein) in combination with a gene editing tool, the first and second donor DNAs, and the recombinase (or nucleic acid encoding same), see FIG. 10. The same proteins can be used as part of the outer shell of a subject nanoparticle in a similar manner as a targeting ligand, e.g., for the purpose of biasing differentiation in target cells that receive the nanoparticle. For example, the following signaling proteins (e.g., extracellular signaling proteins) can be used for the following purposes: for driving differentiation of a hematopoietic stem cell toward a common lymphoid progenitor cell lineage: IL-7 (see, e.g., NCBI Gene ID 3574); for driving differentiation of a hematopoietic stem cell toward a common myeloid progenitor cell lineage: IL-3 (see, e.g., NCBI Gene ID 3562), GM-CSF (see, e.g., NCBI Gene ID 1437), and/or M-CSF (see, e.g., NCBI Gene ID 1435); for driving differentiation of a common lymphoid progenitor cell toward a B-cell fate: IL-3, IL-4 (see, e.g., NCBI Gene ID: 3565), and/or IL-7; for driving differentiation of a common lymphoid progenitor cell toward a Natural Killer Cell fate: IL-15 (see, e.g., NCBI Gene ID 3600); for driving differentiation of a common lymphoid progenitor cell toward a T-cell fate: IL-2 (see, e.g., NCBI Gene ID 3558), IL-7, and/or Notch (see, e.g., NCBI Gene IDs 4851, 4853, 4854, 4855); for driving differentiation of a common lymphoid progenitor cell toward a dendritic cell fate: Flt-3 ligand (see, e.g., NCBI Gene ID 2323); for driving differentiation of a common myeloid progenitor cell toward a dendritic cell fate: Flt-3 ligand, GM-CSF, and/or TNF-alpha (see, e.g., NCBI Gene ID 7124); for driving differentiation of a common myeloid progenitor cell toward a granulocyte-macrophage progenitor cell lineage: GM-CSF; for driving differentiation of a common myeloid progenitor cell toward a megakaryocyte-erythroid progenitor cell lineage: IL-3, SCF (see, e.g., NCBI Gene ID 4254), and/or Tpo (see, e.g., NCBI Gene ID 7173); for driving differentiation of a megakaryocyte-erythroid progenitor cell toward a megakaryocyte fate: IL-3, IL-6 (see, e.g., NCBI Gene ID 3569), SCF, and/or Tpo; for driving differentiation of a megakaryocyte-erythroid progenitor cell toward a erythrocyte fate: erythropoietin (see, e.g., NCBI Gene ID 2056); for driving differentiation of a megakaryocyte toward a platelet fate: IL-11 (see, e.g., NCBI Gene ID 3589) and/or Tpo; for driving differentiation of a granulocyte-macrophage progenitor cell toward a monocyte lineage: GM-CSF and/or M-CSF; for driving differentiation of a granulocyte-macrophage progenitor cell toward a myeloblast lineage: GM-CSF; for driving differentiation of a monocyte toward a monocyte-derived dendritic cell fate: Flt-3 ligand, GM-CSF, IFN-alpha (see, e.g., NCBI Gene ID 3439), and/or IL-4; for driving differentiation of a monocyte toward a macrophage fate: IFN-gamma, IL-6, IL-10 (see, e.g., NCBI Gene ID 3586), and/or M-CSF; for driving differentiation of a myeloblast toward a neutrophil fate: G-CSF (see, e.g., NCBI Gene ID 1440), GM-CSF, IL-6, and/or SCF; for driving differentiation of a myeloblast toward a eosinophil fate: GM-CSF, IL-3, and/or IL-5 (see, e.g., NCBI Gene ID 3567); and for driving differentiation of a myeloblast toward a basophil fate: G-CSF, GM-CSF, and/or IL-3.

Examples of proteins that can be delivered (e.g., as protein and/or a nucleic acid such as DNA or RNA encoding the protein) in combination with the other payloads described herein include but are not limited to: SOX17, HEX, OSKM (Oct4/Sox2/Klf4/c-myc), and/or bFGF (e.g., to drive differentiation toward hepatic stem cell lineage); HNF4a (e.g., to drive differentiation toward hepatocyte fate); Poly (I:C), BMP-4, bFGF, and/or 8-Br-cAMP (e.g., to drive differentiation toward endothelial stem cell/progenitor lineage); VEGF (e.g., to drive differentiation toward arterial endothelium fate); Sox-2, Brn4, Myt1I, Neurod2, AsclI (e.g., to drive differentiation toward neural stem cell/progenitor lineage); and BDNF, FCS, Forskolin, and/or SHH (e.g., to drive differentiation neuron, astrocyte, and/or oligodendrocyte fate).

Examples of signaling proteins (e.g., extracellular signaling proteins) that can be delivered (e.g., as protein and/or a nucleic acid such as DNA or RNA encoding the protein) with the other payloads described herein include but are not limited to: cytokines (e.g., IL-2 and/or IL-15, e.g., for activating CD8+ T-cells); ligands and or signaling proteins that modulate one or more of the Notch, Wnt, and/or Smad signaling pathways; SCF; stem cell differentiating factors (e.g. Sox2, Oct3/4, Nanog, Klf4, c-Myc, and the like); and temporary surface marker “tags” and/or fluorescent reporters for subsequent isolation/purification/concentration. For example, a fibroblast may be converted into a neural stem cell via delivery of Sox2, while it will turn into a cardiomyocyte in the presence of Oct3/4 and small molecule “epigenetic resetting factors.” In a patient with Huntington's disease or a CXCR4 mutation, these fibroblasts may respectively encode diseased phenotypic traits associated with neurons and cardiac cells. By delivering gene editing corrections and these factors in a single package, the risk of deleterious effects due to one or more, but not all of the factors/payloads being introduced can be significantly reduced.

Because the timing and/or location of payload release can be controlled (described in more detail elsewhere in this disclosure), the packaging of multiple payloads in the same package (e.g., same nanoparticle) does not preclude one from achieving different release times/rates and/or locations for different payloads. For example the release of the above proteins (and/or a DNAs or mRNAs encoding same) and/or non-coding RNAs can be controlled separately from the release of the one or more gene editing tools that are part of the same package. For example, proteins and/or nucleic acids (e.g., DNAs, mRNAs, non-coding RNAs, miRNAs) that control cell proliferation and/or differentiation can be released earlier than the one or more gene editing tools or can be released later than the one or more gene editing tools. This can be achieved, e.g., by using more than one sheddable layer and/or by using more than one core (e.g., where one core has a different release profile than the other, e.g., uses a different D- to L-isomer ratio, uses a different ESP:ENP:EPP profile, and the like). In this way, a donor and nuclease may be released in a stepwise manner that allows for optimal editing and insertion efficiencies. Likewise, it may in some cases be desirable to release the first donor DNA and the nuclease (or nucleic acid encoding same) prior to releasing the recombinase (or nucleic acid encoding same) and the second donor DNA.

### Nanoparticle Core

The core of a subject nanoparticle can include an anionic polymer composition (e.g., poly(glutamic acid)), a cationic polymer composition (e.g., poly(arginine), a cationic polypeptide composition (e.g., a histone tail peptide), and a payload (e.g., nucleic acid and/or protein payload, e.g., first and second donor DNAs (e.g., one or more of each), a site specific nuclease or a nucleic acid encoding the site-specific nuclease, and a site specific recombinase or a nucleic acid encoding same). In some cases the core is generated by condensation of a cationic amino acid polymer and payload in the presence of an anionic amino acid polymer (and in some cases in the presence of a cationic polypeptide of a cationic polypeptide composition). In some embodiments, condensation of the components that make up the core can mediate increased transfection efficiency compared to conjugates of cationic polymers with a payload. Inclusion of an anionic polymer in a nanoparticle core may prolong the duration of intracellular residence of the nanoparticle and release of payload.

For the cationic and anionic polymer compositions of the core, ratios of D-isomer polymers to L-isomer polymers can be controlled in order to control the timed release of payload, where increased ratio of D-isomer polymers to L-isomer polymers leads to increased stability (reduced payload release rate), which for example can enable longer lasting gene expression from a payload delivered by a subject nanoparticle. In some cases modifying the ratio of D-to-L isomer polypeptides within the nanoparticle core can cause gene expression profiles (e.g., expression of a protein encoded by a payload molecule) to be on the order of from 1-90 days (e.g. from 1-80, 1-70, 1-60, 1-50, 1-40, 1-30, 1-25, 1-20, 1-15, 1-10, 3-90, 3-80, 3-70, 3-60, 3-50, 3-40, 3-30, 3-25, 3-20, 3-15, 3-10, 5-90, 5-80, 5-70, 5-60, 5-50, 5-40, 5-30, 5-25, 5-20, 5-15, or 5-10 days). The control of payload release (e.g., when delivering a gene editing tool), can be particularly effective for performing genomic edits e.g., in some cases where homology-directed repair is desired.

In some embodiments, a nanoparticle includes a core and a sheddable layer encapsulating the core, where the core includes: (a) an anionic polymer composition; (b) a cationic polymer composition; (c) a cationic polypeptide composition; and (d) a nucleic acid and/or protein payload, where one of (a) and (b) includes a D-isomer polymer of an amino acid, and the other of (a) and (b) includes an L-isomer polymer of an amino acid, and where the ratio of the D-isomer polymer to the L-isomer polymer is in a range of from 10:1 to 1.5:1 (e.g., from 8:1 to 1.5:1, 6:1 to 1.5:1, 5:1 to 1.5:1, 4:1 to 1.5:1, 3:1 to 1.5:1, 2:1 to 1.5:1, 10:1 to 2:1; 8:1 to 2:1, 6:1 to 2:1, 5:1 to 2:1, 10:1 to 3:1; 8:1 to 3:1, 6:1 to 3:1, 5:1 to 3:1, 10:1 to 4:1; 4:1 to 2:1, 6:1 to 4:1, or 10:1 to 5:1), or from 1:1.5 to 1:10 (e.g., from 1:1.5 to 1:8, 1:1.5 to 1:6, 1:1.5 to 1:5, 1:1.5 to 1:4, 1:1.5 to 1:3, 1:1.5 to 1:2, 1:2 to 1:10, 1:2 to 1:8, 1:2 to 1:6, 1:2 to 1:5, 1:2 to 1:4, 1:2 to 1:3, 1:3 to 1:10, 1:3 to 1:8, 1:3 to 1:6, 1:3 to 1:5, 1:4 to 1:10, 1:4 to 1:8, 1:4 to 1:6, or 1:5 to 1:10). In some such cases, the ratio of the D-isomer polymer to the L-isomer polymer is not 1:1. In some such cases, the anionic polymer composition includes an anionic polymer selected from poly(D-glutamic acid) (PDEA) and poly(D-aspartic acid) (PDDA), where (optionally) the cationic polymer composition can include a cationic polymer selected from poly(L-arginine), poly(L-lysine), poly(L-histidine), poly(L-ornithine), and poly(L-citrulline). In some cases the cationic polymer composition comprises a cationic polymer selected from poly(D-arginine), poly(D-lysine), poly(D-histidine), poly(D-ornithine), and poly(D-citrulline), where (optionally) the anionic polymer composition can include an anionic polymer selected from poly(L-glutamic acid) (PLEA) and poly(L-aspartic acid) (PLDA).

In some embodiments, a nanoparticle includes a core and a sheddable layer encapsulating the core, where the core includes: (i) an anionic polymer composition; (ii) a cationic polymer composition; (iii) a cationic polypeptide composition; and (iv) a nucleic acid and/or protein payload, wherein (a) said anionic polymer composition includes polymers of D-isomers of an anionic amino acid and polymers of L-isomers of an anionic amino acid; and/or (b) said cationic polymer composition includes polymers of D-isomers of a cationic amino acid and polymers of L-isomers of a cationic amino acid. In some such cases, the anionic polymer composition comprises a first anionic polymer selected from poly(D-glutamic acid) (PDEA) and poly(D-aspartic acid) (PDDA); and comprises a second anionic polymer selected from poly(L-glutamic acid) (PLEA) and poly(L-aspartic acid) (PLDA). In some cases, the cationic polymer composition comprises a first cationic polymer selected from poly(D-arginine), poly(D-lysine), poly(D-histidine), poly(D-ornithine), and poly(D-citrulline); and comprises a second cationic polymer selected from poly(L-arginine), poly(L-lysine), poly(L-histidine), poly(L-ornithine), and poly(L-citrulline). In some cases, the polymers of D-isomers of an anionic amino acid are present at a ratio, relative to said polymers of L-isomers of an anionic amino acid, in a range of from 10:1 to 1:10. In some cases, the polymers of D-isomers of a cationic amino acid are present at a ratio, relative to said polymers of L-isomers of a cationic amino acid, in a range of from 10:1 to 1:10.

### Nanoparticle Components (Timing)

In some embodiments, timing of payload release can be controlled by selecting particular types of proteins, e.g., as part of the core (e.g., part of a cationic polypeptide composition, part of a cationic polymer composition, and/or part of an anionic polymer composition). For example, it may be desirable to delay payload release for a particular range of time, or until the payload is present at a particular cellular location (e.g., cytosol, nucleus, lysosome, endosome) or under a particular condition (e.g., low pH, high pH, etc.). As such, in some cases a protein is used (e.g., as part of the core) that is susceptible to a specific protein activity (e.g., enzymatic activity), e.g., is a substrate for a specific protein activity (e.g., enzymatic activity), and this is in contrast to being susceptible to general ubiquitous cellular machinery, e.g., general degradation machinery. A protein that is susceptible to a specific protein activity is referred to herein as an ‘enzymatically susceptible protein’ (ESP). Illustrative examples of ESPs include but are not limited to: (i) proteins that are substrates for matrix metalloproteinase (MMP) activity (an example of an extracellular activity), e.g., a protein that includes a motif recognized by an MMP; (ii) proteins that are substrates for cathepsin activity (an example of an intracellular endosomal activity), e.g., a protein that includes a motif recognized by a cathepsin; and (iii) proteins such as histone tails peptides (HTPs) that are substrates for methyltransferase and/or acetyltransferase activity (an example of an intracellular nuclear activity), e.g., a protein that includes a motif that can be enzymatically methylated/de-methylated and/or a motif that can be enzymatically acetylated/de-acetylated. For example, in some cases a nucleic acid payload is condensed with a protein (such as a histone tails peptide) that is a substrate for acetyltransferase activity, and acetylation of the protein causes the protein to release the payload—as such, one can exercise control over payload release by choosing to use a protein that is more or less susceptible to acetylation.

In some cases, a core of a subject nanoparticle includes an enzymatically neutral polypeptide (ENP), which is a polypeptide homopolymer (i.e., a protein having a repeat sequence) where the polypeptide does not have a particular activity and is neutral. For example, unlike NLS sequences and HTPs, both of which have a particular activity, ENPs do not.

In some cases, a core of a subject nanoparticle includes an enzymatically protected polypeptide (EPP), which is a protein that is resistant to enzymatic activity. Examples of PPs include but are not limited to: (i) polypeptides that include D-isomer amino acids (e.g., D-isomer polymers), which can resist proteolytic degradation; and (ii) self-sheltering domains such as a polyglutamine repeat domains (e.g., QQQQQQQQQQ) (SEQ ID NO: 170).

By controlling the relative amounts of susceptible proteins (ESPs), neutral proteins (ENPs), and protected proteins (EPPs), that are part of a subject nanoparticle (e.g., part of the nanoparticle core), one can control the release of payload. For example, use of more ESPs can in general lead to quicker release of payload than use of more EPPs. In addition, use of more ESPs can in general lead to release of payload that depends upon a particular set of conditions/circumstances, e.g., conditions/circumstances that lead to activity of proteins (e.g., enzymes) to which the ESP is susceptible.

### Anionic Polymer Composition of a Nanoparticle

An anionic polymer composition can include one or more anionic amino acid polymers. For example, in some cases a subject anionic polymer composition includes a polymer selected from: poly(glutamic acid)(PEA), poly(aspartic acid)(PDA), and a combination thereof. In some cases a given anionic amino acid polymer can include a mix of aspartic and glutamic acid residues. Each polymer can be present in the composition as a polymer of L-isomers or D-isomers, where D-isomers are more stable in a target cell because they take longer to degrade. Thus, inclusion of D-isomer poly(amino acids) in the nanoparticle core delays degradation of the core and subsequent payload release. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases an anionic polymer composition of a subject nanoparticle includes polymers of D-isomers and polymers of L-isomers of an anionic amino acid polymer (e.g., poly(glutamic acid)(PEA) and poly(aspartic acid)(PDA)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases an anionic polymer composition includes a first anionic polymer (e.g., amino acid polymer) that is a polymer of D-isomers (e.g., selected from poly(D-glutamic acid) (PDEA) and poly(D-aspartic acid) (PDDA)); and includes a second anionic polymer (e.g., amino acid polymer) that is a polymer of L-isomers (e.g., selected from poly(L-glutamic acid) (PLEA) and poly(L-aspartic acid) (PLDA)). In some cases the ratio of the first anionic polymer (D-isomers) to the second anionic polymer (L-isomers) is in a range of from 10:1-1:10 (e.g., from 8:1-1:10, 6:1-1:10, 4:1-1:10, 3:1-1:10, 2:1-1:10,1:1-1:10,10:1-1:8,8:1-1:8,6:1-1:8,4:1-1:8,3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, an anionic polymer composition of a core of a subject nanoparticle includes (e.g., in addition to or in place of any of the foregoing examples of anionic polymers) a glycosaminoglycan, a glycoprotein, a polysaccharide, poly(mannuronic acid), poly(guluronic acid), heparin, heparin sulfate, chondroitin, chondroitin sulfate, keratan, keratan sulfate, aggrecan, poly(glucosamine), or an anionic polymer that comprises any combination thereof.

In some embodiments, an anionic polymer within the core can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases an anionic polymer includes poly(glutamic acid) with a molecular weight of approximately 15 kDa.

In some cases, an anionic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes a cysteine residue. In some cases the anionic amino acid polymer includes cysteine residue on the N- and/or C-terminus. In some cases the anionic amino acid polymer includes an internal cysteine residue.

In some cases, an anionic amino acid polymer includes (and/or is conjugated to) a nuclear localization signal (NLS) (described in more detail below). Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

In some cases, an anionic polymer is added prior to a cationic polymer when generating a subject nanoparticle core.

### Cationic Polymer Composition of a Nanoparticle

A cationic polymer composition can include one or more cationic amino acid polymers. For example, in some cases a subject cationic polymer composition includes a polymer selected from: poly(arginine)(PR), poly(lysine)(PK), poly(histidine)(PH), poly(ornithine), poly(citrulline), and a combination thereof. In some cases a given cationic amino acid polymer can include a mix of arginine, lysine, histidine, ornithine, and citrulline residues (in any convenient combination). Each polymer can be present in the composition as a polymer of L-isomers or D-isomers, where D-isomers are more stable in a target cell because they take longer to degrade. Thus, inclusion of D-isomer poly(amino acids) in the nanoparticle core delays degradation of the core and subsequent payload release. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases a cationic polymer composition of a subject nanoparticle includes polymers of D-isomers and polymers of L-isomers of an cationic amino acid polymer (e.g., poly(arginine)(PR), poly(lysine)(PK), poly(histidine)(PH), poly(ornithine), poly(citrulline)). In some cases the D- to L-isomer ratio is in a range of from 10:1-1:10 (e.g., from 8:1-1:10, 6:1-1:10, 4:1-1:10, 3:1-1:10, 2:1-1:10,1:1-1:10,10:1-1:8,8:1-1:8,6:1-1:8,4:1-1:8,3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases a cationic polymer composition includes a first cationic polymer (e.g., amino acid polymer) that is a polymer of D-isomers (e.g., selected from poly(D-arginine), poly(D-lysine), poly(D-histidine), poly(D-ornithine), and poly(D-citrulline)); and includes a second cationic polymer (e.g., amino acid polymer) that is a polymer of L-isomers (e.g., selected from poly(L-arginine), poly(L-lysine), poly(L-histidine), poly(L-ornithine), and poly(L-citrulline)). In some cases the ratio of the first cationic polymer (D-isomers) to the second cationic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, an cationic polymer composition of a core of a subject nanoparticle includes (e.g., in addition to or in place of any of the foregoing examples of cationic polymers) poly(ethylenimine), poly(amidoamine) (PAMAM), poly(aspartamide), polypeptoids (e.g., for forming “spiderweb”-like branches for core condensation), a charge-functionalized polyester, a cationic polysaccharide, an acetylated amino sugar, chitosan, or a cationic polymer that comprises any combination thereof (e.g., in linear or branched forms).

In some embodiments, an cationic polymer within the core can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases an cationic polymer includes poly(L-arginine), e.g., with a molecular weight of approximately 29 kDa. As another example, in some cases a cationic polymer includes linear poly(ethylenimine) with a molecular weight of approximately 25 kDa (PEI). As another example, in some cases a cationic polymer includes branched poly(ethylenimine) with a molecular weight of approximately 10 kDa. As another example, in some cases a cationic polymer includes branched poly(ethylenimine) with a molecular weight of approximately 70 kDa. In some cases a cationic polymer includes PAMAM.

In some cases, a cationic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes a cysteine residue. In some cases the cationic amino acid polymer includes cysteine residue on the N- and/or C-terminus. In some cases the cationic amino acid polymer includes an internal cysteine residue.

In some cases, a cationic amino acid polymer includes (and/or is conjugated to) a nuclear localization signal (NLS) (described in more detail below). Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes (and/or is conjugated to) one or more (e.g., two or more, three or more, or four or more) NLSs. In some cases the cationic amino acid polymer includes an NLS on the N- and/or C-terminus. In some cases the cationic amino acid polymer includes an internal NLS.

### Cationic Polypeptide Composition of a Nanoparticle

In some embodiments the cationic polypeptide composition of a nanoparticle can mediate stability, subcellular compartmentalization, and/or payload release. As one example, fragments of the N-terminus of histone proteins, referred to generally as histone tail peptides, within a subject nanoparticle core are in some case not only capable of being deprotonated by various histone modifications, such as in the case of histone acetyltransferase-mediated acetylation, but may also mediate effective nuclear-specific unpackaging of components (e.g., a payload) of a nanoparticle core. In some cases a cationic polypeptide composition includes a histone and/or histone tail peptide (e.g., a cationic polypeptide can be a histone and/or histone tail peptide). In some cases a cationic polypeptide composition includes an NLS-containing peptide (e.g., a cationic polypeptide can be an NLS-containing peptide). In some cases, a cationic polypeptide composition includes one or more NLS-containing peptides separated by cysteine residues to facilitate crosslinking. In some cases a cationic polypeptide composition includes a peptide that includes a mitochondrial localization signal (e.g., a cationic polypeptide can be a peptide that includes a mitochondrial localization signal).

### Sheddable Layer (Sheddable Coat) of a Nanoparticle

In some embodiments, a subject nanoparticle includes a sheddable layer (also referred to herein as a “transient stabilizing layer”) that surrounds (encapsulates) the core. In some cases a subject sheddable layer can protect the payload before and during initial cellular uptake. For example, without a sheddable layer, much of the payload can be lost during cellular internalization. Once in the cellular environment, a sheddable layer ‘sheds’ (e.g., the layer can be pH- and/or or glutathione-sensitive), exposing the components of the core.

In some cases a subject sheddable layer includes silica. In some cases, when a subject nanoparticle includes a sheddable layer (e.g., of silica), greater intracellular delivery efficiency can be observed despite decreased probability of cellular uptake. Without wishing to be bound by any particular theory, coating a nanoparticle core with a sheddable layer (e.g., silica coating) can seal the core, stabilizing it until shedding of the layer, which leads to release of the payload (e.g., upon processing in the intended subcellular compartment). Following cellular entry through receptor-mediated endocytosis, the nanoparticle sheds its outermost layer, the sheddable layer degrades in the acidifying environment of the endosome or reductive environment of the cytosol, and exposes the core, which in some cases exposes localization signals such as nuclear localization signals (NLSs) and/or mitochondrial localization signals. Moreover, nanoparticle cores encapsulated by a sheddable layer can be stable in serum and can be suitable for administration in vivo.

Any desired sheddable layer can be used, and one of ordinary skill in the art can take into account where in the target cell (e.g., under what conditions, such as low pH) they desire the payload to be released (e.g., endosome, cytosol, nucleus, lysosome, and the like). Different sheddable layers may be more desirable depending on when, where, and/or under what conditions it would be desirable for the sheddable coat to shed (and therefore release the payload). For example, a sheddable layer can be acid labile. In some cases the sheddable layer is an anionic sheddable layer (an anionic coat). In some cases the sheddable layer comprises silica, a peptoid, a polycysteine, and/or a ceramic (e.g., a bioceramic). In some cases the sheddable includes one or more of: calcium, manganese, magnesium, iron (e.g., the sheddable layer can be magnetic, e.g., Fe₃MnO₂), and lithium. Each of these can include phosphate or sulfate. As such, in some cases the sheddable includes one or more of: calcium phosphate, calcium sulfate, manganese phosphate, manganese sulfate, magnesium phosphate, magnesium sulfate, iron phosphate, iron sulfate, lithium phosphate, and lithium sulfate; each of which can have a particular effect on how and/or under which conditions the sheddable layer will ‘shed.’ Thus, in some cases the sheddable layer includes one or more of: silica, a peptoid, a polycysteine, a ceramic (e.g., a bioceramic), calcium, calcium phosphate, calcium sulfate, calcium oxide, hydroxyapatite, manganese, manganese phosphate, manganese sulfate, manganese oxide, magnesium, magnesium phosphate, magnesium sulfate, magnesium oxide, iron, iron phosphate, iron sulfate, iron oxide, lithium, lithium phosphate, and lithium sulfate (in any combination thereof) (e.g., the sheddable layer can be a coating of silica, peptoid, polycysteine, a ceramic (e.g., a bioceramic), calcium phosphate, calcium sulfate, manganese phosphate, manganese sulfate, magnesium phosphate, magnesium sulfate, iron phosphate, iron sulfate, lithium phosphate, lithium sulfate, or a combination thereof). In some cases the sheddable layer includes silica (e.g., the sheddable layer can be a silica coat). In some cases the sheddable layer includes an alginate gel.

In some cases different release times for different payloads are desirable. For example, in some cases it is desirable to release a payload early (e.g., within 0.5-7 days of contacting a target cell) and in some cases it is desirable to release a payload late (e.g., within 6 days-30 days of contacting a target cell). For example, in some cases it may be desirable to release a payload (e.g., a first donor DNA and a gene editing tool such as a CRISPR/Cas guide RNA, a DNA molecule encoding said CRISPR/Cas guide RNA, a CRISPR/Cas RNA-guided polypeptide, and/or a nucleic acid molecule encoding said CRISPR/Cas RNA-guided polypeptide) within 0.5-7 days of contacting a target cell (e.g., within 0.5-5 days, 0.5-3 days, 1-7 days, 1-5 days, or 1-3 days of contacting a target cell). In some cases it may be desirable to release a payload (e.g., a second Donor DNA molecule and a recombinase or a nucleic acid encoding same) within 6-40 days of contacting a target cell (e.g., within 6-30, 6-20, 6-15, 7-40, 7-30, 7-20, 7-15, 9-40, 9-30, 9-20, or 9-15 days of contacting a target cell). In some cases release times can be controlled by delivering nanoparticles having different payloads at different times. In some cases release times can be controlled by delivering nanoparticles at the same time (as part of different formulations or as part of the same formulation), where the components of the nanoparticle are designed to achieve the desired release times. For example, one may use a sheddable layer that degrades faster or slower, core components that are more or less resistant to degradation, core components that are more or less susceptible to de-condensation, etc.—and any or all of the components can be selected in any convenient combination to achieve the desired timing.

In some cases it is desirable for the payloads to be released at different times. This can be achieved in a number of different ways. For example, a nanoparticle can have more than one core, where one core is made with components that can release the payload early (e.g., within 0.5-7 days of contacting a target cell, e.g., within 0.5-5 days, 0.5-3 days, 1-7 days, 1-5 days, or 1-3 days of contacting a target cell) (e.g., a first donor DNA and/or a genome editing tool such as a ZFP or nucleic acid encoding the ZFP, a TALE or a nucleic acid encoding the TALE, a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like) and the other is made with components that can release the payload (e.g., a second Donor DNA molecule and/or a recombinase—or nucleic acid encoding same) later (e.g., within 6-40 days of contacting a target cell, e.g., within 6-30, 6-20, 6-15, 7-40, 7-30, 7-20, 7-15, 9-40, 9-30, 9-20, or 9-15 days of contacting a target cell).

As another example, a nanoparticle can include more than one sheddable layer, where the outer sheddable layer is shed (releasing a payload) prior to an inner sheddable layer being shed (releasing another payload). In some cases, the inner payload is a first donor DNA molecule and one or more gene editing tools (e.g., a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like) and the outer payload is a second donor DNA and a sequence specific recombinase (or nucleic encoding same). The inner and outer payloads can be any desired payload and either or both can include, for example, one or more siRNAs and/or one or more mRNAs. As such, in some cases a nanoparticle can have more than one sheddable layer and can be designed to release one payload early (e.g., within 0.5-7 days of contacting a target cell, e.g., within 0.5-5 days, 0.5-3 days, 1-7 days, 1-5 days, or 1-3 days of contacting a target cell) (e.g., a donor DNA and/or a genome editing tool such as a ZFP or nucleic acid encoding the ZFP, a TALE or a nucleic acid encoding the TALE, a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like), and another payload (e.g., a second Donor DNA molecule and/or a recombinase) later (e.g., within 6-40 days of contacting a target cell, e.g., within 6-30, 6-20, 6-15, 7-40, 7-30, 7-20, 7-15, 9-40, 9-30, 9-20, or 9-15 days of contacting a target cell).

In some embodiments (e.g., in embodiments described above), time of altered gene expression can be used as a proxy for the time of payload release. As an illustrative example, if one desires to determine if a payload has been released by day 12, one can assay for the desired result of nanoparticle delivery on day 12. For example, if the desired result was to express a protein of interest, e.g., by inserting a DNA sequence encoding the protein of interest, then the expression of the protein of interest can be assayed/monitored to determine if the payload has been released. As yet another example, if the desired result was to alter the genome of the target cell, e.g., via cleaving genomic DNA and/or inserting a sequence of a donor DNA molecule, the expression from the targeted locus and/or the presence of genomic alterations can be assayed/monitored to determine if the payload has been released.

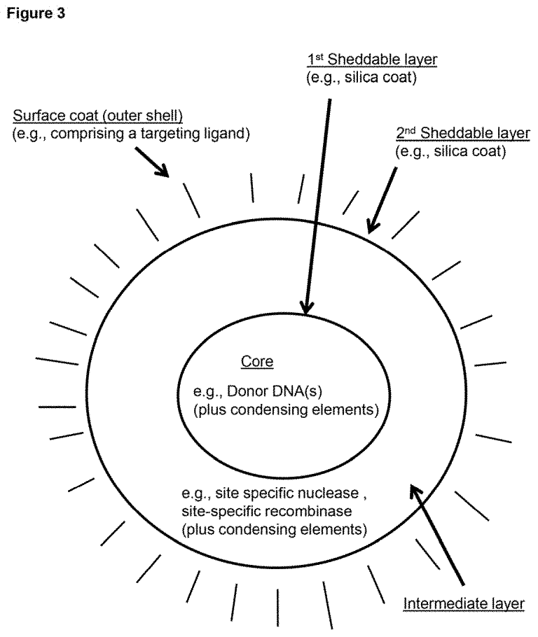

As such, in some cases a sheddable layer provides for a staged release of nanoparticle components. For example, in some cases, a nanoparticle has more than one (e.g., two, three, or four) sheddable layers. For example, for a nanoparticle with two sheddable layers, such a nanoparticle can have, from inner-most to outer-most: a core, e.g., with a first payload; a first sheddable layer, an intermediate layer e.g., with a second payload; and a second sheddable layer surrounding the intermediate layer (see, e.g., FIG. 3). Such a configuration (multiple sheddable layers) facilitates staged release of various desired payloads. As a further illustrative example, a nanoparticle with two sheddable layers (as described above) can include a donor DNA and/or one or more desired gene editing tools in the core (e.g., one or more of: a Donor DNA molecule, a CRISPR/Cas guide RNA, a DNA encoding a CRISPR/Cas guide RNA, and the like), and another desired gene editing tool in the intermediate layer (e.g., a second donor DNA and recombinase or a nucleic acid encoding same)—in any desired combination.

Alternative packaging (e.g., lipid formulations)

In some embodiments, a subject core (e.g., including any combination of components and/or configurations described above) is part of a lipid-based delivery system, e.g., a cationic lipid delivery system (see, e.g., Chesnoy and Huang, Annu Rev Biophys Biomol Struct. 2000, 29:27-47; Hirko et al., Curr Med Chem. 2003 Jul. 10(14):1185-93; and Liu et al., Curr Med Chem. 2003 Jul. 10(14):1307-15). In some cases a subject core (e.g., including any combination of components and/or configurations described above) is not surrounded by a sheddable layer. As noted above a core can include an anionic polymer composition (e.g., poly(glutamic acid)), a cationic polymer composition (e.g., poly(arginine), a cationic polypeptide composition (e.g., a histone tail peptide), and a payload (e.g., nucleic acid and/or protein payload).

In some cases in which the core is part of a lipid-based delivery system, the core is designed with timed and/or positional (e.g., environment-specific) release in mind. For example, in some cases the core includes ESPs, ENPs, and/or EPPs, and in some such cases these components are present at ratios such that payload release is delayed until a desired condition (e.g., cellular location, cellular condition such as pH, presence of a particular enzyme, and the like) is encountered by the core (e.g., described above). In some such embodiments the core includes polymers of D-isomers of an anionic amino acid and polymers of L-isomers of an anionic amino acid, and in some cases the polymers of D- and L-isomers are present, relative to one another, within a particular range of ratios (e.g., described above). In some cases the core includes polymers of D-isomers of a cationic amino acid and polymers of L-isomers of a cationic amino acid, and in some cases the polymers of D- and L-isomers are present, relative to one another, within a particular range of ratios (e.g., described above). In some cases the core includes polymers of D-isomers of an anionic amino acid and polymers of L-isomers of a cationic amino acid, and in some cases the polymers of D- and L-isomers are present, relative to one another, within a particular range of ratios (e.g., described above). In some cases the core includes polymers of L-isomers of an anionic amino acid and polymers of D-isomers of a cationic amino acid, and in some cases the polymers of D- and L-isomers are present, relative to one another, within a particular range of ratios (e.g., described elsewhere herein). In some cases the core includes a protein that includes an NLS (e.g., described elsewhere herein). In some cases the core includes an HTP (e.g., described elsewhere herein).

Cationic lipids are nonviral vectors that can be used for gene delivery and have the ability to condense plasmid DNA. After synthesis of N-[1-(2,3-dioleyloxy)propyl]-N,N,N-trimethylammonium chloride for lipofection, improving molecular structures of cationic lipids has been an active area, including head group, linker, and hydrophobic domain modifications. Modifications have included the use of multivalent polyamines, which can improve DNA binding and delivery via enhanced surface charge density, and the use of sterol-based hydrophobic groups such as 3B-[N—(N′,N′-dimethylaminoethane)-carbamoyl] cholesterol, which can limit toxicity. Helper lipids such as dioleoyl phosphatidylethanolamine (DOPE) can be used to improve transgene expression via enhanced liposomal hydrophobicity and hexagonal inverted-phase transition to facilitate endosomal escape. In some cases a lipid formulation includes one or more of: DLin-DMA, DLin-K-DMA, DLin-KC2-DMA, DLin-MC3-DMA, 98N12-5, C12-200, a cholesterol a PEG-lipid, a lipidopolyamine, dexamethasone-spermine (DS), and disubstituted spermine (D₂S) (e.g., resulting from the conjugation of dexamethasone to polyamine spermine). DLin-DMA, DLin-K-DMA, DLin-KC2-DMA, 98N12-5, C12-200 and DLin-MC3-DMA can be synthesized by methods outlined in the art (see, e.g., Heyes et. al, J. Control Release, 2005, 107, 276-287; Semple et. al, Nature Biotechnology, 2010, 28, 172-176; Akinc et. al, Nature Biotechnology, 2008, 26, 561-569; Love et. al, PNAS, 2010, 107, 1864-1869; international patent application publication WO2010054401; all of which are hereby incorporated by reference in their entirety.

Examples of various lipid-based delivery systems include, but are not limited to those described in the following publications: international patent publication No. WO2016081029; U.S. patent application publication Nos. US20160263047 and US20160237455; and U.S. Pat. Nos. 9,533,047; 9,504,747; 9,504,651; 9,486,538; 9,393,200; 9,326,940; 9,315,828; and 9,308,267; all of which are hereby incorporated by reference in their entirety.

As such, in some cases a subject core is surrounded by a lipid (e.g., a cationic lipid such as a LIPOFECTAMINE transfection reagent). In some cases a subject core is present in a lipid formulation (e.g., a lipid nanoparticle formulation). A lipid formulation can include a liposome and/or a lipoplex. A lipid formulation can include a Spontaneous Vesicle Formation by Ethanol Dilution (SNALP) liposome (e.g., one that includes cationic lipids together with neutral helper lipids which can be coated with polyethylene glycol (PEG) and/or protamine).

A lipid formulation can be a lipidoid-based formulation. The synthesis of lipidoids has been extensively described and formulations containing these compounds can be included in a subject lipid formulation (see, e.g., Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; and Siegwart et al., Proc Natl Acad Sci USA. 2011 108:12996-3001; all of which are incorporated herein by reference in their entirety). In some cases a subject lipid formulation can include one or more of (in any desired combination): 1,2-Dioleoyl-sn-glycero-3-phosphatidylcholine (DOPC); 1,2-Dioleoyl-sn-glycero-3-phosphatidylethanolamine (DOPE); N-[1-(2,3-Dioleyloxy)prophyl]N,N,N-trimethylammonium chloride (DOTMA); 1,2-Dioleoyloxy-3-trimethylammonium-propane (DOTAP); Dioctadecylamidoglycylspermine (DOGS); N-(3-Aminopropyl)-N,N-dimethyl-2,3-bis(dodecyloxy)-1 (GAP-DLRIE); propanaminium bromide; cetyltrimethylammonium bromide (CTAB); 6-Lauroxyhexyl ornithinate (LHON); 1-(2,3-Dioleoyloxypropyl)-2,4,6-trimethylpyridinium (20c); 2,3-Dioleyloxy-N-[2(sperminecarboxamido-ethyl]-N,N-dimethyl-1 (DOSPA); propanaminium trifluoroacetate; 1,2-Dioleyl-3-trimethylammonium-propane (DOPA); N-(2-Hydroxyethyl)-N,N-dimethyl-2,3-bis(tetradecyloxy)-1 (MDRIE); propanaminium bromide; dimyristooxypropyl dimethyl hydroxyethyl ammonium bromide (DMRI); 3.beta.-[N—(N′,N′-Dimethylaminoethane)-carbamoyl]cholesterol DC-Chol; bis-guanidium-tren-cholesterol (BGTC); 1,3-Diodeoxy-2-(6-carboxy-spermyl)-propylamide (DOSPER); Dimethyloctadecylammonium bromide (DDAB); Dioctadecylamidoglicylspermidin (DSL); rac-[(2,3-Dioctadecyloxypropyl)(2-hydroxyethyl)]-dimethylammonium (CLIP-1); chloride rac-[2(2,3-Dihexadecyloxypropyl (CLIP-6); oxymethyloxy)ethyl]trimethylammonium bromide; ethyldimyristoylphosphatidylcholine (EDMPC); 1,2-Distearyloxy-N,N-dimethyl-3-aminopropane (DSDMA); 1,2-Dimyristoyl-trimethylammonium propane (DMTAP); O,O′-Dimyristyl-N-lysyl aspartate (DMKE); 1,2-Distearoyl-sn-glycero-3-ethylphosphocholine (DSEPC); N-Palmitoyl D-erythro-sphingosyl carbamoyl-spermine (CCS); N-t-Butyl-NO-tetradecyl-3-tetradecylaminopropionamidine; diC14-amidine; octadecenolyoxy[ethyl-2-heptadecenyl-3 hydroxyethyl] imidazolinium (DOTIM); chloride N1-Cholesteryloxycarbonyl-3,7-diazanonane-1,9-diamine (CDAN); 2-[3-[bis(3-aminopropyl)amino]propylamino]-N-[2-[di(tetradecyl)amino]-2-oxoethyl]acetamide (RPR209120); ditetradecylcarbamoylme-ethyl-acetamide; 1,2-dilinoleyloxy-3-dimethylaminopropane (DLinDMA); 2,2-dilinoleyl-4-dimethylaminoethyl[1,3]-dioxolane; DLin-KC2-DMA; dilinoleyl-methyl-4-dimethylaminobutyrate; DLin-MC3-DMA; DLin-K-DMA; 98N12-5; C12-200; a cholesterol; a PEG-lipid; a lipiopolyamine; dexamethasone-spermine (DS); and disubstituted spermine (D25).

### Surface Coat (Outer Shell) of a Nanoparticle

In some cases, the sheddable layer (the coat), is itself coated by an additional layer, referred to herein as an “outer shell,” “outer coat,” or “surface coat.” A surface coat can serve multiple different functions. For example, a surface coat can increase delivery efficiency and/or can target a subject nanoparticle to a particular cell type. The surface coat can include a peptide, a polymer, or a ligand-polymer conjugate. The surface coat can include a targeting ligand. For example, an aqueous solution of one or more targeting ligands (with or without linker domains) can be added to a coated nanoparticle suspension (suspension of nanoparticles coated with a sheddable layer). For example, in some cases the final concentration of protonated anchoring residues (of an anchoring domain) is between 25 and 300 μM. In some cases, the process of adding the surface coat yields a monodispersed suspension of particles with a mean particle size between 50 and 150 nm and a zeta potential between 0 and −10 mV.

In some cases, the surface coat interacts electrostatically with the outermost sheddable layer. For example, in some cases, a nanoparticle has two sheddable layers (e.g., from inner-most to outer-most: a core, e.g., with a first payload; a first sheddable layer, an intermediate layer e.g., with a second payload; and a second sheddable layer surrounding the intermediate layer), and the outer shell (surface coat) can interact with (e.g., electrostatically) the second sheddable layer. In some cases, a nanoparticle has only one sheddable layer (e.g., an anionic silica layer), and the outer shell can in some cases electrostatically interact with the sheddable layer.

Thus, in cases where the sheddable layer (e.g., outermost sheddable layer) is anionic (e.g., in some cases where the sheddable layer is a silica coat), the surface coat can interact electrostatically with the sheddable layer if the surface coat includes a cationic component. For example, in some cases the surface coat includes a delivery molecule in which a targeting ligand is conjugated to a cationic anchoring domain. The cationic anchoring domain interacts electrostatically with the sheddable layer and anchors the delivery molecule to the nanoparticle. Likewise, in cases where the sheddable layer (e.g., outermost sheddable layer) is cationic, the surface coat can interact electrostatically with the sheddable layer if the surface coat includes an anionic component.

In some embodiments, the surface coat includes a cell penetrating peptide (CPP). In some cases, a polymer of a cationic amino acid can function as a CPP (also referred to as a ‘protein transduction domain’—PTD), which is a term used to refer to a polypeptide, polynucleotide, carbohydrate, or organic or inorganic compound that facilitates traversing a lipid bilayer, micelle, cell membrane, organelle membrane, or vesicle membrane. A PTD attached to another molecule (e.g., embedded in and/or interacting with a sheddable layer of a subject nanoparticle), which can range from a small polar molecule to a large macromolecule and/or a nanoparticle, facilitates the molecule traversing a membrane, for example going from extracellular space to intracellular space, or cytosol to within an organelle (e.g., the nucleus).

Examples of CPPs include but are not limited to a minimal undecapeptide protein transduction domain (corresponding to residues 47-57 of HIV-1 TAT comprising YGRKKRRQRRR (SEQ ID NO: 160); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 161); Transportan GWTLNSAGYLLGKINLKALAALAKKIL (SEQ ID NO: 162); KALAWEAKLAKALAKALAKHLAKALAKALKCEA (SEQ ID NO: 163); and RQIKIWFQNRRMKWKK (SEQ ID NO: 164). Example CPPs include but are not limited to: YGRKKRRQRRR (SEQ ID NO: 160), RKKRRQRRR (SEQ ID NO: 165), an arginine homopolymer of from 3 arginine residues to 50 arginine residues, RKKRRQRR (SEQ ID NO: 166), YARAAARQARA (SEQ ID NO: 167), THRLPRRRRRR (SEQ ID NO: 168), and GGRRARRRRRR (SEQ ID NO: 169). In some embodiments, the CPP is an activatable CPP (ACPP) (Aguilera et al. (2009) lntegr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane

In some cases a CPP can be added to the nanoparticle by contacting a coated core (a core that is surrounded by a sheddable layer) with a composition (e.g., solution) that includes the CPP. The CPP can then interact with the sheddable layer (e.g., electrostatically).

In some cases, the surface coat includes a polymer of a cationic amino acid (e.g., a poly(arginine) such as poly(L-arginine) and/or poly(D-arginine), a poly(lysine) such as poly(L-lysine) and/or poly(D-lysine), a poly(histidine) such as poly(L-histidine) and/or poly(D-histidine), a poly(ornithine) such as poly(L-ornithine) and/or poly(D-ornithine), poly(citrulline) such as poly(L-citrulline) and/or poly(D-citrulline), and the like). As such, in some cases the surface coat includes poly(arginine), e.g., poly(L-arginine).

In some embodiments, the surface coat includes a heptapeptide such as selank (TKPRPGP—SEQ ID NO: 147) (e.g., N-acetyl selank) and/or semax (MEHFPGP—SEQ ID NO: 148) (e.g., N-acetyl semax). As such, in some cases the surface coat includes selank (e.g., N-acetyl selank). In some cases the surface coat includes semax (e.g., N-acetyl semax).

In some embodiments the surface coat includes a delivery molecule. A delivery molecule includes a targeting ligand and in some cases the targeting ligand is conjugated to an anchoring domain (e.g. a cationic anchoring domain or anionic anchoring domain). In some cases a targeting ligand is conjugated to an anchoring domain (e.g. a cationic anchoring domain or anionic anchoring domain) via an intervening linker.

### Multivalent Surface Coat

In some cases the surface coat includes any one or more of (in any desired combination): (i) one or more of the above described polymers, (ii) one or more targeting ligands, one or more CPPs, and one or more heptapeptides. For example, in some cases a surface coat can include one or more (e.g., two or more, three or more) targeting ligands, but can also include one or more of the above described cationic polymers. In some cases a surface coat can include one or more (e.g., two or more, three or more) targeting ligands, but can also include one or more CPPs. Further, a surface coat may include any combination of glycopeptides to promote stealth functionality, that is, to prevent serum protein adsorption and complement activity. This may be accomplished through Azide-alkyne click chemistry, coupling a peptide containing propargyl modified residues to azide containing derivatives of sialic acid, neuraminic acid, and the like.

In some cases, a surface coat includes a combination of targeting ligands that provides for targeted binding to CD34 and heparin sulfate proteoglycans. For example, poly(L-arginine) can be used as part of a surface coat to provide for targeted binding to heparin sulfate proteoglycans. As such, in some cases, after surface coating a nanoparticle with a cationic polymer (e.g., poly(L-arginine)), the coated nanoparticle is incubated with hyaluronic acid, thereby forming a zwitterionic and multivalent surface.

In some embodiments, the surface coat is multivalent. A multivalent surface coat is one that includes two or more targeting ligands (e.g., two or more delivery molecules that include different ligands). An example of a multimeric (in this case trimeric) surface coat (outer shell) is one that includes the targeting ligands stem cell factor (SCF) (which targets c-Kit receptor, also known as CD117), CD70 (which targets CD27), and SH2 domain-containing protein 1A (SH2D1A) (which targets CD150). For example, in some cases, to target hematopoietic stem cells (HSCs) [KLS (c-Kit⁺ Lin⁻ Sca-1⁺) and CD27⁺/IL-7Ra⁻/CD150⁺/CD34⁻], a subject nanoparticle includes a surface coat that includes a combination of the targeting ligands SCF, CD70, and SH2 domain-containing protein 1A (SH2D1A), which target c-Kit, CD27, and CD150, respectively (see, e.g., Table 1). In some cases, such a surface coat can selectively target HSPCs and long-term HSCs (c-Kit+/Lin−/Sca-1+/CD27+/IL-7Ra−/CD150+/CD34−) over other lymphoid and myeloid progenitors.

In some example embodiments, all three targeting ligands (SCF, CD70, and SH2D1A) are anchored to the nanoparticle via fusion to a cationic anchoring domain (e.g., a poly-histidine such as 6H, a poly-arginine such as 9R, and the like). For example, (1) the targeting polypeptide SCF (which targets c-Kit receptor) can include XMEGICRNRVTNNVKDVTKLVANLPKDYMITLKYVPGMDVLPSHCWISEMVVQLSDSLTDLLDKFS NISEGLSNYSIIDKLVNIVDDLVECVKENSSKDLKKSFKSPEPRLFTPEEFFRIFNRSIDAFKDFVVAS ETSDCVVSSTLSPEKDSRVSVTKPFMLPPVAX (SEQ ID NO: 194), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence; (2) the targeting polypeptide CD70 (which targets CD27) can include XPEEGSGCSVRRRPYGCVLRAALVPLVAGLVICLVVCIQRFAQAQQQLPLESLGWDVAELQLNHT GPQQDPRLYWQGGPALGRSFLHGPELDKGQLRIHRDGIYMVHIQVTLAICSSTTASRHHPTTLAV GICSPASRSISLLRLSFHQGCTIASQRLTPLARGDTLCTNLTGTLLPSRNTDETFFGVQVWVRPX (SEQ ID NO: 195), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence; and (3) the targeting polypeptide SH2D1A (which targets CD150) can include XSSGLVPRGSHMDAVAVYHGKISRETGEKLLLATGLDGSYLLRDSESVPGVYCLCVLYHGYIYTYR VSQTETGSWSAETAPGVHKRYFRKIKNLISAFQKPDQGIVIPLQYPVEKKSSARSTQGTTGIREDP DVCLKAP (SEQ ID NO: 196), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence (e.g., such as MGSSXSSGLVPRGSHMDAVAVYHGKISRETGEKLLLATGLDGSYLLRDSESVPGVYCLCVLYHGY IYTYRVSQTETGSWSAETAPGVHKRYFRKIKNLISAFQKPDQGIVIPLQYPVEKKSSARSTQGTTGI REDPDVCLKAP (SEQ ID NO: 197)).

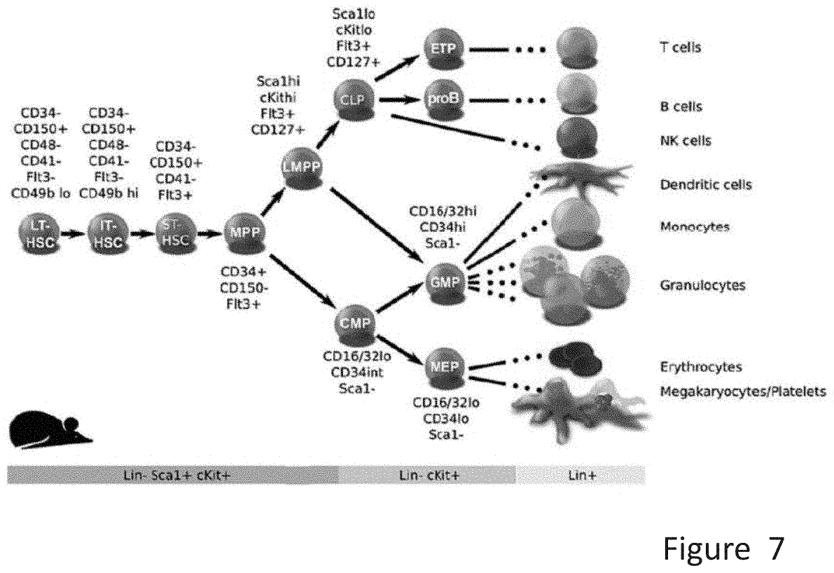

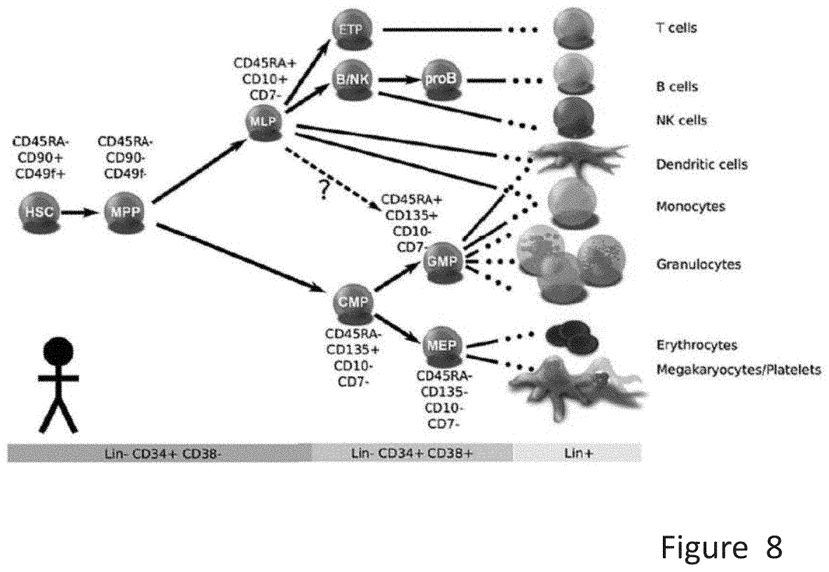

As noted above, nanoparticles of the disclosure can include multiple targeting ligands (as part of a surface coat) in order to target a desired cell type, or in order to target a desired combination of cell types. Examples of cells of interest within the mouse and human hematopoietic cell lineages are depicted in FIGS. 7-8, along with markers that have been identified for those cells. For example, various combinations of cell surface markers of interest include, but are not limited to: [Mouse] (i) CD150; (ii) Sca1, cKit, CD150; (iii) CD150 and CD49b; (iv) Sca1, cKit, CD150, and CD49b; (v) CD150 and Flt3; (vi) Sca1, cKit, CD150, and Flt3; (vii) Flt3 and CD34; (viii) Flt3, CD34, Sca1, and cKit; (ix) Flt3 and CD127; (x) Sca1, cKit, Flt3, and CD127; (xi) CD34; (xii) cKit and CD34; (xiii) CD16/32 and CD34; (xiv) cKit, CD16/32, and CD34; and (xv) cKit; and [Human] (i) CD90 and CD49f; (ii) CD34, CD90, and CD49f; (iii) CD34; (iv) CD45RA and CD10; (v) CD34, CD45RA, and CD10; (vi) CD45RA and CD135; (vii) CD34, CD38, CD45RA, and CD135; (viii) CD135; (ix) CD34, CD38, and CD135; and (x) CD34 and CD38. Thus, in some cases a surface coat includes one or more targeting ligands that provide targeted binding to a surface protein or combination of surface proteins selected from: [Mouse] (i) CD150; (ii) Sca1, cKit, CD150; (iii) CD150 and CD49b; (iv) Sca1, cKit, CD150, and CD49b; (v) CD150 and Flt3; (vi) Sca1, cKit, CD150, and Flt3; (vii) Flt3 and CD34; (viii) Flt3, CD34, Sca1, and cKit; (ix) Flt3 and CD127; (x) Sca1, cKit, Flt3, and CD127; (xi) CD34; (xii) cKit and CD34; (xiii) CD16/32 and CD34; (xiv) cKit, CD16/32, and CD34; and (xv) cKit; and [Human] (i) CD90 and CD49f; (ii) CD34, CD90, and CD49f; (iii) CD34; (iv) CD45RA and CD10; (v) CD34, CD45RA, and CD10; (vi) CD45RA and CD135; (vii) CD34, CD38, CD45RA, and CD135; (viii) CD135; (ix) CD34, CD38, and CD135; and (x) CD34 and CD38. Because a subject nanoparticle can include more than one targeting ligand, and because some cells include overlapping markers, multiple different cell types can be targeted using combinations of surface coats, e.g., in some cases a surface coat may target one specific cell type while in other cases a surface coat may target more than one specific cell type (e.g., 2 or more, 3 or more, 4 or more cell types). For example, any combination of cells within the hematopoietic lineage can be targeted. As an illustrative example, targeting CD34 (using a targeting ligand that provides for targeted binding to CD34) can lead to nanoparticle delivery of a payload to several different cells within the hematopoietic lineage (see, e.g., FIGS. 7-8).

### Delivery Molecules

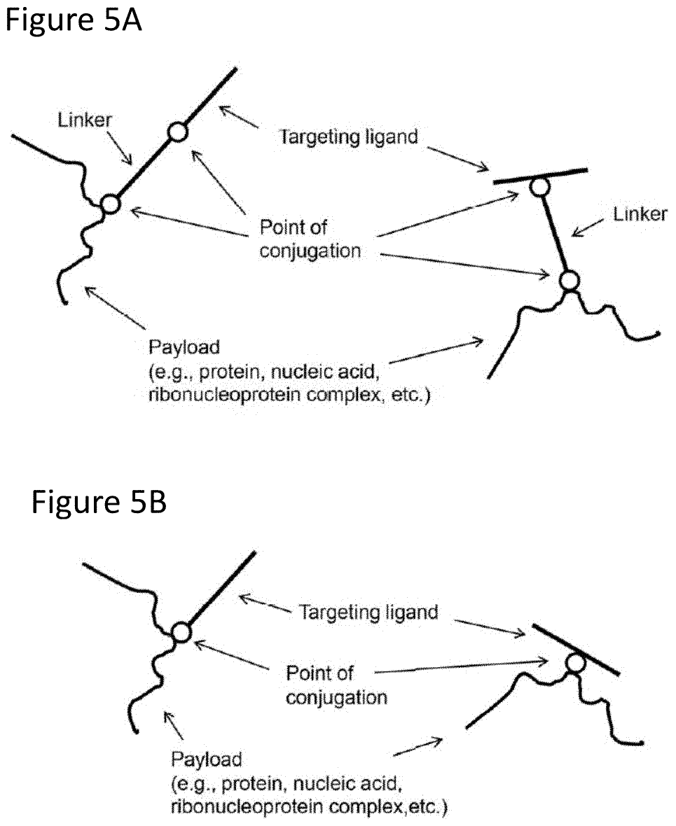

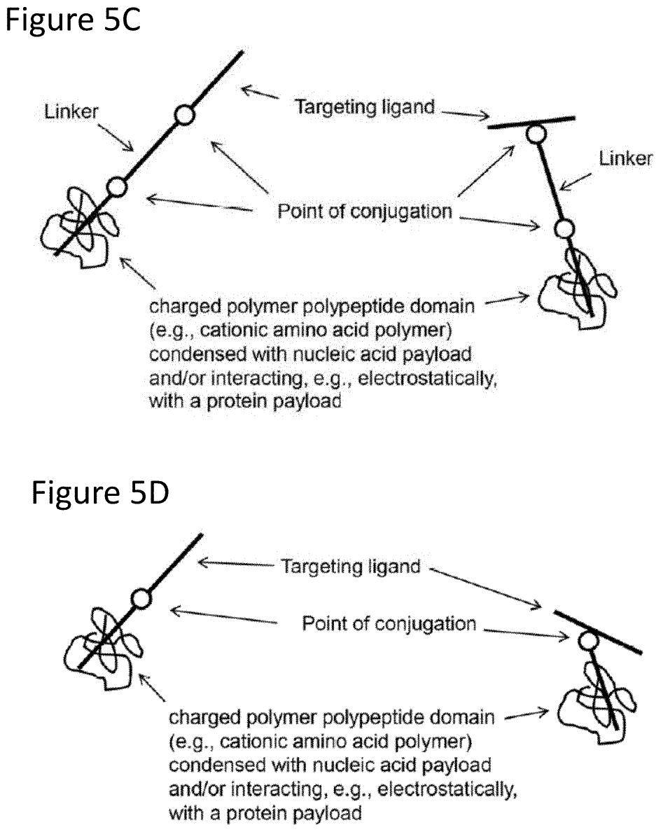

Provided are delivery molecules that include a targeting ligand (a peptide) conjugated to (i) a protein or nucleic acid payload, or (ii) a charged polymer polypeptide domain. The targeting ligand provides for (i) targeted binding to a cell surface protein, and in some cases (ii) engagement of a long endosomal recycling pathway. In some cases when the targeting ligand is conjugated to a charged polymer polypeptide domain, the charged polymer polypeptide domain interacts with (e.g., is condensed with) a nucleic acid payload and/or a protein payload. In some cases the targeting ligand is conjugated via an intervening linker. Refer to FIGS. 5A-D for examples of different possible conjugation strategies (i.e., different possible arrangements of the components of a subject delivery molecule). In some cases, the targeting ligand provides for targeted binding to a cell surface protein, but does not necessarily provide for engagement of a long endosomal recycling pathway. Thus, also provided are delivery molecules that include a targeting ligand (e.g., peptide targeting ligand) conjugated to a protein or nucleic acid payload, or conjugated to a charged polymer polypeptide domain, where the targeting ligand provides for targeted binding to a cell surface protein (but does not necessarily provide for engagement of a long endosomal recycling pathway).

In some cases, the delivery molecules disclosed herein are designed such that a nucleic acid or protein payload reaches its extracellular target (e.g., by providing targeted biding to a cell surface protein) and is preferentially not destroyed within lysosomes or sequestered into ‘short’ endosomal recycling endosomes. Instead, delivery molecules of the disclosure can provide for engagement of the ‘long’ (indirect/slow) endosomal recycling pathway, which can allow for endosomal escape and/or or endosomal fusion with an organelle.

For example, in some cases, β-arrestin is engaged to mediate cleavage of seven-transmembrane GPCRs (McGovern et al., Handb Exp Pharmacol. 2014; 219:341-59; Goodman et al., Nature. 1996 Oct. 3; 383(6599):447-50; Zhang et al., J Biol Chem. 1997 Oct. 24; 272(43):27005-14) and/or single-transmembrane receptor tyrosine kinases (RTKs) from the actin cytoskeleton (e.g., during endocytosis), triggering the desired endosomal sorting pathway. Thus, in some embodiments the targeting ligand of a delivery molecule of the disclosure provides for engagement of 8-arrestin upon binding to the cell surface protein (e.g., to provide for signaling bias and to promote internalization via endocytosis following orthosteric binding).

### Charged Polymer Polypeptide Domain

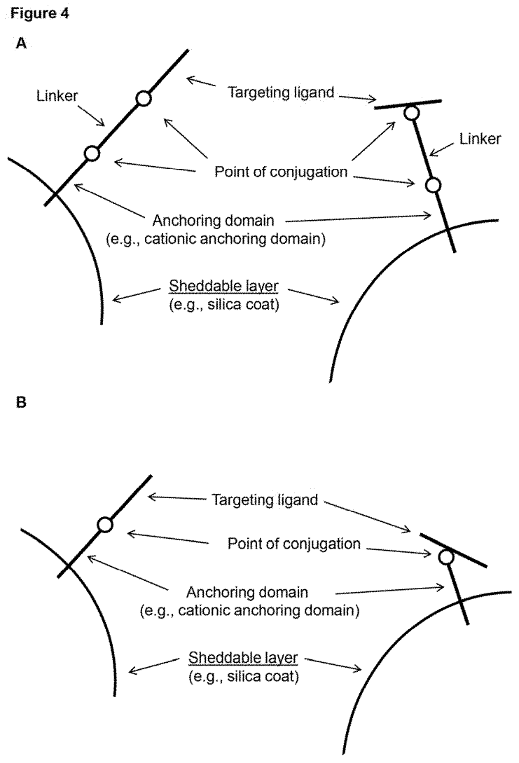

In some case a targeting ligand (e.g., of a subject delivery molecule) is conjugated to a charged polymer polypeptide domain (an anchoring domain such as a cationic anchoring domain or an anionic anchoring domain) (see e.g., FIG. 4 and FIGS. 5A-D). Charged polymer polypeptide domains can include repeating residues (e.g., cationic residues such as arginine, lysine, histidine). In some cases, a charged polymer polypeptide domain (an anchoring domain) has a length in a range of from 3 to 30 amino acids (e.g., from 3-28, 3-25, 3-24, 3-20, 4-30, 4-28, 4-25, 4-24, or 4-20 amino acids; or e.g., from 4-15, 4-12, 5-30, 5-28, 5-25, 5-20, 5-15, 5-12 amino acids). In some cases, a charged polymer polypeptide domain (an anchoring domain) has a length in a range of from 4 to 24 amino acids. In some cases, a charged polymer polypeptide domain (an anchoring domain) has a length in a range of from 5 to 10 amino acids. Suitable examples of a charged polymer polypeptide domain include, but are not limited to: RRRRRRRRR (9R)(SEQ ID NO: 15) and HHHHHH (6H)(SEQ ID NO: 16).

A charged polymer polypeptide domain (a cationic anchoring domain, an anionic anchoring domain) can be any convenient charged domain (e.g., cationic charged domain). For example, such a domain can be a histone tail peptide (HTP) (described elsewhere herein in more detail). In some cases a charged polymer polypeptide domain includes a histone and/or histone tail peptide (e.g., a cationic polypeptide can be a histone and/or histone tail peptide). In some cases a charged polymer polypeptide domain includes an NLS-containing peptide (e.g., a cationic polypeptide can be an NLS-containing peptide). In some cases a charged polymer polypeptide domain includes a peptide that includes a mitochondrial localization signal (e.g., a cationic polypeptide can be a peptide that includes a mitochondrial localization signal).

In some cases, a charged polymer polypeptide domain of a subject delivery molecule is used as a way for the delivery molecular to interact with (e.g., interact electrostatically, e.g., for condensation) the payload (e.g., nucleic acid payload and/or protein payload).

In some cases, a charged polymer polypeptide domain of a subject delivery molecule is used as an anchor to coat the surface of a nanoparticle with the delivery molecule, e.g., so that the targeting ligand is used to target the nanoparticle to a desired cell/cell surface protein (see e.g., FIG. 4). Thus, in some cases, the charged polymer polypeptide domain interacts electrostatically with a charged stabilization layer of a nanoparticle. For example, in some cases a nanoparticle includes a core (e.g., including a nucleic acid, protein, and/or ribonucleoprotein complex payload) that is surrounded by a stabilization layer (e.g., a silica, peptoid, polycysteine, or calcium phosphate coating). In some cases, the stabilization layer has a negative charge and a positively charged polymer polypeptide domain can therefore interact with the stabilization layer, effectively anchoring the delivery molecule to the nanoparticle and coating the nanoparticle surface with a subject targeting ligand (see, e.g., FIG. 4). In some cases, the stabilization layer has a positive charge and a negatively charged polymer polypeptide domain can therefore interact with the stabilization layer, effectively anchoring the delivery molecule to the nanoparticle and coating the nanoparticle surface with a subject targeting ligand. Conjugation can be accomplished by any convenient technique and many different conjugation chemistries will be known to one of ordinary skill in the art. In some cases the conjugation is via sulfhydryl chemistry (e.g., a disulfide bond). In some cases the conjugation is accomplished using amine-reactive chemistry. In some cases, the targeting ligand and the charged polymer polypeptide domain are conjugated by virtue of being part of the same polypeptide.

In some cases a charged polymer polypeptide domain (cationic) can include a polymer selected from: poly(arginine)(PR), poly(lysine)(PK), poly(histidine)(PH), poly(ornithine), poly(citrulline), and a combination thereof. In some cases a given cationic amino acid polymer can include a mix of arginine, lysine, histidine, ornithine, and citrulline residues (in any convenient combination). Polymers can be present as a polymer of L-isomers or D-isomers, where D-isomers are more stable in a target cell because they take longer to degrade. Thus, inclusion of D-isomer poly(amino acids) delays degradation (and subsequent payload release). The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the release kinetics and enzymatic susceptibility to degradation and payload release.

In some cases a cationic polymer includes D-isomers and L-isomers of an cationic amino acid polymer (e.g., poly(arginine)(PR), poly(lysine)(PK), poly(histidine)(PH), poly(ornithine), poly(citrulline)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases a cationic polymer includes a first cationic polymer (e.g., amino acid polymer) that is a polymer of D-isomers (e.g., selected from poly(D-arginine), poly(D-lysine), poly(D-histidine), poly(D-ornithine), and poly(D-citrulline)); and includes a second cationic polymer (e.g., amino acid polymer) that is a polymer of L-isomers (e.g., selected from poly(L-arginine), poly(L-lysine), poly(L-histidine), poly(L-ornithine), and poly(L-citrulline)). In some cases the ratio of the first cationic polymer (D-isomers) to the second cationic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, a cationic polymer includes (e.g., in addition to or in place of any of the foregoing examples of cationic polymers) poly(ethylenimine), poly(amidoamine) (PAMAM), poly(aspartamide), polypeptoids (e.g., for forming “spiderweb”-like branches for core condensation), a charge-functionalized polyester, a cationic polysaccharide, an acetylated amino sugar, chitosan, or a cationic polymer that includes any combination thereof (e.g., in linear or branched forms).

In some embodiments, an cationic polymer can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases a cationic polymer includes poly(L-arginine), e.g., with a molecular weight of approximately 29 kDa. As another example, in some cases a cationic polymer includes linear poly(ethylenimine) with a molecular weight of approximately 25 kDa (PEI). As another example, in some cases a cationic polymer includes branched poly(ethylenimine) with a molecular weight of approximately 10 kDa. As another example, in some cases a cationic polymer includes branched poly(ethylenimine) with a molecular weight of approximately 70 kDa. In some cases a cationic polymer includes PAMAM.

In some cases, a cationic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes a cysteine residue. In some cases the cationic amino acid polymer includes cysteine residue on the N- and/or C-terminus. In some cases the cationic amino acid polymer includes an internal cysteine residue.

In some cases, a cationic amino acid polymer includes (and/or is conjugated to) a nuclear localization signal (NLS) (described in more detail below). Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) includes one or more (e.g., two or more, three or more, or four or more) NLSs. In some cases the cationic amino acid polymer includes an NLS on the N- and/or C-terminus. In some cases the cationic amino acid polymer includes an internal NLS.

In some cases, the charged polymer polypeptide domain is condensed with a nucleic acid payload and/or a protein payload (see e.g., FIGS. 5A-D). In some cases, the charged polymer polypeptide domain interacts electrostatically with a protein payload. In some cases, the charged polymer polypeptide domain is co-condensed with silica, salts, and/or anionic polymers to provide added endosomal buffering capacity, stability, and, e.g., optional timed release. In some cases, a charged polymer polypeptide domain of a subject delivery molecule is a stretch of repeating cationic residues (such as arginine, lysine, and/or histidine), e.g., in some 4-25 amino acids in length or 4-15 amino acids in length. Such a domain can allow the delivery molecule to interact electrostatically with an anionic sheddable matrix (e.g., a co-condensed anionic polymer). Thus, in some cases, a subject charged polymer polypeptide domain of a subject delivery molecule is a stretch of repeating cationic residues that interacts (e.g., electrostatically) with an anionic sheddable matrix and with a nucleic acid and/or protein payload. Thus, in some cases a subject delivery molecule interacts with a payload (e.g., nucleic acid and/or protein) and is present as part of a composition with an anionic polymer (e.g., co-condenses with the payload and with an anionic polymer).

The anionic polymer of an anionic sheddable matrix (i.e., the anionic polymer that interacts with the charged polymer polypeptide domain of a subject delivery molecule) can be any convenient anionic polymer/polymer composition. Examples include, but are not limited to: poly(glutamic acid) (e.g., poly(D-glutamic acid) (PDE), poly(L-glutamic acid) (PLE), both PDE and PLE in various desired ratios, etc.) In some cases, PDE is used as an anionic sheddable matrix. In some cases, PLE is used as an anionic sheddable matrix (anionic polymer). In some cases, PDE is used as an anionic sheddable matrix (anionic polymer). In some cases, PLE and PDE are both used as an anionic sheddable matrix (anionic polymer), e.g., in a 1:1 ratio (50% PDE, 50% PLE).

Anionic Polymer

An anionic polymer can include one or more anionic amino acid polymers. For example, in some cases a subject anionic polymer composition includes a polymer selected from: poly(glutamic acid)(PEA), poly(aspartic acid)(PDA), and a combination thereof. In some cases a given anionic amino acid polymer can include a mix of aspartic and glutamic acid residues. Each polymer can be present in the composition as a polymer of L-isomers or D-isomers, where D-isomers are more stable in a target cell because they take longer to degrade. Thus, inclusion of D-isomer poly(amino acids) can delay degradation and subsequent payload release. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases an anionic polymer composition includes polymers of D-isomers and polymers of L-isomers of an anionic amino acid polymer (e.g., poly(glutamic acid)(PEA) and poly(aspartic acid)(PDA)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases an anionic polymer composition includes a first anionic polymer (e.g., amino acid polymer) that is a polymer of D-isomers (e.g., selected from poly(D-glutamic acid) (PDEA) and poly(D-aspartic acid) (PDDA)); and includes a second anionic polymer (e.g., amino acid polymer) that is a polymer of L-isomers (e.g., selected from poly(L-glutamic acid) (PLEA) and poly(L-aspartic acid) (PLDA)). In some cases the ratio of the first anionic polymer (D-isomers) to the second anionic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, an anionic polymer composition includes (e.g., in addition to or in place of any of the foregoing examples of anionic polymers) a glycosaminoglycan, a glycoprotein, a polysaccharide, poly(mannuronic acid), poly(guluronic acid), heparin, heparin sulfate, chondroitin, chondroitin sulfate, keratan, keratan sulfate, aggrecan, poly(glucosamine), or an anionic polymer that comprises any combination thereof.

In some embodiments, an anionic polymer can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases an anionic polymer includes poly(glutamic acid) with a molecular weight of approximately 15 kDa.

In some cases, an anionic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes a cysteine residue. In some cases the anionic amino acid polymer includes cysteine residue on the N- and/or C-terminus. In some cases the anionic amino acid polymer includes an internal cysteine residue.

In some cases, an anionic amino acid polymer includes (and/or is conjugated to) a nuclear localization signal (NLS) (described in more detail below). Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

In some cases, an anionic polymer is conjugated to a targeting ligand.

### Linker

In some embodiments a targeting ligand is conjugated to an anchoring domain (e.g., a cationic anchoring domain, an anionic anchoring domain) or to a payload via an intervening linker. The linker can be a protein linker or non-protein linker. A linker can in some cases aid in stability, prevent complement activation, and/or provide flexibility to the ligand relative to the anchoring domain.

Conjugation of a targeting ligand to a linker or a linker to an anchoring domain can be accomplished in a number of different ways. In some cases the conjugation is via sulfhydryl chemistry (e.g., a disulfide bond, e.g., between two cysteine residues). In some cases the conjugation is accomplished using amine-reactive chemistry. In some cases, a targeting ligand includes a cysteine residue and is conjugated to the linker via the cysteine residue; and/or an anchoring domain includes a cysteine residue and is conjugated to the linker via the cysteine residue. In some cases, the linker is a peptide linker and includes a cysteine residue. In some cases, the targeting ligand and a peptide linker are conjugated by virtue of being part of the same polypeptide; and/or the anchoring domain and a peptide linker are conjugated by virtue of being part of the same polypeptide.

In some cases, a subject linker is a polypeptide and can be referred to as a polypeptide linker. It is to be understood that while polypeptide linkers are contemplated, non-polypeptide linkers (chemical linkers) are used in some cases. For example, in some embodiments the linker is a polyethylene glycol (PEG) linker. Suitable protein linkers include polypeptides of between 4 amino acids and 60 amino acids in length (e.g., 4-50, 4-40, 4-30, 4-25, 4-20, 4-15, 4-10, 6-60, 6-50, 6-40, 6-30, 6-25, 6-20, 6-15, 6-10, 8-60, 8-50, 8-40, 8-30, 8-25, 8-20, or 8-15 amino acids in length).

In some embodiments, a subject linker is rigid (e.g., a linker that includes one or more proline residues). One non-limiting example of a rigid linker is GAPGAPGAP (SEQ ID NO: 17). In some cases, a polypeptide linker includes a C residue at the N- or C-terminal end. Thus, in some case a rigid linker is selected from: GAPGAPGAPC (SEQ ID NO: 18) and CGAPGAPGAP (SEQ ID NO: 19).

Peptide linkers with a degree of flexibility can be used. Thus, in some cases, a subject linker is flexible. The linking peptides may have virtually any amino acid sequence, bearing in mind that flexible linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)ₙ, glycine-serine polymers (including, for example, (GS)ₙ, GSGGSₙ (SEQ ID NO: 20), GGSGGSₙ (SEQ ID NO: 21), and GGGSₙ (SEQ ID NO: 22), where n is an integer of at least one), glycine-alanine polymers, alanine-serine polymers. Example linkers can comprise amino acid sequences including, but not limited to, GGSG (SEQ ID NO: 23), GGSGG (SEQ ID NO: 24), GSGSG (SEQ ID NO: 25), GSGGG (SEQ ID NO: 26), GGGSG (SEQ ID NO: 27), GSSSG (SEQ ID NO: 28), and the like. The ordinarily skilled artisan will recognize that design of a peptide conjugated to any elements described above can include linkers that are all or partially flexible, such that the linker can include a flexible linker as well as one or more portions that confer less flexible structure. Additional examples of flexible linkers include, but are not limited to: GGGGGSGGGGG (SEQ ID NO: 29) and GGGGGSGGGGS (SEQ ID NO: 30). As noted above, in some cases, a polypeptide linker includes a C residue at the N- or C-terminal end. Thus, in some cases a flexible linker includes an amino acid sequence selected from: GGGGGSGGGGGC (SEQ ID NO: 31), CGGGGGSGGGGG (SEQ ID NO: 32), GGGGGSGGGGSC (SEQ ID NO: 33), and CGGGGGSGGGGS (SEQ ID NO: 34).

In some cases, a subject polypeptide linker is endosomolytic. Endosomolytic polypeptide linkers include but are not limited to: KALA (SEQ ID NO: 35) and GALA (SEQ ID NO: 36). As noted above, in some cases, a polypeptide linker includes a C residue at the N- or C-terminal end. Thus, in some cases a subject linker includes an amino acid sequence selected from: CKALA (SEQ ID NO: 37), KALAC (SEQ ID NO: 38), CGALA (SEQ ID NO: 39), and GALAC (SEQ ID NO: 40).

### Illustrative Examples of Sulfhydryl Coupling Reactions

(e.g., for conjugation via sulfhydryl chemistry, e.g., using a cysteine residue)
(e.g., for conjugating a targeting ligand or glycopeptide to a linker, conjugating a targeting ligand or glycopeptide to an anchoring domain (e.g., cationic anchoring domain), conjugating a linker to an anchoring domain (e.g., cationic anchoring domain), and the like)

Disulfide Bond

Cysteine residues in the reduced state, containing free sulfhydryl groups, readily form disulfide bonds with protected thiols in a typical disulfide exchange reaction.

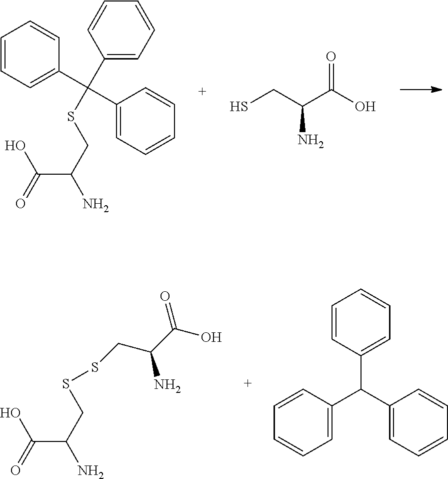

Thioether/Thioester Bond

Sulfhydryl groups of cysteine react with maleimide and acyl halide groups, forming stable thioether and thioester bonds respectively.

Maleimide

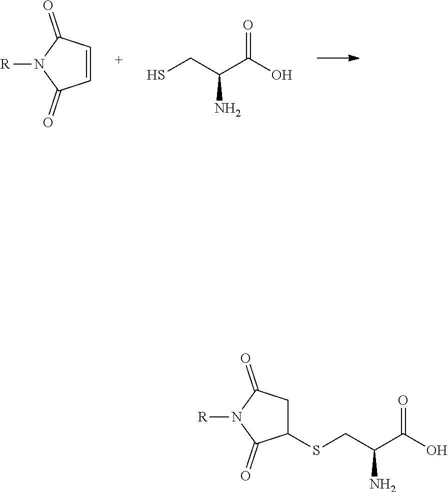

Acyl Halide

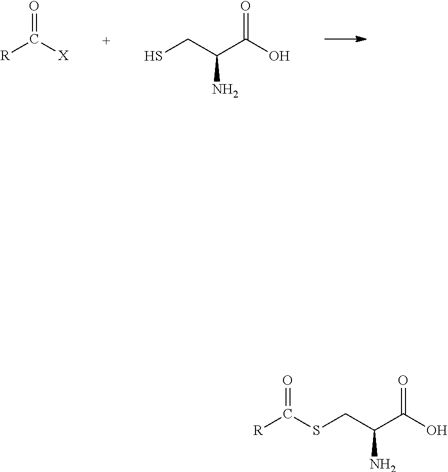

Azide-alkyne Cycloaddition

This conjugation is facilitated by chemical modification of the cysteine residue to contain an alkyne bond, or by the use of an L-propargyl amino acid derivative (e.g., L-propargyl cysteine—pictured below) in synthetic peptide preparation (e.g., solid phase synthesis). Coupling is then achieved by means of Cu promoted click chemistry.

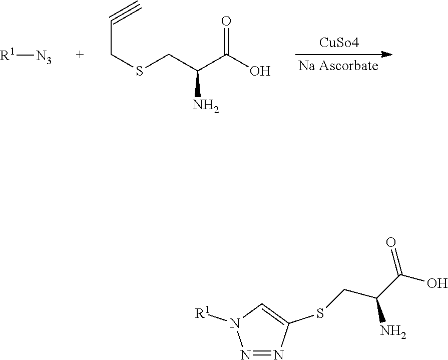

### Examples of Targeting Ligands

Examples of targeting ligands include, but are not limited to, those that include the following amino acid sequences:

SCF (targets/binds to c-Kit receptor)
(SEQ ID NO: 184)
EGICRNRVTNNVKDVTKLVANLPKDYMITLKYVPGMDVLPSHCWISE
MVVQLSDSLTDLLDKFSNISEGLSNYSIIDKLVNIVDDLVECVKENS
SKDLKKSFKSPEPRLFTPEEFFRIFNRSIDAFKDFVVASETSDCVVS
STLSPEKDSRVSVTKPFMLPPVA;
CD70 (targets/binds to CD27)
(SEQ ID NO: 185)
PEEGSGCSVRRRPYGCVLRAALVPLVAGLVICLVVCIQRFAQAQQQL
PLESLGWDVAELQLNHTGPQQDPRLYWQGGPALGRSFLHGPELDKGQ
LRIHRDGIYMVHIQVTLAICSSTTASRHHPTTLAVGICSPASRSISL
LRLSFHQGCTIASQRLTPLARGDTLCTNLTGTLLPSRNTDETFFGVQ
WVRP;
and
SH2 domain-containing protein 1A (SH2D1A)
(targets/binds to CD150)
(SEQ ID NO: 186)
SSGLVPRGSHMDAVAVYHGKISRETGEKLLLATGLDGSYLLRDSESV
PGVYCLCVLYHGYIYTYRVSQTETGSWSAETAPGVHKRYFRKIKNLI
SAFQKPDQGIVIPLQYPVEKKSSARSTQGTTGIREDPDVCLKAP

Thus, non-limiting examples of targeting ligands (which can be used alone or in combination with other targeting ligands) include:

9R-SCF
(SEQ ID NO: 189)
RRRRRRRRRMEGICRNRVTNNVKDVTKLVANLPKDYMITLKYVPGMDVLPS
HCWISEMVVQLSDSLTDLLDKFSNISEGLSNYSIIDKLVNIVDDLVECVKE
NSSKDLKKSFKSPEPRLFTPEEFFRIFNRSIDAFKDFVVASETSDCVVSST
LSPEKDSRVSVTKPFMLPPVA
9R-CD70
(SEQ ID NO: 190)
RRRRRRRRRPEEGSGCSVRRRPYGCVLRAALVPLVAGLVICLVVCIQRFAQ
AQQQLPLESLGWDVAELQLNHTGPQQDPRLYWQGGPALGRSFLHGPELDKG
QLRIHRDGIYMVHIQVTLAICSSTTASRHHPTTLAVGICSPASRSISLLRL
SFHQGCTIASQRLTPLARGDTLCTNLTGTLLPSRNTDETFFGVQVVVRP
CD70-9R
(SEQ ID NO: 191)
PEEGSGCSVRRRPYGCVLRAALVPLVAGLVICLVVCIQRFAQAQQQLPLES
LGWDVAELQLNHTGPQQDPRLYWQGGPALGRSFLHGPELDKGQLRIHRDGI
YMVHIQVTLAICSSTTASRHHPTTLAVGICSPASRSISLLRLSFHQGCTIA
SQRLTPLARGDTLCTNLTGTLLPSRNTDETFFGVQVVVRPRRRRRRRRR
6H-SH2D1A
(SEQ ID NO: 192)
MGSSHHHHHHSSGLVPRGSHMDAVAVYHGKISRETGEKLLLATGLDGSYLL
RDSESVPGVYCLCVLYHGYIYTYRVSQTETGSWSAETAPGVHKRYFRKIKN
LISAFQKPDQGIVIPLQYPVEKKSSARSTQGTTGIREDPDVCLKAP
6H-SH2D1A
(SEQ ID NO: 193)
RRRRRRRRRSSGLVPRGSHMDAVAVYHGKISRETGEKLLLATGLDGSYLLR
DSESVPGVYCLCVLYHGYIYTYRVSQTETGSWSAETAPGVHKRYFRKIKNL
ISAFQKPDQGIVIPLQYPVEKKSSARSTQGTTGIREDPDVCLKAP
Illustrative examples of delivery molecules and
components (0a) Cysteine conjugation anchor 1
(CCA1)[anchoring domain (e.g., cationic anchoring
domain)-linker (GAPGAPGAP)-cysteine]
(SEQ ID NO: 41)
RRRRRRRRR GAPGAPGAP C
(0b) Cysteine conjugation anchor 2 (CCA2)
[cysteine-linker (GAPGAPGAP)-anchoring domain
(e.g., cationic anchoring domain)
(SEQ ID NO: 42)
C GAPGAPGAP RRRRRRRRR
(1a) α5β1 ligand
[anchoring domain (e.g., cationic anchoring
domain)-linker (GAPGAPGAP)-Targeting ligand]
(SEQ ID NO: 45)
RRRRRRRRR GAPGAPGAP RRETAWA
(1b) α5β1 ligand
[Targeting ligand-linker (GAPGAPGAP)-anchoring
domain (e.g., cationic anchoring domain)]
(SEQ ID NO: 46)
RRETAWA GAPGAPGAP RRRRRRRRR
(1c) α5β1 ligand-Cys left
(SEQ ID NO: 19)
CGAPGAPGAP
Note: This can be conjugated to CCA1 (see above)
either via sulfhydryl chemistry (e.g., a disulfide
bond) or amine-reactive chemistry.
(1d) α5β1 ligand-Cys right
(SEQ ID NO: 18)
GAPGAPGAPC
Note: This can be conjugated to CCA2 (see above)
either via sulfhydryl chemistry (e.g., a disulfide
bond) or amine-reactive chemistry.
(2a) RGD α5β1 ligand
[anchoring domain (e.g., cationic anchoring
domain)-linker (GAPGAPGAP)-Targeting ligand]
(SEQ ID NO: 47)
RRRRRRRRR GAPGAPGAP RGD
(2b) RGD a5b1 ligand
[Target ligand-linker (GAPGAPGAP)-anchoring domain
(e.g., cationic anchoring domain)]
(SEQ ID NO: 48)
RGD GAPGAPGAP RRRRRRRRR
(2c) RGD ligand-Cys left
(SEQ ID NO: 49)
CRGD
Note: This can be conjugated to CCA1 (see above)
either via sulfhydryl chemistry (e.g., a disulfide
bond) or amine-reactive chemistry.
(2d) RGD ligand-Cys right
(SEQ ID NO: 50)
RGDC
Note: This can be conjugated to CCA2 (see above)
either via sulfhydryl chemistry (e.g., a disulfide
bond) or amine-reactive chemistry.
(3a) Transferrin ligand
[anchoring domain (e.g., cationic anchoring
domain)-linker (GAPGAPGAP)-Targeting ligand]
(SEQ ID NO: 51)
RRRRRRRRR GAPGAPGAP THRPPMWSPVWP
(3b) Transferrin ligand
[Target ligand-linker (GAPGAPGAP)-anchoring domain
(e.g., cationic anchoring domain)]
(SEQ ID NO: 52)
THRPPMWSPVWP GAPGAPGAP RRRRRRRRR
(3c) Transferrin ligand-Cys left
(SEQ ID NO: 53)
CTHRPPMWSPVWP
(SEQ ID NO: 54)
CPTHRPPMWSPVWP
Note: This can be conjugated to CCA1 (see above)
either via sulfhydryl chemistry (e.g., a disulfide
bond) or amine-reactive chemistry.
(3d) Transferrin ligand-Cys right
(SEQ ID NO: 55)
THRPPMWSPVWPC
Note: This can be conjugated to CCA2 (see above)
eithervia sulfhydryl chemistry (e.g., a disulfide
bond) or amine-reactive chemistry.
(4a) E-selectin ligand [1-21]
[anchoring domain (e.g., cationic anchoring
domain)-linker (GAPGAPGAP)-Targeting ligand]
(SEQ ID NO: 56)
RRRRRRRRR GAPGAPGAP MIASQFLSALTLVLLIKESGA
(4b) E-selectin ligand [1-21]
[Target ligand-linker (GAPGAPGAP)-anchoring domain
(e.g., cationic anchoring domain)]
(SEQ ID NO: 57)
MIASQFLSALTLVLLIKESGA GAPGAPGAP RRRRRRRRR
(4c) E-selectin ligand [1-21]-Cys left
(SEQ ID NO: 58)
CMIASQFLSALTLVLLIKESGA
Note: This can be conjugated to CCA1 (see above)
either via sulfhydryl chemistry (e.g., a disulfide
bond) or amine-reactive chemistry.
(4d) E-selectin ligand [1-21]-Cys right
(SEQ ID NO: 59)
MIASQFLSALTLVLLIKESGAC
Note: This can be conjugated to CCA2 (see above)
either via sulfhydryl chemistry (e.g., a disulfide
bond) or amine-reactive chemistry.
(5a) FGF fragment [26-47]
[anchoring domain (e.g., cationic anchoring
domain)-linker (GAPGAPGAP)-Targeting ligand]
(SEQ ID NO: 60)
RRRRRRRRR GAPGAPGAP KNGGFFLRIHPDGRVDGVREKS
Note: This can be conjugated to CCA1 (see above)
either via sulfhydryl chemistry (e.g., a disulfide
bond) or amine-reactive chemistry.
(5b) FGF fragment [26-47
[Target ligand-linker (GAPGAPGAP)-anchoring domain
(e.g., cationic anchoring domain)]
(SEQ ID NO: 61)
KNGGFFLRIHPDGRVDGVREKS GAPGAPGAP RRRRRRRRR
Note: This can be conjugated to CCA1 (see above)
either via sulfhydryl chemistry (e.g., a disulfide
bond) or amine-reactive chemistry.
(5c) FGF fragment [25-47]-Cys on left is native
(SEQ ID NO: 43)
CKNGGFFLRIHPDGRVDGVREKS
Note: This can be conjugated to CCA1 (see above)
either via sulfhydryl chemistry (e.g., a disulfide
bond) or amine-reactive chemistry.
(5d) FGF fragment [26-47]-Cys right
(SEQ ID NO: 44)
KNGGFFLRIHPDGRVDGVREKSC
Note: This can be conjugated to CCA2 (see above)
either via sulfhydryl chemistry (e.g., a disulfide
bond) or amine-reactive chemistry.
(6a) Exendin (S11C) [1-39]
(SEQ ID NO: 2)
HGEGTFTSDLCKQMEEEAVRLFIEWLKNGGPSSGAPPPS
Note: This can be conjugated to CCA1 (see above)
either via sulfhydryl chemistry (e.g., a disulfide
bond) or amine-reactive chemistry.

### Targeting Ligand

A variety of targeting ligands (e.g., as part of a subject delivery molecule, e.g., as part of a nanoparticle) can be used and numerous different targeting ligands are envisioned. In some embodiments the targeting ligand is a fragment (e.g., a binding domain) of a wild type protein. For example, in some cases a peptide targeting ligand of a subject delivery molecule can have a length of from 4-50 amino acids (e.g., from 4-40, 4-35, 4-30, 4-25, 4-20, 4-15, 5-50, 5-40, 5-35, 5-30, 5-25, 5-20, 5-15, 7-50, 7-40, 7-35, 7-30, 7-25, 7-20, 7-15, 8-50, 8-40, 8-35, 8-30, 8-25, 8-20, or 8-15 amino acids). The targeting ligand can be a fragment of a wild type protein, but in some cases has a mutation (e.g., insertion, deletion, substitution) relative to the wild type amino acid sequence (i.e., a mutation relative to a corresponding wild type protein sequence). For example, a targeting ligand can include a mutation that increases or decreases binding affinity with a target cell surface protein.

In some cases the targeting ligand is an antigen-binding region of an antibody (F(ab)). In some cases the targeting ligand is an ScFv. “Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. For a review of scFv see Pluckthun, in The Pharmacology of Monoclonal Antibodies, vol. 113, Rosenburg and Moore eds., Springer-Verlag, New York, pp. 269-315 (1994).

In some cases a targeting ligand includes a viral glycoprotein, which in some cases binds to ubiquitous surface markers such as heparin sulfate proteoglycans, and may induce micropinocytosis (and/or macropinocytosis) in some cell populations through membrane ruffling associated processes. Poly(L-arginine) is another example targeting ligand that can also be used for binding to surface markers such as heparin sulfate proteoglycans.

In some cases a targeting ligand is coated upon a particle surface (e.g., nanoparticle surface) either electrostatically or utilizing covalent modifications to the particle surface or one or more polymers on the particle surface. In some cases, a targeting ligand can include a mutation that adds a cysteine residue, which can facilitate conjugation to a linker and/or an anchoring domain (e.g., cationic anchoring domain). For example, cysteine can be used for crosslinking (conjugation) via sulfhydryl chemistry (e.g., a disulfide bond) and/or amine-reactive chemistry.

In some cases, a targeting ligand includes an internal cysteine residue. In some cases, a targeting ligand includes a cysteine residue at the N- and/or C-terminus. In some cases, in order to include a cysteine residue, a targeting ligand is mutated (e.g., insertion or substitution), e.g., relative to a corresponding wild type sequence. As such, any of the targeting ligands described herein can be modified by inserting and/or substituting in a cysteine residue (e.g., internal, N-terminal, C-terminal insertion of or substitution with a cysteine residue).

By “corresponding” wild type sequence is meant a wild type sequence from which the subject sequence was or could have been derived (e.g., a wild type protein sequence having high sequence identity to the sequence of interest). In some cases, a “corresponding” wild type sequence is one that has 85% or more sequence identity (e.g., 90% or more, 92% or more, 95% or more, 97% or more, 98% or more, 99% or more, 99.5% or more, or 100% sequence identity) over the amino acid stretch of interest. For example, for a targeting ligand that has one or more mutations (e.g., substitution, insertion) but is otherwise highly similar to a wild type sequence, the amino acid sequence to which it is most similar may be considered to be a corresponding wild type amino acid sequence.

A corresponding wild type protein/sequence does not have to be 100% identical (e.g., can be 85% or more identical, 90% or more identical, 95% or more identical, 98% or more identical, 99% or more identical, etc.) (outside of the position(s) that is modified), but the targeting ligand and corresponding wild type protein (e.g., fragment of a wild protein) can bind to the intended cell surface protein, and retain enough sequence identity (outside of the region that is modified) that they can be considered homologous. The amino acid sequence of a “corresponding” wild type protein sequence can be identified/evaluated using any convenient method (e.g., using any convenient sequence comparison/alignment software such as BLAST, MUSCLE, T-COFFEE, etc.).

Examples of targeting ligands that can be used as part of a surface coat (e.g., as part of a delivery molecule of a surface coat) include, but are not limited to, those listed in Table 1. Examples of targeting ligands that can be used as part of a subject delivery molecule include, but are not limited to, those listed in Table 3 (many of the sequences listed in Table 3 include the targeting ligand (e.g., SNRWLDVK for row 2) (SEQ ID NO: 298) conjugated to a cationic polypeptide domain, e.g., 9R, 6R, etc., via a linker (e.g., GGGGSGGGGS) (SEQ ID NO: 30). Examples of amino acid sequences that can be included in a targeting ligand include, but are not limited to: NPKLTRMLTFKFY (SEQ ID NO: 296) (IL2), TSVGKYPNTGYYGD (SEQ ID NO: 297) (CD3), SNRWLDVK (Siglec) (SEQ ID NO: 298), EKFILKVRPAFKAV (SEQ ID NO: 299) (SCF); EKFILKVRPAFKAV (SEQ ID NO: 299) (SCF), EKFILKVRPAFKAV (SEQ ID NO: 299) (SCF), SNYSIIDKLVNIVDDLVECVKENS (SEQ ID NO: 302) (cKit), and Ac-SNYSAibADKAibANAibADDAibAEAibAKENS (SEQ ID NO: 303) (cKit). Thus in some cases a targeting ligand includes an amino acid sequence that has 85% or more (e.g., 90% or more, 95% or more, 98% or more, 99% or more, or 100%) sequence identity with NPKLTRMLTFKFY (SEQ ID NO: 296) (IL2), TSVGKYPNTGYYGD (SEQ ID NO: 297) (CD3), SNRWLDVK (Siglec) (SEQ ID NO: 298), EKFILKVRPAFKAV (SEQ ID NO: 299) (SCF); EKFILKVRPAFKAV (SEQ ID NO: 299) (SCF), EKFILKVRPAFKAV (SEQ ID NO: 299) (SCF), or SNYSIIDKLVNIVDDLVECVKENS (SEQ ID NO: 302) (cKit).

TABLE 1
Examples of Targeting ligands

| Cell Surface | | | |
|---|---|---|---|
| Protein | Targeting Ligand | Sequence | SEQ ID NO: |
| Family B GPCR | Exendin | HGEGTFTSDLSKQMEEEAVRLFIEWLKNGGPSSG | 1 |
| | | APPPS | |
| | Exendin (S11C) | HGEGTFTSDLCKQMEEEAVRLFIEWLKNGGPSSG | 2 |
| | | APPPS | |
| FGF receptor | FGF fragment | KRLYCKNGGFFLRIHPDGRVDGVREKSDPHIKLQL | 3 |
| | | QAEERGVVSIKGVCANRYLAMKEDGRLLASKCVT | |
| | | DECFFFERLESNNYNTY | |
| | FGF fragment | KNGGFFLRIHPDGRVDGVREKS | 4 |
| | FGF fragment | HFKDPK | 5 |
| | FGF fragment | LESNNYNT | 6 |
| | E-selectin | MIASQFLSALTLVLLIKESGA | 7 |
| | L-selectin | MVFPWRCEGTYWGSRNILKLWVVVTLLCCDFLIHH | 8 |
| | | GTHC | |
| | | MIFPWKCQSTQRDLWNIFKLWGVVTMLCCDFLAH | 9 |
| | | HGTDC | |
| | | MIFPWKCQSTQRDLWNIFKLWGVVTMLCC | 10 |
| P-selectin | PSGL-1 | MAVGASGLEGDKMAGAMPLQLLLLLILLGPGNSL | 271 |
| | (SELPLG) | QLWDTWADEAEKALGPLLARDRRQATEYEYLDY | |
| | | DFLPETEPPEMLRNSTDTTPLTGPGTPESTTVEPA | |
| | | ARRSTGLDAGGAVTELTTELANMGNLSTDSAAME | |
| | | IQTTQPAATEAQTTQPVPTEAQTTPLAATEAQTTR | |
| | | LTATEAQTTPLAATEAQTTPPAATEAQTTQPTGLE | |
| | | AQTTAPAAMEAQTTAPAAMEAQTTPPAAMEAQTT | |
| | | QTTAMEAQTTAPEATEAQTTQPTATEAQTTPLAA | |
| | | MEALSTEPSATEALSMEPTTKRGLFIPFSVSSVTH | |
| | | KGIPMAASNLSVNYPVGAPDHISVKQCLLAILILAL | |
| | | VATIFFVCTVVLAVRLSRKGHMYPVRNYSPTEMV | |
| | | CISSLLPDGGEGPSATANGGLSKAKSPGLTPEPR | |
| | | EDREGDDLTLHSFLP | |
| E-selectin | ESL-1 | MAACGRVRRMFRLSAALHLLLLFAAGAEKLPGQG | 272 |
| | (GLG1) | VHSQGQGPGANFVSFVGQAGGGGPAGQQLPQL | |
| | | PQSSQLQQQQQQQQQQQQPQPPQPPFPAGGPP | |
| | | ARRGGAGAGGGWKLAEEESCREDVTRVCPKHT | |
| | | WSNNLAVLECLQDVREPENEISSDCNHLLWNYKL | |
| | | NLTTDPKFESVAREVCKSTITEIKECADEPVGKGY | |
| | | MVSCLVDHRGNITEYQCHQYITKMTAIIFSDYRLIC | |
| | | GFMDDCKNDINILKCGSIRLGEKDAHSQGEVVSCL | |
| | | EKGLVKEAEEREPKIQVSELCKKAILRVAELSSDD | |
| | | FHLDRHLYFACRDDRERFCENTQAGEGRVYKCLF | |
| | | NHKFEESMSEKCREALTTRQKLIAQDYKVSYSLAK | |
| | | SCKSDLKKYRCNVENLPRSREARLSYLLMCLESA | |
| | | VHRGRQVSSECQGEMLDYRRMLMEDFSLSPEIIL | |
| | | SCRGEIEHHCSGLHRKGRTLHCLMKVVRGEKGNL | |
| | | GMNCQQALQTLIQETDPGADYRIDRALNEACESVI | |
| | | QTACKHIRSGDPMILSCLMEHLYTEKMVEDCEHR | |
| | | LLELQYFISRDWKLDPVLYRKCQGDASRLCHTHG | |
| | | WNETSEFMPQGAVFSCLYRHAYRTEEQGRRLSR | |
| | | ECRAEVQRILHQRAMDVKLDPALQDKCLIDLGKW | |
| | | CSEKTETGQELECLQDHLDDLVVECRDIVGNLTEL | |
| | | ESEDIQIEALLMRACEPIIQNFCHDVADNQIDSGDL | |
| | | MECLIQNKHQKDMNEKCAIGVTHFQLVQMKDFRF | |
| | | SYKFKMACKEDVLKLCPNIKKKVDVVICLSTTVRN | |
| | | DTLQEAKEHRVSLKCRRQLRVEELEMTEDIRLEP | |
| | | DLYEACKSDIKNFCSAVQYGNAQIIECLKENKKQL | |
| | | STRCHQKVFKLQETEMMDPELDYTLMRVCKQMIK | |
| | | RFCPEADSKTMLQCLKQNKNSELMDPKCKQMITK | |
| | | RQITQNTDYRLNPMLRKACKADIPKFCHGILTKAK | |
| | | DDSELEGQVISCLKLRYADQRLSSDCEDQIRIIIQE | |
| | | SALDYRLDPQLQLHCSDEISSLCAEEAAAQEQTG | |
| | | QVEECLKVNLLKIKTELCKKEVLNMLKESKADIFVD | |
| | | PVLHTACALDIKHHCAAITPGRGRQMSCLMEALE | |
| | | DKRVRLQPECKKRLNDRIEMWSYAAKVAPADGFS | |
| | | DLAMQVMTSPSKNYILSVISGSICILFLIGLMCGRIT | |
| | | KRVTRELKDRLQYRSETMAYKGLVWSQDVTGSP | |
| | | A | |
| | PSGL-1 (SELPLG) | See above | 271 |
| | CD44 | MDKFVWWHAAWGLCLVPLSLAQIDLNITCRFAGVF | 273 |
| | | HVEKNGRYSISRTEAADLCKAFNSTLPTMAQMEK | |
| | | ALSIGFETCRYGFIEGHVVIPRIHPNSICAANNTGV | |
| | | YILTSNTSQYDTYCFNASAPPEEDCTSVTDLPNAF | |
| | | DGPITITIVNRDGTRYVQKGEYRTNPEDIYPSNPTD | |
| | | DDVSSGSSSERSSTSGGYIFYTFSTVHPIPDEDSP | |
| | | WITDSTDRIPATTLMSTSATATETATKRQETWDW | |
| | | FSWLFLPSESKNHLHTTTQMAGTSSNTISAGWEP | |
| | | NEENEDERDRHLSFSGSGIDDDEDFISSTISTTPR | |
| | | AFDHTKQNQDVVTQWNPSHSNPEVLLQTTTRMTD | |
| | | VDRNGTTAYEGNWNPEAHPPLIHHEHHEEEETPH | |
| | | STSTIQATPSSTTEETATQKEQWFGNRWHEGYR | |
| | | QTPKEDSHSTTGTAAASAHTSHPMQGRTTPSPE | |
| | | DSSVVTDFFNPISHPMGRGHQAGRRMDMDSSHSI | |
| | | TLQPTANPNTGLVEDLDRTGPLSMTTQQSNSQSF | |
| | | STSHEGLEEDKDHPTTSTLTSSNRNDVTGGRRDP | |
| | | NHSEGSTTLLEGYTSHYPHTKESRTFIPVTSAKTG | |
| | | SFGVTAVTVGDSNSNVNRSLSGDQDTFHPSGGS | |
| | | HTTHGSESDGHSHGSQEGGANTTSGPIRTPQIPE | |
| | | WLIILASLLALALILAVCIAVNSRRRCGQKKKLVI | |
| | | NSGNGAVEDRKPSGLNGEASKSQEMVHLVNKESSE | |
| | | TPDQFMTADETRNLQNVDMKIGV | |
| | DR3 | MEQRPRGCAAVAAALLLVLLGARAQGGTRSPRC | 274 |
| | (TNFRSF25) | DCAGDFHKKIGLFCCRGCPAGHYLKAPCTEPCGN | |
| | | STCLVCPQDTFLAWENHHNSECARCQACDEQAS | |
| | | QVALENCSAVADTRCGCKPGWFVECQVSQCVSS | |
| | | SPFYCQPCLDCGALHRHTRLLCSRRDTDCGTCLP | |
| | | GFYEHGDGCVSCPTPPPSLAGAPWGAVQSAVPL | |
| | | SVAGGRVGVFVVVQVLLAGLVVPLLLGATLTYTYR | |
| | | HCWPHKPLVTADEAGMEALTPPPATHLSPLDSAH | |
| | | TLLAPPDSSEKICTVQLVGNSVVTPGYPETQEALC | |
| | | PQVTWSWDQLPSRALGPAAAPTLSPESPAGSPA | |
| | | MMLQPGPQLYDVMDAVPARRWKEFVRTLGLREA | |
| | | EIEAVEVEIGRFRDQQYEMLKRWRQQQPAGLGA | |
| | | VYAALERMGLDGCVEDLRSRLQRGP | |
| | LAMP1 | MAAPGSARRPLLLLLLLLLLGLMHCASAAMFMVK | 275 |
| | | NGNGTACIMANFSAAFSVNYDTKSGPKNMTFDLP | |
| | | SDATVVLNRSSCGKENTSDPSLVIAFGRGHTLTLN | |
| | | FTRNATRYSVQLMSFVYNLSDTHLFPNASSKEIKT | |
| | | VESITDIRADIDKKYRCVSGTQVHMNNVTVTLHDA | |
| | | TIQAYLSNSSFSRGETRCEQDRPSPTTAPPAPPS | |
| | | PSPSPVPKSPSVDKYNVSGTNGTCLLASMGLQLN | |
| | | LTYERKDNTTVTRLLNINPNKTSASGSCGAHLVTL | |
| | | ELHSEGTTVLLFQFGMNASSSRFFLQGIQLNTILP | |
| | | DARDPAFKAANGSLRALQATVGNSYKCNAEEHV | |
| | | RVTKAFSVNIFKVVVVQAFKVEGGQFGSVEECLLD | |
| | | ENSMLIPIAVGGALAGLVLIVLIAYLVGRKRSHAGY | |
| | | QTI | |
| | LAMP2 | MVCFRLFPVPGSGLVLVCLVLGAVRSYALELNLTD | 276 |
| | | SENATCLYAKWQMNFTVRYETTNKTYKTVTISDH | |
| | | GTVTYNGSICGDDQNGPKIAVQFGPGFSWIANFT | |
| | | KAASTYSIDSVSFSYNTGDNTTFPDAEDKGILTVD | |
| | | ELLARIPLNDLFRCNSLSTLEKNDVVQHYWDVLV | |
| | | QAFVQNGTVSTNEFLCDKDKTSTVAPTIHTTVPSP | |
| | | TTTPTPKEKPEAGTYSVNNGNDTCLLATMGLQLNI | |
| | | TQDKVASVININPNTTHSTGSCRSHTALLRLNSSTI | |
| | | KYLDFVFAVKNENRFYLKEVNISMYLVNGSVFSIA | |
| | | NNNLSYWDAPLGSSYMCNKEQTVSVSGAFQINTF | |
| | | DLRVQPFNVTQGKYSTAQDCSADDDNFLVPIAVG | |
| | | AALAGVLILVLLAYFIGLKHHHAGYEQF | |
| | Mac2-BP | MTPPRLFWVVVLLVAGTQGVNDGDMRLADGGAT | 277 |
| | (galectin 3 binding | NQGRVEIFYRGQWGTVCDNLWDLTDASVVCRAL | |
| | protein) | GFENATQALGRAAFGQGSGPIMLDEVQCTGTEAS | |
| | (LGALS3BP) | LADCKSLGWLKSNCRHERDAGVVCTNETRSTHTL | |
| | | DLSRELSEALGQIFDSQRGCDLSISVNVQGEDALG | |
| | | FCGHTVILTANLEAQALWKEPGSNVTMSVDAECV | |
| | | PMVRDLLRYFYSRRIDITLSSVKCFHKLASAYGAR | |
| | | QLQGYCASLFAILLPQDPSFQMPLDLYAYAVATGD | |
| | | ALLEKLCLQFLAWNFEALTQAEAWPSVPTDLLQLL | |
| | | LPRSDLAVPSELALLKAVDTWSWGERASHEEVEG | |
| | | LVEKIRFPMMLPEELFELQFNLSLYWSHEALFQKK | |
| | | TLQALEFHTVPFQLLARYKGLNLTEDTYKPRIYTSP | |
| | | TWSAFVTDSSWSARKSQLVYQSRRGPLVKYSSD | |
| | | YFQAPSDYRYYPYQSFQTPQHPSFLFQDKRVSW | |
| | | SLVYLPTIQSCWNYGFSCSSDELPVLGLTKSGGS | |
| | | DRTIAYENKALMLCEGLFVADVTDFEGWKAAIPSA | |
| | | LDTNSSKSTSSFPCPAGHFNGFRTVIRPFYLTNSS | |
| | | GVD | |
| Transferrin | Transferrin ligand | THRPPMWSPVWP | 11 |
| receptor | | | |
| α5β1 integrin | α5βligand | RRETAWA | 12 |
| | | RGD | |
| | | RGDGW | 181 |
| integrin | Integrin binding | (Ac)-GCGYGRGDSPG-(NH2) | 188 |
| | peptide | GCGYGRGDSPG | 182 |
| α5β3 integrin | a5β3 ligand | DGARYCRGDCFDG | 187 |
| | rabies virus | YTIWMPENPRPGTPCDIFTNSRGKRASNGGGG | 183 |
| | glycoprotein(RVG) | | |
| c-Kit receptor | stem cell factor | EGICRNRVTNNVKDVTKLVANLPKDYMITLKYVPG | 184 |
| (CD117) | (SCF) | MDVLPSHCWISEMVVQLSDSLTDLLDKFSNISEGL | |
| | | SNYSIIDKLVNIVDDLVECVKENSSKDLKKSFKSPE | |
| | | PRLFTPEEFFRIFNRSIDAFKDFVVASETSDCVVSS | |
| | | TLSPEKDSRVSVTKPFMLPPVA | |
| CD27 | CD70 | PEEGSGCSVRRRPYGCVLRAALVPLVAGLVICLV | 185 |
| | | VCIQRFAQAQQQLPLESLGWDVAELQLNHTGPQ | |
| | | QDPRLYWQGGPALGRSFLHGPELDKGQLRIHRD | |
| | | GIYMVHIQVTLAICSSTTASRHHPTTLAVGICSPAS | |
| | | RSISLLRLSFHQGCTIASQRLTPLARGDTLCTNLTG | |
| | | TLLPSRNTDETFFGVQVVVRP | |
| CD150 | SH2 domain- | SSGLVPRGSHMDAVAVYHGKISRETGEKLLLATG | 186 |
| | containing protein | LDGSYLLRDSESVPGVYCLCVLYHGYIYTYRVSQT | |
| | 1A (SH2D1A) | ETGSWSAETAPGVHKRYFRKIKNLISAFQKPDQGI | |
| | | VIPLQYPVEKKSSARSTQGTTGIREDPDVCLKAP | |
| IL2R | IL2 | NPKLTRMLTFKFY | 296 |
| CD3 | Cde3-epsilon | NFYLYRA-NH2 | 320 |
| CD8 | peptide-HLA-A*2402 | RYPLTFGWCF-NH2 | 321 |
| CD28 | CD80 | VVLKYEKDAFKR | 322 |
| CD28 | CD86 | ENLVLNE | 323 |

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12. In some cases, a targeting ligand includes the amino acid sequence RGD and/or the amino acid sequence set forth in any one of SEQ ID NOs: 1-12. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12.

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12 and 181-187. In some cases, a targeting ligand includes the amino acid sequence RGD and/or the amino acid sequence set forth in any one of SEQ ID NOs: 1-12 and 181-187. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12 and 181-187.

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12, 181-187, and 271-277. In some cases, a targeting ligand includes the amino acid sequence RGD and/or the amino acid sequence set forth in any one of SEQ ID NOs: 1-12, 181-187, and 271-277. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12, 181-187, and 271-277.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187, and 271-277. In some cases, a targeting ligand includes the amino acid sequence set forth in any one of SEQ ID NOs: 181-187, and 271-277. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187, and 271-277.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187. In some cases, a targeting ligand includes the amino acid sequence set forth in any one of SEQ ID NOs: 181-187. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 271-277. In some cases, a targeting ligand includes the amino acid sequence set forth in any one of SEQ ID NOs: 271-277. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 271-277.

The terms “targets” and “targeted binding” are used herein to refer to specific binding. The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, a ligand specifically binds to a particular receptor relative to other available receptors). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity correlates with a lower K_{d}.

In some cases, the targeting ligand provides for targeted binding to a cell surface protein selected from a family B G-protein coupled receptor (GPCR), a receptor tyrosine kinase (RTK), a cell surface glycoprotein, and a cell-cell adhesion molecule. Consideration of a ligand's spatial arrangement upon receptor docking can be used to accomplish a desired functional selectivity and endosomal sorting biases, e.g., so that the structure function relationship between the ligand and the target is not disrupted due to the conjugation of the targeting ligand to the payload or anchoring domain (e.g., cationic anchoring domain). For example, conjugation to a nucleic acid, protein, ribonucleoprotein, or anchoring domain (e.g., cationic anchoring domain) could potentially interfere with the binding cleft(s).

Thus, in some cases, where a crystal structure of a desired target (cell surface protein) bound to its ligand is available (or where such a structure is available for a related protein), one can use 3D structure modeling and sequence threading to visualize sites of interaction between the ligand and the target. This can facilitate, e.g., selection of internal sites for placement of substitutions and/or insertions (e.g., of a cysteine residue).

As an example, in some cases, the targeting ligand provides for binding to a family B G protein coupled receptor (GPCR) (also known as the ‘secretin-family’). In some cases, the targeting ligand provides for binding to both an allosteric-affinity domain and an orthosteric domain of the family B GPCR to provide for the targeted binding and the engagement of long endosomal recycling pathways, respectively.

G-protein-coupled receptors (GPCRs) share a common molecular architecture (with seven putative transmembrane segments) and a common signaling mechanism, in that they interact with G proteins (heterotrimeric GTPases) to regulate the synthesis of intracellular second messengers such as cyclic AMP, inositol phosphates, diacylglycerol and calcium ions. Family B (the secretin-receptor family or ‘family 2’) of the GPCRs is a small but structurally and functionally diverse group of proteins that includes receptors for polypeptide hormones and molecules thought to mediate intercellular interactions at the plasma membrane (see e.g., Harmar et al., Genome Biol. 2001; 2(12):REVIEWS3013). There have been important advances in structural biology as relates to members of the secretin-receptor family, including the publication of several crystal structures of their N-termini, with or without bound ligands, which work has expanded the understanding of ligand binding and provides a useful platform for structure-based ligand design (see e.g., Poyner et al., Br J Pharmacol. 2012 May; 166(1):1-3).

For example, one may desire to use a subject delivery molecule to target the pancreatic cell surface protein GLP1R (e.g., to target ß-islets) using the Exendin-4 ligand, or a derivative thereof (e.g., a cysteine substituted Exendin-4 targeting ligand such as that presented as SEQ ID NO: 2). Because GLP1R is abundant within the brain and pancreas, a targeting ligand that provides for targeting binding to GLP1R can be used to target the brain and pancreas. Thus, targeting GLP1R facilitates methods (e.g., treatment methods) focused on treating diseases (e.g., via delivery of one or more gene editing tools) such as Huntington's disease (CAG repeat expansion mutations), Parkinson's disease (LRRK2 mutations), ALS (SOD1 mutations), and other CNS diseases. Targeting GLP1R also facilitates methods (e.g., treatment methods) focused on delivering a payload to pancreatic β-islets for the treatment of diseases such as diabetes mellitus type I, diabetes mellitus type II, and pancreatic cancer (e.g., via delivery of one or more gene editing tools).

When targeting GLP1R using a modified version of exendin-4, an amino acid for cysteine substitution and/or insertion (e.g., for conjugation to a nucleic acid payload) can be identified by aligning the Exendin-4 amino acid sequence, which is HGEGTFTSDLSKQMEEEAVRLFIEWLKNGGPSSGAPPPS (SEQ ID NO. 1), to crystal structures of glucagon-GCGR (4ERS) and GLP1-GLP1R-ECD complex (PDB: 310L), using PDB 3 dimensional renderings, which may be rotated in 3D space in order to anticipate the direction that a cross-linked complex must face in order not to disrupt the two binding clefts. When a desirable cross-linking site (e.g., site for substitution/insertion of a cysteine residue) of a targeting ligand (that targets a family B GPCR) is sufficiently orthogonal to the two binding clefts of the corresponding receptor, high-affinity binding may occur as well as concomitant long endosomal recycling pathway sequestration (e.g., for optimal payload release). The cysteine substitution at amino acid positions 10, 11, and/or 12 of SEQ ID NO: 1 confers bimodal binding and specific initiation of a Gs-biased signaling cascade, engagement of beta arrestin, and receptor dissociation from the actin cytoskeleton. In some cases, this targeting ligand triggers internalization of the nanoparticle via receptor-mediated endocytosis, a mechanism that is not engaged via mere binding to the GPCR's N-terminal domain without concomitant orthosteric site engagement (as is the case with mere binding of the affinity strand, Exendin-4 [31-39]).

In some cases, a subject targeting ligand includes an amino acid sequence having 85% or more (e.g., 90% or more, 95% or more, 98% or more, 99% or more, or 100%) identity to the exendin-4 amino acid sequence (SEQ ID NO: 1). In some such cases, the targeting ligand includes a cysteine substitution or insertion at one or more of positions corresponding to L10, S11, and K12 of the amino acid sequence set forth in SEQ ID NO: 1. In some cases, the targeting ligand includes a cysteine substitution or insertion at a position corresponding to S11 of the amino acid sequence set forth in SEQ ID NO: 1. In some cases, a subject targeting ligand includes an amino acid sequence having the exendin-4 amino acid sequence (SEQ ID NO: 1). In some cases, the targeting ligand is conjugated (with or without a linker) to an anchoring domain (e.g., a cationic anchoring domain).

As another example, in some cases a targeting ligand according to the present disclosure provides for binding to a receptor tyrosine kinase (RTK) such as fibroblast growth factor (FGF) receptor (FGFR). Thus in some cases the targeting ligand is a fragment of an FGF (i.e., comprises an amino acid sequence of an FGF). In some cases, the targeting ligand binds to a segment of the RTK that is occupied during orthosteric binding (e.g., see the examples section below). In some cases, the targeting ligand binds to a heparin-affinity domain of the RTK. In some cases, the targeting ligand provides for targeted binding to an FGF receptor and comprises an amino acid sequence having 85% or more sequence identity (e.g., 90% or more, 95% or more, 97% or more, 98% or more, 99% or more, 99.5% or more, or 100% sequence identity) with the amino acid sequence KNGGFFLRIHPDGRVDGVREKS (SEQ ID NO: 4). In some cases, the targeting ligand provides for targeted binding to an FGF receptor and comprises the amino acid sequence set forth as SEQ ID NO: 4.

In some cases, small domains (e.g., 5-40 amino acids in length) that occupy the orthosteric site of the RTK may be used to engage endocytotic pathways relating to nuclear sorting of the RTK (e.g., FGFR) without engagement of cell-proliferative and proto-oncogenic signaling cascades, which can be endemic to the natural growth factor ligands. For example, the truncated bFGF (tbFGF) peptide (a.a.30-115), contains a bFGF receptor binding site and a part of a heparin-binding site, and this peptide can effectively bind to FGFRs on a cell surface, without stimulating cell proliferation. The sequences of tbFGF are KRLYCKNGGFFLRIHPDGRVDGVREKSDPHIKLQLQAEERGVVSIKGVCANRYLAMKEDGRLLAS KCVTDECFFFERLESNNYNTY (SEQ ID NO: 13) (see, e.g., Cai et al., Int J Pharm. 2011 Apr. 15; 408(1-2):173-82).

In some cases, the targeting ligand provides for targeted binding to an FGF receptor and comprises the amino acid sequence HFKDPK (SEQ ID NO: 5) (see, e.g., the examples section below). In some cases, the targeting ligand provides for targeted binding to an FGF receptor, and comprises the amino acid sequence LESNNYNT (SEQ ID NO: 6) (see, e.g., the examples section below).

In some cases, a targeting ligand according to the present disclosure provides for targeted binding to a cell surface glycoprotein. In some cases, the targeting ligand provides for targeted binding to a cell-cell adhesion molecule. For example, in some cases, the targeting ligand provides for targeted binding to CD34, which is a cell surface glycoprotein that functions as a cell-cell adhesion factor, and which is protein found on hematopoietic stem cells (e.g., of the bone marrow). In some cases, the targeting ligand is a fragment of a selectin such as E-selectin, L-selectin, or P-selectin (e.g., a signal peptide found in the first 40 amino acids of a selectin). In some cases a subject targeting ligand includes sushi domains of a selectin (e.g., E-selectin, L-selectin, P-selectin).

In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIASQFLSALTLVLLIKESGA (SEQ ID NO: 7). In some cases, the targeting ligand comprises the amino acid sequence set forth as SEQ ID NO: 7. In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MVFPWRCEGTYWGSRNILKLWVWVTLLCCDFLIHHGTHC (SEQ ID NO: 8). In some cases, the targeting ligand comprises the amino acid sequence set forth as SEQ ID NO: 8. In some cases, targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIFPWKCQSTQRDLWNIFKLWGWTMLCCDFLAHHGTDC (SEQ ID NO: 9). In some cases, targeting ligand comprises the amino acid sequence set forth as SEQ ID NO: 9. In some cases, targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIFPWKCQSTQRDLWNIFKLWGVVTMLCC (SEQ ID NO: 10). In some cases, targeting ligand comprises the amino acid sequence set forth as SEQ ID NO: 10.

Fragments of selectins that can be used as a subject targeting ligand (e.g., a signal peptide found in the first 40 amino acids of a selectin) can in some cases attain strong binding to specifically-modified sialomucins, e.g., various Sialyl Lewisx modifications/O-sialylation of extracellular CD34 can lead to differential affinity for P-selectin, L-selectin and E-selectin to bone marrow, lymph, spleen and tonsillar compartments. Conversely, in some cases a targeting ligand can be an extracellular portion of CD34. In some such cases, modifications of sialylation of the ligand can be utilized to differentially target the targeting ligand to various selectins.

In some cases, a targeting ligand according to the present disclosure provides for targeted binding to E-selectin. E-selectin can mediate the adhesion of tumor cells to endothelial cells and ligands for E-selectin can play a role in cancer metastasis. As an example, P-selectin glycoprotein-1 (PSGL-1) (e.g., derived from human neutrophils) can function as a high-efficiency ligand for E-selectin (e.g., expressed by the endothelium), and a subject targeting ligand can therefore in some cases include the PSGL-1 amino acid sequence (or a fragment thereof the binds to E-selectin). As another example, E-selectin ligand-1 (ESL-1) can bind E-selectin and a subject targeting ligand can therefore in some cases include the ESL-1 amino acid sequence (or a fragment thereof the binds to E-selectin). In some cases, a targeting ligand with the PSGL-1 and/or ESL-1 amino acid sequence (or a fragment thereof the binds to E-selectin) bears one or more sialyl Lewis modifications in order to bind E-selectin. As another example, in some cases CD44, death receptor-3 (DR3), LAMP1, LAMP2, and Mac2-BP can bind E-selectin and a subject targeting ligand can therefore in some cases include the amino acid sequence (or a fragment thereof the binds to E-selectin) of any one of: CD44, death receptor-3 (DR3), LAMP1, LAMP2, and Mac2-BP.

In some cases, a targeting ligand according to the present disclosure provides for targeted binding to P-selectin. In some cases PSGL-1 can provide for such targeted binding. In some cases a subject targeting ligand can therefore in some cases include the PSGL-1 amino acid sequence (or a fragment thereof the binds to P-selectin). In some cases, a targeting ligand with the PSGL-1 amino acid sequence (or a fragment thereof the binds to P-selectin) bears one or more sialyl Lewis modifications in order to bind P-selectin.

In some cases, a targeting ligand according to the present disclosure provides for targeted binding to a target selected from: CD3, CD28, CD90, CD45f, CD34, CD80, CD86, CD19, CD20, CD22, CD47, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL-2, IL-7, IL-10, IL-12, IL-15, IL-18, TNFα, IFNγ, TGF-β, and α5β1.

In some cases, a targeting ligand according to the present disclosure provides for targeted binding to a transferrin receptor. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence THRPPMWSPVWP (SEQ ID NO: 11). In some cases, targeting ligand comprises the amino acid sequence set forth as SEQ ID NO: 11.

In some cases, a targeting ligand according to the present disclosure provides for targeted binding to an integrin (e.g., α5β1 integrin). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence RRETAWA (SEQ ID NO: 12). In some cases, targeting ligand comprises the amino acid sequence set forth as SEQ ID NO: 12. In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence RGDGW (SEQ ID NO: 181). In some cases, targeting ligand comprises the amino acid sequence set forth as SEQ ID NO: 181. In some cases, the targeting ligand comprises the amino acid sequence RGD.

In some cases, a targeting ligand according to the present disclosure provides for targeted binding to an integrin. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence GCGYGRGDSPG (SEQ ID NO: 182). In some cases, the targeting ligand comprises the amino acid sequence set forth as SEQ ID NO: 182. In some cases such a targeting ligand is acetylated on the N-terminus and/or amidated (NH2) on the C-terminus.

In some cases, a targeting ligand according to the present disclosure provides for targeted binding to an integrin (e.g., α5β3 integrin). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence DGARYCRGDCFDG (SEQ ID NO: 187). In some cases, the targeting ligand comprises the amino acid sequence set forth as SEQ ID NO: 187.

In some embodiments, a targeting ligand used to target the brain includes an amino acid sequence from rabies virus glycoprotein (RVG) (e.g., YTIWMPENPRPGTPCDIFTNSRGKRASNGGGG (SEQ ID NO: 183)). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 183. As for any of targeting ligand (as described elsewhere herein), RVG can be conjugated and/or fused to an anchoring domain (e.g., 9R peptide sequence). For example, a subject delivery molecule used as part of a surface coat of a subject nanoparticle can include the sequence YTIWMPENPRPGTPCDIFTNSRGKRASNGGGGRRRRRRRRR (SEQ ID NO: 180).

In some cases, a targeting ligand according to the present disclosure provides for targeted binding to c-Kit receptor. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 184. In some cases, the targeting ligand comprises the amino acid sequence set forth as SEQ ID NO: 184.

In some cases, a targeting ligand according to the present disclosure provides for targeted binding to CD27. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 185. In some cases, the targeting ligand comprises the amino acid sequence set forth as SEQ ID NO: 185.

In some cases, a targeting ligand according to the present disclosure provides for targeted binding to CD150. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 186. In some cases, the targeting ligand comprises the amino acid sequence set forth as SEQ ID NO: 186.

In some embodiments, a targeting ligand provides for targeted binding to KLS CD27+/IL-7Ra−/CD150+/CD34− hematopoietic stem and progenitor cells (HSPCs). For example, a gene editing tool(s) (described elsewhere herein) can be introduced in order to disrupt expression of a BCL11a transcription factor and consequently generate fetal hemoglobin. As another example, the beta-globin (HBB) gene may be targeted directly to correct the altered E7V substitution with a corresponding homology-directed repair donor DNA molecule. As one illustrative example, a CRISPR/Cas RNA-guided polypeptide (e.g., Cas9, CasX, CasY, Cpf1) can be delivered with an appropriate guide RNA such that it will bind to loci in the HBB gene and create double-stranded or single-stranded breaks in the genome, initiating genomic repair. In some cases, a Donor DNA molecule (single stranded or double stranded) is introduced (as part of a payload) and is release for 14-30 days while a guide RNA/CRISPR/Cas protein complex (a ribonucleoprotein complex) can be released over the course of from 1-7 days.

In some embodiments, a targeting ligand provides for targeted binding to CD4+ or CD8+ T-cells, hematopoietic stem and progenitor cells (HSPCs), or peripheral blood mononuclear cells (PBMCs), in order to modify the T-cell receptor. For example, a gene editing tool(s) (described elsewhere herein) can be introduced in order to modify the T-cell receptor. The T-cell receptor may be targeted directly and substituted with a corresponding donor DNA molecule for a novel T-cell receptor. As one example, a CRISPR/Cas RNA-guided polypeptide (e.g., Cas9, CasX, CasY, Cpf1) can be delivered with an appropriate guide RNA such that it will bind to loci in the TCR gene and create double-stranded or single-stranded breaks in the genome, initiating insertion of a first donor DNA (that has one or more target sequences for a sequence specific recombinase), and a nucleotide sequence of interest is inserted from the second donor DNA by a recombinase that recognizes the target sequence(s) that was inserted by the first donor DNA. In some cases, a Donor DNA molecule (single stranded or double stranded) is introduced (as part of a payload). It would be evident to skilled artisans that other CRISPR guide RNA and donor sequences, targeting beta-globin, CCR5, the T-cell receptor, or any other gene of interest, and/or other expression vectors may be employed in accordance with the present disclosure.

In some embodiments, a targeting ligand is a nucleic acid aptamer. In some embodiments, a targeting ligand is a peptoid.

Also provided are delivery molecules with two different peptide sequences that together constitute a targeting ligand. For example, in some cases a targeting ligand is bivalent (e.g., heterobivalent). In some cases, cell-penetrating peptides and/or heparin sulfate proteoglycan binding ligands are used as heterobivalent endocytotic triggers along with any of the targeting ligands of this disclosure. A heterobivalent targeting ligand can include an affinity sequence from one of targeting ligand and an orthosteric binding sequence (e.g., one known to engage a desired endocytic trafficking pathway) from a different targeting ligand.

### Anchoring Domain

In some embodiments, a delivery molecule includes a targeting ligand conjugated to an anchoring domain (e.g., cationic anchoring domain, an anionic anchoring domain). In some cases a subject delivery vehicle includes a payload that is condensed with and/or interacts electrostatically the anchoring domain (e.g., a delivery molecule can be the delivery vehicle used to deliver the payload). In some cases the surface coat of a nanoparticle includes such a delivery molecule with an anchoring domain, and in some such cases the payload is in the core (interacts with the core) of such a nanoparticle. See the above section describing charged polymer polypeptide domains for additional details related to anchoring domains.

### Histone Tail Peptide (HTPs)

In some embodiments a cationic polypeptide composition of a subject nanoparticle includes a histone peptide or a fragment of a histone peptide, such as an N-terminal histone tail (e.g., a histone tail of an H1, H2 (e.g., H2A, H2AX, H2B), H3, or H4 histone protein). A tail fragment of a histone protein is referred to herein as a histone tail peptide (HTP). Because such a protein (a histone and/or HTP) can condense with a nucleic acid payload as part of the core of a subject nanoparticle, a core that includes one or more histones or HTPs (e.g., as part of the cationic polypeptide composition) is sometimes referred to herein as a nucleosome-mimetic core. Histones and/or HTPs can be included as monomers, and in some cases form dimers, trimers, tetramers and/or octamers when condensing a nucleic acid payload into a nanoparticle core. In some cases HTPs are not only capable of being deprotonated by various histone modifications, such as in the case of histone acetyltransferase-mediated acetylation, but may also mediate effective nuclear-specific unpackaging of components of the core (e.g., release of a payload). Trafficking of a core that includes a histone and/or HTP may be reliant on alternative endocytotic pathways utilizing retrograde transport through the Golgi and endoplasmic reticulum. Furthermore, some histones include an innate nuclear localization sequence and inclusion of an NLS in the core can direct the core (including the payload) to the nucleus of a target cell.

In some embodiments a subject cationic polypeptide composition includes a protein having an amino acid sequence of an H2A, H2AX, H2B, H3, or H4 protein. In some cases a subject cationic polypeptide composition includes a protein having an amino acid sequence that corresponds to the N-terminal region of a histone protein. For example, the fragment (an HTP) can include the first 5, 10, 15, 20, 25, 30, 35, 40, 45, or 50 N-terminal amino acids of a histone protein. In some cases, a subject HTP includes from 5-50 amino acids (e.g., from 5-45, 5-40, 5-35, 5-30, 5-25, 5-20, 8-50, 8-45, 8-40, 8-35, 8-30, 10-50, 10-45, 10-40, 10-35, or 10-30 amino acids) from the N-terminal region of a histone protein. In some cases a subject a cationic polypeptide includes from 5-150 amino acids (e.g., from 5-100, 5-50, 5-35, 5-30, 5-25, 5-20, 8-150, 8-100, 8-50, 8-40, 8-35, 8-30, 10-150, 10-100, 10-50, 10-40, 10-35, or 10-30 amino acids).

In some cases a cationic polypeptide (e.g., a histone or HTP, e.g., H1, H2, H2A, H2AX, H2B, H3, or H4) of a cationic polypeptide composition includes a post-translational modification (e.g., in some cases on one or more histidine, lysine, arginine, or other complementary residues). For example, in some cases the cationic polypeptide is methylated (and/or susceptible to methylation/demethylation), acetylated (and/or susceptible to acetylation/deacetylation), crotonylated (and/or susceptible to crotonylation/decrotonylation), ubiquitinylated (and/or susceptible to ubiquitinylation/deubiquitinylation), phosphorylated (and/or susceptible to phosphorylation/dephosphorylation), SUMOylated (and/or susceptible to SUMOylation/deSUMOylation), farnesylated (and/or susceptible to farnesylation/defarnesylation), sulfated (and/or susceptible to sulfation/desulfation) or otherwise post-translationally modified. In some cases a cationic polypeptide (e.g., a histone or HTP, e.g., H1, H2, H2A, H2AX, H2B, H3, or H4) of a cationic polypeptide composition is p300/CBP substrate (e.g., see example HTPs below, e.g., SEQ ID NOs: 129-130). In some cases a cationic polypeptide (e.g., a histone or HTP, e.g., H1, H2, H2A, H2AX, H2B, H3, or H4) of a cationic polypeptide composition includes one or more thiol residues (e.g., can include a cysteine and/or methionine residue) that is sulfated or susceptible to sulfation (e.g., as a thiosulfate sulfurtransferase substrate). In some cases a cationic polypeptide (e.g., a histone or HTP, e.g., H1, H2, H2A, H2AX, H2B, H3, or H4) of a cationic polypeptide is amidated on the C-terminus. Histones H2A, H2B, H3, and H4 (and/or HTPs) may be monomethylated, dimethylated, or trimethylated at any of their lysines to promote or suppress transcriptional activity and alter nuclear-specific release kinetics.

A cationic polypeptide can be synthesized with a desired modification or can be modified in an in vitro reaction. Alternatively, a cationic polypeptide (e.g., a histone or HTP) can be expressed in a cell population and the desired modified protein can be isolated/purified. In some cases the cationic polypeptide composition of a subject nanoparticle includes a methylated HTP, e.g., includes the HTP sequence of H3K4(Me3)—includes the amino acid sequence set forth as SEQ ID NO: 75 or 88). In some cases a cationic polypeptide (e.g., a histone or HTP, e.g., H1, H2, H2A, H2AX, H2B, H3, or H4) of a cationic polypeptide composition includes a C-terminal amide.

Examples of Histones and HTPs

Examples Include but are not Limited to the Following Sequences:

H2A
(SEQ ID NO: 62)
SGRGKQGGKARAKAKTRSSR [1-20]
(SEQ ID NO: 63)
SGRGKQGGKARAKAKTRSSRAGLQFPVGRVHRLLRKGGG [1-39]
(SEQ ID NO: 64)
MSGRGKQGGKARAKAKTRSSRAGLQFPVGRVHRLLRKGNYAERVGAGAPV
YLAAVLEYLTAEILELAGNAARDNKKTRIIPRHLQLAIRNDEELNKLLGK
VTIAQGGVLPNIQAVLLPKKTESHHKAKGK [1-130]
H2AX
(SEQ ID NO: 65)
CKATQASQEY [1-143]
(SEQ ID NO: 66)
KKTSATVGPKAPSGGKKATQASQEY [KK 120-129]
(SEQ ID NO: 67)
MSGRGKTGGKARAKAKSRSSRAGLQFPVGRVHRLLRKGHYAERVGAGAPV
YLAAVLEYLTAEILELAGNAARDNKKTRIIPRHLQLAIRNDEELNKLLGG
VTIAQGGVLPNIQAVLLPKKTSATVGPKAPSGGKKATQASQEY [1-143]
H2B
(SEQ ID NO: 68)
PEPA-K(cr)-SAPAPK [1-11 H2BK5(cr)]
[cr: crotonylated (crotonylation)]
(SEQ ID NO: 69)
PEPAKSAPAPK [1-11]
(SEQ ID NO: 70)
AQKKDGKKRKRSRKE [21-35]
(SEQ ID NO: 71)
MPEPAKSAPAPKKGSKKAVTKAQKKDGKKRKRSRKESYSIYVYKVLKQVH
PDTGISSKAMGIMNSFVNDIFERIAGEASRLAHYNKRSTITSREIQTAVR
LLLPGELAKHAVSEGTKAVTKYTSSK[1-126]
H3
(SEQ ID NO: 72)
ARTKQTAR [1-8]
(SEQ ID NO: 73)
ART-K(Me1)-QTARKS [1-8 H3K4(Me1)]
(SEQ ID NO: 74)
ART-K(Me2)-QTARKS [1-8 H3K4(Me2)]
(SEQ ID NO: 75)
ART-K(Me3)-QTARKS [1-8 H3K4(Me3)]
(SEQ ID NO: 76)
ARTKQTARK-pS-TGGKA [1-15 H3pS10]
(SEQ ID NO: 77)
ARTKQTARKSTGGKAPRKWC-NH2 [1-18 WC, amide]
(SEQ ID NO: 78)
ARTKQTARKSTGG-K(Ac)-APRKQ [1-19 H3K14(Ac)]
(SEQ ID NO: 79)
ARTKQTARKSTGGKAPRKQL [1-20]
(SEQ ID NO: 80)
ARTKQTAR-K(Ac)-STGGKAPRKQL [1-20 H3K9(Ac)]
(SEQ ID NO: 81)
ARTKQTARKSTGGKAPRKQLA [1-21]
(SEQ ID NO: 82)
ARTKQTAR-K(Ac)-STGGKAPRKQLA [1-21 H3K9(Ac)]
(SEQ ID NO: 83)
ARTKQTAR-K(Me2)-STGGKAPRKQLA [1-21 H3K9(Me1)]
(SEQ ID NO: 84)
ARTKQTAR-K(Me2)-STGGKAPRKQLA [1-21 H3K9(Me2)]
(SEQ ID NO: 85)
ARTKQTAR-K(Me2)-STGGKAPRKQLA [1-21 H3K9(Me3)]
(SEQ ID NO: 86)
ART-K(Me1)-QTARKSTGGKAPRKQLA [1-21 H3K4(Me1)]
(SEQ ID NO: 87)
ART-K(Me2)-QTARKSTGGKAPRKQLA [1-21 H3K4(Me2)]
(SEQ ID NO: 88)
ART-K(Me3)-QTARKSTGGKAPRKQLA [1-21 H3K4(Me3)]
(SEQ ID NO: 89)
ARTKQTAR-K(Ac)-pS-TGGKAPRKQLA [1-21 H3K9(Ac), pS10]
(SEQ ID NO: 90)
ART-K(Me3)-QTAR-K(Ac)-pS-TGGKAPRKQLA
[1-21 H3K4(Me3), K9(Ac), pS10]
(SEQ ID NO: 91)
ARTKQTARKSTGGKAPRKQLAC [1-21 Cys]
(SEQ ID NO: 92)
ARTKQTAR-K(Ac)-STGGKAPRKQLATKA [1-24 H3K9(Ac)]
(SEQ ID NO: 93)
ARTKQTAR-K(Me3)-STGGKAPRKQLATKA [1-24 H3K9(Me3)]
(SEQ ID NO: 94)
ARTKQTARKSTGGKAPRKQLATKAA [1-25]
(SEQ ID NO: 95)
ART-K(Me3)-QTARKSTGGKAPRKQLATKAA [1-25 H3K4(Me3)]
(SEQ ID NO: 96)
TKQTAR-K(Me1)-STGGKAPR [3-17 H3K9(Me1)]
(SEQ ID NO: 97)
TKQTAR-K(Me2)-STGGKAPR [3-17 H3K9(Me2)]
(SEQ ID NO: 98)
TKQTAR-K(Me3)-STGGKAPR [3-17 H3K9(Me3)]
(SEQ ID NO: 99)
KSTGG-K(Ac)-APRKQ [9-19 H3K14(Ac)
(SEQ ID NO: 100)
QTARKSTGGKAPRKQLASK [5-23]
(SEQ ID NO: 101)
APRKQLATKAARKSAPATGGVKKPH [15-39]
(SEQ ID NO: 102)
ATKAARKSAPATGGVKKPHRYRPG [21-44]
(SEQ ID NO: 103)
KAARKSAPA [23-31]
(SEQ ID NO: 104)
KAARKSAPATGG [23-34]
(SEQ ID NO: 105)
KAARKSAPATGGC [23-34 Cys]
(SEQ ID NO: 106)
KAAR-K(Ac)-SAPATGG [H3K27(Ac)]
(SEQ ID NO: 107)
KAAR-K(Me1)-SAPATGG [H3K27(Me1)]
(SEQ ID NO: 108)
KAAR-K(Me2)-SAPATGG [H3K27(Me2)]
(SEQ ID NO: 109)
KAAR-K(Me3)-SAPATGG [H3K27(Me3)]
(SEQ ID NO: 110)
AT-K(Ac)-AARKSAPSTGGVKKPHRYRPG [21-44 H3K23(Ac)]
(SEQ ID NO: 111)
ATKAARK-pS-APATGGVKKPHRYRPG [21-44 pS28]
(SEQ ID NO: 112)
ARTKQTARKSTGGKAPRKQLATKAARKSAPATGGV [1-35]
(SEQ ID NO: 113)
STGGV-K(Me1)-KPHRY [31-41 H3K36(Me1)]
(SEQ ID NO: 114)
STGGV-K(Me2)-KPHRY [31-41 H3K36(Me2)]
(SEQ ID NO: 115)
STGGV-K(Me3)-KPHRY [31-41 H3K36(Me3)]
(SEQ ID NO: 116)
GTVALREIRRYQ-K(Ac)-STELLIR [44-63 H3K56(Ac)]
(SEQ ID NO: 117)
ARTKQTARKSTGGKAPRKQLATKAARKSAPATGGVKKPHR
YRPGTVALRE [1-50]
(SEQ ID NO: 118)
TELLIRKLPFQRLVREIAQDF-K(Me1)-TDLRFQSAAI
[H3K79(Me1)]
(SEQ ID NO: 119)
EIAQDFKTDLR [73-83
(SEQ ID NO: 120)
EIAQDF-K(Ac)-TDLR [73-83 H3K79(Ac)]
(SEQ ID NO: 121)
EIAQDF-K(Me3)-TDLR [73-83 H3K79(Me3)]
(SEQ ID NO: 122)
RLVREIAQDFKTDLRFQSSAV [69-89]
(SEQ ID NO: 123)
RLVREIAQDFK-(Me1)-TDLRFQSSAV
[69-89 H3K79(Me1), amide]
(SEQ ID NO: 124)
RLVREIAQDFK-(Me2)-TDLRFQSSAV
[69-89 H3K79(Me2), amide]
(SEQ ID NO: 125)
RLVREIAQDFK-(Me3)-TDLRFQSSAV
[69-89 H3K79(Me3), amide]
(SEQ ID NO: 126)
KRVTIMPKDIQLARRIRGERA [116-136]
(SEQ ID NO: 127)
MARTKQTARKSTGGKAPRKQLATKVARKSAPATGGVKKPHRYRPGT
VALREIRRYQKSTELLIRKLPFQRLMREIAQDFKTDLRFQSSAVMA
LQEACESYLVGLFEDTNLCVIHAKRVTIMPKDIQLARRIRGERA
[1-136]
H4
(SEQ ID NO: 128)
SGRGKGG [1-7]
(SEQ ID NO: 129)
RGKGGKGLGKGA [4-12]
(SEQ ID NO: 130)
SGRGKGGKGLGKGGAKRHRKV [1-21]
(SEQ ID NO: 131)
KGLGKGGAKRHRKVLRDNWC-NH2 [8-25 WC, amide]
(SEQ ID NO: 132)
SGRG-K(Ac)-GG-K(Ac)-GLG-K(Ac)-GGA-K(Ac)
RHRKVLRDNGSGSK [1-25 H4K5, 8, 12, 16(Ac)]
(SEQ ID NO: 133)
SGRGKGGKGLGKGGAKRHRK-NH2
[1-20 H4 PRMT7(protein arginine methyltransferase 7)
Substrate, M1]
(SEQ ID NO: 134)
SGRG-K(Ac)-GGKGLGKGGAKRHRK [1-20 H4K5 (Ac)]
(SEQ ID NO: 135)
SGRGKGG-K(Ac)-GLGKGGAKRHRK [1-20 H4K8 (Ac)]
(SEQ ID NO: 136)
SGRGKGGKGLG-K(Ac)-GGAKRHRK [1-20 H4K12 (Ac)]
(SEQ ID NO: 137)
SGRGKGGKGLGKGGA-K(Ac)-RHRK [1-20 H4K16 (Ac)]
(SEQ ID NO: 138)
KGLGKGGAKRHRKVLRDNWC-NH2 [1-25 WC, amide]
(SEQ ID NO: 139)
MSGRGKGGKGLGKGGAKRHRKVLRDNIQGITKPAIRRLARRGGVKRI
SGLIYEETRGVLKVFLENVIRDAVTYTEHAKRKTVTAMDVVYALKRQ
GRTLYGFGG [1-103]

As such, a cationic polypeptide of a subject cationic polypeptide composition can include an amino acid sequence having the amino acid sequence set forth in any of SEQ ID NOs: 62-139. In some cases a cationic polypeptide of subject a cationic polypeptide composition includes an amino acid sequence having 80% or more sequence identity (e.g., 85% or more, 90% or more, 95% or more, 98% or more, 99% or more, or 100% sequence identity) with the amino acid sequence set forth in any of SEQ ID NOs: 62-139. In some cases a cationic polypeptide of subject a cationic polypeptide composition includes an amino acid sequence having 90% or more sequence identity (e.g., 95% or more, 98% or more, 99% or more, or 100% sequence identity) with the amino acid sequence set forth in any of SEQ ID NOs: 62-139. The cationic polypeptide can include any convenient modification, and a number of such contemplated modifications are discussed above, e.g., methylated, acetylated, crotonylated, ubiquitinylated, phosphorylated, SUMOylated, farnesylated, sulfated, and the like.

In some cases a cationic polypeptide of a cationic polypeptide composition includes an amino acid sequence having 80% or more sequence identity (e.g., 85% or more, 90% or more, 95% or more, 98% or more, 99% or more, or 100% sequence identity) with the amino acid sequence set forth in SEQ ID NO: 94. In some cases a cationic polypeptide of a cationic polypeptide composition includes an amino acid sequence having 95% or more sequence identity (e.g., 98% or more, 99% or more, or 100% sequence identity) with the amino acid sequence set forth in SEQ ID NO: 94. In some cases a cationic polypeptide of a cationic polypeptide composition includes the amino acid sequence set forth in SEQ ID NO: 94. In some cases a cationic polypeptide of a cationic polypeptide composition includes the sequence represented by H3K4(Me3) (SEQ ID NO: 95), which comprises the first 25 amino acids of the human histone 3 protein, and tri-methylated on the lysine 4 (e.g., in some cases amidated on the C-terminus).

In some embodiments a cationic polypeptide (e.g., a histone or HTP, e.g., H1, H2, H2A, H2AX, H2B, H3, or H4) of a cationic polypeptide composition includes a cysteine residue, which can facilitate conjugation to: a cationic (or in some cases anionic) amino acid polymer, a linker, an NLS, and/or other cationic polypeptides (e.g., in some cases to form a branched histone structure). For example, a cysteine residue can be used for crosslinking (conjugation) via sulfhydryl chemistry (e.g., a disulfide bond) and/or amine-reactive chemistry. In some cases the cysteine residue is internal. In some cases the cysteine residue is positioned at the N-terminus and/or C-terminus. In some cases, a cationic polypeptide (e.g., a histone or HTP, e.g., H1, H2, H2A, H2AX, H2B, H3, or H4) of a cationic polypeptide composition includes a mutation (e.g., insertion or substitution) that adds a cysteine residue. Examples of HTPs that include a cysteine include but are not limited to:

(SEQ ID NO: 140)
CKATQASQEY-from H2AX
(SEQ ID NO: 141)
ARTKQTARKSTGGKAPRKQLAC-from H3
(SEQ ID NO: 142)
ARTKQTARKSTGGKAPRKWC
(SEQ ID NO: 143)
KAARKSAPATGGC-from H3
(SEQ ID NO: 144)
KGLGKGGAKRHRKVLRDNWC-from H4
(SEQ ID NO: 145)
MARTKQTARKSTGGKAPRKQLATKVARKSAPATGGVKKPHRYRPGTVALR
EIRRYQKSTELLIRKLPFQRLMREIAQDFKTDLRFQSSAVMALQEACESY
LVGLFEDTNLCVIHAKRVTIMPKDIQLARRIRGERA-from H3

In some embodiments a cationic polypeptide (e.g., a histone or HTP, e.g., H1, H2, H2A, H2AX, H2B, H3, or H4) of a cationic polypeptide composition is conjugated to a cationic (and/or anionic) amino acid polymer of the core of a subject nanoparticle. As an example, a histone or HTP can be conjugated to a cationic amino acid polymer (e.g., one that includes poly(lysine)), via a cysteine residue, e.g., where the pyridyl disulfide group(s) of lysine(s) of the polymer are substituted with a disulfide bond to the cysteine of a histone or HTP.

### Modified/Branching Structure

In some embodiments a cationic polypeptide of a subject a cationic polypeptide composition has a linear structure. In some embodiments a cationic polypeptide of a subject a cationic polypeptide composition has a branched structure.

For example, in some cases, a cationic polypeptide (e.g., HTPs, e.g., HTPs with a cysteine residue) is conjugated (e.g., at its C-terminus) to the end of a cationic polymer (e.g., poly(L-arginine), poly(D-lysine), poly(L-lysine), poly(D-lysine)), thus forming an extended linear polypeptide. In some cases, one or more (two or more, three or more, etc.) cationic polypeptides (e.g., HTPs, e.g., HTPs with a cysteine residue) are conjugated (e.g., at their C-termini) to the end(s) of a cationic polymer (e.g., poly(L-arginine), poly(D-lysine), poly(L-lysine), poly(D-lysine)), thus forming an extended linear polypeptide. In some cases the cationic polymer has a molecular weight in a range of from 4,500-150,000 Da).

As another example, in some cases, one or more (two or more, three or more, etc.) cationic polypeptides (e.g., HTPs, e.g., HTPs with a cysteine residue) are conjugated (e.g., at their C-termini) to the side-chains of a cationic polymer (e.g., poly(L-arginine), poly(D-lysine), poly(L-lysine), poly(D-lysine)), thus forming a branched structure (branched polypeptide). Formation of a branched structure by components of the nanoparticle core (e.g., components of a subject cationic polypeptide composition) can in some cases increase the amount of core condensation (e.g., of a nucleic acid payload) that can be achieved. Thus, in some cases it is desirable to used components that form a branched structure. Various types of branches structures are of interest, and examples of branches structures that can be generated (e.g., using subject cationic polypeptides such as HTPs, e.g., HTPs with a cysteine residue; peptoids, polyamides, and the like) include but are not limited to: brush polymers, webs (e.g., spider webs), graft polymers, star-shaped polymers, comb polymers, polymer networks, dendrimers, and the like.

In some cases, a branched structure includes from 2-30 cationic polypeptides (e.g., HTPs) (e.g., from 2-25, 2-20, 2-15, 2-10, 2-5, 4-30, 4-25, 4-20, 4-15, or 4-10 cationic polypeptides), where each can be the same or different than the other cationic polypeptides of the branched structure. In some cases the cationic polymer has a molecular weight in a range of from 4,500-150,000 Da). In some cases, 5% or more (e.g., 10% or more, 20% or more, 25% or more, 30% or more, 40% or more, or 50% or more) of the side-chains of a cationic polymer (e.g., poly(L-arginine), poly(D-lysine), poly(L-lysine), poly(D-lysine)) are conjugated to a subject cationic polypeptide (e.g., HTP, e.g., HTP with a cysteine residue). In some cases, up to 50% (e.g., up to 40%, up to 30%, up to 25%, up to 20%, up to 15%, up to 10%, or up to 5%) of the side-chains of a cationic polymer (e.g., poly(L-arginine), poly(D-lysine), poly(L-lysine), poly(D-lysine)) are conjugated to a subject cationic polypeptide (e.g., HTP, e.g., HTP with a cysteine residue). Thus, an HTP can be branched off of the backbone of a polymer such as a cationic amino acid polymer.

In some cases formation of branched structures can be facilitated using components such as peptoids (polypeptoids), polyamides, dendrimers, and the like. For example, in some cases peptoids (e.g., polypeptoids) are used as a component of a nanoparticle core, e.g., in order to generate a web (e.g., spider web) structure, which can in some cases facilitate condensation of the nanoparticle core.

One or more of the natural or modified polypeptide sequences herein may be modified with terminal or intermittent arginine, lysine, or histidine sequences. In one embodiment, each polypeptide is included in equal amine molarities within a nanoparticle core. In this embodiment, each polypeptide's C-terminus can be modified with 5R (5 arginines). In some embodiments, each polypeptide's C-terminus can be modified with 9R (9 arginines). In some embodiments, each polypeptide's N-terminus can be modified with 5R (5 arginines). In some embodiments, each polypeptide's N-terminus can be modified with 9R (9 arginines). In some cases, an H2A, H2B, H3 and/or H4 histone fragment (e.g., HTP) are each bridged in series with a FKFL Cathepsin B proteolytic cleavage domain or RGFFP Cathepsin D proteolytic cleavage domain. In some cases, an H2A, H2B, H3 and/or H4 histone fragment (e.g., HTP) can be bridged in series by a 5R (5 arginines), 9R (9 arginines), 5K (5 lysines), 9K (9 lysines), 5H (5 histidines), or 9H (9 histidines) cationic spacer domain. In some cases, one or more H2A, H2B, H3 and/or H4 histone fragments (e.g., HTPs) are disulfide-bonded at their N-terminus to protamine.

To illustrate how to generate a branched histone structure, example methods of preparation are provided. One example of such a method includes the following: covalent modification of equimolar ratios of Histone H2AX [134-143], Histone H3 [1-21 Cys], Histone H3 [23-34 Cys], Histone H4 [8-25 WC] and SV40 T-Ag-derived NLS can be performed in a reaction with 10% pyridyl disulfide modified poly(L-Lysine) [MW=5400, 18000, or 45000 Da; n=30, 100, or 250]. In a typical reaction, a 29 μL aqueous solution of 700 μM Cys-modified histone/NLS (20 nmol) can be added to 57 μL of 0.2 M phosphate buffer (pH 8.0). Second, 14 μL of 100 μM pyridyl disulfide protected poly(lysine) solution can then be added to the histone solution bringing the final volume to 100 μL with a 1:2 ratio of pyridyl disulfide groups to Cysteine residues. This reaction can be carried out at room temperature for 3 h. The reaction can be repeated four times and degree of conjugation can be determined via absorbance of pyridine-2-thione at 343 nm.

As another example, covalent modification of a 0:1, 1:4, 1:3, 1:2, 1:1, 1:2, 1:3, 1:4, or 1:0 molar ratio of Histone H3 [1-21 Cys] peptide and Histone H3 [23-34 Cys] peptide can be performed in a reaction with 10% pyridyl disulfide modified poly(L-Lysine) or poly(L-Arginine) [MW=5400, 18000, or 45000 Da; n=30, 100, or 250]. In a typical reaction, a 29 μL aqueous solution of 700 μM Cys-modified histone (20 nmol) can be added to 57 μL of 0.2 M phosphate buffer (pH 8.0). Second, 14 μL of 100 μM pyridyl disulfide protected poly(lysine) solution can then be added to the histone solution bringing the final volume to 100 μL with a 1:2 ratio of pyridyl disulfide groups to Cysteine residues. This reaction can be carried out at room temperature for 3 h. The reaction can be repeated four times and degree of conjugation can be determined via absorbance of pyridine-2-thione at 343 nm.

In some cases, an anionic polymer is conjugated to a targeting ligand.

### Nuclear Localization Sequence (NLS)

In some embodiments a cationic polypeptide (e.g., a histone or HTP, e.g., H1, H2, H2A, H2AX, H2B, H3, or H4) of a cationic polypeptide composition includes (and/or is conjugated to) one or more (e.g., two or more, three or more, or four or more) nuclear localization sequences (NLSs). Thus in some cases the cationic polypeptide composition of a subject nanoparticle includes a peptide that includes an NLS. In some cases a histone protein (or an HTP) of a subject nanoparticle includes one or more (e.g., two or more, three or more) natural nuclear localization signals (NLSs). In some cases a histone protein (or an HTP) of a subject nanoparticle includes one or more (e.g., two or more, three or more) NLSs that are heterologous to the histone protein (i.e., NLSs that do not naturally occur as part of the histone/HTP, e.g., an NLS can be added by humans). In some cases the HTP includes an NLS on the N- and/or C-terminus.

In some embodiments a cationic amino acid polymer (e.g., poly(arginine)(PR), poly(lysine)(PK), poly(histidine)(PH), poly(ornithine), poly(citrulline), poly(D-arginine)(PDR), poly(D-lysine)(PDK), poly(D-histidine)(PDH), poly(D-ornithine), poly(D-citrulline), poly(L-arginine)(PLR), poly(L-lysine)(PLK), poly(L-histidine)(PLH), poly(L-ornithine), or poly(L-citrulline)) of a cationic polymer composition includes (and/or is conjugated to) one or more (e.g., two or more, three or more, or four or more) NLSs. In some cases the cationic amino acid polymer includes an NLS on the N- and/or C-terminus. In some cases the cationic amino acid polymer includes an internal NLS.

In some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), or poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

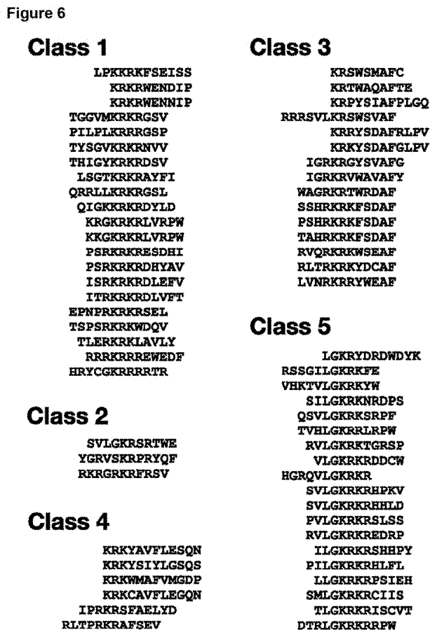

Any convenient NLS can be used (e.g., conjugated to a histone, an HTP, a cationic amino acid polymer, an anionic amino acid polymer, and the like). Examples include, but are not limited to Class 1 and Class 2 ‘monopartite NLSs’, as well as NLSs of Classes 3-5 (see, e.g., FIG. 6, which is adapted from Kosugi et al., J Biol Chem. 2009 Jan. 2; 284(1):478-85). In some cases, an NLS has the formula: (K/R) (K/R) X₁₀₋₁₂(K/R)₃₋₅. In some cases, an NLS has the formula: K(K/R)X(K/R).

In some embodiments a cationic polypeptide of a cationic polypeptide composition includes one more (e.g., two or more, three or more, or four or more) NLSs. In some cases the cationic polypeptide is not a histone protein or histone fragment (e.g., is not an HTP). Thus, in some cases the cationic polypeptide of a cationic polypeptide composition is an NLS-containing peptide.

In some cases, the NLS-containing peptide includes a cysteine residue, which can facilitate conjugation to: a cationic (or in some cases anionic) amino acid polymer, a linker, histone protein for HTP, and/or other cationic polypeptides (e.g., in some cases as part of a branched histone structure). For example, a cysteine residue can be used for crosslinking (conjugation) via sulfhydryl chemistry (e.g., a disulfide bond) and/or amine-reactive chemistry. In some cases the cysteine residue is internal. In some cases the cysteine residue is positioned at the N-terminus and/or C-terminus. In some cases, an NLS-containing peptide of a cationic polypeptide composition includes a mutation (e.g., insertion or substitution) (e.g., relative to a wild type amino acid sequence) that adds a cysteine residue.

Examples of NLSs that can be used as an NLS-containing peptide (or conjugated to any convenient cationic polypeptide such as an HTP or cationic polymer or cationic amino acid polymer or anionic amino acid polymer) include but are not limited to (some of which include a cysteine residue):

```
  (SEQ ID NO: 151)
PKKKRKV (T-ag NLS)
(SEQ ID NO: 152)
PKKKRKVEDPYC-5V40 T-Ag-derived NLS
(SEQ ID NO: 153)
PKKKRKVGPKKKRKVGPKKKRKVGPKKKRKVGC (NLS SV40)
(SEQ ID NO: 154)
CYGRKKRRQRRR-N-terminal cysteine of cysteine-TAT
(SEQ ID NO: 155)
CSIPPEVKFNKPFVYLI
  (SEQ ID NO: 156)
DRQIKIWFQNRRMKWKK
(SEQ ID NO: 157)
PKKKRKVEDPYC-C-term cysteine of an SV40
T-Ag-derived NLS
(SEQ ID NO: 158)
PAAKRVKLD [cMyc NLS]
```

For non-limiting examples of NLSs that can be used, see, e.g., Kosugi et al., J Biol Chem. 2009 Jan. 2; 284(1):478-85, e.g., see FIG. 6 of this disclosure.

### Mitochondrial Localization Signal

In some embodiments a cationic polypeptide (e.g., a histone or HTP, e.g., H1, H2, H2A, H2AX, H2B, H3, or H4), an anionic polymer, and/or a cationic polymer of a subject nanoparticle includes (and/or is conjugated to) one or more (e.g., two or more, three or more, or four or more) mitochondrial localization sequences. Any convenient mitochondrial localization sequence can be used. Examples of mitochondrial localization sequences include but are not limited to: PEDEIWLPEPESVDVPAKPISTSSMMMP (SEQ ID NO: 149), a mitochondrial localization sequence of SDHB, mono/di/triphenylphosphonium or other phosphoniums, VAMP 1A, VAMP 1B, the 67 N-terminal amino acids of DGAT2, and the 20 N-terminal amino acids of Bax.

### Delivery

As noted above, in some embodiments a subject method includes introducing a delivery vehicle (e.g., a nanoparticle, a delivery molecule, etc.) into a target cell, which can in some cases be accomplished by contacting the target cell with the delivery vehicle. If the target cell is in vivo, the introducing can be accomplished by administering the delivery vehicle to an individual. A subject delivery vehicle (e.g., nanoparticle, delivery molecule, etc.) can be delivered to any desired target cell, e.g., any desired eukaryotic cell.

In some cases the target cell is in vitro (e.g., the cell is in culture), e.g., the cell can be a cell of an established tissue culture cell line. In some cases the target cell is ex vivo (e.g., the cell is a primary cell (or a recent descendant) isolated from an individual, e.g. a patient). In some cases, the target cell is in vivo and is therefore inside of (part of) an organism.

The components described herein, e.g., as payloads of a delivery vehicle, may be introduced to a subject (i.e., administered to an individual) via any convenient route—examples include but are not limited to: systemic, local, parenteral, subcutaneous (s.c.), intravenous (i.v.), intracranial (i.c.), intraspinal, intraocular, intradermal (i.d.), intramuscular (i.m.), intralymphatic or into spinal fluid. The components/delivery vehicle may be introduced by injection (e.g., systemic injection, direct local injection, local injection into or near a tumor and/or a site of tumor resection, etc.), catheter, or the like. Examples of methods for local delivery (e.g., delivery to a tumor and/or cancer site) include, e.g., by bolus injection, e.g. by a syringe, e.g. into a joint, tumor, or organ, or near a joint, tumor, or organ; e.g., by continuous infusion, e.g. by cannulation, e.g. with convection (see e.g. US Application No. 20070254842, incorporated here by reference).

The number of administrations of treatment to a subject may vary. Introducing a delivery vehicle, into an individual may be a one-time event; but in certain situations, such treatment may elicit improvement for a limited period of time and require an on-going series of repeated treatments. In other situations, multiple administrations of a delivery vehicle may be required before an effect is observed. As will be readily understood by one of ordinary skill in the art, the exact protocols depend upon the disease or condition, the stage of the disease and parameters of the individual being treated.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of a delivery vehicle is an amount that is sufficient, when administered to the individual, to palliate, ameliorate, stabilize, reverse, prevent, slow or delay the progression of a disease state/ailment.

An example therapeutic intervention is one that creates resistance to HIV infection in addition to ablating any retroviral DNA that has been integrated into the host genome. T-cells are directly affected by HIV and thus a hybrid blood targeting strategy for CD34+ and CD45+ cells may be explored. For example, an effective therapeutic intervention may include simultaneously targeting HSCs and T-cells and delivering an ablation (and replacement sequence) to the CCR5-Δ32 and gag/rev/pol genes through multiple guided nucleases (e.g., within a single particle).

In some cases, the target cell is a mammalian cell (e.g., a rodent cell, a mouse cell, a rat cell, an ungulate cell, a cow cell, a sheep cell, a pig cell, a horse cell, a camel cell, a rabbit cell, a canine (dog) cell, a feline (cat) cell, a primate cell, a non-human primate cell, a human cell). Any cell type can be targeted, and in some cases specific targeting of particular cells depends on the presence of targeting ligands (e.g., as part of a surface coat of a nanoparticle, as part of a delivery molecule, etc), where the targeting ligands provide for targeting binding to a particular cell type. For example, cells that can be targeted include but are not limited to bone marrow cells, hematopoietic stem cells (HSCs), long-term HSCs, short-term HSCs, hematopoietic stem and progenitor cells (HSPCs), peripheral blood mononuclear cells (PBMCs), myeloid progenitor cells, lymphoid progenitor cells, T-cells, B-cells (e.g., via targeting CD19, CD20, CD22), NKT cells, NK cells, dendritic cells, monocytes, granulocytes, erythrocytes, megakaryocytes, mast cells, basophils, eosinophils, neutrophils, macrophages (e.g., via targeting CD47 via SIRPα-mimetic peptides), erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, muscle cells, skeletal muscle cells, cardiac muscle cells, hepatic cells, fat cells, intestinal cells, cells of the colon, and cells of the stomach.

Examples of various applications (e.g., for targeting neurons, cells of the pancreas, hematopoietic stem cells and multipotent progenitors, etc.) are discussed above, e.g., in the context of targeting ligands. For example, Hematopoietic stem cells and multipotent progenitors can be targeted for gene editing (e.g., insertion) in vivo. Even editing 1% of bone marrow cells in vivo (approximately 15 billion cells) would target more cells than an ex vivo therapy (approximately 10 billion cells). As another example, pancreatic cells (e.g., β islet cells) can be targeted, e.g., to treat pancreatic cancer, to treat diabetes, etc. As another example, somatic cells in the brain such as neurons can be targeted (e.g., to treat indications such as Huntington's disease, Parkinson's (e.g., LRRK2 mutations), and ALS (e.g., SOD1 mutations)). In some cases this can be achieved through direct intracranial injections.

As another example, endothelial cells and cells of the hematopoietic system (e.g., megakaryocytes and/or any progenitor cell upstream of a megakaryocyte such as a megakaryocyte-erythroid progenitor cell (MEP), a common myeloid progenitor cell (CMP), a multipotent progenitor cell (MPP), a hematopoietic stem cells (HSC), a short term HSC (ST-HSC), an IT-HSC, a long term HSC (LT-HSC)—see, e.g., FIGS. 7-8) can be targeted with a subject nanoparticle (or subject viral or non-viral delivery vehicle) to treat Von Willebrand's disease. For example, a cell (e.g., an endothelial cell, a megakaryocyte and/or any progenitor cell upstream of a megakaryocyte such as an MEP, a CMP, an MPP, an HSC such as an ST-HSC, an IT-HSC, and/or an LT-HSC) harboring a mutation in the gene encoding von Willebrand factor (VWF) can be targeted (in vitro, ex vivo, in vivo) in order to edit (and correct) the mutated gene, e.g., by introducing a replacement sequence (e.g., via delivery of a donor DNA). In some of the above cases (e.g., in cases related to treating Von Willebrand's disease, in cases related to targeting a cell harboring a mutation in the gene encoding VWF), a subject targeting ligand provides for targeted binding to E-selectin.

Methods and compositions of this disclosure can be used to treat any number of diseases, including any disease that is linked to a known causative mutation, e.g., a mutation in the genome. For example, methods and compositions of this disclosure can be used to treat sickle cell disease, ß thalassemia, HIV, myelodysplastic syndromes, JAK2-mediated polycythemia vera, JAK2-mediated primary myelofibrosis, JAK2-mediated leukemia, and various hematological disorders. As additional non-limiting examples, the methods and compositions of this disclosure can also be used for B-cell antibody generation, immunotherapies (e.g., delivery of a checkpoint blocking reagent), and stem cell differentiation applications.

In some embodiments, a targeting ligand provides for targeted binding to KLS CD27+/IL-7Ra−/CD150+/CD34− hematopoietic stem and progenitor cells (HSPCs). For example, the beta-globin (HBB) gene may be targeted directly to correct the altered E7V substitution with an appropriate donor DNA molecule (of an insert donor composition). As one illustrative example, a CRISPR/Cas RNA-guided polypeptide (e.g., Cas9, CasX, CasY, Cpf1) can be delivered with an appropriate guide RNA(s) such that it will bind to loci in the HBB gene and create a cut in the genome, initiating insertion of a sequence from an introduced first donor DNA (target donor composition). The target site(s) produced by said insertion provide a target for a site-specific recombinase, which catalyzes insertion of a nucleotide sequence of interest from a second donor DNA. In some cases, a Donor DNA molecule (single stranded or double stranded) is introduced (as part of a payload) and is release for 14-30 days while a guide RNA/CRISPR/Cas protein complex (a ribonucleoprotein complex) can be released over the course of from 1-7 days.

In some embodiments, a targeting ligand provides for targeted binding to CD4+ or CD8+ T-cells, hematopoietic stem and progenitor cells (HSPCs), or peripheral blood mononuclear cells (PBMCs), in order to modify the T-cell receptor. For example, a gene editing tool(s) (described elsewhere herein) can be introduced in order to modify the T-cell receptor. The T-cell receptor may be targeted directly and substituted with a corresponding homology-directed repair donor DNA molecule for a novel T-cell receptor. As one example, a CRISPR/Cas RNA-guided polypeptide (e.g., Cas9, CasX, CasY, Cpf1) can be delivered with an appropriate guide RNA(s) such that it will bind to loci in the HBB gene and create a cut in the genome, initiating insertion of a sequence from an introduced first donor DNA (target donor composition). The target site(s) produced by said insertion provide a target for a site-specific recombinase, which catalyzes insertion of a nucleotide sequence of interest from a second donor DNA. It would be evident to skilled artisans that other CRISPR guide RNA and donor sequences, targeting beta-globin, CCR5, the T-cell receptor, or any other gene of interest, and/or other expression vectors may be employed in accordance with the present disclosure.

In some cases, a subject method is used to target a locus that encodes a T cell receptor (TCR), which in some cases has nearly 100 domains and as many as 1,000,000 base pairs with the constant region separated from the V(D)J regions by ˜100,000 base pairs or more. In some cases insertion of the donor DNA occurs within a nucleotide sequence that encodes a T cell receptor (TCR) protein. In some such cases the sequence of the donor DNA (of the insert donor composition) that is inserted into the genome encodes amino acids of a CDR1, CDR2, or CDR3 region of the TCR protein. See, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21.

In some cases a subject method is used to insert genes while placing them under the control of (in operable linkage with) specific enhancers as a fail-safe to genome engineering. If the insertion fails, the enhancer is disrupted leading to the subsequent gene and any possible indels being unlikely to express. If the gene insertion succeeds, a new gene can be inserted with a stop codon at its end, which is particularly useful for multi-part genes such as the TCR locus. In some cases, the subject methods can be used to insert a chimeric antigen receptor (CAR) or other construct into a T-cell, or to cause a B-cell to create a specific antibody or alternative to an antibody (such as a nanobody, shark antibody, etc.).

In some cases the sequence of the donor DNA (of the insert donor composition) that is inserted into the genome encodes a chimeric antigen receptor (CAR). In some such cases, insertion of the donor DNA results in operable linkage of the nucleotide sequence encoding the CAR to an endogenous T-cell promoter (i.e., expression of the CAR will be under the control of an endogenous promoter). In some cases the sequence of the donor DNA (of the insert donor composition) that is inserted into the genome includes a nucleotide sequence that is operably linked to a promoter and encodes a chimeric antigen receptor (CAR)—and thus the inserted CAR will be under the control of the promoter that was present on the donor DNA.

In some cases the sequence of the donor DNA (of the insert donor composition) that is inserted into the genome includes a nucleotide sequence encoding a cell-specific targeting ligand that is membrane bound and presented extracellularly. In some cases, insertion of said donor DNA results in operable linkage of the nucleotide sequence encoding the cell-specific targeting ligand to an endogenous promoter. In some cases the sequence of the donor DNA (of the insert donor composition) that is inserted into the genome includes a promoter operably linked to the sequence that encodes a cell-specific targeting ligand that is membrane bound and presented extracellularly—and therefore, after insertion of the sequence, expression of the membrane bound targeting ligand will be under the control of the promoter that was present on the donor DNA.

In some embodiments, insertion of a sequence of the donor DNA (of the insert donor composition) occurs within a nucleotide sequence that encodes a T cell receptor (TCR) Alpha or Delta subunit. In some cases, insertion of a sequence of the donor DNA (of the insert donor composition) occurs within a nucleotide sequence that encodes a TCR Beta or Gamma subunit. In some cases a subject method and/or composition includes two donor DNAs. In some such cases insertion of one sequence of the donor DNA (of the insert donor composition) occurs within a nucleotide sequence that encodes a T cell receptor (TCR) Alpha or Delta subunit and insertion of the sequence of the other donor DNA (of the insert donor composition) occurs within a nucleotide sequence that encodes a T cell receptor (TCR) Beta or Gamma subunit.

In some embodiments, insertion of a sequence of the donor DNA (of the insert donor composition) occurs within a nucleotide sequence that encodes a T cell receptor (TCR) Alpha or Delta subunit constant region. In some cases insertion of a sequence of the donor DNA (of the insert donor composition) occurs within a nucleotide sequence that encodes a T cell receptor (TCR) Beta or Gamma subunit constant region. In some cases a subject method and/or composition includes two donor DNAs. In some such cases insertion of one sequence of the donor DNA (of the insert donor composition) occurs within a nucleotide sequence that encodes a T cell receptor (TCR) Alpha or Delta subunit constant region and insertion of the sequence of the other donor DNA (of the insert donor composition) occurs within a nucleotide sequence that encodes a T cell receptor (TCR) Beta or Gamma subunit constant region.

In some embodiments, insertion of a sequence of the donor DNA (of the insert donor composition) occurs within a nucleotide sequence that functions as a T cell receptor (TCR) Alpha or Delta subunit promoter. In some cases insertion of a sequence of the donor DNA (of the insert donor composition) occurs within a nucleotide sequence that functions as a T cell receptor (TCR) Beta or Gamma subunit promoter. In some cases a subject method and/or composition includes two donor DNAs. In some such cases insertion of one sequence of the donor DNA (of the insert donor composition) occurs within a nucleotide sequence that functions as a T cell receptor (TCR) Alpha or Delta subunit promoter and insertion of the sequence of the other donor DNA (of the insert donor composition) occurs within a nucleotide sequence that functions as a T cell receptor (TCR) Beta or Gamma subunit promoter.

In some embodiments, insertion of a sequence of the donor DNA (of the insert donor composition) occurs within a nucleotide sequence that encodes a T cell receptor (TCR) Alpha or Gamma subunit. In some cases, insertion of a sequence of the donor DNA (of the insert donor composition) occurs within a nucleotide sequence that encodes a TCR Beta or Delta subunit. In some cases a subject method and/or composition includes two donor DNAs. In some such cases insertion of one sequence of the donor DNA (of the insert donor composition) occurs within a nucleotide sequence that encodes a T cell receptor (TCR) Alpha or Gamma subunit and insertion of the sequence of the other donor DNA (of the insert donor composition) occurs within a nucleotide sequence that encodes a T cell receptor (TCR) Beta or Delta subunit.

In some embodiments, insertion of a sequence of the donor DNA (of the insert donor composition) occurs within a nucleotide sequence that encodes a T cell receptor (TCR) Alpha or Gamma subunit constant region. In some cases insertion of a sequence of the donor DNA (of the insert donor composition) occurs within a nucleotide sequence that encodes a T cell receptor (TCR) Beta or Delta subunit constant region. In some cases a subject method and/or composition includes two donor DNAs. In some such cases insertion of one sequence of the donor DNA (of the insert donor composition) occurs within a nucleotide sequence that encodes a T cell receptor (TCR) Alpha or Gamma subunit constant region and insertion of the sequence of the other donor DNA (of the insert donor composition) occurs within a nucleotide sequence that encodes a T cell receptor (TCR) Beta or Delta subunit constant region.

In some embodiments, insertion of a sequence of the donor DNA (of the insert donor composition) occurs within a nucleotide sequence that functions as a T cell receptor (TCR) Alpha or Gamma subunit promoter. In some cases insertion of a sequence of the donor DNA (of the insert donor composition) occurs within a nucleotide sequence that functions as a T cell receptor (TCR) Beta or Delta subunit promoter. In some cases a subject method and/or composition includes two donor DNAs. In some such cases insertion of one sequence of the donor DNA (of the insert donor composition) occurs within a nucleotide sequence that functions as a T cell receptor (TCR) Alpha or Gamma subunit promoter and insertion of the sequence of the other donor DNA (of the insert donor composition) occurs within a nucleotide sequence that functions as a T cell receptor (TCR) Beta or Delta subunit promoter.

In some embodiment, insertion of a sequence of the donor DNA (of the insert donor composition) results in operable linkage of the inserted sequence with a T cell receptor (TCR) Alpha, Beta, Gamma or Delta endogenous promoter. In some cases, the inserted sequence includes a protein-coding nucleotide sequence that is operably linked to a TCR Alpha, Beta, Gamma or Delta promoter such that after insertion, the protein-coding sequence will remain operably linked to (under the control of) the promoter present in the donor DNA. In some cases insertion of said sequence results in operable linkage of the inserted sequence (e.g., a protein-coding nucleotide sequence such as a CAR, TCR-alpha, TCR-beta, TCR-gamma, or TCR-Delta sequence) with a CD3 or CD28 promoter. In some cases the inserted sequence of the donor DNA (of the insert donor composition) includes a protein-coding nucleotide sequence that is operably linked to a promoter (e.g., a T-cell specific promoter). In some cases insertion of the sequence of the donor DNA (of the insert donor composition) results in operable linkage of the inserted sequence with an endogenous promoter (e.g., a stem cell specific or somatic cell specific endogenous promoter). In some cases the inserted sequence of the donor DNA (of the insert donor composition) includes a nucleotide sequence that encodes a reporter protein (e.g., fluorescent protein such as GFP, RFP, YFP, CFP, a near-IR and/or far red reporter protein, etc., e.g., for evaluating gene editing efficiency). In some cases the inserted sequence includes a protein-coding nucleotide sequence (e.g., one that encodes all or a portion of a TCR protein) that does not have introns.

In some cases a subject method (and/or subject compositions) can be used for insertion of sequence for applications such as insertion of fluorescent reporters (e.g., a fluorescent protein such green fluorescent protein (GFP)/red fluorescent protein (RFP)/near-IR/far-red, and the like), e.g., into the C- and/or N-termini of any encoded protein of interest such as transmembrane proteins.

### Co-Delivery (not Necessarily a Nanoparticle of the Disclosure)

As noted above, one advantage of delivering multiple payloads as part of the same package (delivery vehicle) is that the efficiency of each payload is not diluted. In some embodiments a first donor DNA, one or more site specific nucleases (or one or more nucleic acids encoding same), a second donor DNA, and a site specific recombinase (or a nucleic acid encoding same) are payloads of the same delivery vehicle. In some embodiments, a donor DNA and/or one or more gene editing tools (e.g., as described elsewhere herein) is delivered in combination with (e.g., as part of the same package/delivery vehicle, where the delivery vehicle does not need to be a nanoparticle of the disclosure) a protein (and/or a DNA or mRNA encoding same) and/or a non-coding RNA that increases genomic editing efficiency. In some embodiments, one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls cell division and/or differentiation. For example, in some cases one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls cell division. In some cases one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls differentiation. In some cases, one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that biases the cell DNA repair machinery.

As noted above, in some cases the delivery vehicle does not need to be a nanoparticle of the disclosure. For example, in some cases the delivery vehicle is viral and in some cases the delivery vehicle is non-viral. Examples of non-viral delivery systems include materials that can be used to co-condense multiple nucleic acid payloads, or combinations of protein and nucleic acid payloads. Examples include, but are not limited to: (1) lipid based particles such as zwitterionic or cationic lipids, and exosome or exosome-derived vesicles; (2) inorganic/hybrid composite particles such as those that include ionic complexes co-condensed with nucleic acids and/or protein payloads, and complexes that can be condensed from cationic ionic states of Ca, Mg, Si, Fe and physiological anions such as O²⁻, OH, PO₄³⁻, SO₄²⁻; (3) carbohydrate delivery vehicles such as cyclodextrin and/or alginate; (4) polymeric and/or co-polymeric complexes such as poly(amino-acid) based electrostatic complexes, poly(Amido-Amine), and cationic poly(B-Amino Ester); and (5) virus like particles (e.g., protein and nucleic acid based). Examples of viral delivery systems include but are not limited to: AAV, adenoviral, retroviral, and lentiviral.

### Examples of Payloads for Co-Delivery

In some embodiments the payload components described herein can be delivered in combination with (e.g., as part of the same package/delivery vehicle, where the delivery vehicle does not need to be a nanoparticle of the disclosure) one or more of: SCF (and/or a DNA or mRNA encoding SCF), HoxB4 (and/or a DNA or mRNA encoding HoxB4), BCL-XL (and/or a DNA or mRNA encoding BCL-XL), SIRT6 (and/or a DNA or mRNA encoding SIRT6), a nucleic acid molecule (e.g., an siRNA and/or an LNA) that suppresses miR-155, a nucleic acid molecule (e.g., an siRNA, an shRNA, a microRNA) that reduces ku70 expression, and a nucleic acid molecule (e.g., an siRNA, an shRNA, a microRNA) that reduces ku80 expression.

For examples of microRNAs (delivered as RNAs or as DNA encoding the RNAs) that can be delivered together, see FIG. 9. For example, the following microRNAs can be used for the following purposes: for blocking differentiation of a pluripotent stem cell toward ectoderm lineage: miR-430/427/302; for blocking differentiation of a pluripotent stem cell toward endoderm lineage: miR-109 and/or miR-24; for driving differentiation of a pluripotent stem cell toward endoderm lineage: miR-122 and/or miR-192; for driving differentiation of an ectoderm progenitor cell toward a keratinocyte fate: miR-203; for driving differentiation of a neural crest stem cell toward a smooth muscle fate: miR-145; for driving differentiation of a neural stem cell toward a glial cell fate and/or toward a neuron fate: miR-9 and/or miR-124a; for blocking differentiation of a mesoderm progenitor cell toward a chondrocyte fate: miR-199a; for driving differentiation of a mesoderm progenitor cell toward an osteoblast fate: miR-296 and/or miR-2861; for driving differentiation of a mesoderm progenitor cell toward a cardiac muscle fate: miR-1; for blocking differentiation of a mesoderm progenitor cell toward a cardiac muscle fate: miR-133; for driving differentiation of a mesoderm progenitor cell toward a skeletal muscle fate: miR-214, miR-206, miR-1 and/or miR-26a; for blocking differentiation of a mesoderm progenitor cell toward a skeletal muscle fate: miR-133, miR-221, and/or miR-222; for driving differentiation of a hematopoietic progenitor cell toward differentiation: miR-223; for blocking differentiation of a hematopoietic progenitor cell toward differentiation: miR-128a and/or miR-181a; for driving differentiation of a hematopoietic progenitor cell toward a lymphoid progenitor cell: miR-181; for blocking differentiation of a hematopoietic progenitor cell toward a lymphoid progenitor cell: miR-146; for blocking differentiation of a hematopoietic progenitor cell toward a myeloid progenitor cell: miR-155, miR-24a, and/or miR-17; for driving differentiation of a lymphoid progenitor cell toward a T cell fate: miR-150; for blocking differentiation of a myeloid progenitor cell toward a granulocyte fate: miR-223; for blocking differentiation of a myeloid progenitor cell toward a monocyte fate: miR-17-5p, miR-20a, and/or miR-106a; for blocking differentiation of a myeloid progenitor cell toward a red blood cell fate: miR-150, miR-155, miR-221, and/or miR-222; and for driving differentiation of a myeloid progenitor cell toward a red blood cell fate: miR-451 and/or miR-16.

For examples of signaling proteins (e.g., extracellular signaling proteins) that can be delivered together with the components described herein (e.g., first and second donor DNAs, one or more gene editing tools (e.g., as described elsewhere herein), and a sequence specific recombinase—or a nucleic acid encoding same), see FIG. 10. For example, the following signaling proteins (e.g., extracellular signaling proteins) (e.g., delivered as protein or as a nucleic acid such as DNA or RNA encoding the protein) can be used for the following purposes: for driving differentiation of a hematopoietic stem cell toward a common lymphoid progenitor cell lineage: IL-7; for driving differentiation of a hematopoietic stem cell toward a common myeloid progenitor cell lineage: IL-3, GM-CSF, and/or M-CSF; for driving differentiation of a common lymphoid progenitor cell toward a B-cell fate: IL-3, IL-4, and/or IL-7; for driving differentiation of a common lymphoid progenitor cell toward a Natural Killer Cell fate: IL-15; for driving differentiation of a common lymphoid progenitor cell toward a T-cell fate: IL-2, IL-7, and/or Notch; for driving differentiation of a common lymphoid progenitor cell toward a dendritic cell fate: Flt-3 ligand; for driving differentiation of a common myeloid progenitor cell toward a dendritic cell fate: Flt-3 ligand, GM-CSF, and/or TNF-alpha; for driving differentiation of a common myeloid progenitor cell toward a granulocyte-macrophage progenitor cell lineage: GM-CSF; for driving differentiation of a common myeloid progenitor cell toward a megakaryocyte-erythroid progenitor cell lineage: IL-3, SCF, and/or Tpo; for driving differentiation of a megakaryocyte-erythroid progenitor cell toward a megakaryocyte fate: IL-3, IL-6, SCF, and/or Tpo; for driving differentiation of a megakaryocyte-erythroid progenitor cell toward a erythrocyte fate: erythropoietin; for driving differentiation of a megakaryocyte toward a platelet fate: IL-11 and/or Tpo; for driving differentiation of a granulocyte-macrophage progenitor cell toward a monocyte lineage: GM-CSF and/or M-CSF; for driving differentiation of a granulocyte-macrophage progenitor cell toward a myeloblast lineage: GM-CSF; for driving differentiation of a monocyte toward a monocyte-derived dendritic cell fate: Flt-3 ligand, GM-CSF, IFN-alpha, and/or IL-4; for driving differentiation of a monocyte toward a macrophage fate: IFN-gamma, IL-6, IL-10, and/or M-CSF; for driving differentiation of a myeloblast toward a neutrophil fate: G-CSF, GM-CSF, IL-6, and/or SCF; for driving differentiation of a myeloblast toward a eosinophil fate: GM-CSF, IL-3, and/or IL-5; and for driving differentiation of a myeloblast toward a basophil fate: G-CSF, GM-CSF, and/or IL-3.

Examples of proteins that can be delivered (e.g., as protein and/or a nucleic acid such as DNA or RNA encoding the protein) together with the components described herein include but are not limited to: SOX17, HEX, OSKM (Oct4/Sox2/Klf4/c-myc), and/or bFGF (e.g., to drive differentiation toward hepatic stem cell lineage); HNF4a (e.g., to drive differentiation toward hepatocyte fate); Poly (I:C), BMP-4, bFGF, and/or 8-Br-cAMP (e.g., to drive differentiation toward endothelial stem cell/progenitor lineage); VEGF (e.g., to drive differentiation toward arterial endothelium fate); Sox-2, Brn4, Myt11, Neurod2, Ascl1 (e.g., to drive differentiation toward neural stem cell/progenitor lineage); and BDNF, FCS, Forskolin, and/or SHH (e.g., to drive differentiation neuron, astrocyte, and/or oligodendrocyte fate).

Examples of signaling proteins (e.g., extracellular signaling proteins) that can be delivered (e.g., as protein and/or a nucleic acid such as DNA or RNA encoding the protein) together with the components described herein include but are not limited to: cytokines (e.g., IL-2 and/or IL-15, e.g., for activating CD8+ T-cells); ligands and or signaling proteins that modulate one or more of the Notch, Wnt, and/or Smad signaling pathways; SCF; stem cell programming factors (e.g. Sox2, Oct3/4, Nanog, Klf4, c-Myc, and the like); and temporary surface marker “tags” and/or fluorescent reporters for subsequent isolation/purification/concentration. For example, a fibroblast may be converted into a neural stem cell via delivery of Sox2, while it will turn into a cardiomyocyte in the presence of Oct3/4 and small molecule “epigenetic resetting factors.” In a patient with Huntington's disease or a CXCR4 mutation, these fibroblasts may respectively encode diseased phenotypic traits associated with neurons and cardiac cells. By delivering gene editing corrections and these factors in a single package, the risk of deleterious effects due to one or more, but not all of the factors/payloads being introduced can be significantly reduced.

Applications include in vivo approaches wherein a cell death cue may be conditional upon a gene edit not being successful, and cell differentiation/proliferation/activation is tied to a tissue/organ-specific promoter and/or exogenous factor. A diseased cell receiving a gene edit may activate and proliferate, but due to the presence of another promoter-driven expression cassette (e.g. one tied to the absence of tumor suppressor such as p21 or p53), those cells will subsequently be eliminated. The cells expressing desired characteristics, on the other hand, may be triggered to further differentiate into the desired downstream lineages.

### Kits

Also within the scope of the disclosure are kits. For example, in some cases a subject kit can include one or more of (in any combination): (i) a first donor DNA (described elsewhere herein); (ii) one or more site specific nucleases (or one or more nucleic acids encoding same) such as a ZFN pair, a TALEN pair, a nickase Ca9, a Cpf1, etc.; (iii) a second donor DNA (described elsewhere herein); (iv) a sequence specific recombinase (or nucleic acid encoding same); (v) a targeting ligand, (vi) a linker, (vii) a targeting ligand conjugated to a linker, (viii) a targeting ligand conjugated to an anchoring domain (e.g., with or without a linker), (ix) an agent for use as a sheddable layer (e.g., silica), (x) an additional payload, e.g., an siRNA or a transcription template for an siRNA or shRNA; a gene editing tool, and the like, (xi) a polymer that can be used as a cationic polymer, (xii) a polymer that can be used as an anionic polymer, (xiii) a polypeptide that can be used as a cationic polypeptide, e.g., one or more HTPs, and (xiv) a subject viral or non-viral delivery vehicle. In some cases, a subject kit can include instructions for use. Kits typically include a label indicating the intended use of the contents of the kit. The term label includes any writing, or recorded material, e.g., computer-readable media, supplied on or with the kit, or which otherwise accompanies the kit.

### First Illustrative Example of Nanoparticle Synthesis

Procedures were performed within a sterile, dust free environment (BSL-II hood). Gastight syringes were sterilized with 70% ethanol before rinsing 3 times with filtered nuclease free water, and were stored at 4° C. before use. Surfaces were treated with RNAse inhibitor prior to use.

Nanoparticle Core

A first solution (an anionic solution) was prepared by combining the appropriate amount of payload (in this case plasmid DNA (EGFP-N1 plasmid) with an aqueous mixture (an ‘anionic polymer composition’) of poly(D-glutamic Acid) and poly(L-glutamic acid). This solution was diluted to the proper volume with 10 mM Tris-HCl at pH 8.5. A second solution (a cationic solution), which was a combination of a ‘cationic polymer composition’ and a ‘cationic polypeptide composition’, was prepared by diluting a concentrated solution containing the appropriate amount of condensing agents to the proper volume with 60 mM HEPES at pH 5.5. In this case, the ‘cationic polymer composition’ was poly(L-arginine) and the ‘cationic polypeptide composition’ was 16 μg of H3K4(me3) (tail of histone H3, tri methylated on K4).

Precipitation of nanoparticle cores in batches less than 200 μl can be carried out by dropwise addition of the condensing solution to the payload solution in glass vials or low protein binding centrifuge tubes followed by incubation for 30 minutes at 4° C. For batches greater than 200 μl, the two solutions can be combined in a microfluidic format (e.g., using a standard mixing chip (e.g. Dolomite Micromixer) or a hydrodynamic flow focusing chip). Optimal input flowrates can be determined such that the resulting suspension of nanoparticle cores is monodispersed, exhibiting a mean particle size below 100 nm.

In this case, the two equal volume solutions from above (one of cationic condensing agents and one of anionic condensing agents) were prepared for mixing. For the solution of cationic condensing agents, polymer/peptide solutions were added to one protein low bind tube (eppendorf) and were then diluted with 60 mM HEPES (pH 5.5) to a total volume of 100 μl (as noted above). This solution was kept at room temperature while preparing the anionic solution. For the solution of anionic condensing agents, the anionic solutions were chilled on ice with minimal light exposure. 10 μg of nucleic acid in aqueous solution (roughly 1 μg/μl) and 7 μg of aqueous poly (D-Glutamic Acid) [0.1%] were diluted with 10 mM Tris-HCl (pH 8.5) to a total volume of 100 μl (as noted above).

Each of the two solutions was filtered using a 0.2 micron syringe filter and transferred to its own Hamilton 1 ml Gastight Syringe (Glass, (insert product number). Each syringe was placed on a Harvard Pump 11 Elite Dual Syringe Pump. The syringes were connected to appropriate inlets of a Dolomite Micro Mixer chip using tubing, and the syringe pump was run at 120 μl/min for a 100 μl total volume. The resulting solution included the core composition (which now included nucleic acid payload, anionic components, and cationic components).

### Core Stabilization (Adding a Sheddable Layer)

To coat the core with a sheddable layer, the resulting suspension of nanoparticle cores was then combined with a dilute solution of sodium silicate in 10 mM Tris HCl (pH8.5, 10-500 mM) or calcium chloride in 10 mM PBS (pH 8.5, 10-500 mM), and allowed to incubate for 1-2 hours at room temperature. In this case, the core composition was added to a diluted sodium silicate solution to coat the core with an acid labile coating of polymeric silica (an example of a sheddable layer). To do so, 10 μl of stock Sodium Silicate (Sigma) was first dissolved in 1.99 ml of Tris buffer (10 mM Tris pH=8.5, 1:200 dilution) and was mixed thoroughly. The Silicate solution was filtered using a sterile 0.1 micron syringe filter, and was transferred to a sterile Hamilton Gastight syringe, which was mounted on a syringe pump. The core composition from above was also transferred to a sterile Hamilton Gastight syringe, which was also mounted on the syringe pump. The syringes were connected to the appropriate inlets of a Dolomite Micro Mixer chip using PTFE tubing, and the syringe pump was run at 120 μl/min.

Stabilized (coated) cores can be purified using standard centrifugal filtration devices (100 kDa Amicon Ultra, Millipore) or dialysis in 30 mM HEPES (pH 7.4) using a high molecular weight cutoff membrane. In this case, the stabilized (coated) cores were purified using a centrifugal filtration device. The collected coated nanoparticles (nanoparticle solution) were washed with dilute PBS (1:800) or HEPES and filtered again (the solution can be resuspended in 500 μl sterile dispersion buffer or nuclease free water for storage). Effective silica coating was demonstrated. The stabilized cores had a size of 110.6 nm and zeta potential of −42.1 mV (95%).

### Surface Coat (Outer Shell)

Addition of a surface coat (also referred to as an outer shell), sometimes referred to as “surface functionalization,” was accomplished by electrostatically grafting ligand species (in this case Rabies Virus Glycoprotein fused to a 9-Arg peptide sequence as a cationic anchoring domain—‘RVG9R’) to the negatively charged surface of the stabilized (in this case silica coated) nanoparticles. Beginning with silica coated nanoparticles that were filtered and resuspended in dispersion buffer or water, the final volume of each nanoparticle dispersion was determined, as was the desired amount of polymer or peptide to add such that the final concentration of protonated amine group was at least 75 uM. The desired surface constituents were added and the solution was sonicated for 20-30 seconds prior to incubate for 1 hour. Centrifugal filtration was performed at 300 kDa (the final product can be purified using standard centrifugal filtration devices, e.g., 300-500 kDa from Amicon Ultra Millipore, or dialysis, e.g., in 30 mM HEPES (pH 7.4) using a high molecular weight cutoff membrane), and the final resuspension was in either cell culture media or dispersion buffer. In some cases, optimal outer shell addition yields a monodispersed suspension of particles with a mean particle size between 50 and 150 nm and a zeta potential between 0 and −10 mV. In this case, the nanoparticles with an outer shell had a size of 115.8 nm and a Zeta potential of −3.1 mV (100%).

### Second Illustrative Example of Nanoparticle Synthesis

Nanoparticles were synthesized at room temperature, 37C or a differential of 37C and room temperature between cationic and anionic components. Solutions were prepared in aqueous buffers utilizing natural electrostatic interactions during mixing of cationic and anionic components. At the start, anionic components were dissolved in Tris buffer (30 mM-60 mM; pH=7.4-9) or HEPES buffer (30 mM, pH=5.5) while cationic components were dissolved in HEPES buffer (30 mM-60 mM, pH=5-6.5).

Specifically, payloads (e.g., genetic material (RNA or DNA), genetic material-protein-nuclear localization signal polypeptide complex (ribonucleoprotein), or polypeptide) were reconstituted in a basic, neutral or acidic buffer. For analytical purposes, the payload was manufactured to be covalently tagged with or genetically encode a fluorophore. Wth pDNA payloads, a Cy5-tagged peptide nucleic acid (PNA) specific to AGAGAG tandem repeats was used to fluorescently tag fluorescent reporter vectors and fluorescent reporter-therapeutic gene vectors. A timed-release component that may also serve as a negatively charged condensing species (e.g. poly(glutamic acid)) was also reconstituted in a basic, neutral or acidic buffer. Targeting ligands with a wild-type derived or wild-type mutated targeting peptide conjugated to a linker-anchor sequence were reconstituted in acidic buffer. In the case where additional condensing species or nuclear localization signal peptides were included in the nanoparticle, these were also reconstituted in buffer as 0.03% w/v working solutions for cationic species, and 0.015% w/v for anionic species. Experiments were also conducted with 0.1% w/v working solutions for cationic species and 0.1% w/v for anionic species. All polypeptides, except those complexing with genetic material, were sonicated for ten minutes to improve solubilization.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below in SET A and SET B. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

### Set A

1. A method for inserting a donor sequence into a cell's genome, comprising:
introducing into a cell, a delivery vehicle with a payload comprising:

(a) a nuclease composition, comprising: one or more sequence specific nucleases or one or more nucleic acids that encode the one or more sequence specific nucleases, wherein the one or more sequence specific nucleases cleaves the cell's genome;

(b) a target donor composition, comprising: a first donor DNA, which comprises a nucleotide sequence that is inserted into the cell's genome, wherein insertion of said nucleotide sequence produces, in the cell's genome at the site of insertion, a target sequence for a site-specific recombinase;

(c) a recombinase composition, comprising: the site-specific recombinase, or a nucleic acid encoding the site-specific recombinase, wherein the site-specific recombinase recognizes said target sequence; and

(d) an insert donor composition, comprising: a second donor DNA, which comprises a nucleotide sequence that is inserted into the cell's genome as a result of recognition of said target sequence by the site-specific recombinase.

2. The method of 1, wherein insertion of the nucleotide sequence of the first donor DNA of the target donor composition produces a first target sequence for the site-specific recombinase at a first location in the cell's genome and a second target sequence for the site-specific recombinase at a second location in the cell's genome.
3. The method of 1, wherein the nuclease composition cleaves the cell's genome at two locations, and wherein the target donor composition comprises two of said first donor DNAs, each of which comprises a nucleotide sequence that is inserted into the cell's genome, thereby producing a first target sequence for the site-specific recombinase at a first location in the cell's genome and a second target sequence for the site-specific recombinase at a second location in the cell's genome.
4. The method of 2 or 3, wherein the first and second locations in the cell's genome are separated by 1,000,000 base pairs or less.
5. The method of 2 or 3, wherein the first and second locations in the cell's genome are separated by 100,000 base pairs or less.
6. The method of 2 or 3, wherein the nucleotide sequence, of the insert donor composition, that is inserted into the cell's genome has a length of from 10 base pairs (bp) to 100 kilobase pairs (kbp).
7. The method of any one of 1-6, wherein the second donor DNA comprises two target sequences for the site-specific recombinase, wherein the two target sequences flank the nucleotide sequence that is inserted into the cell's genome.
8. The method of any one of 1-7, wherein the target sequence for the site-specific recombinase is selected from: an attB site, an attP site, an attL site, an attR site, a loxP site, and an FRT site.
9. The method of any one of 1-8, wherein the site-specific recombinase is selected from: ϕC31, ϕC31 RDF, Cre, and FLP.
10. The method of any one of 1-9, wherein at least one of the one or more sequence specific nucleases is selected from: a meganuclease, a homing endonuclease, a zinc finger nuclease (ZFN), and a transcription activator-like effector nuclease (TALEN).
11. The method of any one of 1-9, wherein at least one of the one or more sequence specific nucleases is a Class 2 CRISPR/Cas effector protein.
12. The method of 11, wherein the Class 2 CRISPR/Cas effector protein is selected from Cas9 and cpf1.
13. The method of 11 or 12, wherein the nuclease composition comprises one or more CRISPR/Cas guide nucleic acids or one or more nucleic acids encoding the CRISPR/Cas guide nucleic acids.
14. The method of any one of 1-13, wherein the delivery vehicle is non-viral.
15. The method of any one of 1-14, wherein the delivery vehicle is a nanoparticle.
16. The method of 15, wherein, in addition to the payload, the nanoparticle comprises a core comprising an anionic polymer composition, a cationic polymer composition, and a cationic polypeptide composition.
17. The method of 16, wherein said anionic polymer composition comprises an anionic polymer selected from poly(glutamic acid) and poly(aspartic acid).
18. The method of 16 or 17, wherein said cationic polymer composition comprises a cationic polymer selected from poly(arginine), poly(lysine), poly(histidine), poly(ornithine), and poly(citrulline).
19. The method of any one of 16-18, wherein nanoparticle further comprises a sheddable layer encapsulating the core.
20. The method of 19, wherein the sheddable layer is an anionic coat or a cationic coat.
21. The method of 19 or 20, wherein the sheddable layer comprises one or more of: silica, a peptoid, a polycysteine, calcium, calcium oxide, hydroxyapatite, calcium phosphate, calcium sulfate, manganese, manganese oxide, manganese phosphate, manganese sulfate, magnesium, magnesium oxide, magnesium phosphate, magnesium sulfate, iron, iron oxide, iron phosphate, and iron sulfate.
22. The method of any one of 19-21, wherein the nanoparticle further comprises a surface coat surrounding the sheddable layer.
23. The method of 22, wherein the surface coat comprises a cationic or anionic anchoring domain that interacts electrostatically with the sheddable layer.
24. The method of 22 or 23, wherein the surface coat comprises one or more targeting ligands.
25. The method of 24, wherein the one or more targeting ligands are selected from: a peptide, an ScFv, a F(ab), a nucleic acid aptamer, or a peptoid.
26. The method of 22 or 23, wherein the surface coat comprises one or more targeting ligands selected from the group consisting of: rabies virus glycoprotein (RVG) fragment, ApoE-transferrin, lactoferrin, melanoferritin, ovotransferritin, L-selectin, E-selectin, P-selectin, sialylated peptides, polysialylated O-linked peptides, TPO, EPO, PSGL-1, ESL-1, CD44, death receptor-3 (DR3), LAMP1, LAMP2, Mac2-BP, stem cell factor (SCF), CD70, SH2 domain-containing protein 1A (SH2D1A), a exendin-4, GLP1, RGD, a Transferrin ligand, an FGF fragment, succinic acid, a bisphosphonate, a hematopoietic stem cell chemotactic lipid, sphingosine, ceramide, sphingosine-1-phosphate, ceramide-1-phosphate, and an active targeting fragment of any of the above.
27. The method of 22 or 23, wherein the surface coat comprises one or more targeting ligands that provides for targeted binding to a target selected from: CD3, CD28, CD90, CD45f, CD34, CD80, CD86, CD19, CD20, CD22, CD3-epsilon, CD3-gamma, CD3-delta; TCR Alpha, TCR Beta, TCR gamma, and/or TCR delta constant regions; 4-1 BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL-2, IL-7, IL-10, IL-12, IL-15, IL-18, TNFα, IFNγ, TGF-β, and α5β1.
28. The method of 22 or 23, wherein the surface coat comprises one or more targeting ligands that provides for targeted binding to target cells selected from: bone marrow cells, hematopoietic stem cells (HSCs), hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells, megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, liver cells, muscle cells, skeletal muscle cells, cardiac muscle cells, hepatic cells, fat cells, intestinal cells, cells of the colon, and cells of the stomach.
29. The method of any one of 1-14, wherein the delivery vehicle is a targeting ligand conjugated to the payload, wherein the targeting ligand provides for targeted binding to a cell surface protein.
30. The method of any one of 1-14, wherein the delivery vehicle is a targeting ligand conjugated to a charged polymer polypeptide domain, wherein the targeting ligand provides for targeted binding to a cell surface protein, and wherein the charged polymer polypeptide domain is condensed with a nucleic acid payload and/or is interacting electrostatically with a protein payload.
31. The method of 29 or 30, wherein the targeting ligand is a peptide, an ScFv, a F(ab), a nucleic acid aptamer, or a peptoid.
32. The method of 30, wherein the charged polymer polypeptide domain has a length in a range of from 3 to 30 amino acids.
33. The method of any one of 30-32, wherein the delivery vehicle further comprises an anionic polymer interacting with the payload and the charged polymer polypeptide domain.
34. The method of 33, wherein the anionic polymer is selected from poly(glutamic acid) and poly(aspartic acid).
35. The method of any one of 29-34, wherein the targeting ligand has a length of from 5-50 amino acids.
36. The method of any one of 29-35, wherein the targeting ligand provides for targeted binding to a cell surface protein selected from a family B G-protein coupled receptor (GPCR), a receptor tyrosine kinase (RTK), a cell surface glycoprotein, and a cell-cell adhesion molecule.
37. The method of any one of 29-35, wherein the targeting ligand is selected from the group consisting of: rabies virus glycoprotein (RVG) fragment, ApoE-transferrin, lactoferrin, melanoferritin, ovotransferritin, L-selectin, E-selectin, P-selectin, sialylated peptides, polysialylated O-linked peptides, TPO, EPO, PSGL-1, ESL-1, CD44, death receptor-3 (DR3), LAMP1, LAMP2, Mac2-BP, stem cell factor (SCF), CD70, SH2 domain-containing protein 1A (SH2D1A), a exendin-4, GLP1, RGD, a Transferrin ligand, an FGF fragment, succinic acid, a bisphosphonate, a hematopoietic stem cell chemotactic lipid, sphingosine, ceramide, sphingosine-1-phosphate, ceramide-1-phosphate, and an active targeting fragment of any of the above.
38. The method of any one of 29-35, wherein the targeting ligand provides for targeted binding to a target selected from: CD3, CD28, CD90, CD45f, CD34, CD80, CD86, CD19, CD20, CD22, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL-2, IL-7, IL-10, IL-12, IL-15, IL-18, TNFα, IFNγ, TGF-β, and α5β1.
39. The method of any one of 29-35, wherein the targeting ligand provides for binding to a cell type selected from the group consisting of: bone marrow cells, hematopoietic stem cells (HSCs), long-term HSCs, short-term HSCs, hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, muscle cells, skeletal muscle cells, cardiac muscle cells, hepatic cells, fat cells, intestinal cells, cells of the colon, and cells of the stomach.
40. The method of any one of 1-39, wherein insertion of the nucleotide sequence of the second donor DNA into the cell's genome results in operable linkage of the inserted sequence with an endogenous promoter.
41. The method of 40, wherein the endogenous promoter is selected from the group consisting of: (i) a T-cell specific promoter; (ii) a CD3 promoter; (iii) a CD28 promoter; (iv) a stem cell specific promoter; (v) a somatic cell specific promoter; and (vi) a T cell receptor (TCR) Alpha, Beta, Gamma or Delta promoter.
42. The method of any one of 1-39, wherein the nucleotide sequence, of the insert donor composition, that is inserted includes a protein-coding sequence that is operably linked to a promoter.
43. The method of 42, wherein the promoter is selected from the group consisting of: (i) a T-cell specific promoter; (ii) a CD3 promoter; (iii) a CD28 promoter; (iv) a stem cell specific promoter; (v) a somatic cell specific promoter; and (vi) a T cell receptor (TCR) Alpha, Beta, Gamma or Delta promoter.
44. The method of any one of 1-43, wherein the nucleotide sequence, of the second donor DNA, that is inserted into the cell's genome encodes a T cell receptor (TCR) protein.
45. The method of any one of 1-43, wherein the nucleotide sequence, of the second donor DNA, that is inserted into the cell's genome encodes a CDR1, CDR2, or CDR3 region of a T cell receptor (TCR) protein.
46. The method of any one of 1-43, wherein the nucleotide sequence, of the second donor DNA, that is inserted into the cell's genome encodes a chimeric antigen receptor (CAR).
47. The method of 46, wherein insertion of the nucleotide sequence that encodes the CAR results in operable linkage of the nucleotide sequence that encodes the CAR with an endogenous T-cell specific promoter.
48. The method of any one of 1-43, wherein the nucleotide sequence, of the second donor DNA, that is inserted into the cell's genome encodes a multivalent surface receptor.
49. The method of 48, wherein the cell is a T-cell.
50. The method of 48 or 49, wherein the multivalent surface receptor is a bispecific or trispecific chimeric antigen receptor (CAR) or T cell receptor (TCR).
51. The method of any one of 1-43, wherein the nucleotide sequence, of the second donor DNA, that is inserted into the cell's genome encodes a cell-specific targeting ligand that is membrane bound and presented extracellularly.
52. The method of any one of 1-43, wherein the nucleotide sequence, of the second donor DNA, that is inserted into the cell's genome encodes a reporter protein.
53. The method of 52, wherein the reporter protein is a fluorescent protein.
54. The method of 52 or 53, wherein the nucleotide sequence that encodes the reporter protein is operably linked to a cell-specific or tissue-specific promoter.
55. The method of 52 or 53, wherein the nucleotide sequence that encodes the reporter protein is operably linked to a constitutive promoter.
56. The method of any one of 1-55, wherein the nucleotide sequence, of the second donor DNA, that is inserted into the cell's genome includes a protein-coding nucleotide sequence that does not have introns.
57. The method of 56, wherein the nucleotide sequence that does not have introns encodes all or a portion of a TCR protein.
58. The method of any one of 1-57, wherein the method comprises introducing a first and a second of said delivery vehicles into the cell,

wherein the nucleotide sequence of the second donor DNA of the first delivery vehicle, that is inserted into the cell's genome, encodes a T cell receptor (TCR) Alpha or Delta subunit, and

wherein the nucleotide sequence of the second donor DNA of the second delivery vehicle, that is inserted into the cell's genome, encodes a TCR Beta or Gamma subunit.

59. The method of any one of 1-57, wherein the method comprises introducing a first and a second of said delivery vehicles into the cell,

wherein the nucleotide sequence of the second donor DNA of the first delivery vehicle, that is inserted into the cell's genome, encodes a T cell receptor (TCR) Alpha or Delta subunit constant region, and

wherein the nucleotide sequence of the second donor DNA of the second delivery vehicle, that is inserted into the cell's genome, encodes a TCR Beta or Gamma subunit constant region.

60. The method of any one of 1-57, wherein the method comprises introducing a first and a second of said delivery vehicles into the cell,

wherein the nucleotide sequence of the second donor DNA of the first delivery vehicle is inserted within a nucleotide sequence that functions as a T cell receptor (TCR) Alpha or Delta subunit promoter, and

wherein the nucleotide sequence of the second donor DNA of the second delivery vehicle is inserted within a nucleotide sequence that functions as a TCR Beta or Gamma subunit promoter.

61. The method of any one of 1-60, wherein the cell is a mammalian cell.
62. The method of any one of 1-61, wherein the cell is a human cell.
63. A composition comprising:

(a) a nuclease composition, comprising: one or more sequence specific nucleases or one or more nucleic acids that encode the one or more sequence specific nucleases;

(b) a target donor composition, comprising: a first donor DNA that comprises a target sequence for a site-specific recombinase;

(c) a recombinase composition, comprising: the site-specific recombinase, or a nucleic acid encoding the site-specific recombinase, wherein the site-specific recombinase recognizes said target sequence; and

(d) an insert donor composition, comprising: a second donor DNA, which comprises a nucleotide sequence of interest for insertion into a target cell's genome;

wherein (a), (b), (c), and (d) are payloads as part of the same delivery vehicle.

64. The composition of 63, wherein the delivery vehicle is a nanoparticle.
65. The composition of 64, wherein the nanoparticle comprises a core comprising (a), (b), an anionic polymer composition, a cationic polymer composition, and a cationic polypeptide composition.
66. The composition of 64 or 65, wherein the nanoparticle comprises a targeting ligand that targets the nanoparticle to a cell surface protein.
67. The composition of any one of 63-66, wherein the payloads form one or more deoxyribonucleoprotein complexes or one or more ribo-deoxyribonucleoprotein complexes.
68. The composition of any one of 63-67, wherein the delivery vehicle is a targeting ligand conjugated to a charged polymer polypeptide domain, wherein the targeting ligand provides for targeted binding to a cell surface protein, and wherein the charged polymer polypeptide domain is interacting electrostatically with one or more of the payloads.
69. The composition of 68, wherein the delivery vehicle further comprises an anionic polymer interacting with one or more of the payloads and the charged polymer polypeptide domain.
70. The composition of any one of 63-67, wherein the delivery vehicle is a targeting ligand conjugated one or more of the payloads, wherein the targeting ligand provides for targeted binding to a cell surface protein.
71. The composition of any one of 63-67, herein the delivery vehicle includes a targeting ligand coated upon a water-oil-water emulsion particle, upon an oil-water emulsion micellar particle, upon a multilamellar water-oil-water emulsion particle, upon a multilayered particle, or upon a DNA origami nanobot.
72. The method of any one of 66-71, wherein the targeting ligand is a peptide, an ScFv, a F(ab), a nucleic acid aptamer, or a peptoid.
73. The composition of any one of 63-67, wherein the delivery vehicle is non-viral.

### Set B

1. A method for inserting a donor sequence into a cell's genome, comprising:
introducing into a cell, a delivery vehicle with a payload comprising:

(a) a nuclease composition, comprising: one or more sequence specific nucleases or one or more nucleic acids that encode the one or more sequence specific nucleases, wherein the one or more sequence specific nucleases cleaves the cell's genome;

(b) a target donor composition, comprising: a first donor DNA, which comprises a nucleotide sequence that is inserted into the cell's genome, wherein insertion of said nucleotide sequence produces, in the cell's genome at the site of insertion, a target sequence for a site-specific recombinase;

(c) a recombinase composition, comprising: the site-specific recombinase, or a nucleic acid encoding the site-specific recombinase, wherein the site-specific recombinase recognizes said target sequence; and

(d) an insert donor composition, comprising: a second donor DNA, which comprises a nucleotide sequence that is inserted into the cell's genome as a result of recognition of said target sequence by the site-specific recombinase.

2. The method of 1, wherein insertion of the nucleotide sequence of the first donor DNA of the target donor composition produces a first target sequence for the site-specific recombinase at a first location in the cell's genome and a second target sequence for the site-specific recombinase at a second location in the cell's genome.
3. The method of 1, wherein the nuclease composition cleaves the cell's genome at two locations, and wherein the target donor composition comprises two of said first donor DNAs, each of which comprises a nucleotide sequence that is inserted into the cell's genome, thereby producing a first target sequence for the site-specific recombinase at a first location in the cell's genome and a second target sequence for the site-specific recombinase at a second location in the cell's genome.
4. The method of 2 or 3, wherein the first and second locations in the cell's genome are separated by 1,000,000 base pairs or less.
5. The method of 2 or 3, wherein the first and second locations in the cell's genome are separated by 100,000 base pairs or less.
6. The method of 2 or 3, wherein the nucleotide sequence, of the insert donor composition, that is inserted into the cell's genome has a length of from 10 base pairs (bp) to 100 kilobase pairs (kbp).
7. The method of any one of 1-6, wherein the second donor DNA comprises two target sequences for the site-specific recombinase, wherein the two target sequences flank the nucleotide sequence that is inserted into the cell's genome.
8. The method of any one of 1-7, wherein the target sequence for the site-specific recombinase is selected from: an attB site, an attP site, an attL site, an attR site, a loxP site, and an FRT site.
9. The method of any one of 1-8, wherein the site-specific recombinase is selected from: ϕC31, ϕC31 RDF, Cre, and FLP.
10. The method of any one of 1-9, wherein at least one of the one or more sequence specific nucleases is selected from: a meganuclease, a homing endonuclease, a zinc finger nuclease (ZFN), and a transcription activator-like effector nuclease (TALEN).
11. The method of any one of 1-9, wherein at least one of the one or more sequence specific nucleases is a Class 2 CRISPR/Cas effector protein.
12. The method of 11, wherein the Class 2 CRISPR/Cas effector protein is selected from Cas9 and cpf1.
13. The method of 11 or 12, wherein the nuclease composition comprises one or more CRISPR/Cas guide nucleic acids or one or more nucleic acids encoding the CRISPR/Cas guide nucleic acids.
14. The method of any one of 1-13, wherein the delivery vehicle is non-viral.
15. The method of any one of 1-14, wherein the delivery vehicle is a nanoparticle.
16. The method of 15, wherein, in addition to the payload, the nanoparticle comprises a core comprising an anionic polymer composition, a cationic polymer composition, and a cationic polypeptide composition.
17. The method of 16, wherein said anionic polymer composition comprises an anionic polymer selected from poly(glutamic acid) and poly(aspartic acid).
18. The method of 16 or 17, wherein said cationic polymer composition comprises a cationic polymer selected from poly(arginine), poly(lysine), poly(histidine), poly(ornithine), and poly(citrulline).
19. The method of any one of 16-18, wherein nanoparticle further comprises a sheddable layer encapsulating the core.
20. The method of 19, wherein the sheddable layer is an anionic coat or a cationic coat.
21. The method of 19 or 20, wherein the sheddable layer comprises one or more of: silica, a peptoid, a polycysteine, calcium, calcium oxide, hydroxyapatite, calcium phosphate, calcium sulfate, manganese, manganese oxide, manganese phosphate, manganese sulfate, magnesium, magnesium oxide, magnesium phosphate, magnesium sulfate, iron, iron oxide, iron phosphate, and iron sulfate.
22. The method of any one of 19-21, wherein the nanoparticle further comprises a surface coat surrounding the sheddable layer.
23. The method of 22, wherein the surface coat comprises a cationic or anionic anchoring domain that interacts electrostatically with the sheddable layer.
24. The method of 22 or 23, wherein the surface coat comprises one or more targeting ligands.
25. The method of 24, wherein the one or more targeting ligands are selected from: a peptide, an ScFv, a F(ab), a nucleic acid aptamer, or a peptoid.
26. The method of 22 or 23, wherein the surface coat comprises one or more targeting ligands selected from the group consisting of: rabies virus glycoprotein (RVG) fragment, ApoE-transferrin, lactoferrin, melanoferritin, ovotransferritin, L-selectin, E-selectin, P-selectin, sialylated peptides, polysialylated O-linked peptides, TPO, EPO, PSGL-1, ESL-1, CD44, death receptor-3 (DR3), LAMP1, LAMP2, Mac2-BP, stem cell factor (SCF), CD70, SH2 domain-containing protein 1A (SH2D1A), a exendin-4, GLP1, RGD, a Transferrin ligand, an FGF fragment, succinic acid, a bisphosphonate, a hematopoietic stem cell chemotactic lipid, sphingosine, ceramide, sphingosine-1-phosphate, ceramide-1-phosphate, IL2, CD80, CD86, CD8 epsilon, peptide-HLA-A*2402, and an active targeting fragment of any of the above.
27. The method of 22 or 23, wherein the surface coat comprises one or more targeting ligands that provides for targeted binding to a target selected from: CD3, CD8, CD4, CD28, CD90, CD45f, CD34, CD80, CD86, CD19, CD20, CD22, CD47, CD3-epsilon, CD3-gamma, CD3-delta; TCR Alpha, TCR Beta, TCR gamma, and/or TCR delta constant regions; 4-1 BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL2R, IL7R, IL10R, IL12R, IL15R, IL18R, TNFα, IFNγ, TGF-β, and α5β1.
28. The method of 22 or 23, wherein the surface coat comprises one or more targeting ligands that provides for targeted binding to target cells selected from: bone marrow cells, hematopoietic stem cells (HSCs), hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells, megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, liver cells, muscle cells, skeletal muscle cells, cardiac muscle cells, hepatic cells, fat cells, intestinal cells, cells of the colon, and cells of the stomach.
29. The method of any one of 1-14, wherein the delivery vehicle is a targeting ligand conjugated to the payload, wherein the targeting ligand provides for targeted binding to a cell surface protein.
30. The method of any one of 1-14, wherein the delivery vehicle is a targeting ligand conjugated to a charged polymer polypeptide domain, wherein the targeting ligand provides for targeted binding to a cell surface protein, and wherein the charged polymer polypeptide domain is condensed with a nucleic acid payload and/or is interacting electrostatically with a protein payload.
31. The method of 29 or 30, wherein the targeting ligand is a peptide, an ScFv, a F(ab), a nucleic acid aptamer, or a peptoid.
32. The method of 30, wherein the charged polymer polypeptide domain has a length in a range of from 3 to 30 amino acids.
33. The method of any one of 30-32, wherein the delivery vehicle further comprises an anionic polymer interacting with the payload and the charged polymer polypeptide domain.
34. The method of 33, wherein the anionic polymer is selected from poly(glutamic acid) and poly(aspartic acid).
35. The method of any one of 29-34, wherein the targeting ligand has a length of from 5-50 amino acids.
36. The method of any one of 29-35, wherein the targeting ligand provides for targeted binding to a cell surface protein selected from: a family B G-protein coupled receptor (GPCR), a receptor tyrosine kinase (RTK), a cell surface glycoprotein, and a cell-cell adhesion molecule.
37. The method of any one of 29-35, wherein the targeting ligand is selected from the group consisting of: rabies virus glycoprotein (RVG) fragment, ApoE-transferrin, lactoferrin, melanoferritin, ovotransferritin, L-selectin, E-selectin, P-selectin, sialylated peptides, polysialylated O-linked peptides, TPO, EPO, PSGL-1, ESL-1, CD44, death receptor-3 (DR3), LAMP1, LAMP2, Mac2-BP, stem cell factor (SCF), CD70, SH2 domain-containing protein 1A (SH2D1A), exendin, exendin-S11C, GLP1, RGD, a Transferrin ligand, an FGF fragment, an α5β1 ligand, IL2, Cde3-epsilon, peptide-HLA-A*2402, CD80, CD86, succinic acid, a bisphosphonate, a hematopoietic stem cell chemotactic lipid, sphingosine, ceramide, sphingosine-1-phosphate, ceramide-1-phosphate, and an active targeting fragment of any of the above.
38. The method of any one of 29-35, wherein the targeting ligand provides for targeted binding to a target selected from: CD3, CD28, CD90, CD45f, CD34, CD80, CD86, CD19, CD20, CD22, CD47, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL-2, IL-7, IL-10, IL-12, IL-15, IL-18, TNFα, IFNγ, TGF-β, and α5β1.
39. The method of any one of 29-35, wherein the targeting ligand provides for binding to a cell type selected from the group consisting of: bone marrow cells, hematopoietic stem cells (HSCs), long-term HSCs, short-term HSCs, hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, muscle cells, skeletal muscle cells, cardiac muscle cells, hepatic cells, fat cells, intestinal cells, cells of the colon, and cells of the stomach.
40. The method of any one of 1-39, wherein insertion of the nucleotide sequence of the second donor DNA into the cell's genome results in operable linkage of the inserted sequence with an endogenous promoter.
41. The method of 40, wherein the endogenous promoter is selected from the group consisting of: (i) a T-cell specific promoter; (ii) a CD3 promoter; (iii) a CD28 promoter; (iv) a stem cell specific promoter; (v) a somatic cell specific promoter; (vi) a T cell receptor (TCR) Alpha, Beta, Gamma or Delta promoter; (v) a B-cell specific promoter; (vi) a CD19 promoter; (vii) a CD20 promoter; (viii) a CD22 promoter; (ix) a B29 promoter; and (x) a T-cell or B-cell V(D)J-specific promoter.
42. The method of any one of 1-39, wherein the nucleotide sequence, of the insert donor composition, that is inserted includes a protein-coding sequence that is operably linked to a promoter.
43. The method of 42, wherein the promoter is selected from the group consisting of: (i) a T-cell specific promoter; (ii) a CD3 promoter; (iii) a CD28 promoter; (iv) a stem cell specific promoter; (v) a somatic cell specific promoter; (vi) a T cell receptor (TCR) Alpha, Beta, Gamma or Delta promoter; (v) a B-cell specific promoter; (vi) a CD19 promoter; (vii) a CD20 promoter; (viii) a CD22 promoter; (ix) a B29 promoter; and (x) a T-cell or B-cell V(D)J-specific promoter.
44. The method of any one of 1-43, wherein the nucleotide sequence, of the second donor DNA, that is inserted into the cell's genome encodes (i) a T cell receptor (TCR) protein; (ii) an IgA, IgD, IgE, IgG, or IgM protein; or (iii) the K or A chains of an IgA, IgD, IgE, IgG, or IgM protein.
45. The method of any one of 1-43, wherein the nucleotide sequence, of the second donor DNA, that is inserted into the cell's genome encodes a CDR1, CDR2, or CDR3 region of a T cell receptor (TCR) protein.
46. The method of any one of 1-43, wherein the nucleotide sequence, of the second donor DNA, that is inserted into the cell's genome encodes a chimeric antigen receptor (CAR).
47. The method of 46, wherein insertion of the nucleotide sequence that encodes the CAR results in operable linkage of the nucleotide sequence that encodes the CAR with an endogenous T-cell specific promoter.
48. The method of any one of 1-43, wherein the nucleotide sequence, of the second donor DNA, that is inserted into the cell's genome encodes a multivalent surface receptor.
49. The method of 48, wherein the cell is a T-cell.
50. The method of 48 or 49, wherein the multivalent surface receptor is a bispecific or trispecific chimeric antigen receptor (CAR) or T cell receptor (TCR).
51. The method of any one of 1-43, wherein the nucleotide sequence, of the second donor DNA, that is inserted into the cell's genome encodes a cell-specific targeting ligand that is membrane bound and presented extracellularly.
52. The method of any one of 1-43, wherein the nucleotide sequence, of the second donor DNA, that is inserted into the cell's genome encodes a reporter protein.
53. The method of 52, wherein the reporter protein is a fluorescent protein.
54. The method of 52 or 53, wherein the nucleotide sequence that encodes the reporter protein is operably linked to a cell-specific or tissue-specific promoter.
55. The method of 52 or 53, wherein the nucleotide sequence that encodes the reporter protein is operably linked to a constitutive promoter.
56. The method of any one of 1-55, wherein the nucleotide sequence, of the second donor DNA, that is inserted into the cell's genome includes a protein-coding nucleotide sequence that does not have introns.
57. The method of 56, wherein the nucleotide sequence that does not have introns encodes all or a portion of a TCR protein or an Immunoglobulin.
58. The method of any one of 1-57, wherein the method comprises introducing a first and a second of said delivery vehicles into the cell, wherein:
(1) the nucleotide sequence of the second donor DNA of the first delivery vehicle, that is inserted into the cell's genome, encodes a T cell receptor (TCR) Alpha or Delta subunit, and the nucleotide sequence of the second donor DNA of the second delivery vehicle, that is inserted into the cell's genome, encodes a TCR Beta or Gamma subunit; or
(2) the nucleotide sequence of the second donor DNA of the first delivery vehicle, that is inserted into the cell's genome, encodes a T cell receptor (TCR) Alpha or Gamma subunit, and the nucleotide sequence of the second donor DNA of the second delivery vehicle, that is inserted into the cell's genome, encodes a TCR Beta or Delta subunit; or
(3) the nucleotide sequence of the second donor DNA of the first delivery vehicle, that is inserted into the cell's genome, encodes the K chain of an IgA, IgD, IgE, IgG, or IgM protein, and the nucleotide sequence of the second donor DNA of the second delivery vehicle, that is inserted into the cell's genome, encodes the A chain of an IgA, IgD, IgE, IgG, or IgM protein.
   59. The method of any one of 1-57, wherein the method comprises introducing a first and a second of said delivery vehicles into the cell,

wherein the nucleotide sequence of the second donor DNA of the first delivery vehicle, that is inserted into the cell's genome, encodes a T cell receptor (TCR) Alpha or Delta subunit constant region, and

wherein the nucleotide sequence of the second donor DNA of the second delivery vehicle, that is inserted into the cell's genome, encodes a TCR Beta or Gamma subunit constant region.

60. The method of any one of 1-57, wherein the method comprises introducing a first and a second of said delivery vehicles into the cell, wherein:
(1) the nucleotide sequence of the second donor DNA of the first delivery vehicle is inserted within a nucleotide sequence that functions as a T cell receptor (TCR) Alpha or Delta subunit promoter, and the nucleotide sequence of the second donor DNA of the second delivery vehicle is inserted within a nucleotide sequence that functions as a TCR Beta or Gamma subunit promoter; or
(2) the nucleotide sequence of the second donor DNA of the first delivery vehicle is inserted within a nucleotide sequence that functions as a T cell receptor (TCR) Alpha or Gamma subunit promoter, and the nucleotide sequence of the second donor DNA of the second delivery vehicle is inserted within a nucleotide sequence that functions as a TCR Beta or Delta subunit promoter; or
(3) the nucleotide sequence of the second donor DNA of the first delivery vehicle is inserted within a nucleotide sequence that functions as a promoter for a K chain of an IgA, IgD, IgE, IgG, or IgM protein, and the nucleotide sequence of the second donor DNA of the second delivery vehicle is inserted within a nucleotide sequence that functions as a promoter for a λ chain of an IgA, IgD, IgE, IgG, or IgM protein.
   61. The method of any one of 1-60, wherein the cell is a mammalian cell.
   62. The method of any one of 1-61, wherein the cell is a human cell.
   63. A composition comprising:

(a) a nuclease composition, comprising: one or more sequence specific nucleases or one or more nucleic acids that encode the one or more sequence specific nucleases;

(b) a target donor composition, comprising: a first donor DNA that comprises a target sequence for a site-specific recombinase;

(c) a recombinase composition, comprising: the site-specific recombinase, or a nucleic acid encoding the site-specific recombinase, wherein the site-specific recombinase recognizes said target sequence; and

(d) an insert donor composition, comprising: a second donor DNA, which comprises a nucleotide sequence of interest for insertion into a target cell's genome;

wherein (a), (b), (c), and (d) are payloads as part of the same delivery vehicle.

64. The composition of 63, wherein the delivery vehicle is a nanoparticle.
65. The composition of 64, wherein the nanoparticle comprises a core comprising (a), (b), an anionic polymer composition, a cationic polymer composition, and a cationic polypeptide composition.
66. The composition of 64 or 65, wherein the nanoparticle comprises a targeting ligand that targets the nanoparticle to a cell surface protein.
67. The composition of 66, wherein the cell surface protein is CD47.
68. The composition of 67, wherein the targeting ligand is a SIRPα protein mimetic (which prevents macrophage uptake) (e.g., an external fragment of SIRPα).
69. The composition of 68, wherein the nanoparticle further comprises an endocytosis-triggering ligand.
70. The composition of any one of 63-69, wherein the payloads form one or more deoxyribonucleoprotein complexes or one or more ribo-deoxyribonucleoprotein complexes.
71. The composition of any one of 63-70, wherein the delivery vehicle is a targeting ligand conjugated to a charged polymer polypeptide domain, wherein the targeting ligand provides for targeted binding to a cell surface protein, and wherein the charged polymer polypeptide domain is interacting electrostatically with one or more of the payloads.
72. The composition of 71, wherein the delivery vehicle further comprises an anionic polymer interacting with one or more of the payloads and the charged polymer polypeptide domain.
73. The composition of any one of 63-70, wherein the delivery vehicle is a targeting ligand conjugated one or more of the payloads, wherein the targeting ligand provides for targeted binding to a cell surface protein.
74. The composition of any one of 63-70, herein the delivery vehicle includes a targeting ligand coated upon a water-oil-water emulsion particle, upon an oil-water emulsion micellar particle, upon a multilamellar water-oil-water emulsion particle, upon a multilayered particle, or upon a DNA origami nanobot.
75. The method of any one of 66-71, wherein the targeting ligand is a peptide, an ScFv, a F(ab), a nucleic acid aptamer, or a peptoid.
76. The composition of any one of 63-70, wherein the delivery vehicle is non-viral.

### EXPERIMENTAL

The following examples are put forth so as to provide those of ordinary skill in the art with a complete disclosure and description of how to make and use the present invention, and are not intended to limit the scope of the invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Cellular uptake and phenotype were characterized with flow cytometry and high-content screening, quantifying delivery efficiency and gene editing across various subpopulations of cells.

In these experiments, unstimulated human primary Pan-T Cells (a mixture of CD4+ and CD8+ T-cells) and peripheral blood mononuclear cells (PBMCs) were flash transfected (30 minutes incubation with nanoparticles), washed twice with PBS containing 10 ug/ml heparan sulfate, and analyzed 24 hours later on an Attune N×T flow cytometer. Cells were stained with antibodies specific for CD4 and CD8, and transduction of EGFP-tagged Cas9 was quantified in each subpopulation.

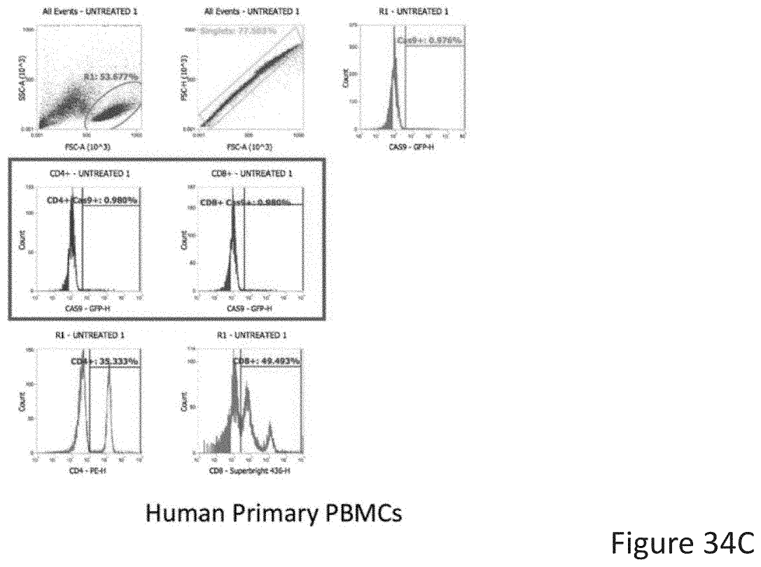

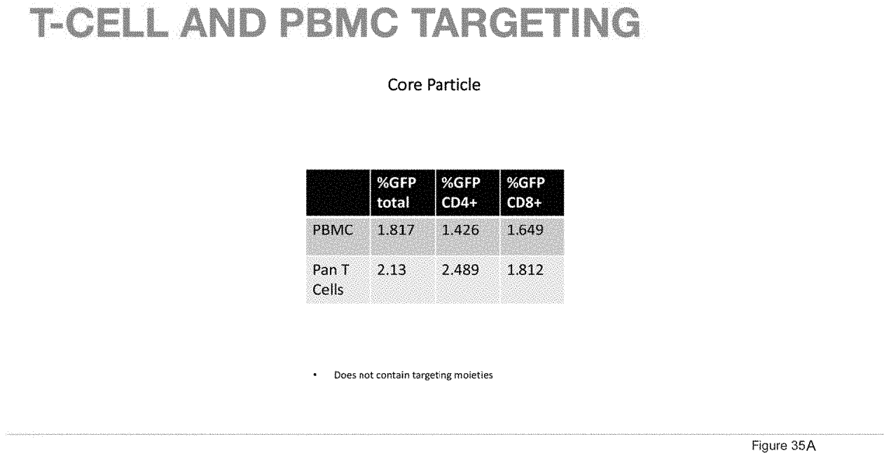

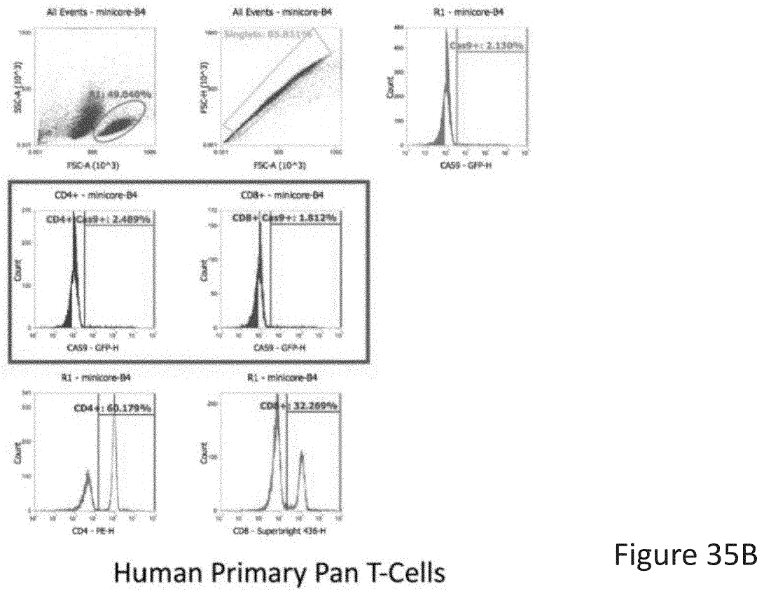

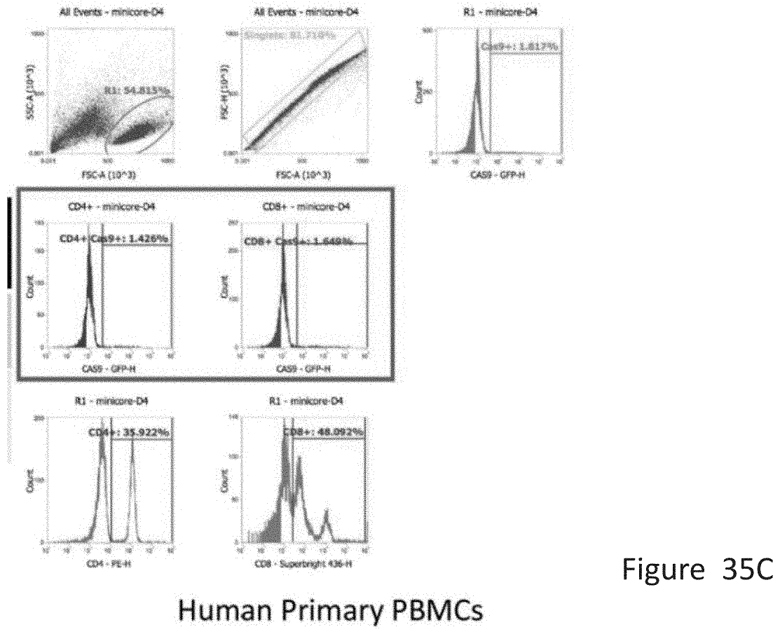

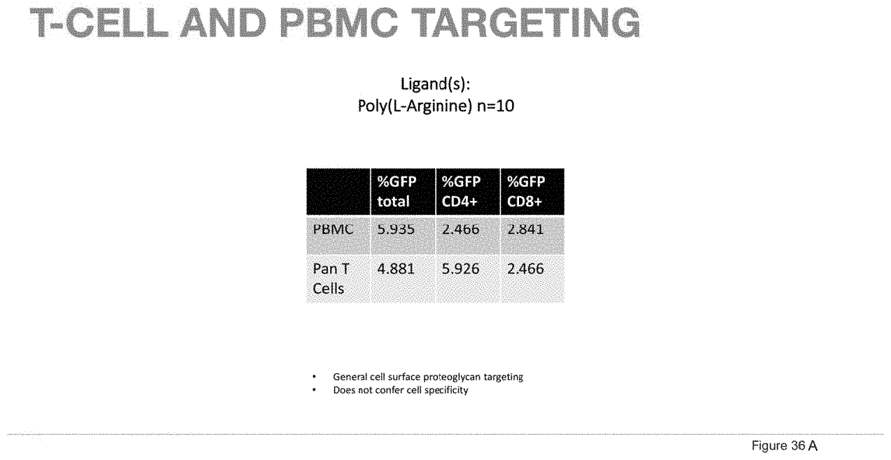

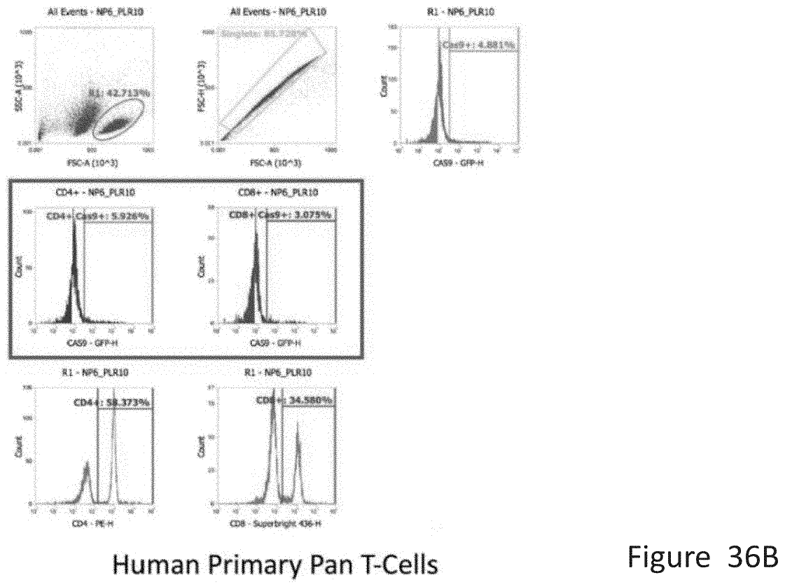

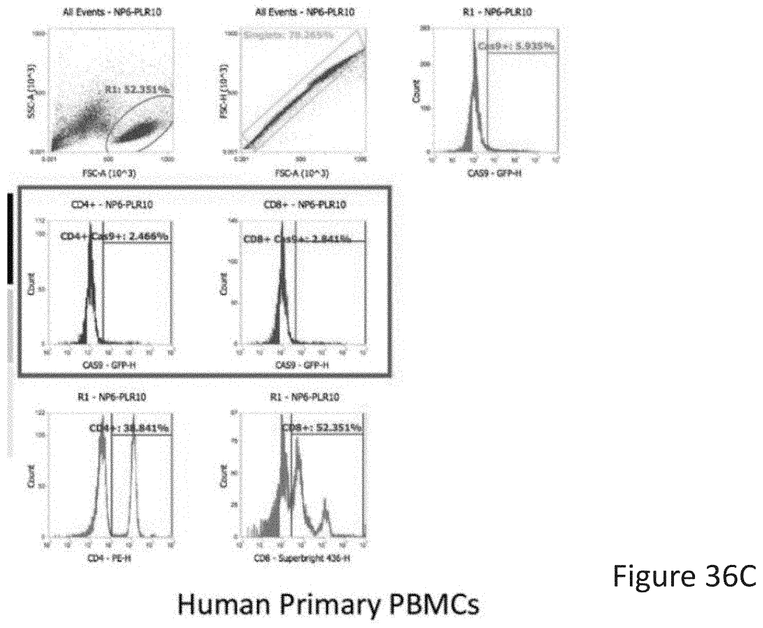

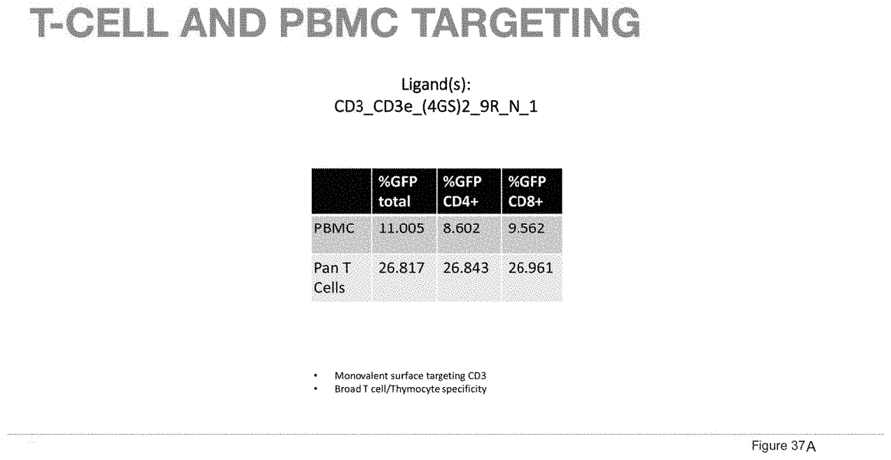

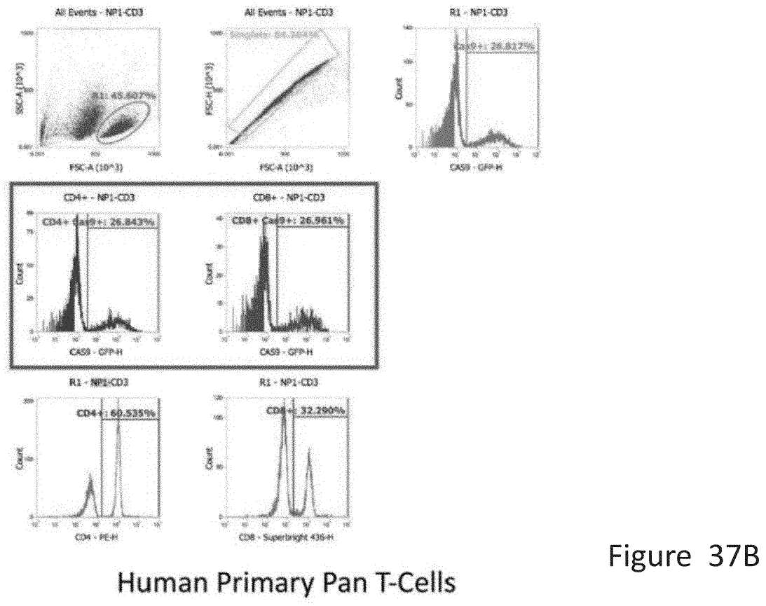

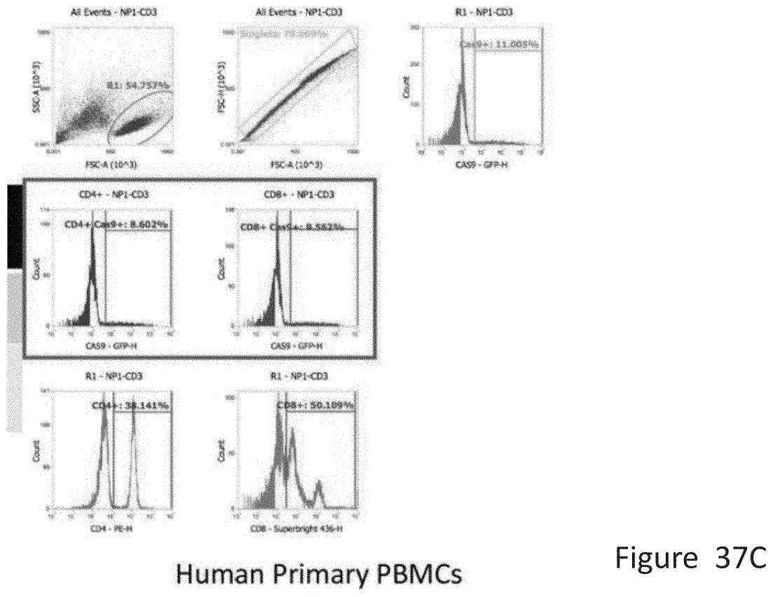

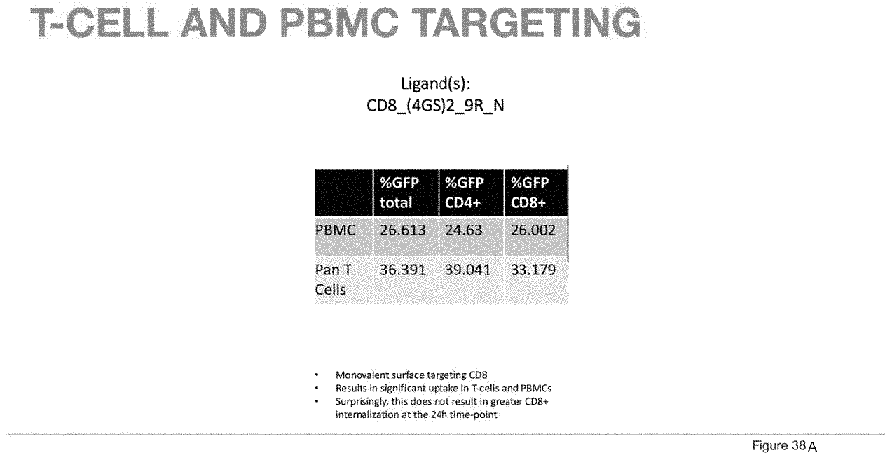

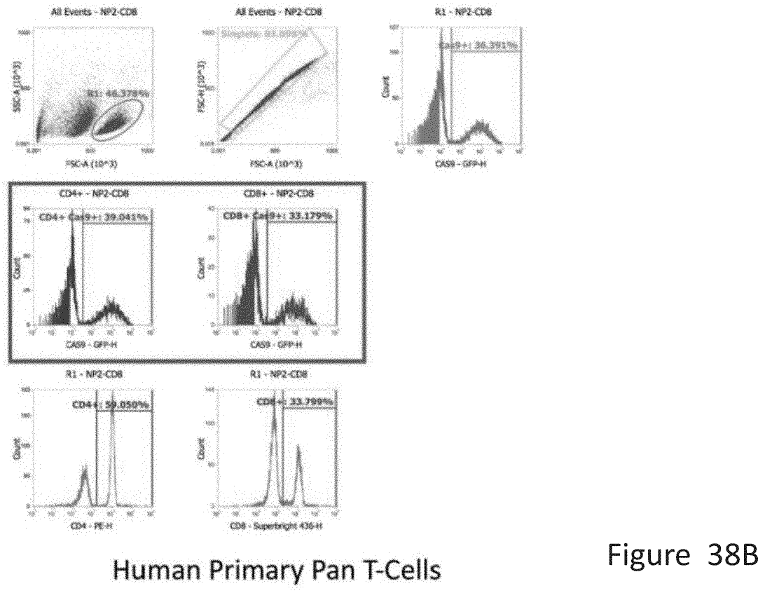

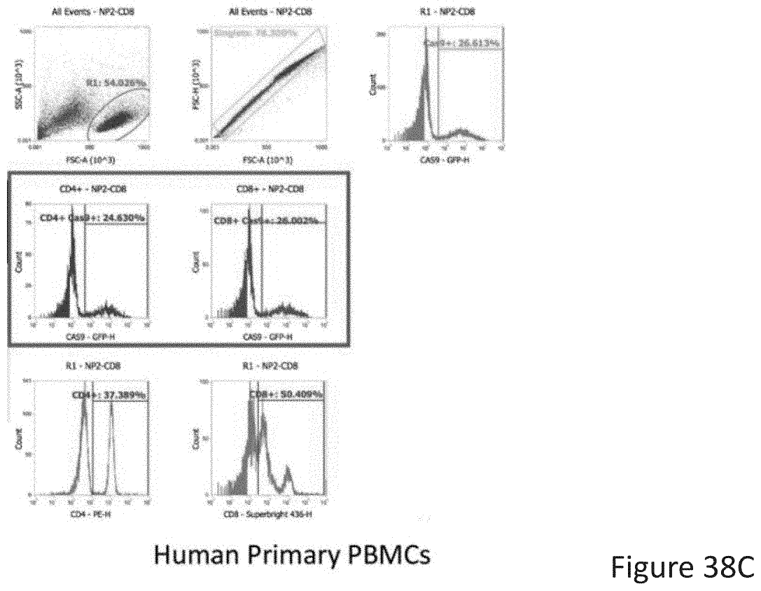

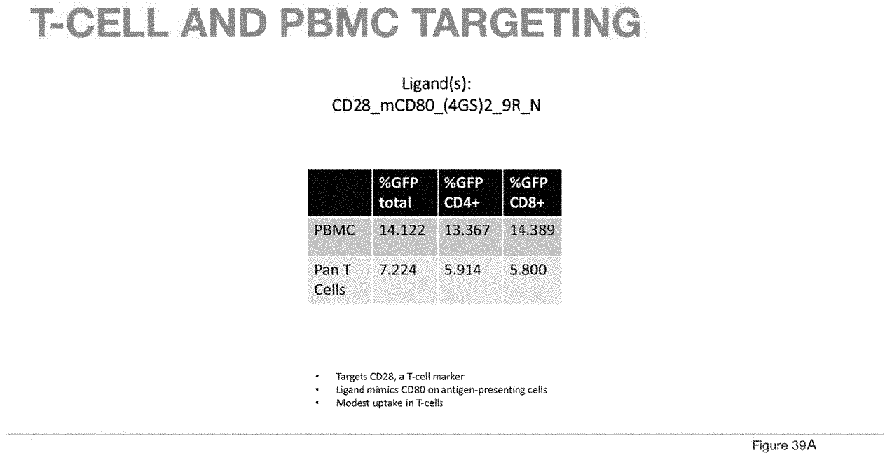

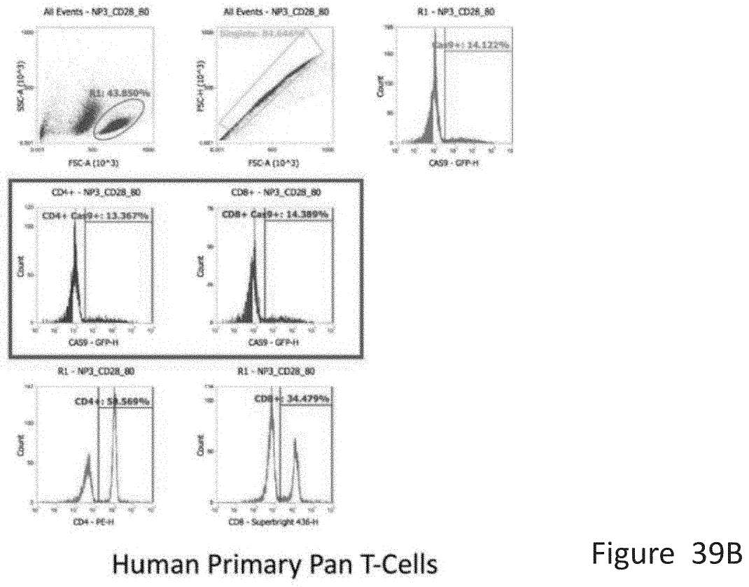

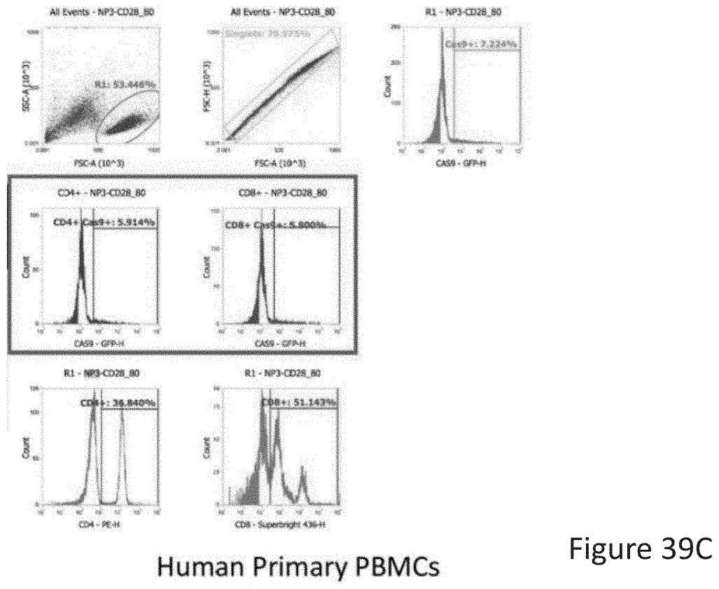

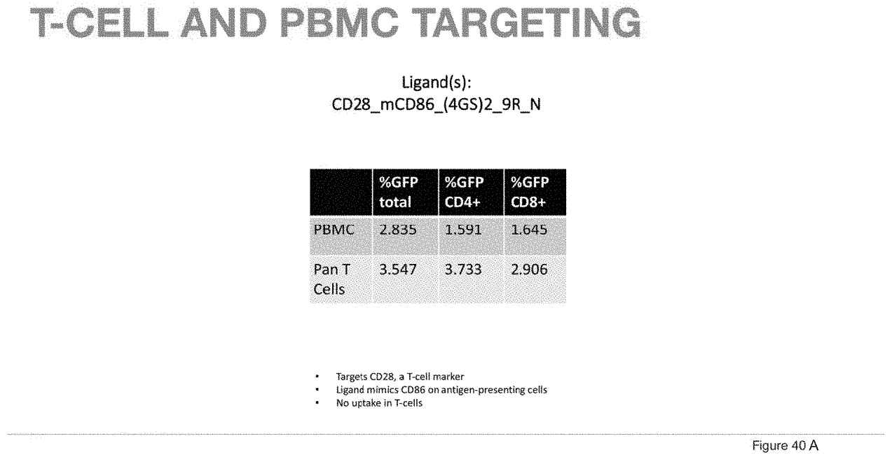

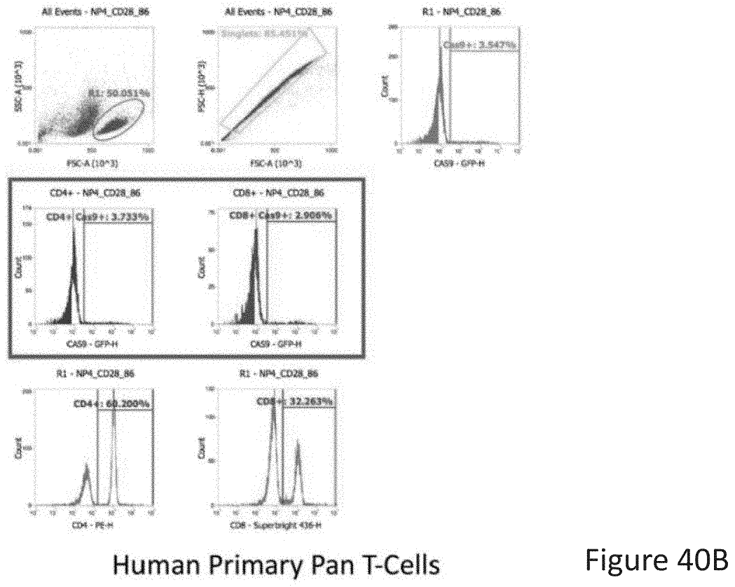

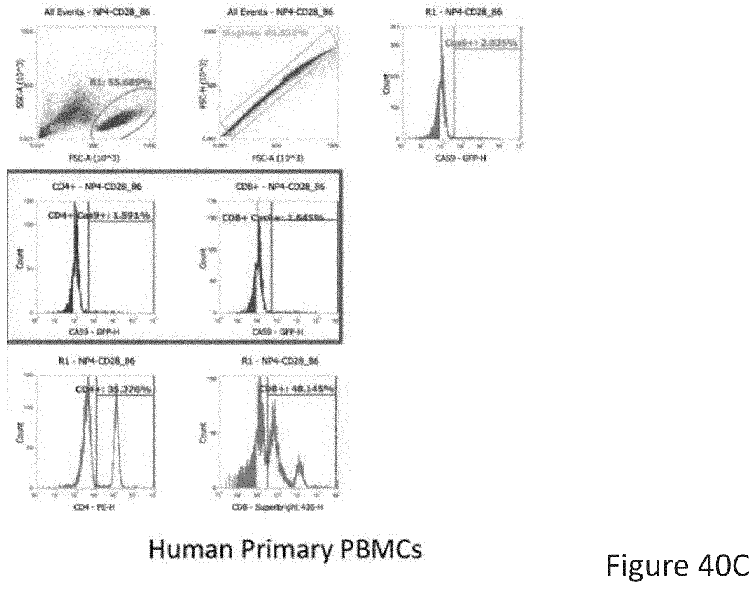

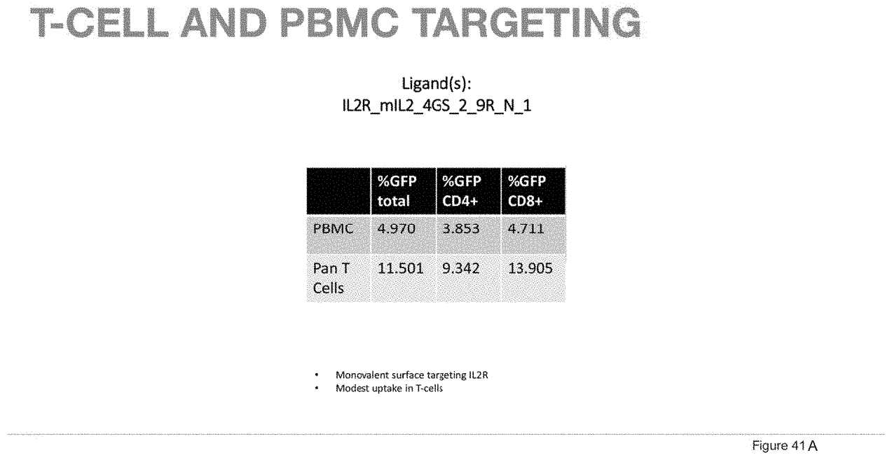

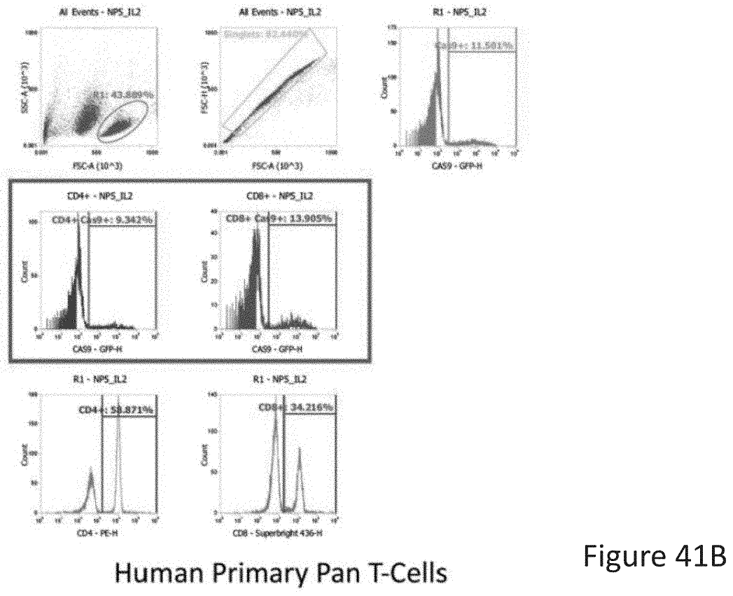

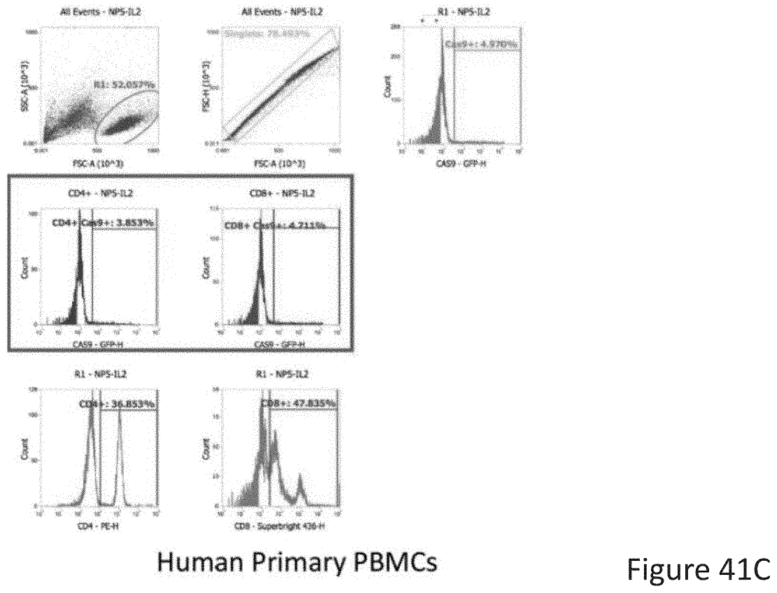

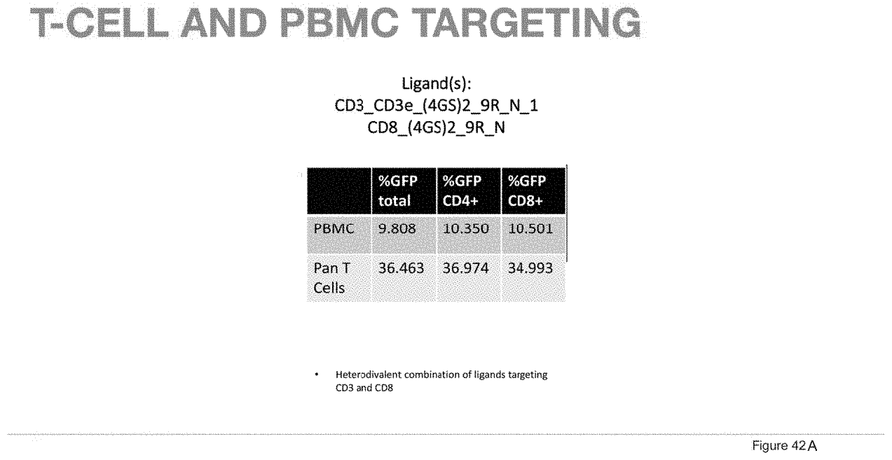

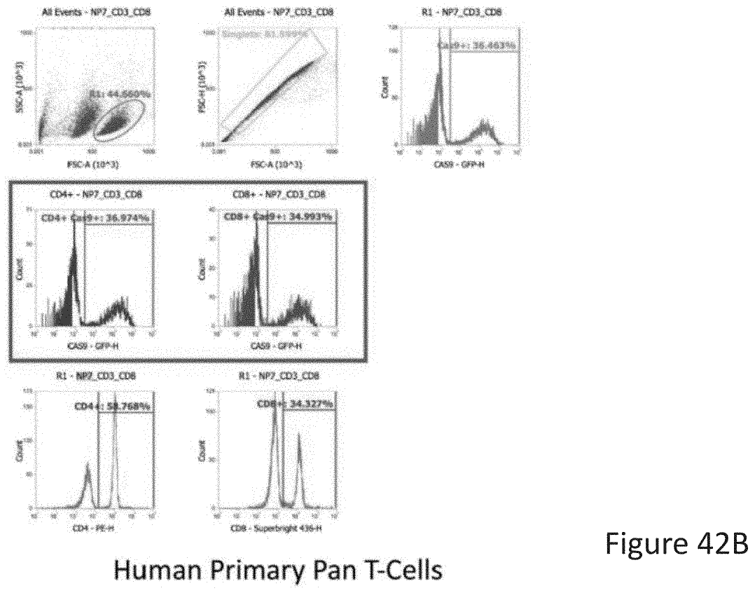

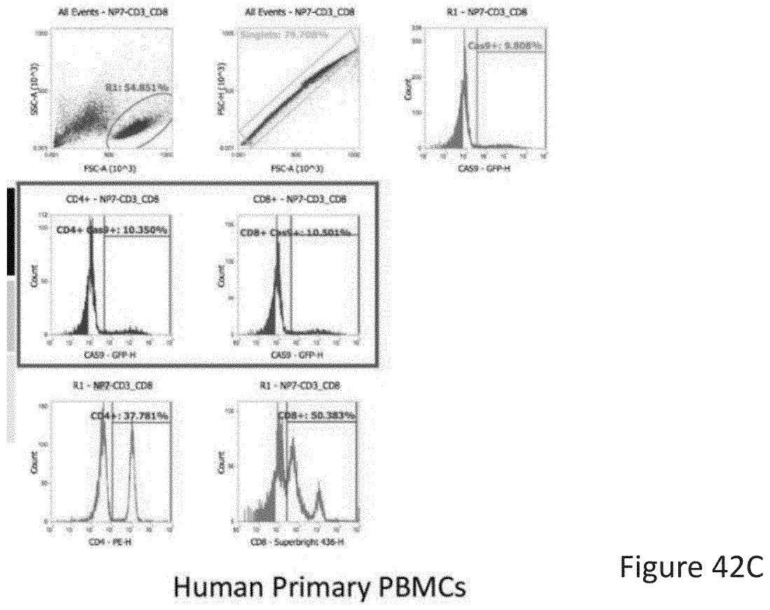

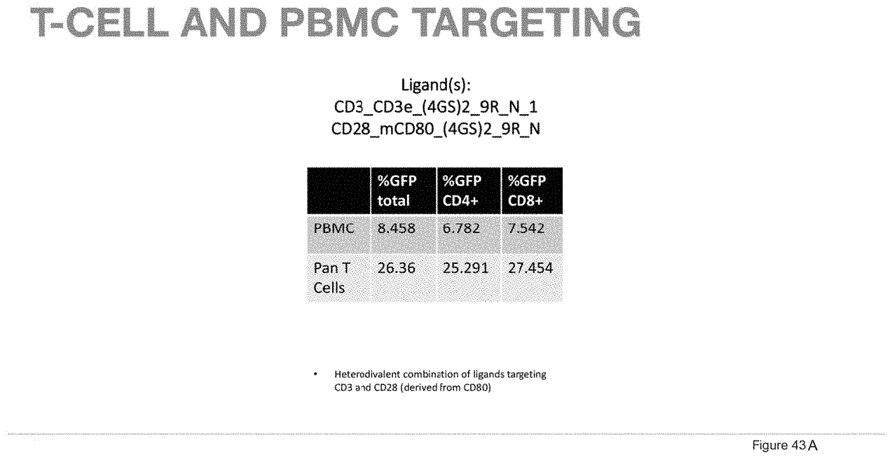

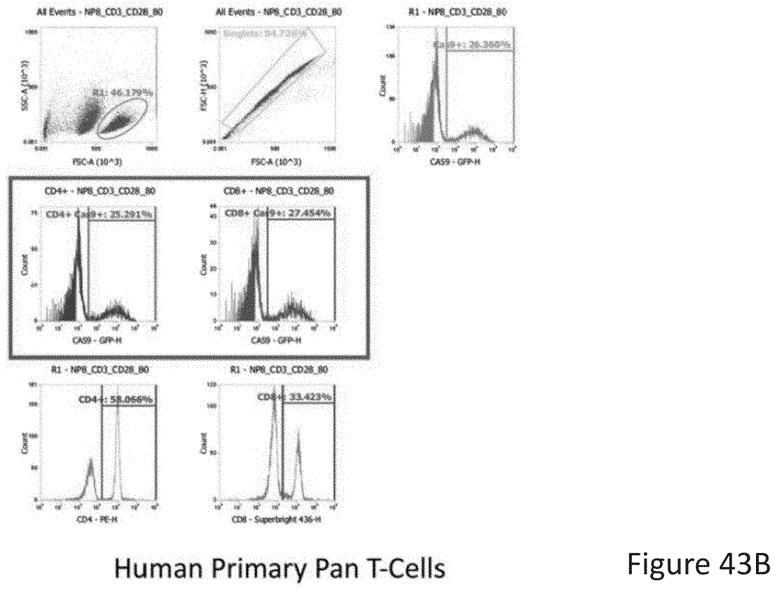

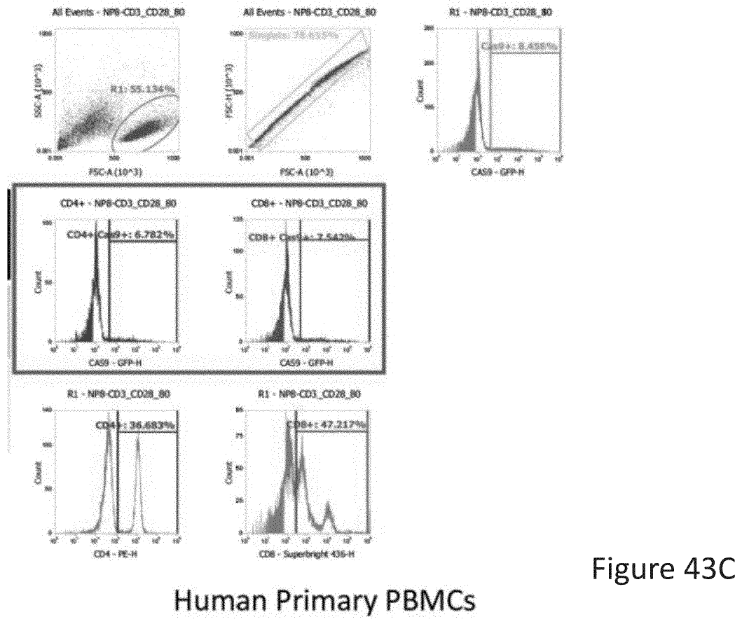

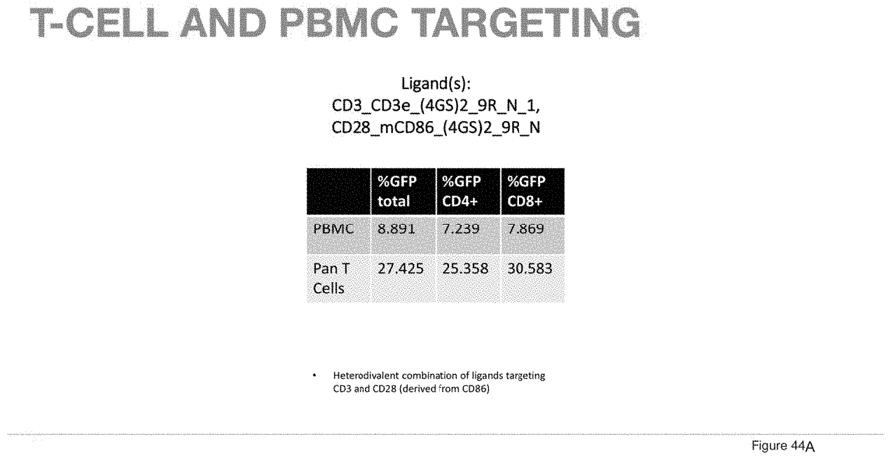

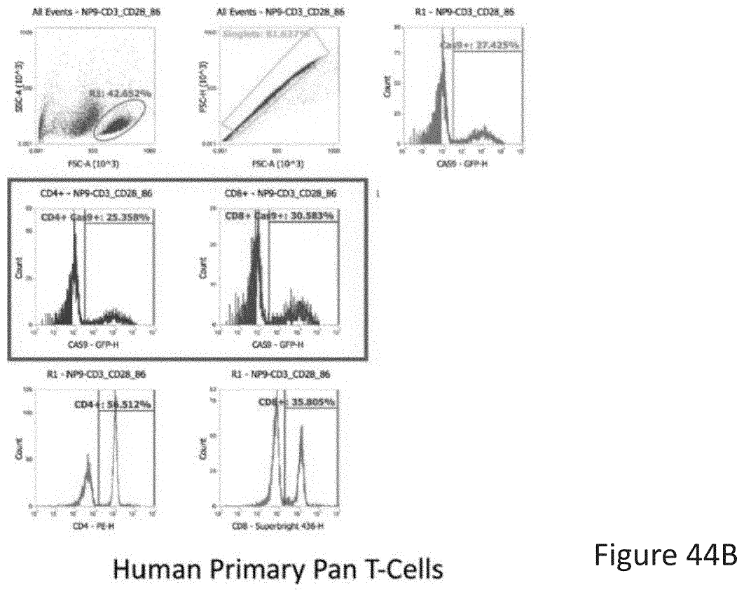

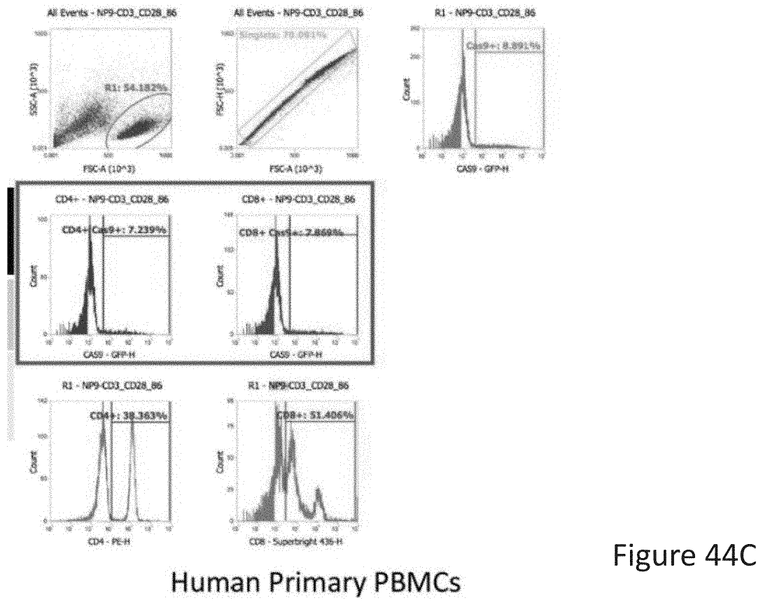

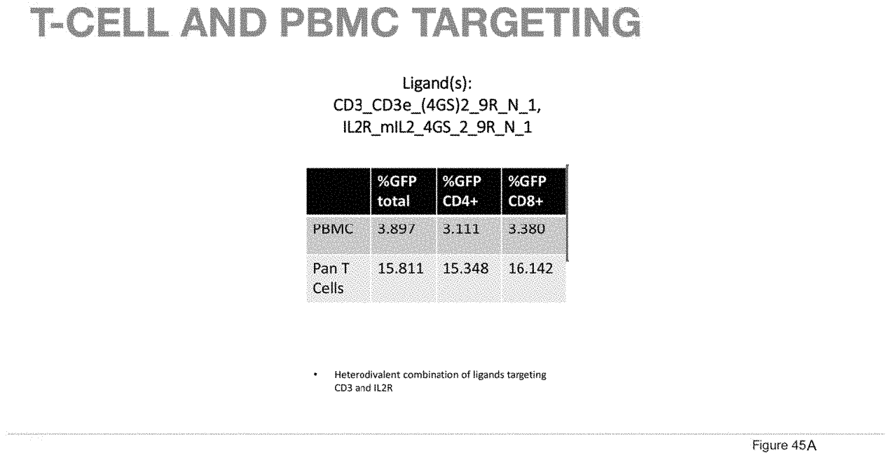

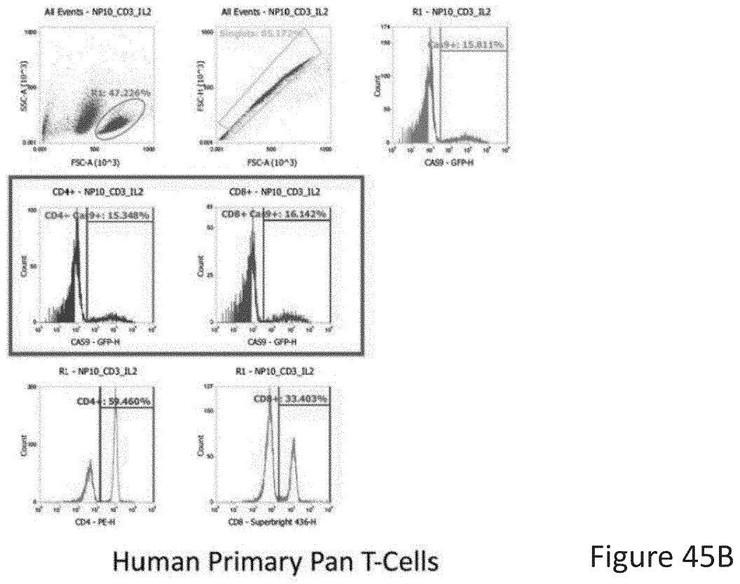

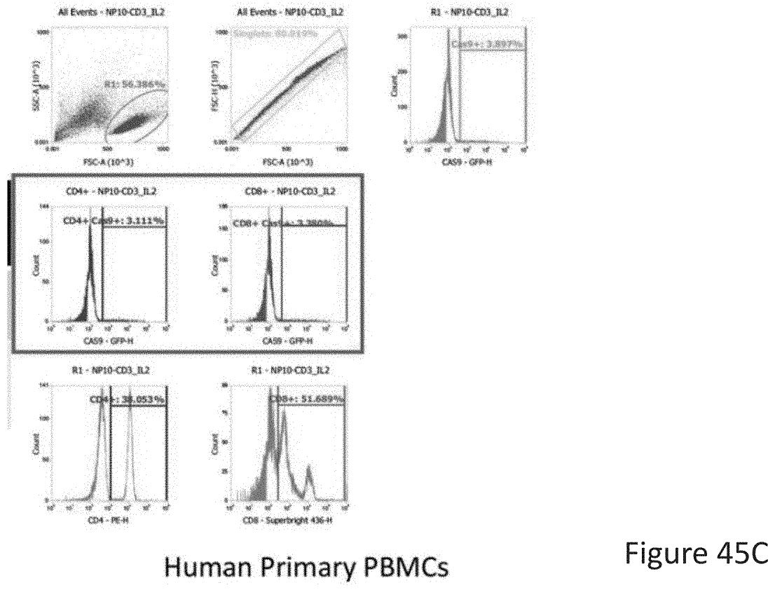

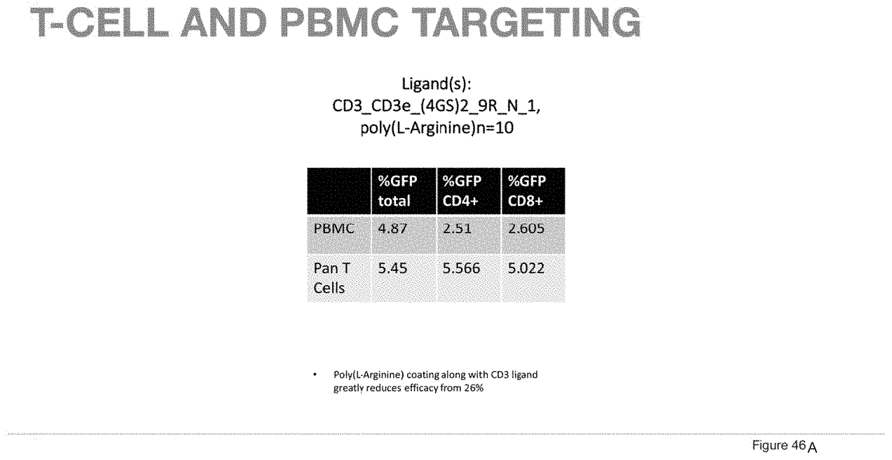

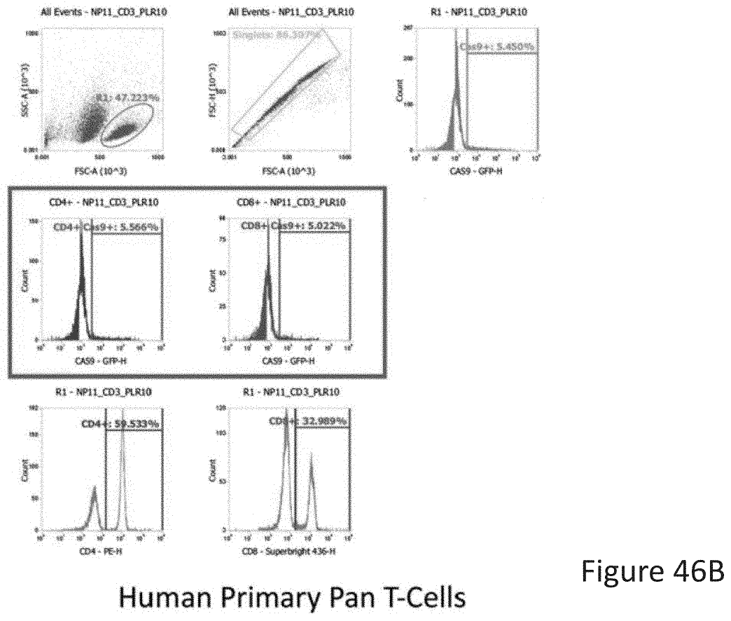

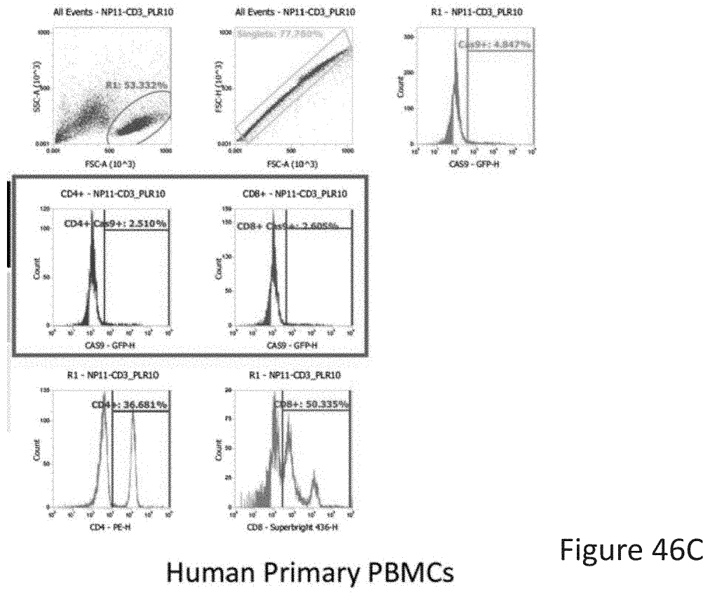

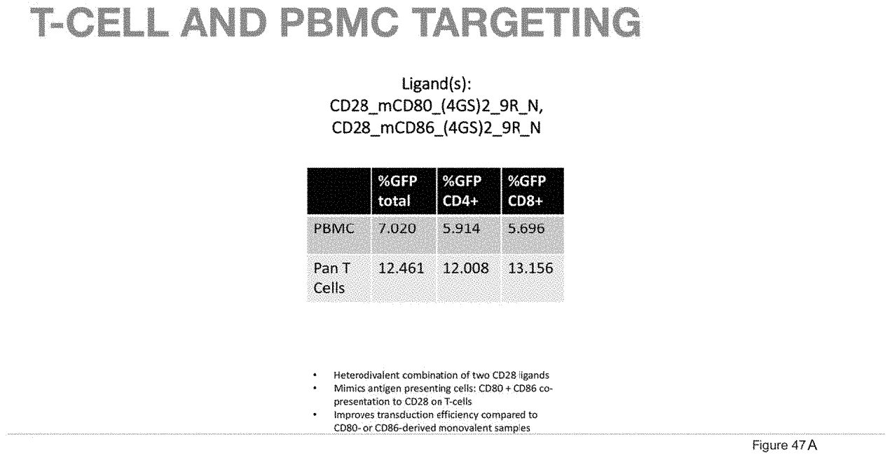

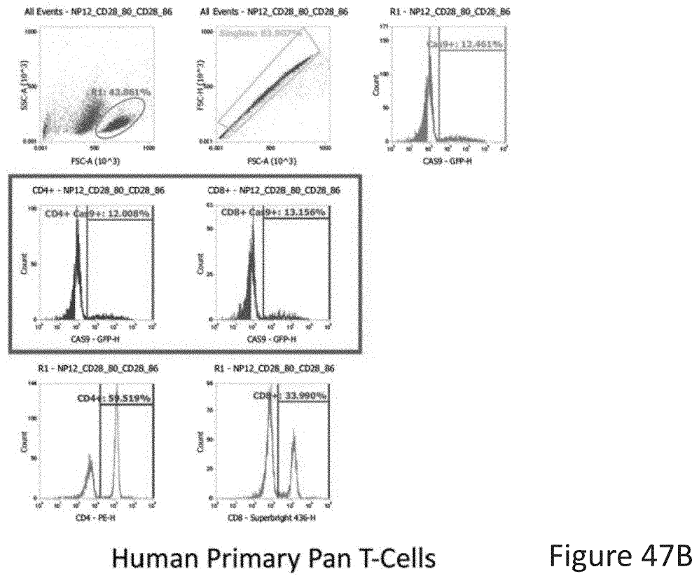

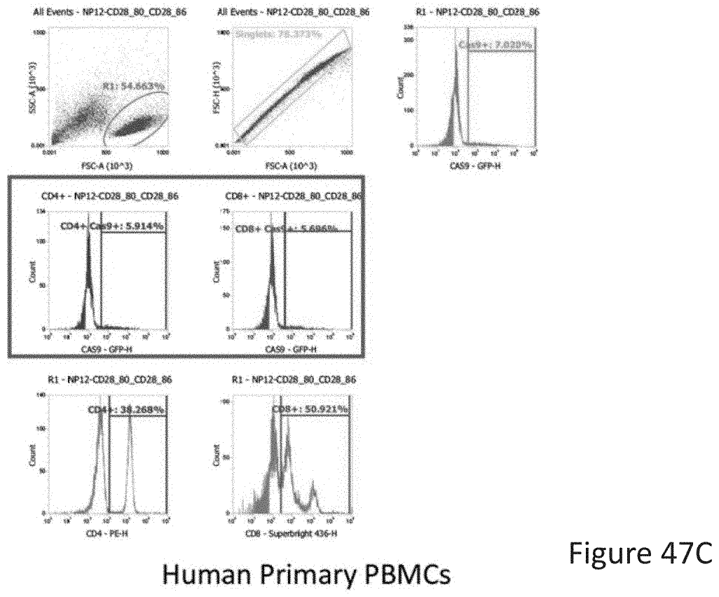

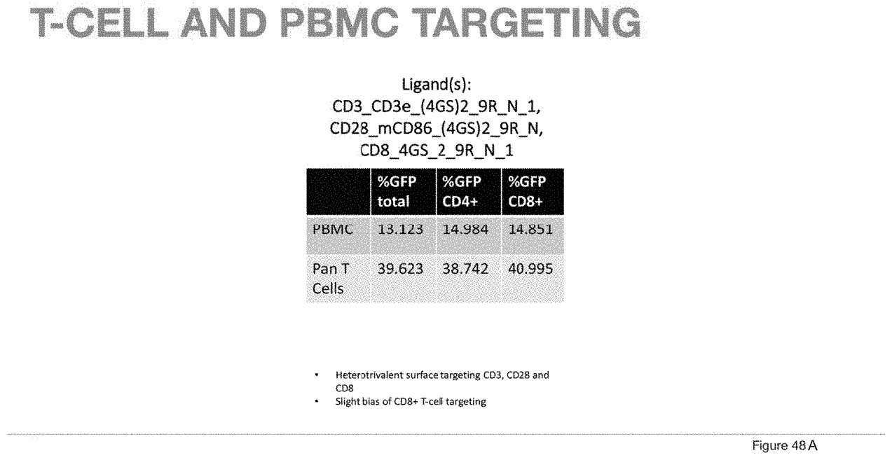

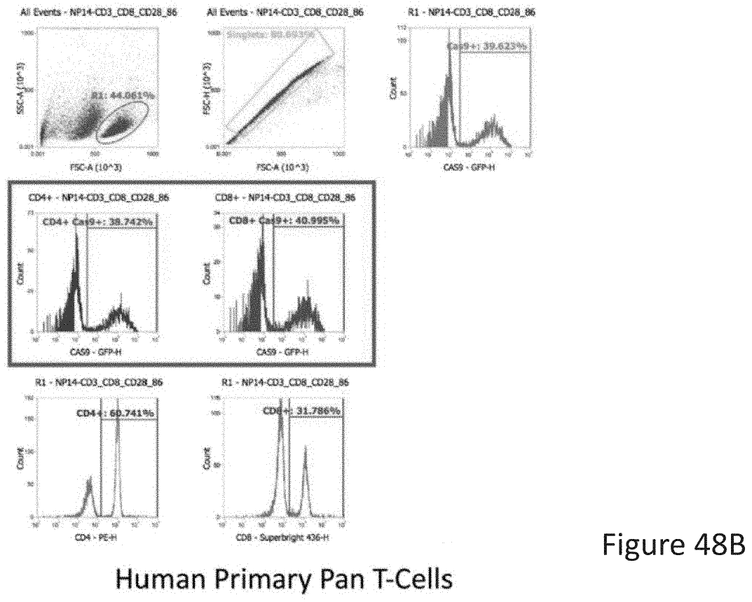

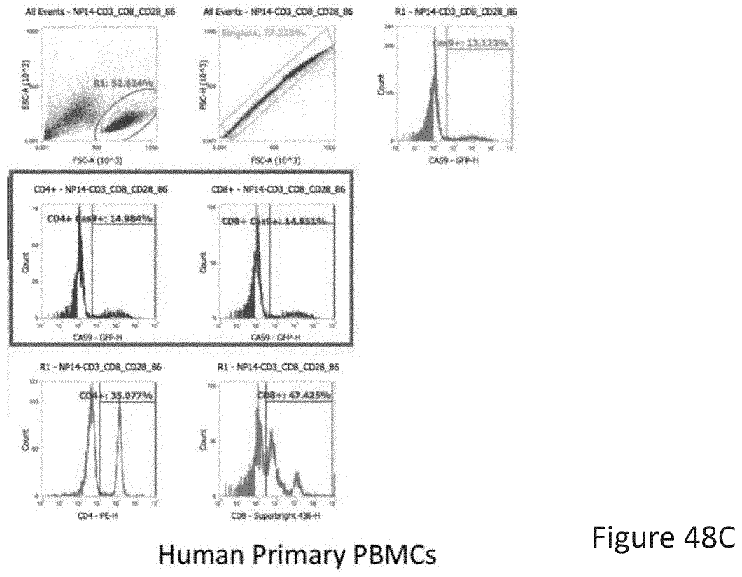

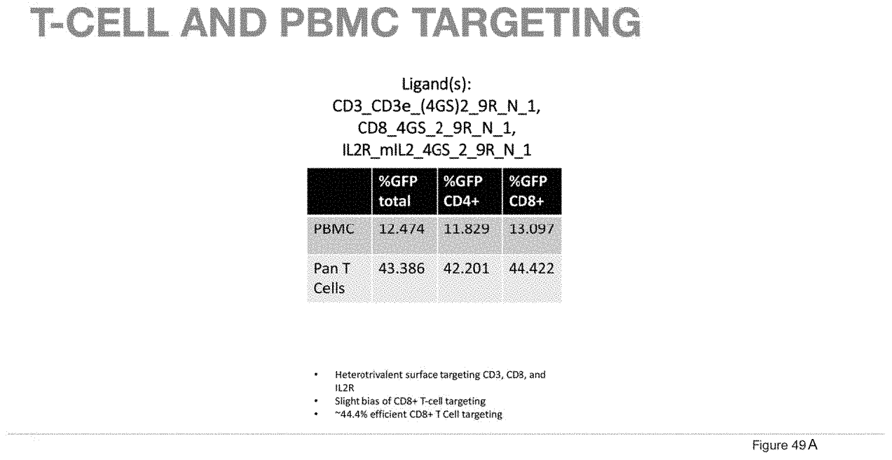

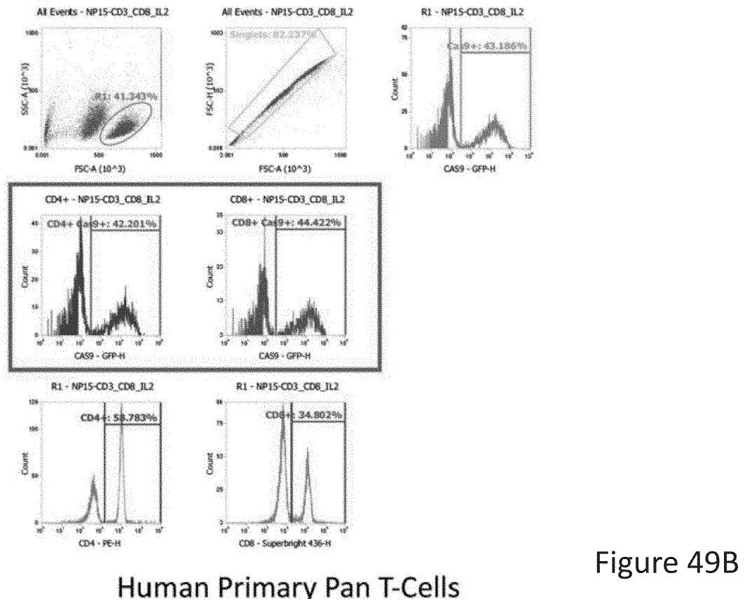

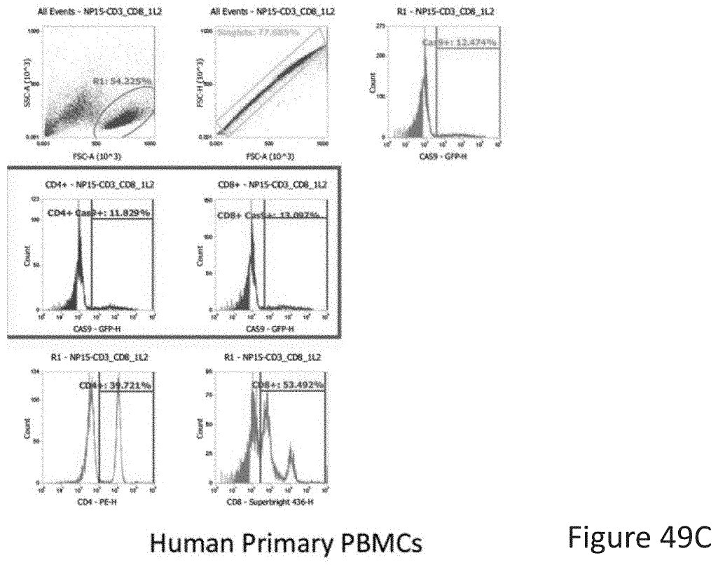

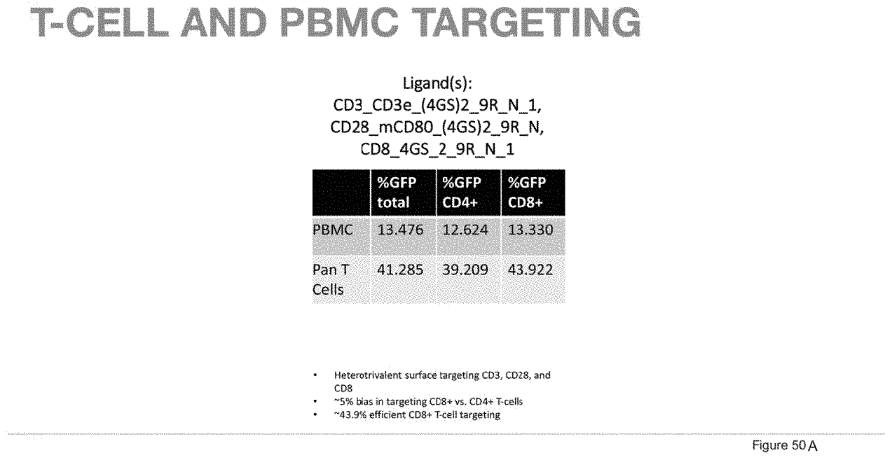

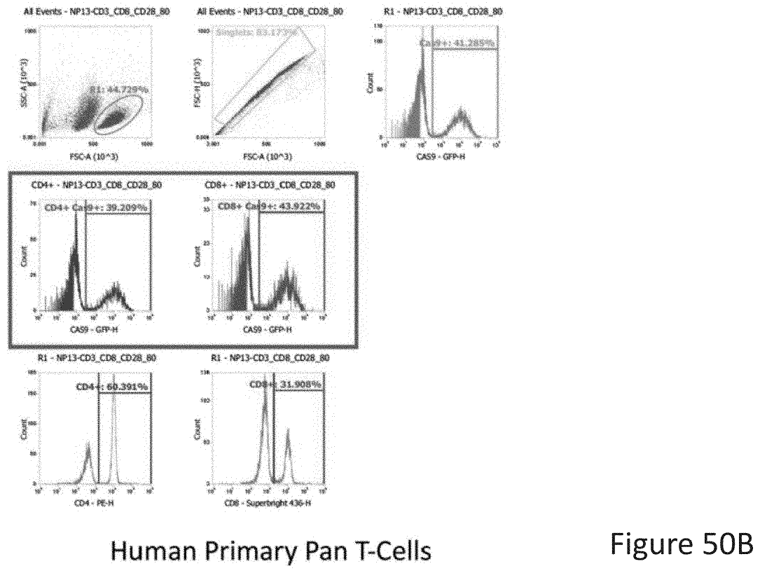

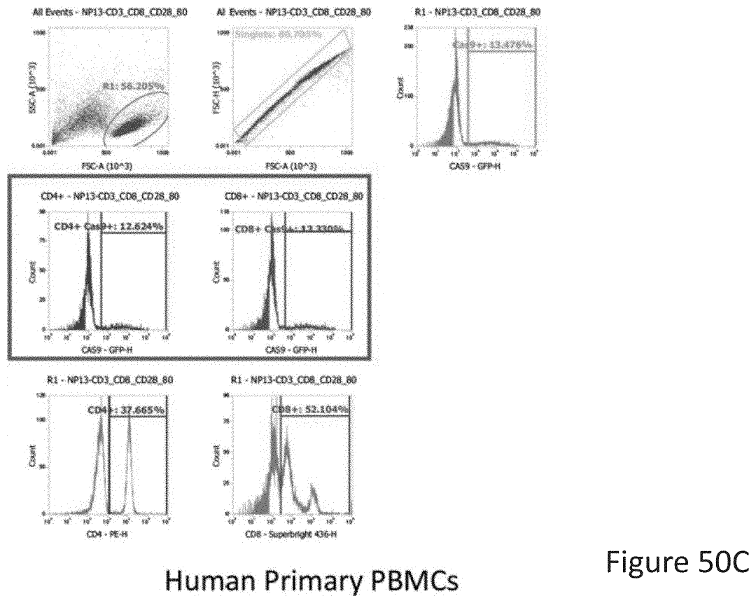

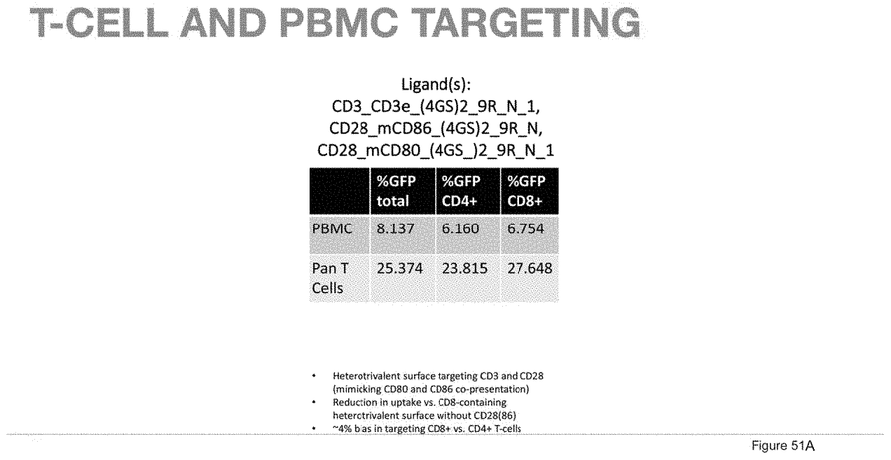

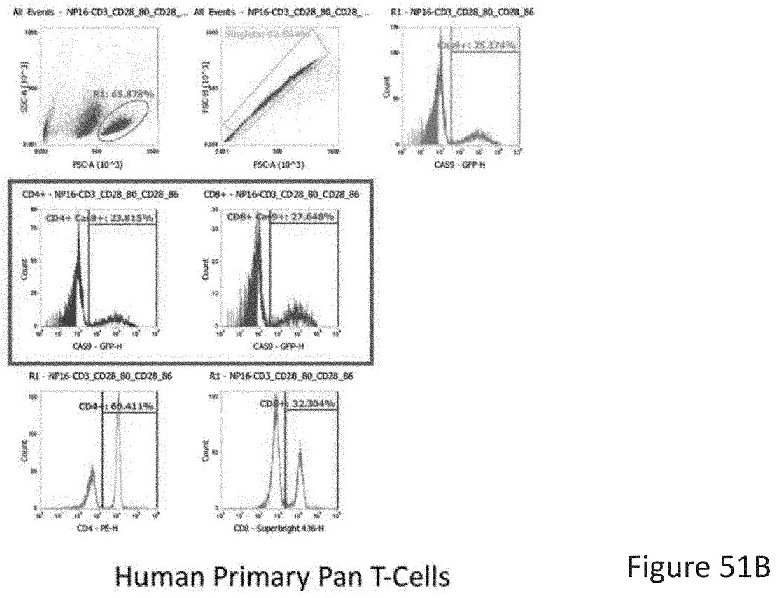

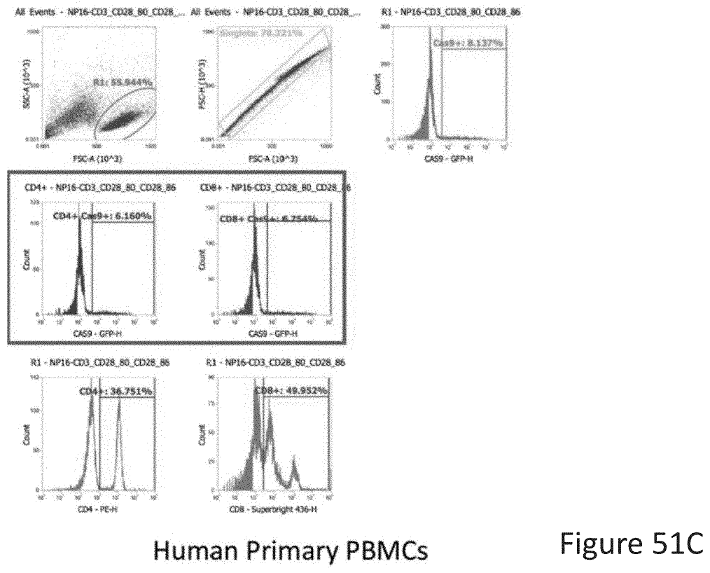

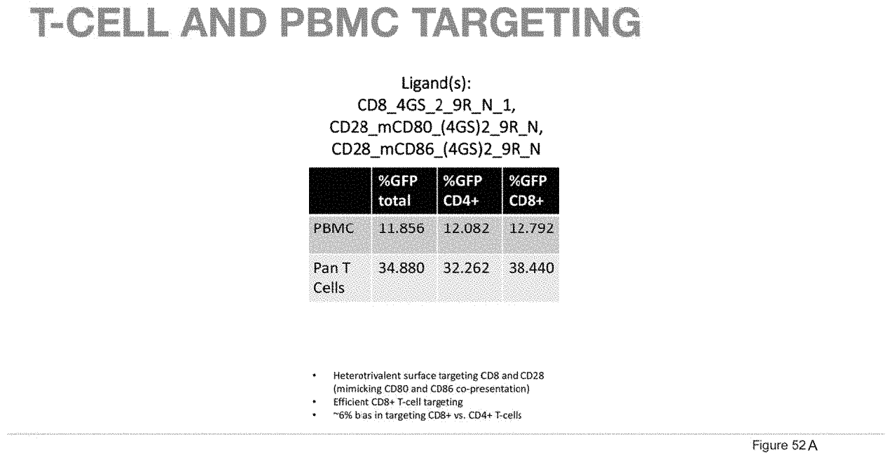

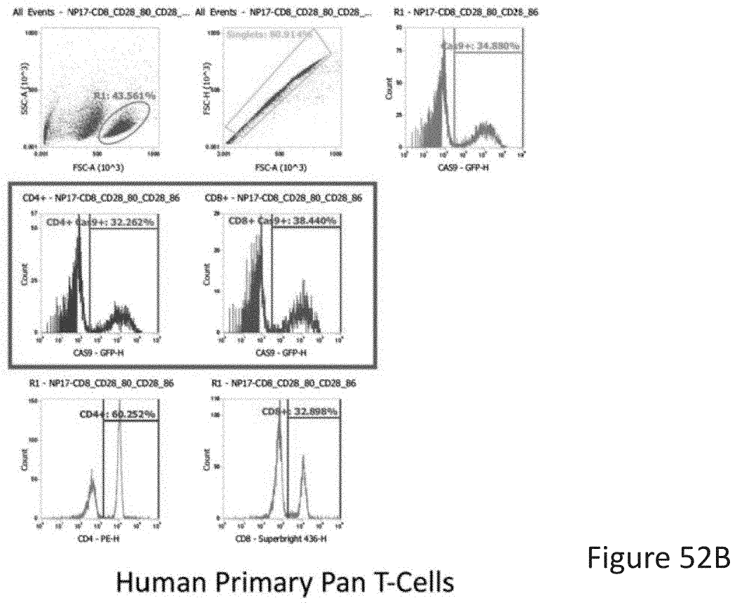

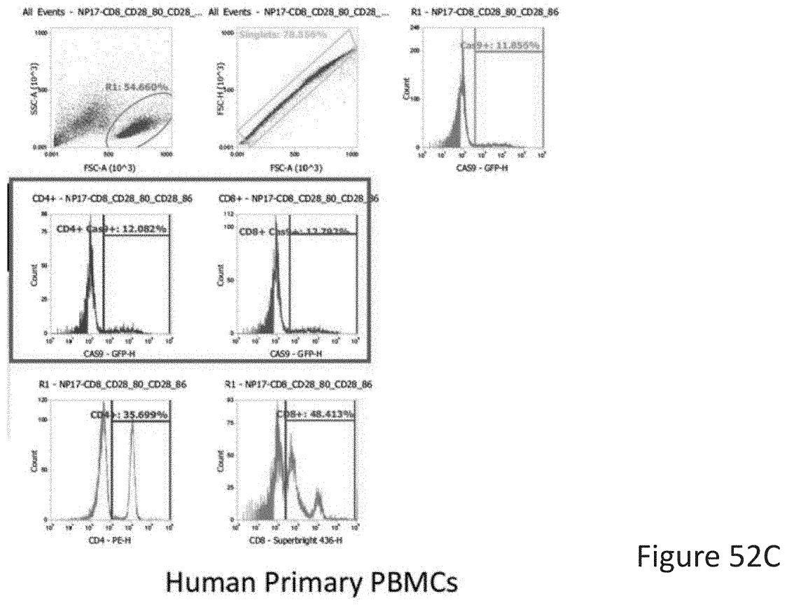

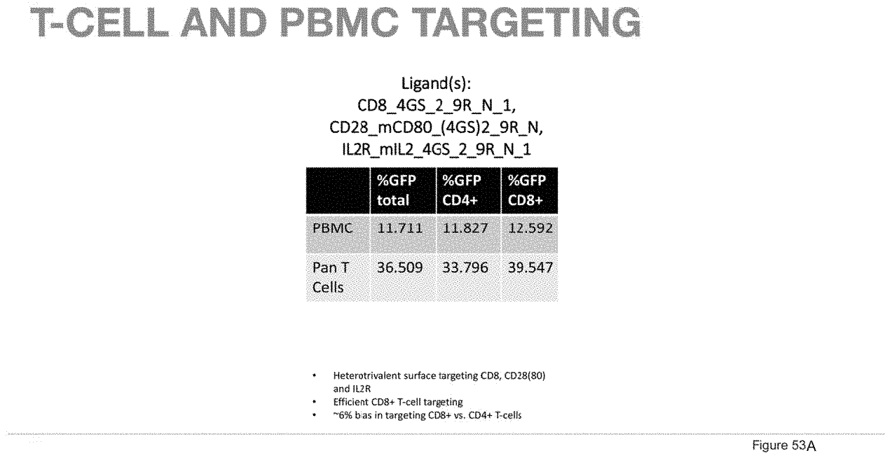

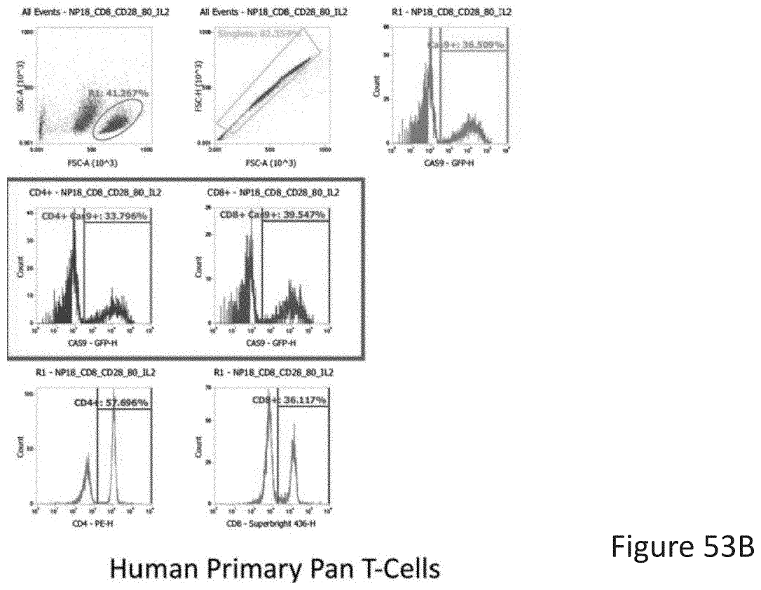

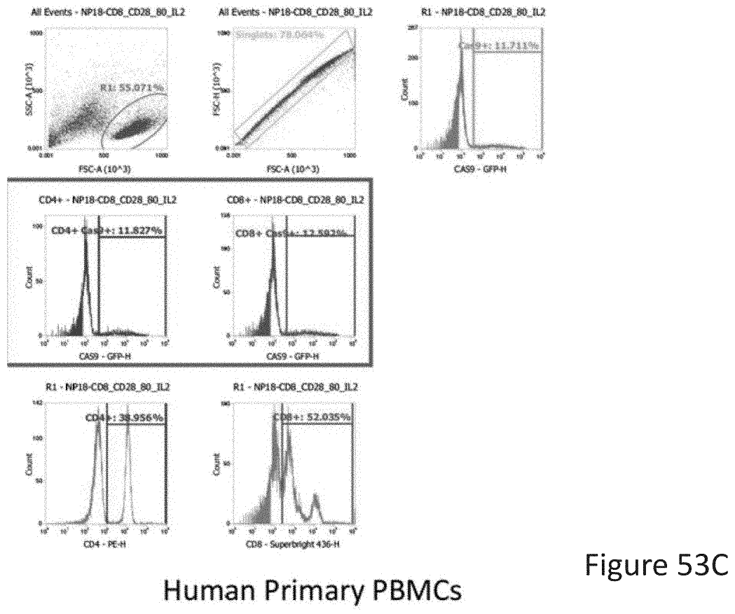

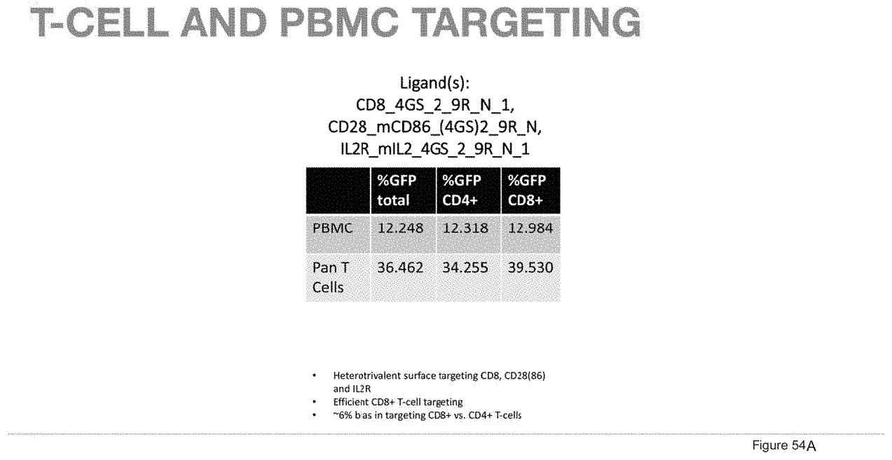

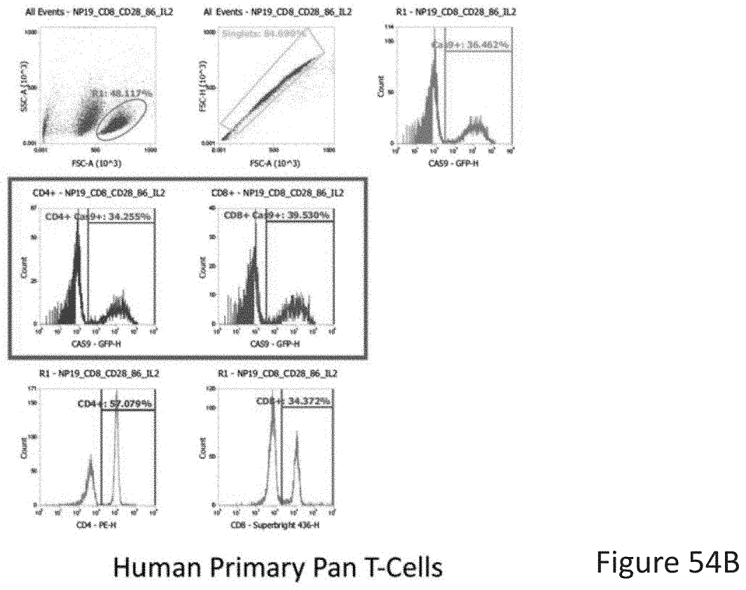

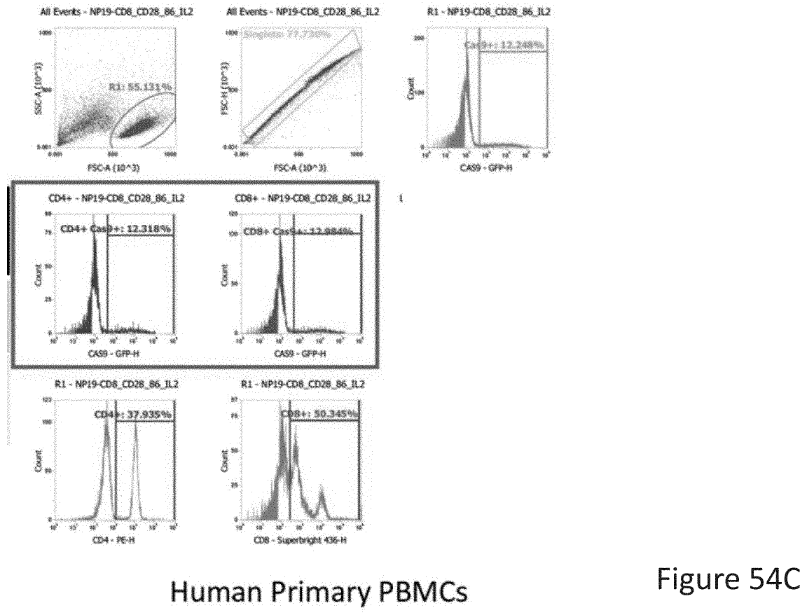

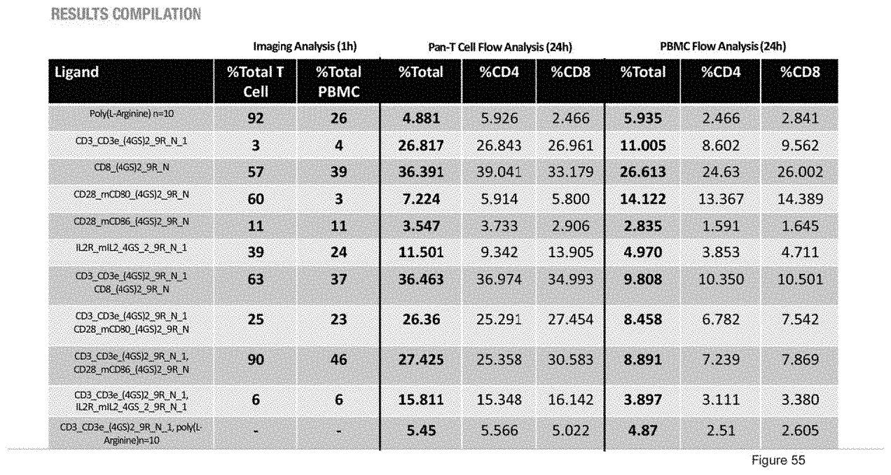

Exemplary data collected from this example is shown in FIGS. 34A-54C.

### Example 2: Multimodal Datasets

Cellular uptake and phenotype were characterized with flow cytometry and high-content screening, quantifying delivery efficiency and gene editing across various subpopulations of cells.

In these experiments, unstimulated human primary Pan-T Cells (a mixture of CD4+ and CD8+ T cells) and peripheral blood mononuclear cells (PBMCs) were flash transfected (30 minutes incubation with nanoparticles), washed twice with PBS containing 10 ug/ml heparan sulfate, and analyzed 24 hours later on an Attune N×T flow cytometer. Cells were stained with antibodies specific for CD4 and CD8, and transduction of EGFP-tagged Cas9 was quantified in each subpopulation. The following tables show comparisons of imaging performed 1 h post-transfection utilizing a BioTek Cytation 5 Imaging Reader with a 40× objective vs. flow cytometry data gathered at 24 h. We believe that 24 h time-points determine cellular internalization, whereas early time-points determine cellular affinity. Unsupervised learning was utilized for determining cellular affinity at the 1 h time-point from images, and imaging data was compared to cellular uptake at the 24 h time-point as assessed via flow cytometry.

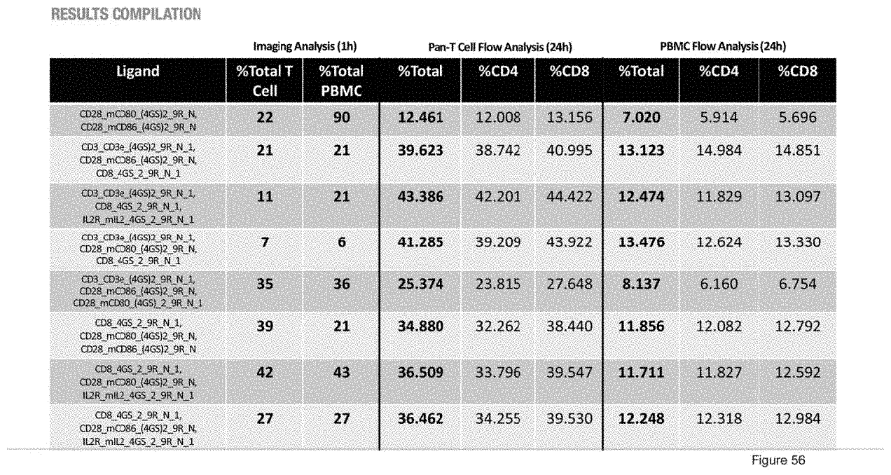

Exemplary data collected from this example is shown in FIGS. 55-56.

### Example 3: Optimization of Nanoparticle Cores for 4/5-Component Delivery

In the following experiments, three rounds of screening were performed to determine an optimal set of core formulations for co-delivery of a CRISPR-Cas9 RNP targeting the GFP or TCR locus, ssDNA for inserting an attP landing site, plasmid encoding a recombinase, and plasmid possessing an attB site for RFP insertion into the attP target locus. These experiments are performed prior to subsequent optimization of targeting ligand densities as detailed in Example 2 in a separate use-case (CRISPR-EGFP RNP delivery only).

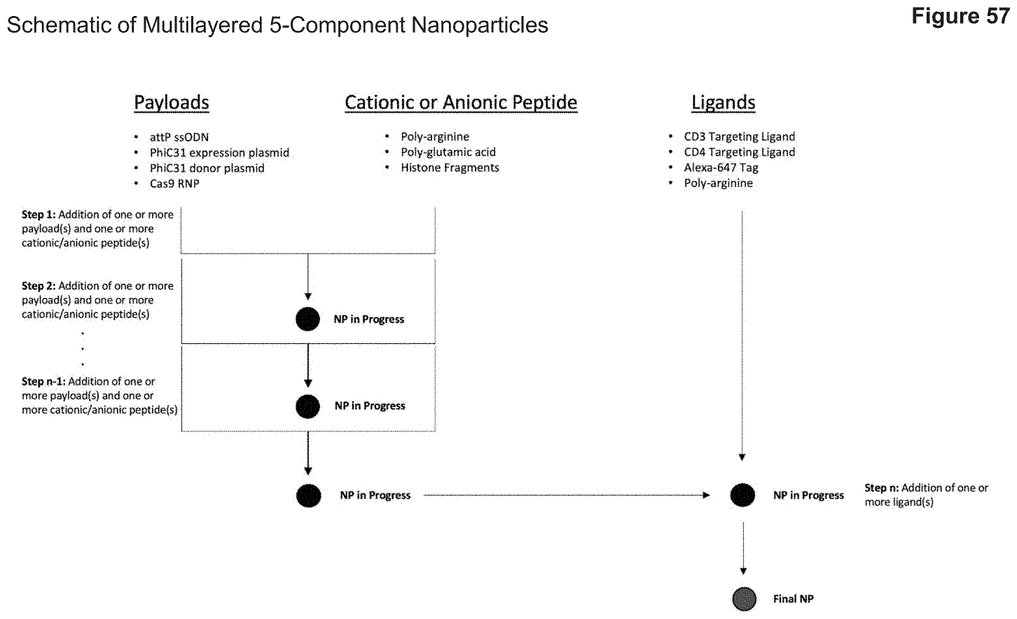

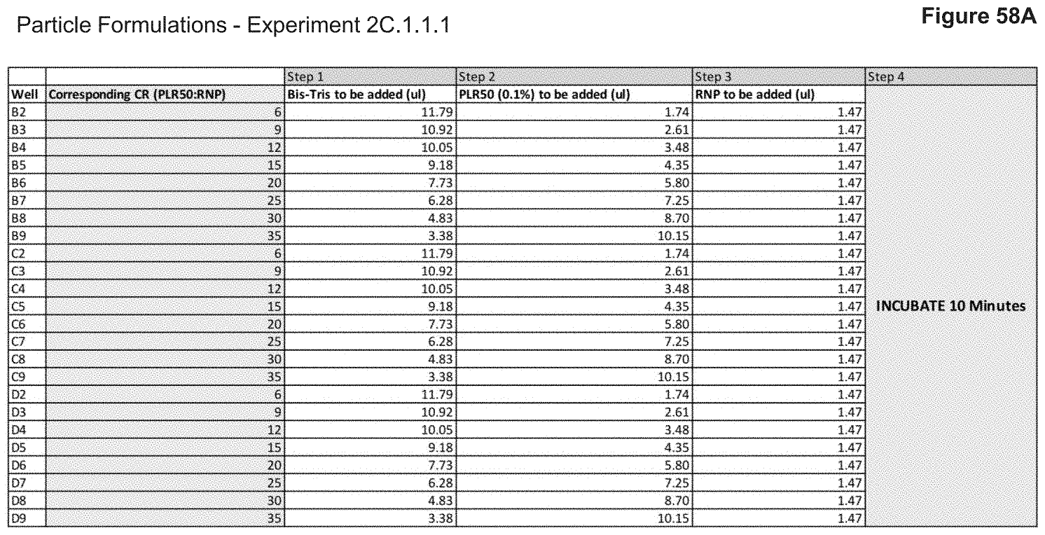

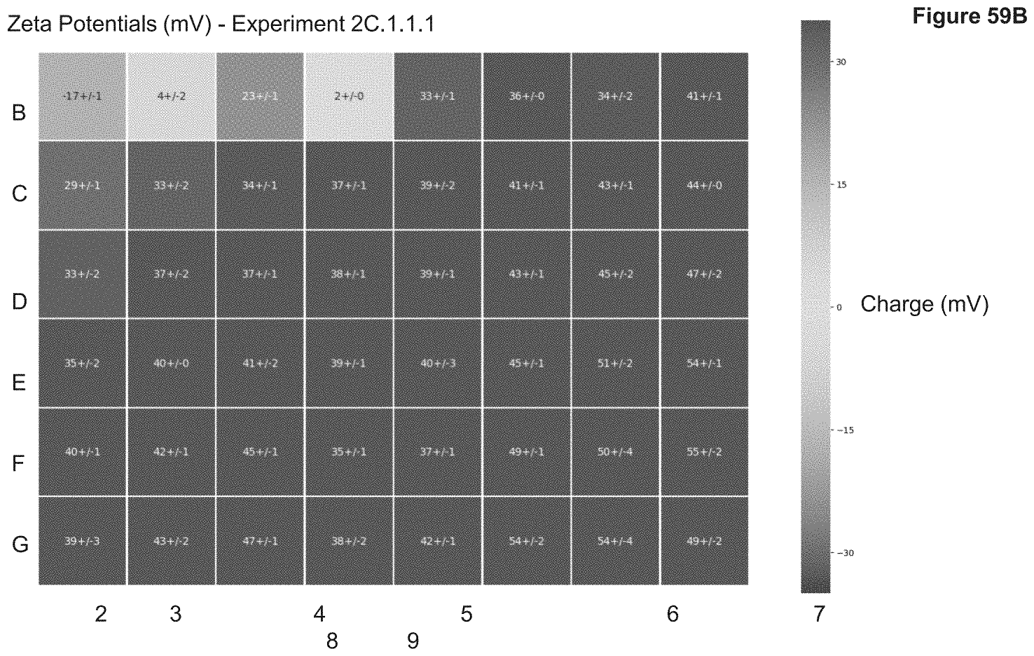

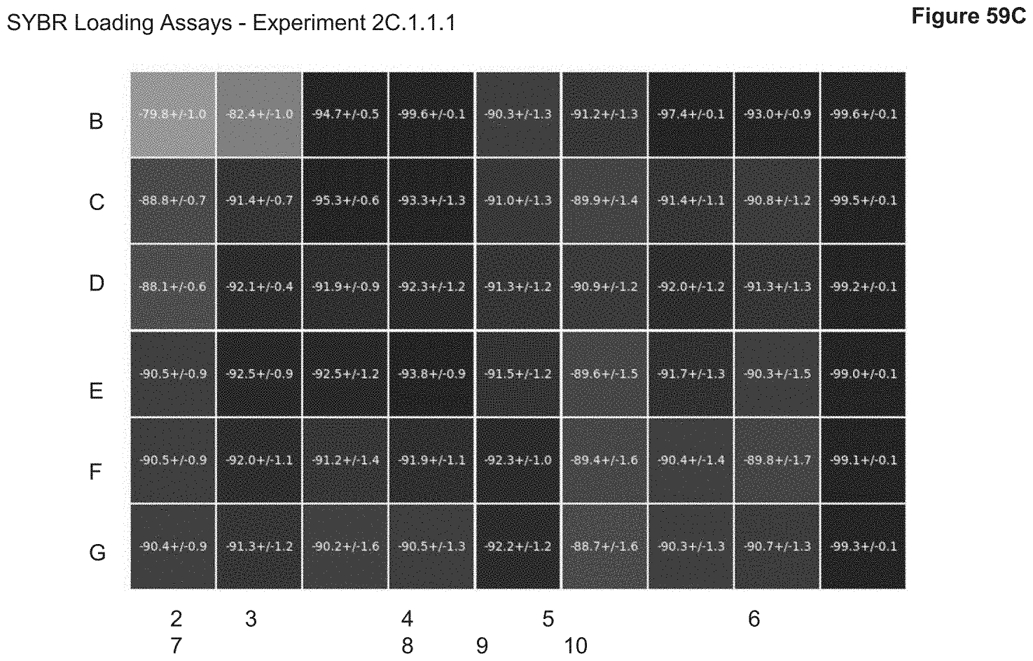

Exemplary data collected from this example is shown in FIGS. 57-62Y.

Physicochemical studies, namely SYBR assays, allow for determining payload condensation indices and subsequently understanding the relative percentage of genetic material that is condensed into a nanoparticle following various co-delivery formulation syntheses. Additionally, various particle embodiments include histone-derived and NLS-decorated sequences that may serve to be “transcriptionally active” substrates for histone-modifying enzymes and acetyl CoA, in addition to serving as crosslinking peptides due to their cysteine modifications throughout their electrostatic chains. In many cases, the most favorable payload condensation indices are closely correlated to particle sizes <200 nm and stable zeta potentials, as well as high degrees of particle uptake and/or gene editing.

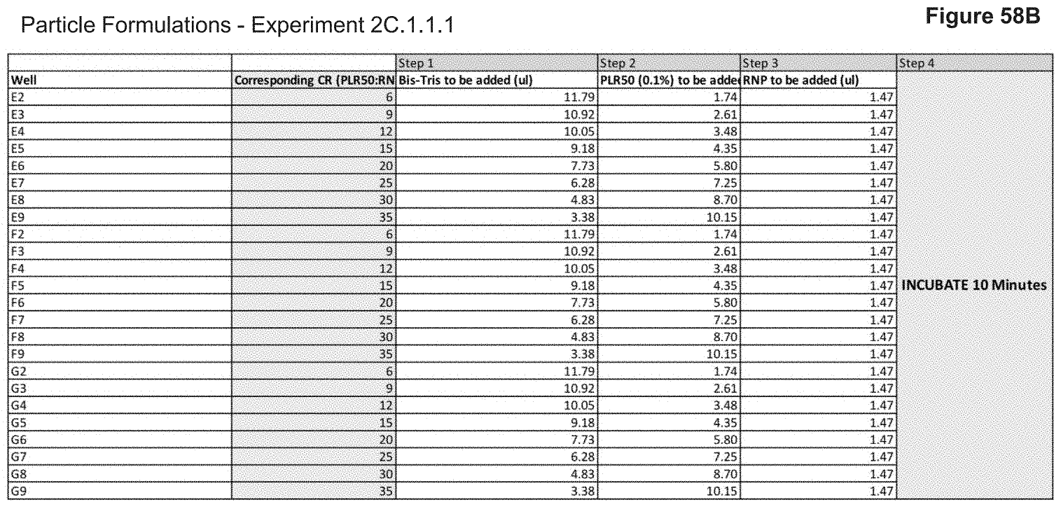

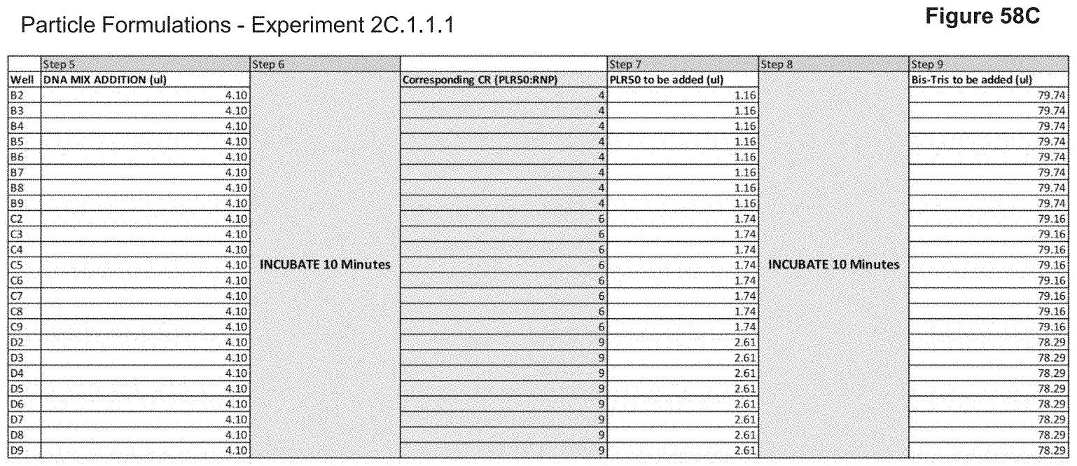

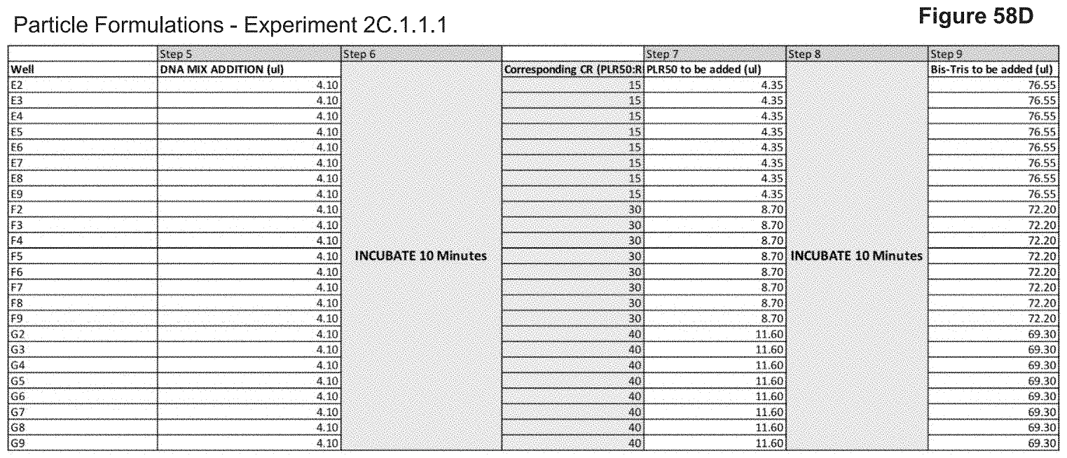

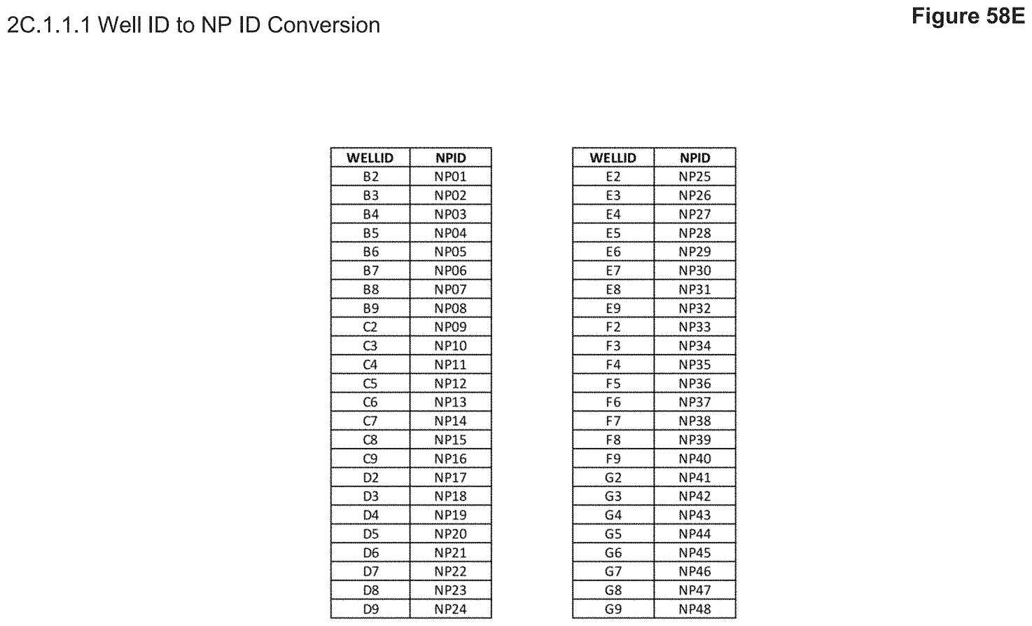

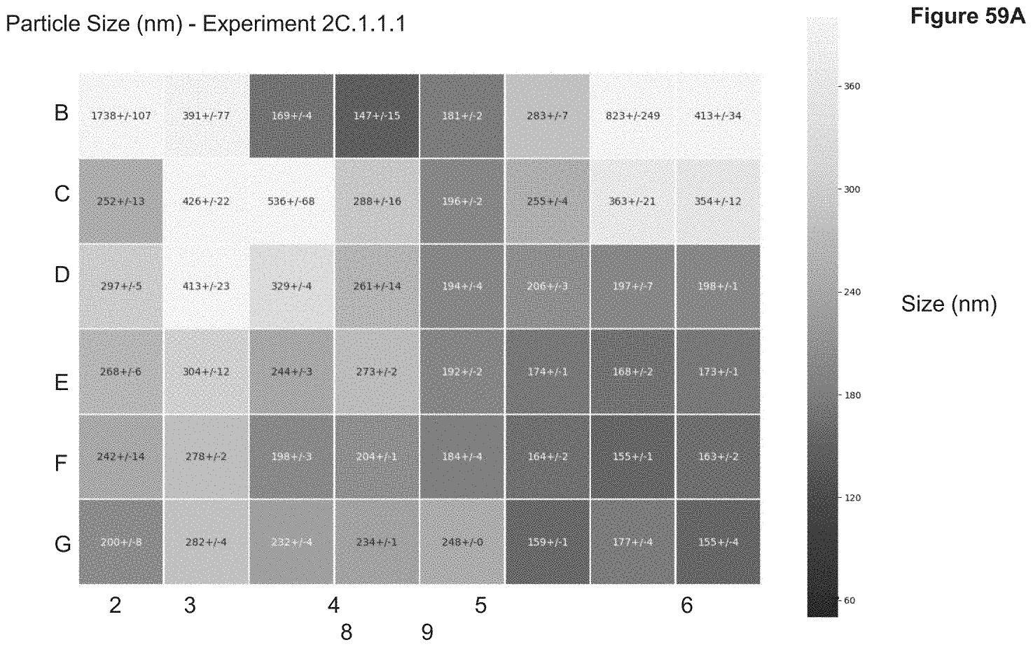

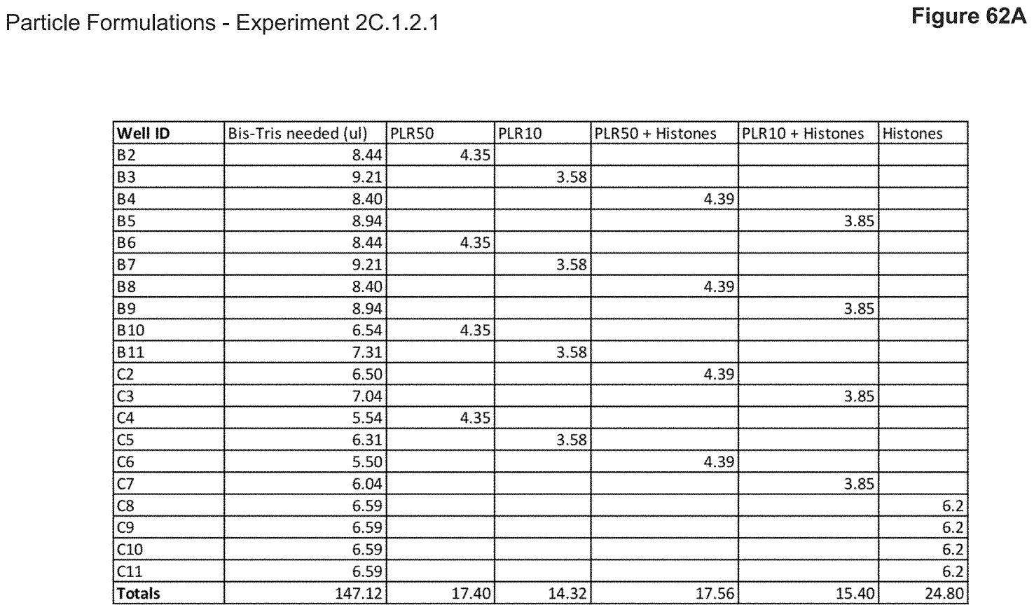

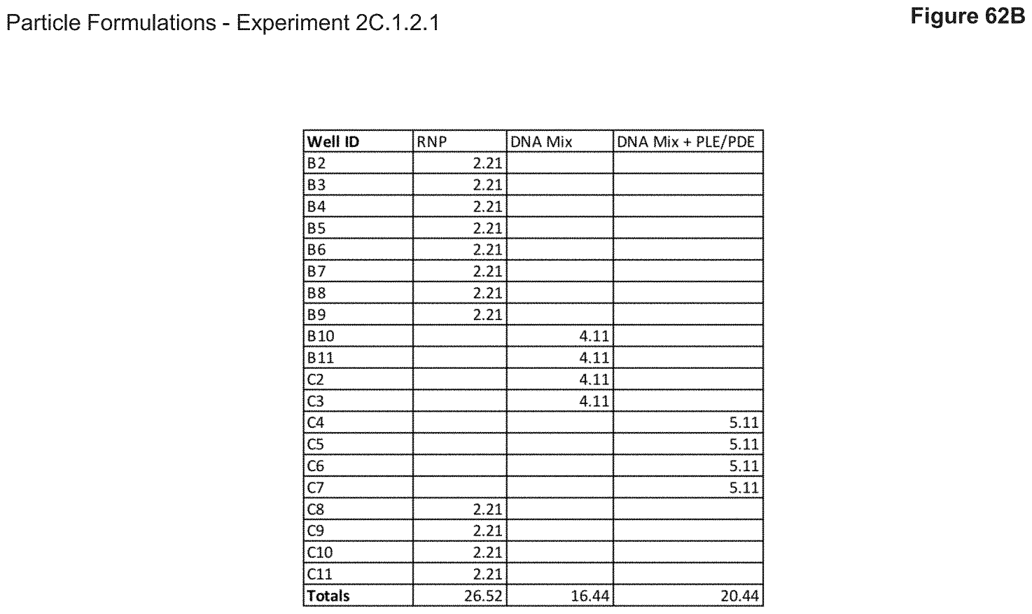

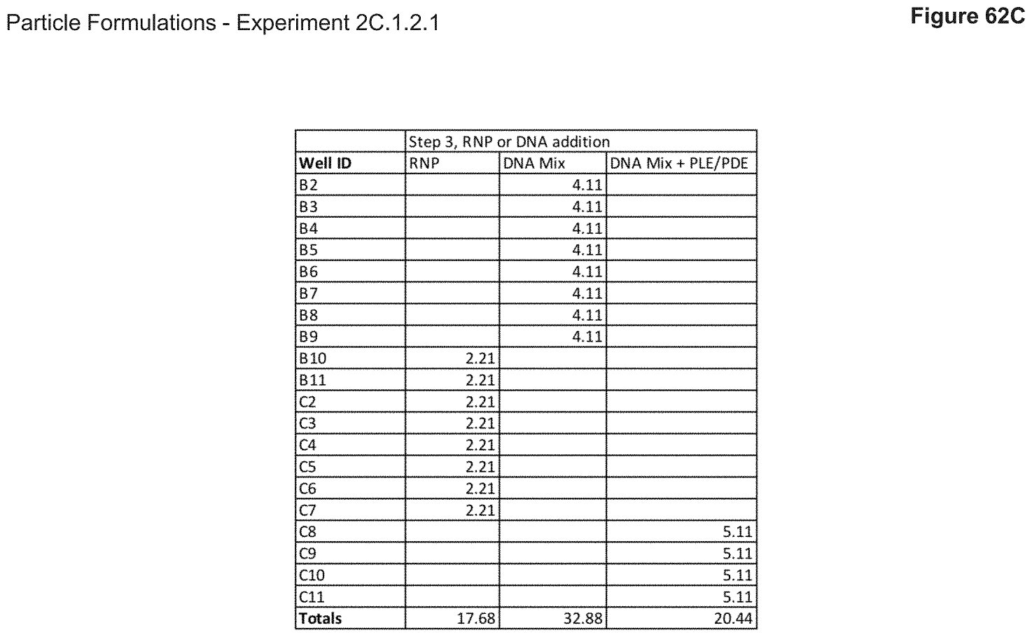

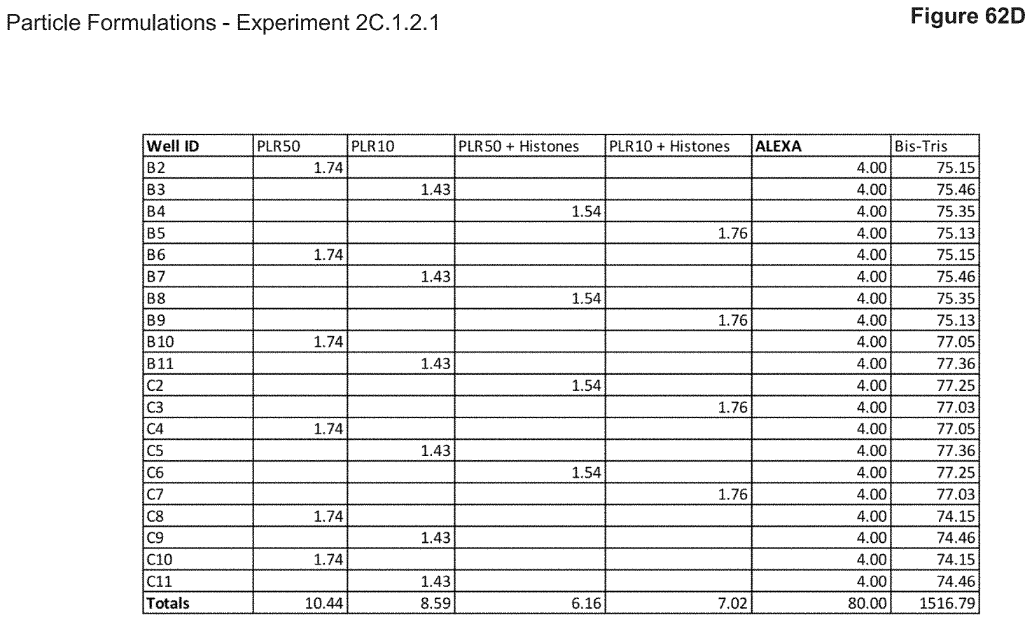

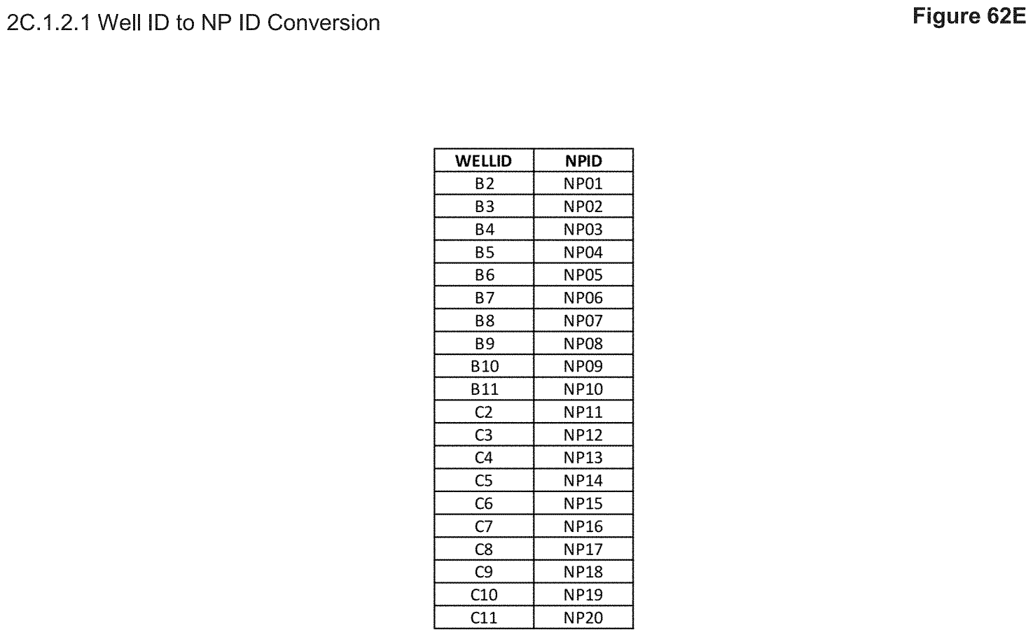

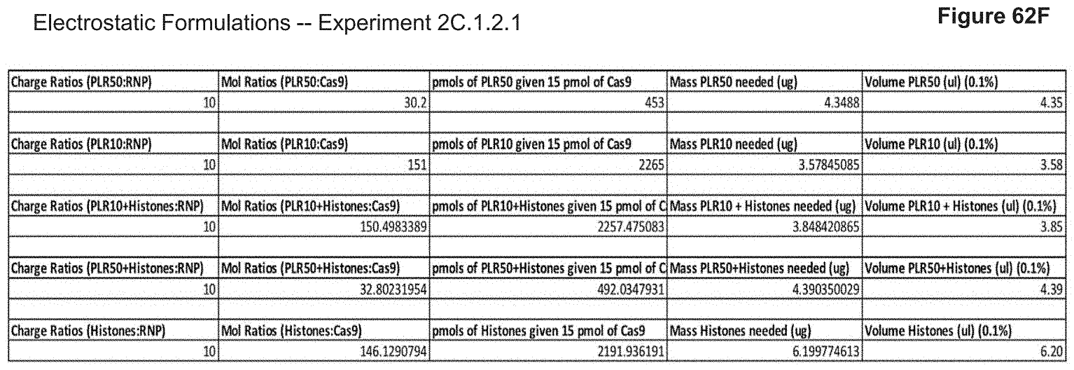

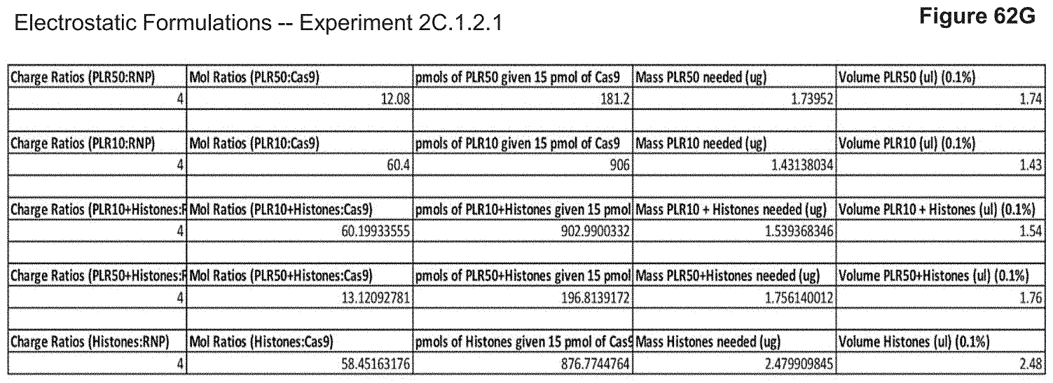

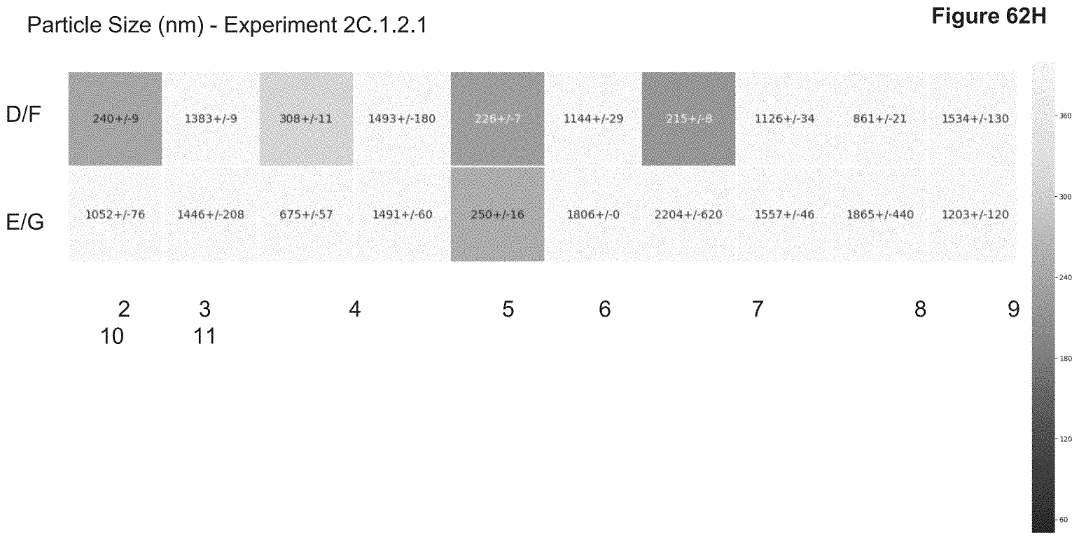

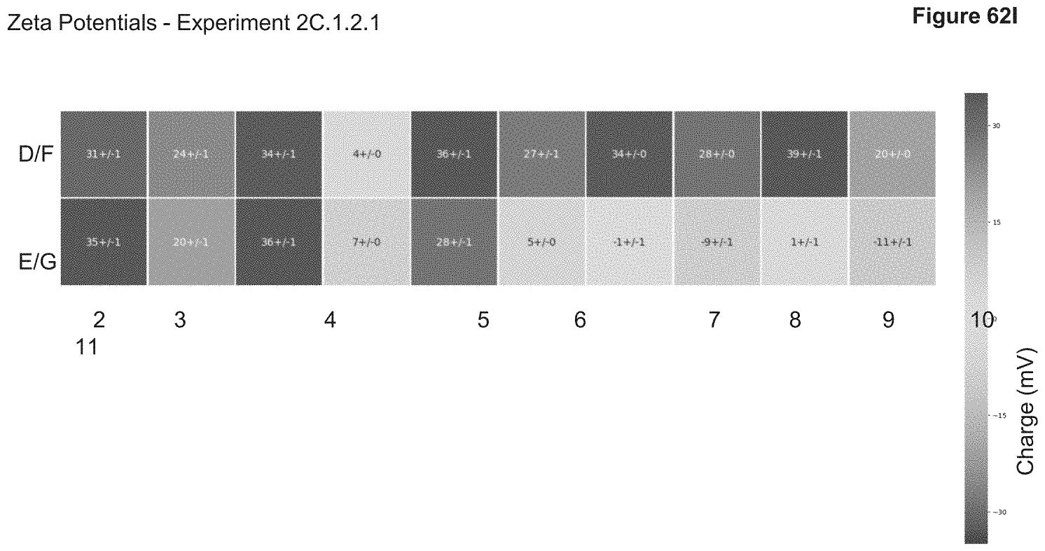

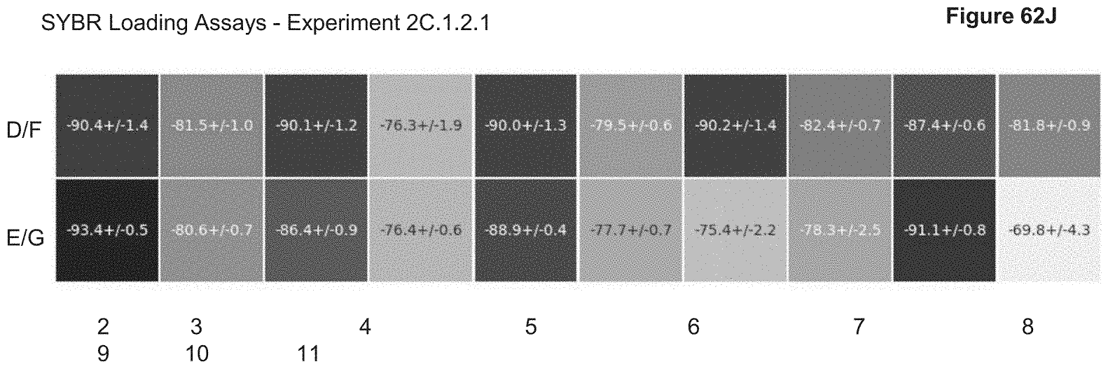

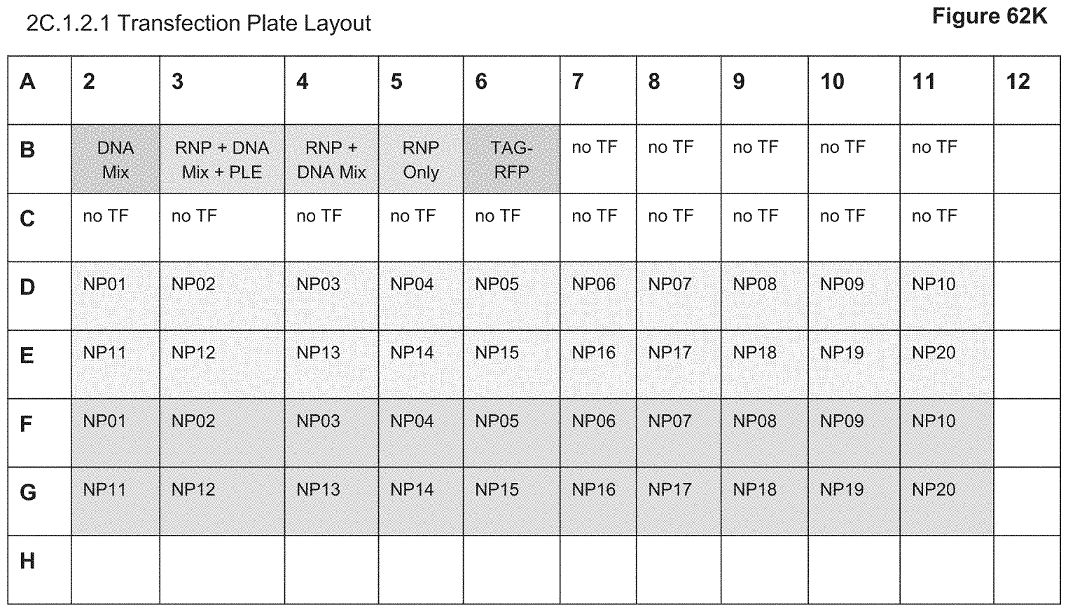

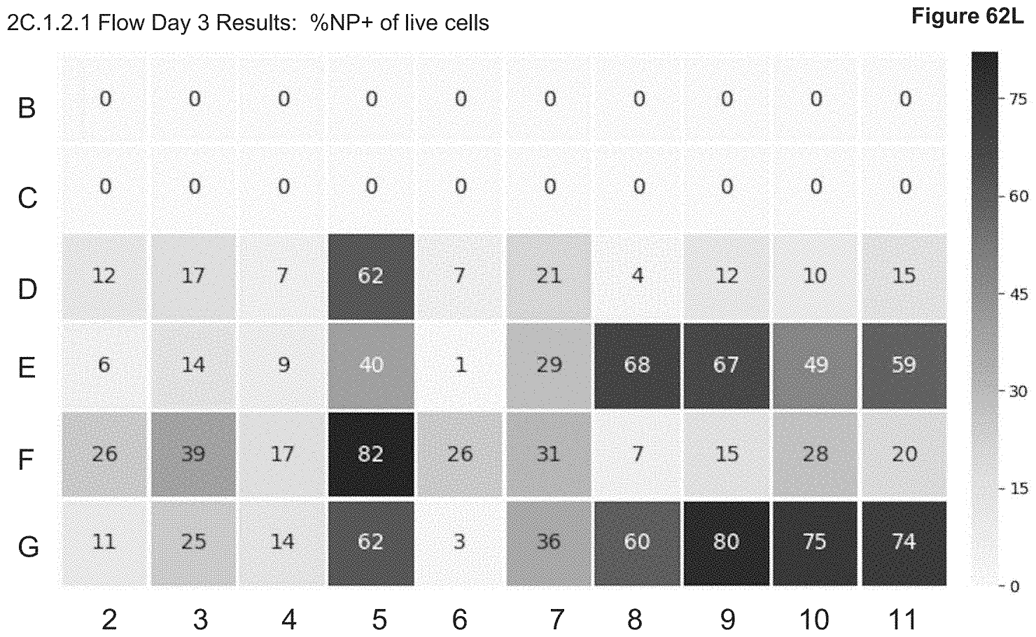

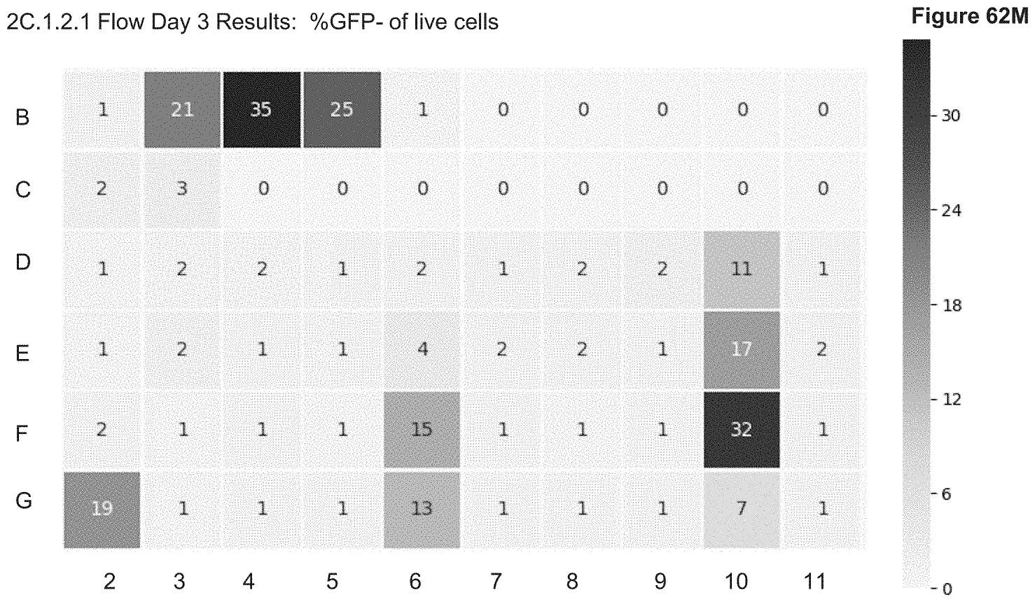

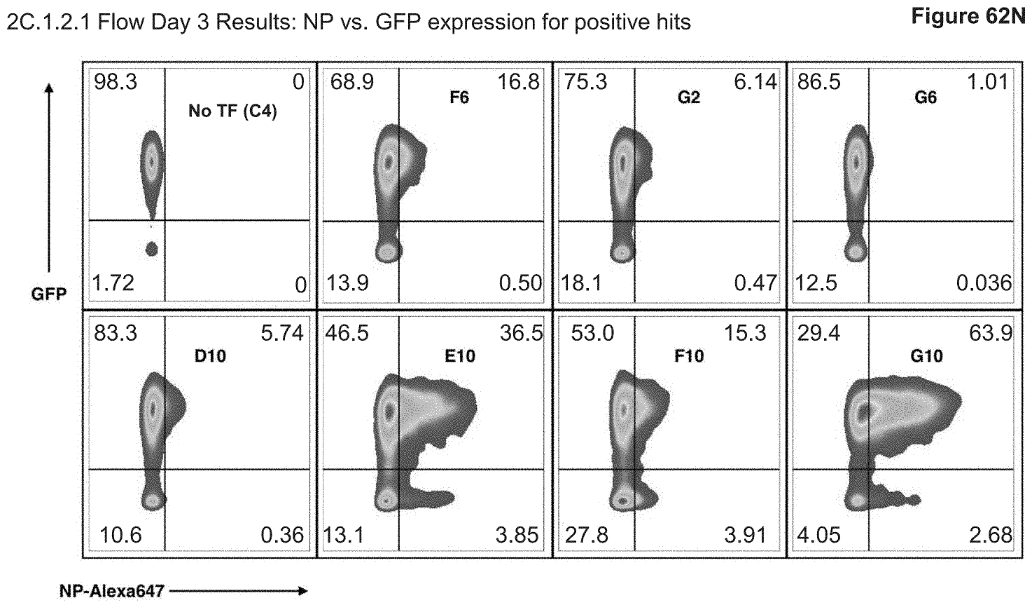

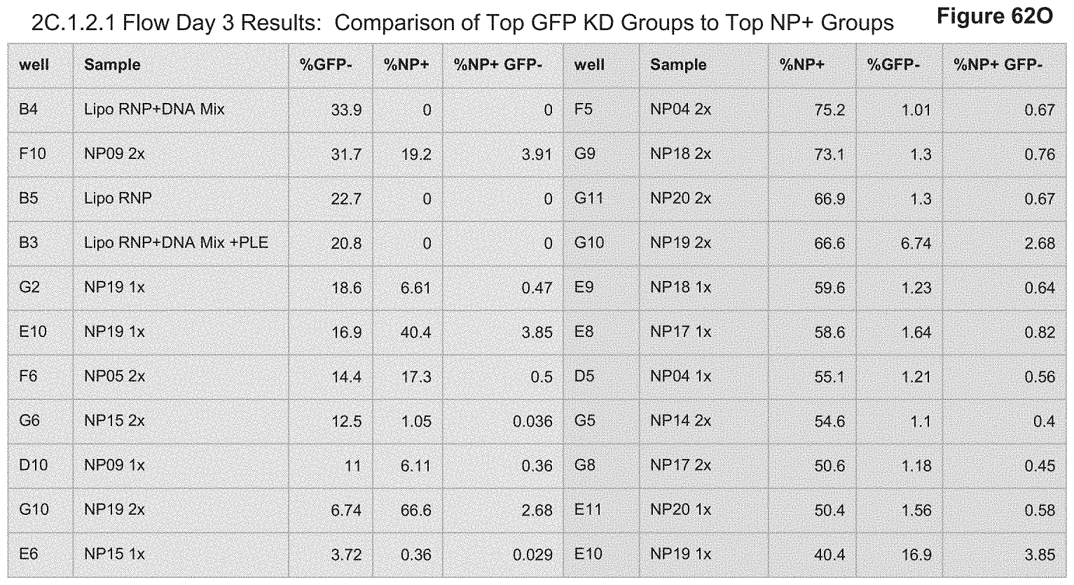

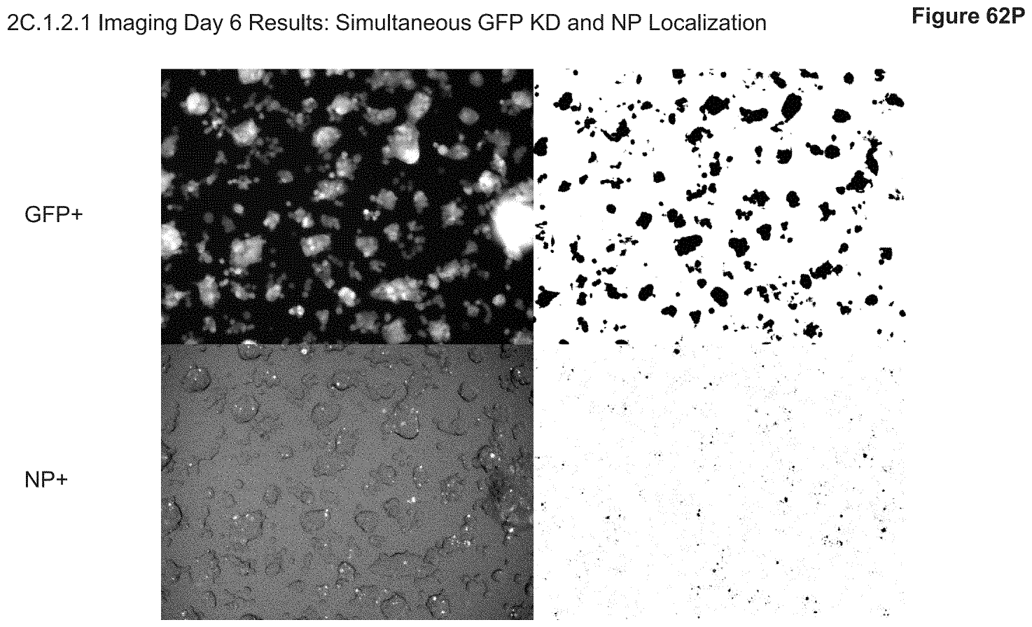

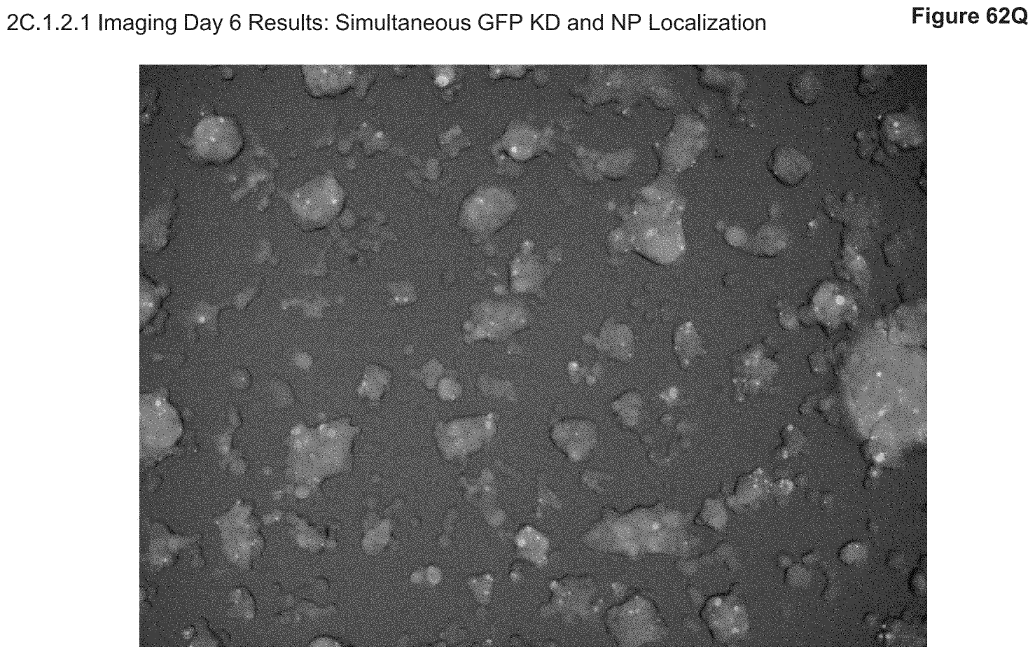

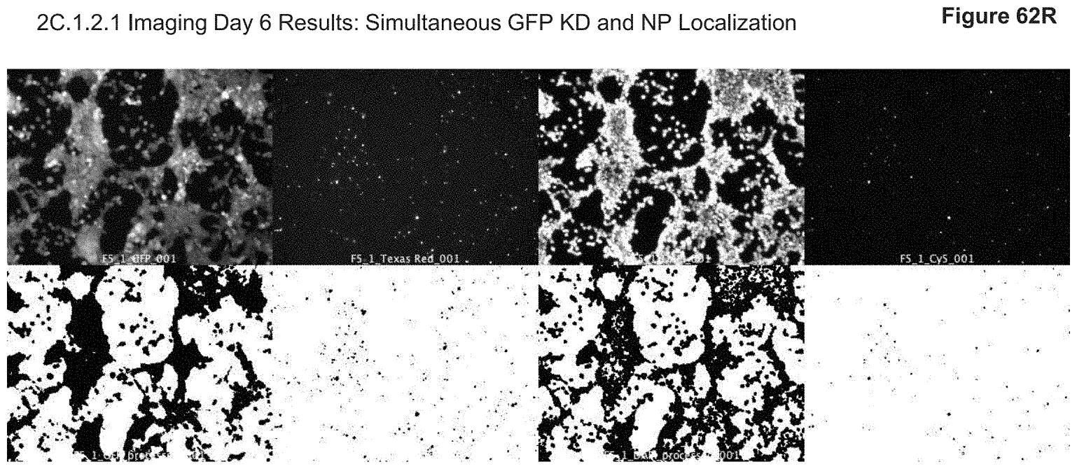

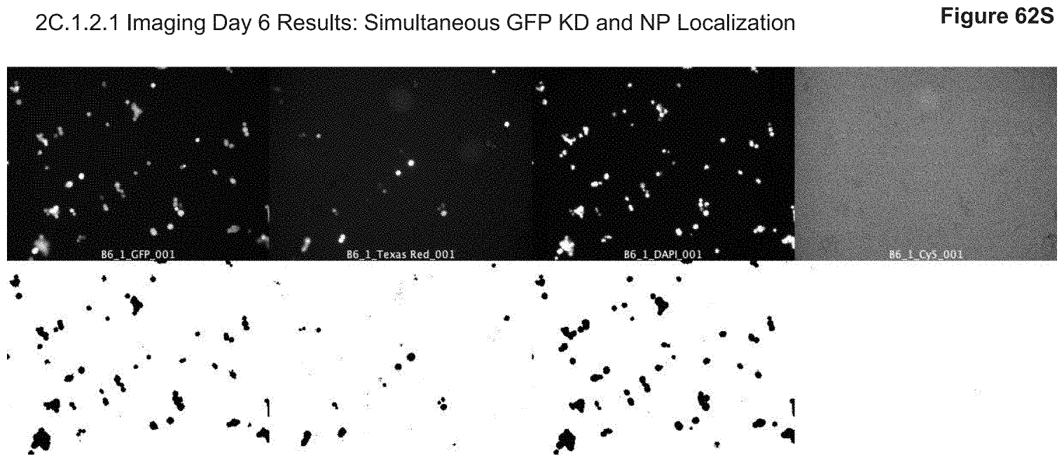

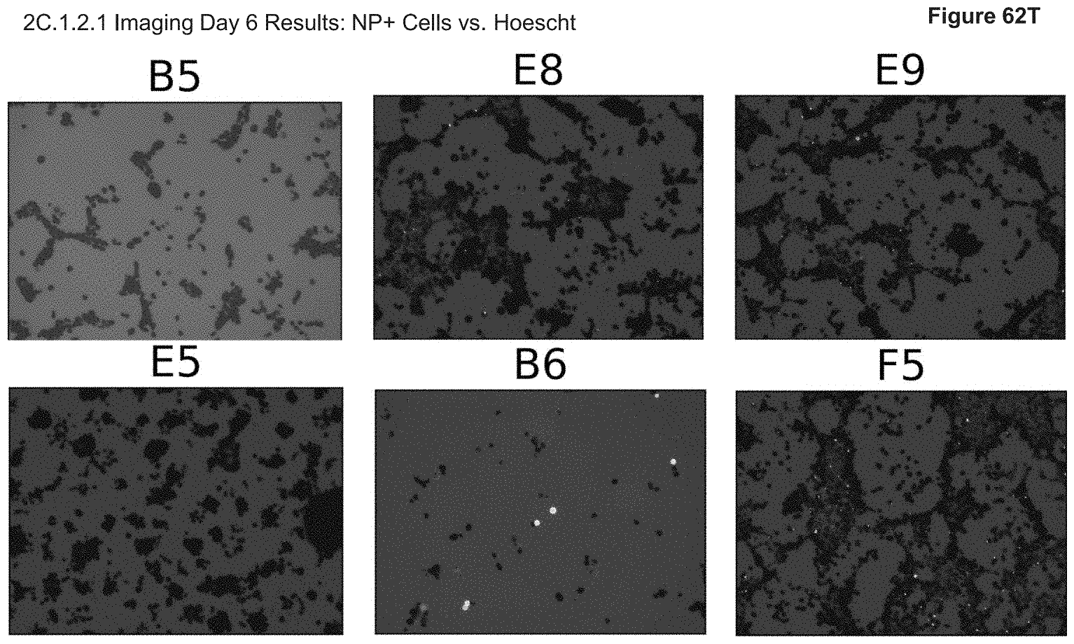

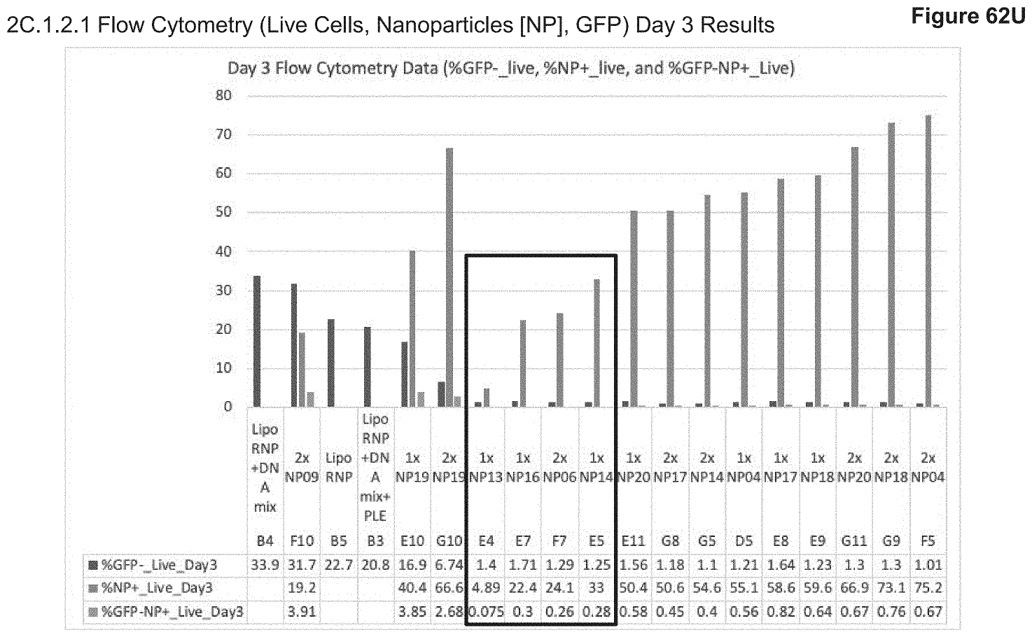

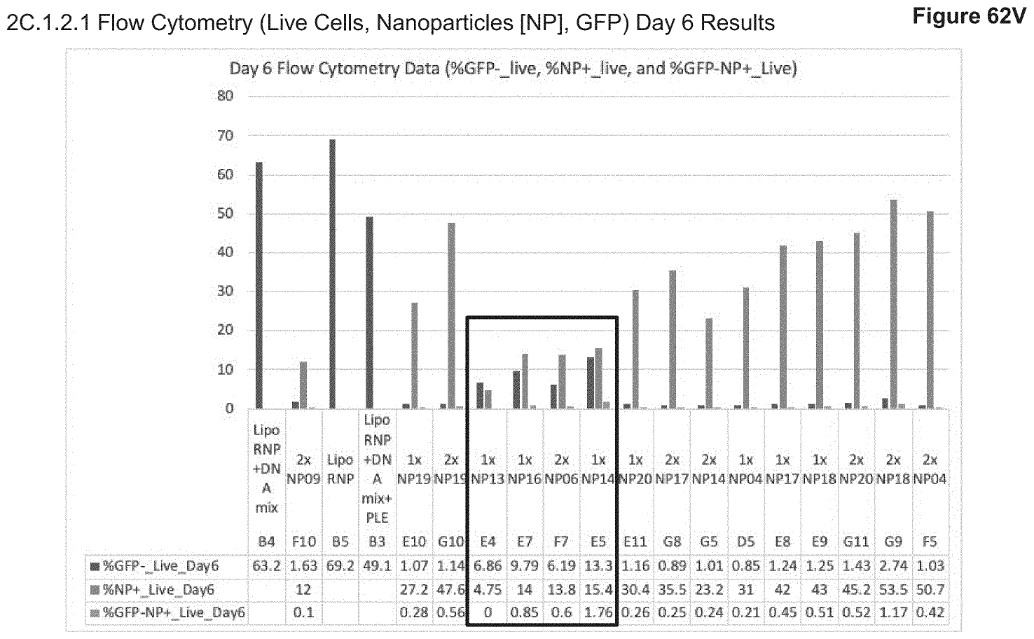

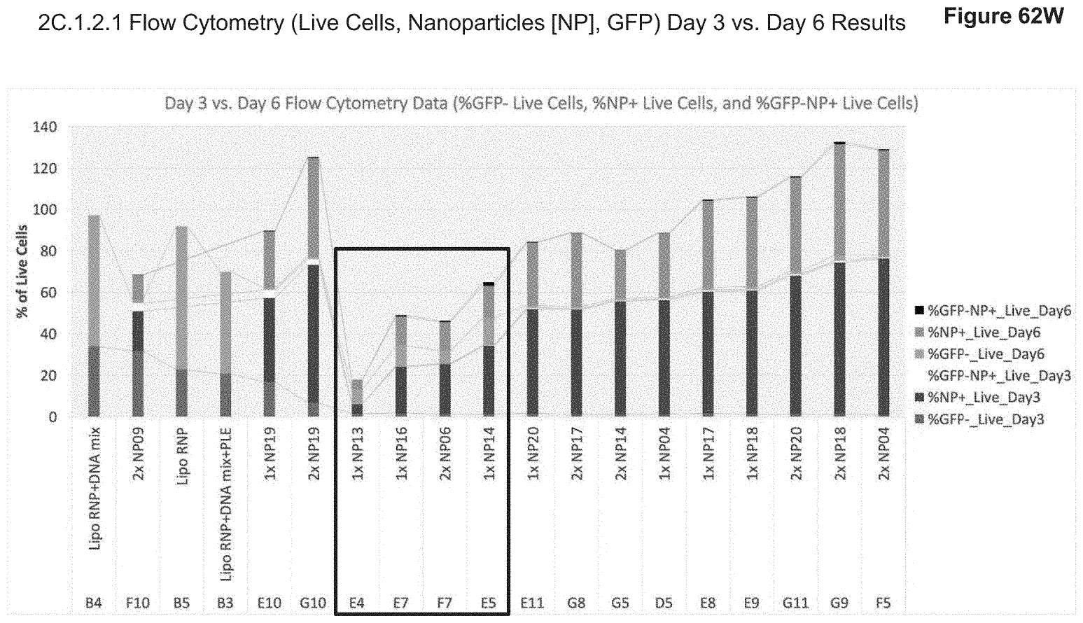

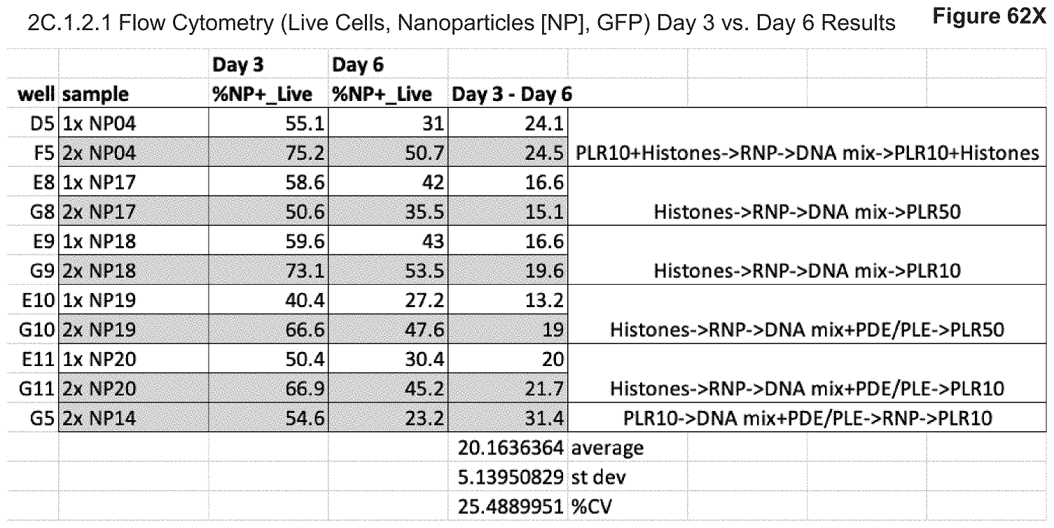

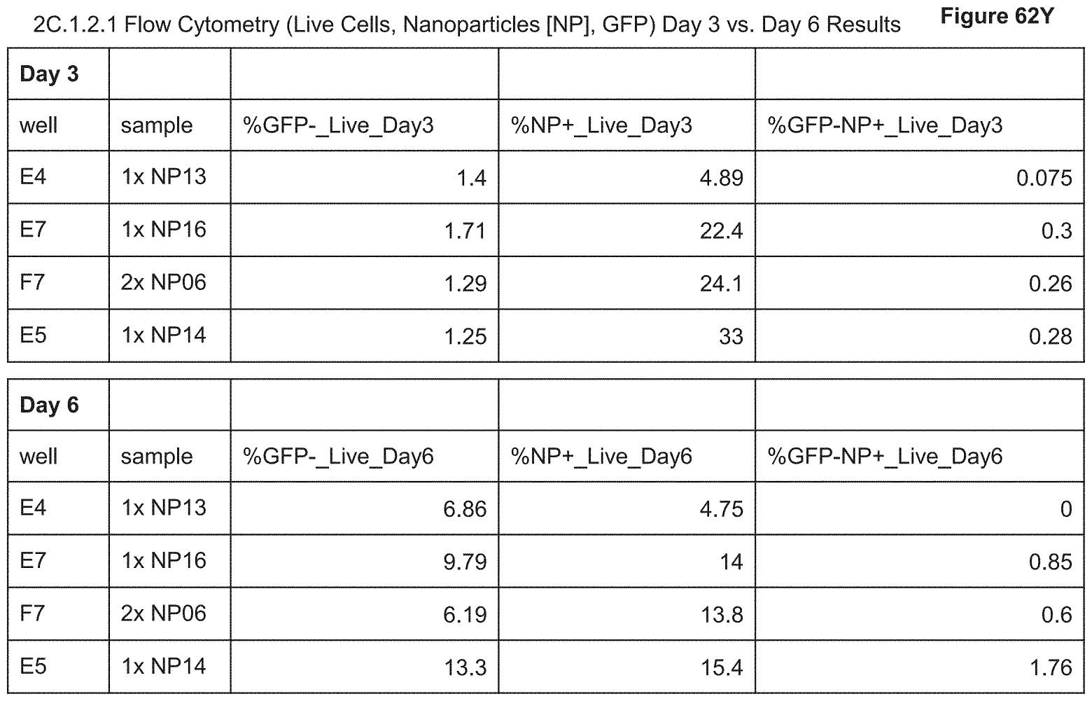

In these illustrative examples, 3 iterative cycles (FIGS. 58A-62Y) were performed to demonstrate that screening of the various electrostatic peptides, without targeting ligands, allows for optimization of nanoparticle “cores” to achieve functional gene editing, loading of all relevant payloads into nanoparticles (efficient SYBR condensation indices of 5-component nanoparticles with CRISPR-Cas9 RNP, ssDNA ODN, and two plasmids), and up to 58.9% transfection efficiencies (corresponding to 3.52% GFP k/d from CRISPR RNP) and 19.8% and 19.6% efficient GFP k/d efficiencies (corresponding to 18.6% particle uptake and 14.6% particle uptake, respectively). The particles in these studies are exceedingly stable and shield of the nucleic acid cargoes efficiently, and lead to efficient cellular targeting with variable subcellular release and functional editing efficiencies. The variability in % live cells that are nanoparticle+ cells between days 3 and 6 (two imaging time-points) is also apparent, whereby inclusion of variable poly(L-glutamic acid) to poly(D-glutamic acid) ratios alongside variable ratios of histone fragments (H2A and H2B as well as an NLS-modified histone fragment) and an endosomolytic, AF647-labeled functional peptide serve to generate various degrees of 1) subcellular release efficiency (NP+ cells vs. edited cells) and/or 2) extended vs. “quick-release” (6d+) residence of nanoparticles within the cellular environment (FIGS. 62U-62Y).

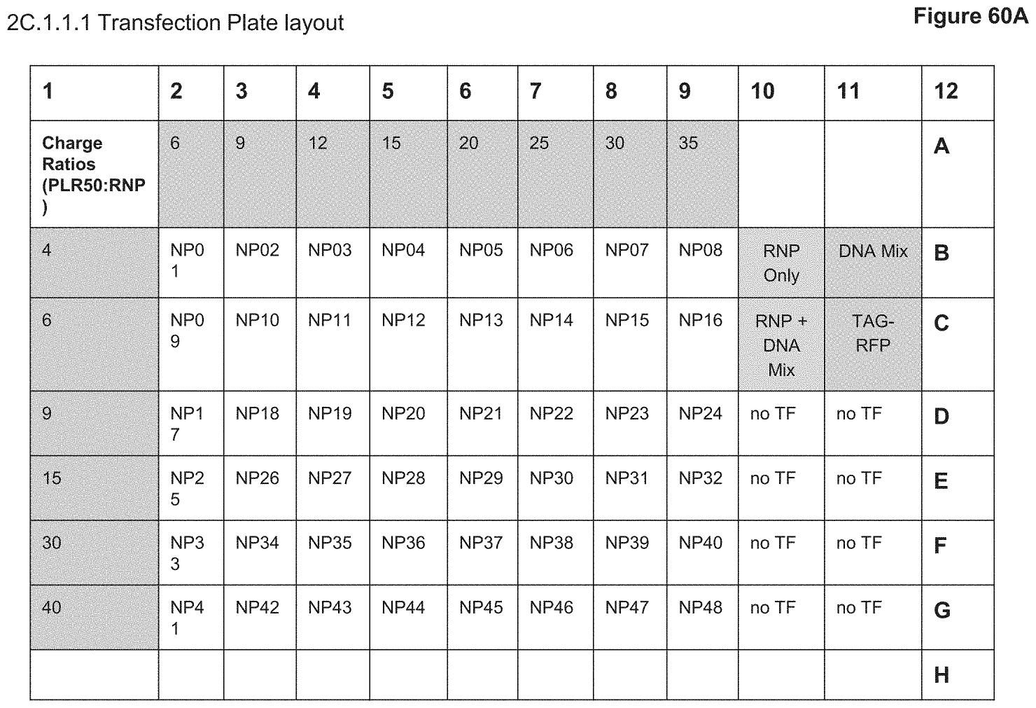

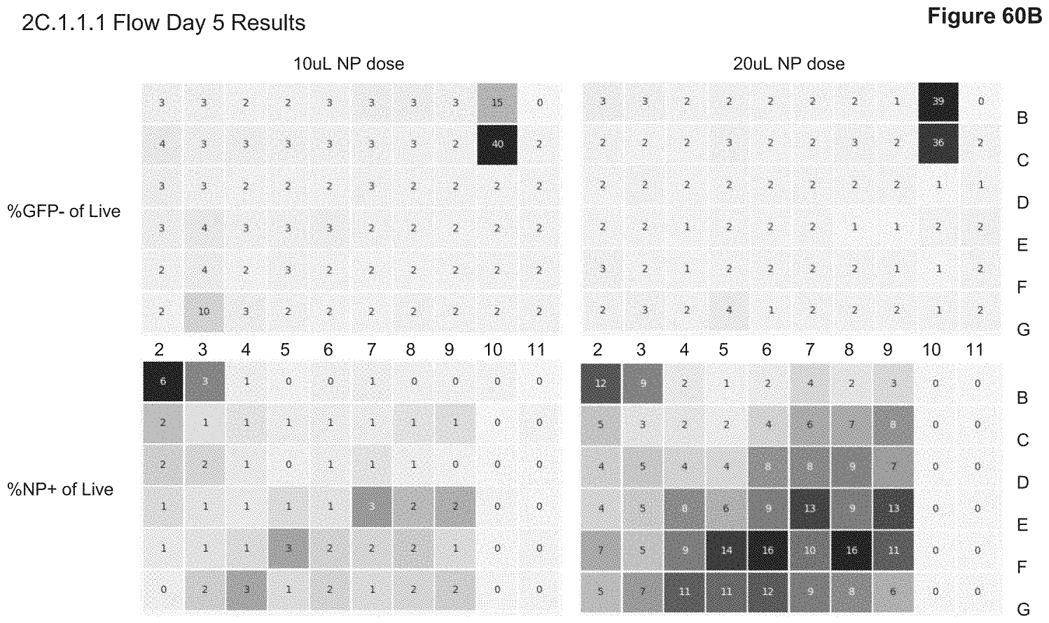

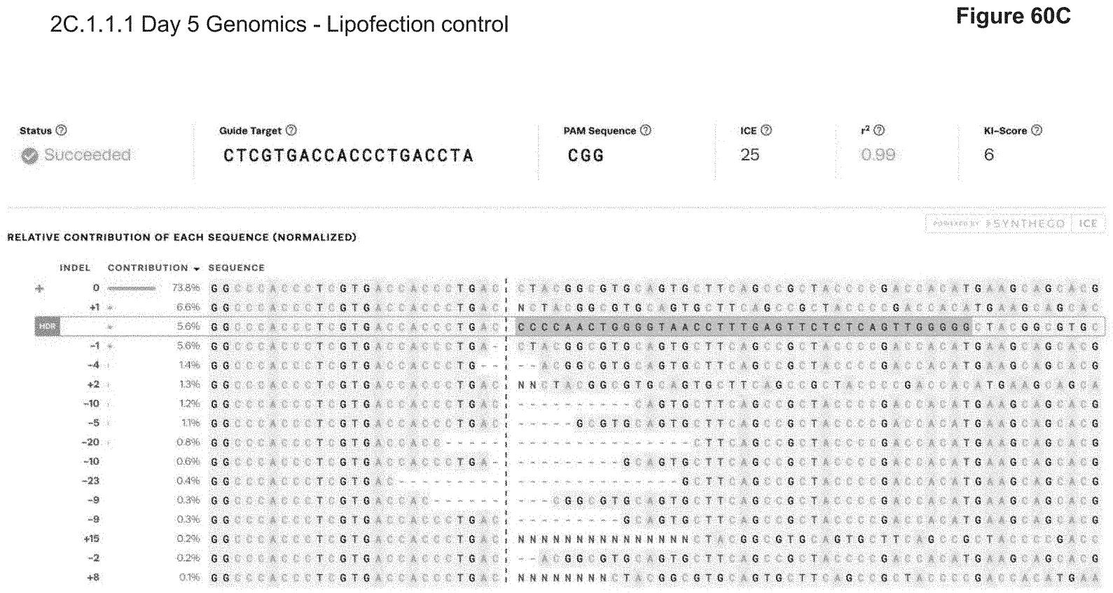

As can be seen from FIG. 60I, many cells are GFP− with nanoparticle transfections, and the percentage of GFP− cells (bottom half of flow plot: y-axis represents GFP intensity and x-axis represents NP-Alexa647 intensity) in representative groups varies considerably in terms of GFP+NP+ cells vs. GFP+NP− cells, presumably due to a) varying condensation efficiencies of the given electrostatic polymers, b) varying subcellular trafficking efficiency of the electrostatic polymers, c) varying compartment-specific nuclear release of the electrostatic polymer-bound payloads, and/or d) varying timed release profiles associated with a given “layer” leading to variability in functional genome editing potential of the given formulation.

### 1. Nanoparticle Synthesis

Peptides were synthesized using standard Fmoc-based solid-phase peptide synthesis (SPPS). Sequences were synthesized from the C- to N-direction. The first Fmoc-protected amino acid was coupled onto NovaPeg Rink amide resin (Millipore Sigma) using 1-[Bis(dimethylamino)methylene]-1H-1,2,3-triazolo[4,5-b]pyridinium 3-oxide hexafluorophosphate (HATU) with N-methyl morpholine (NMM) in dimethyl formamide (DMF). The Fmoc protecting group was removed using 20% 4-methyl piperidine (4PIP) in DMF. Subsequent coupling and deprotection were performed for each amino acid in the peptide polymer. The completed peptide was cleaved from the resin and globally deprotected using 5 mL of a cleavage cocktail (4.5 mL trifluoroacetic acid:250 uL water:250 uL triisopropyl silane) and mixed for 90 minutes. Cleaved peptide was collected by passing the resin and cocktail solution through a disposable column equipped with a frit. Peptide was precipitated from the TFA solution using 50 mL of cold diethyl ether (4° C.). Diethyl ether was removed and crude peptide was washed with additional cold ether (2×50 mL) and dried under a stream of nitrogen gas. Crude peptide was dissolved in 20% acetonitrile (ACN) in water (˜5 mL) and fractionated by reverse-phase chromatography. Purified fractions were combined, frozen, and lyophilized to yield purified peptide as a powder.

The following materials were purchased:

NLS-Cas9-GFP (Genscript Z03393)
NLS-Cas9-NLS (Aldevron 9212)
LL285 (Synthego)-guide sequence:
(SEQ ID NO: 278)
CTCGTGACCACCCTGACCTA
(ref. Glaser et al. 2016)
(SEQ ID NO: 279)
LL295 IDT-
gcgatgccacctacggcaagctgaccctgaagttcatctgcaccaccgg
caagctgcccgtgccctggcccaccctcgtgaccaccctgacCCCCAAC
TGGGGTAACCTTTGAGTTCTCTCAGTTGGGGGctacggcgtgcagtgct
tcagccgctaccccgacca
LL224 (Synthego) guide sequence:
(SEQ ID NO: 280)
AGAGTCTCTCAGCTGGTACA
(ref. Roth et al. 2018)
(SEQ ID NO: 281)
LL294 IDT-
GTCCCACAGATATCCAGAACCCTGACCCTGCCGTGTCCCCAACTGGGGT
AACCTTTGAGTTCTCTCAGTTGGGGGACCAGCTGAGAGACTCTAAATCC
AGTGACAAGTCTGTCTGCCTATTCACCGATTTTGATTCTCAAACAAATG
TGTCACAAAGTAAGGATTC
pLL312 System Biosciences FC200-PA1
pLL313 System Biosciences FC550A-1
pTagRFP-N Evrogen FP142

TABLE 1
Nanoparticle Formulation key

| | | | Molecular |
|---|---|---|---|
| ID | Name | Isomer | Weight (g/mol) |
| en1 | NLS-Cas9-GFP | L | 186229 |
| en4 | NLS-Cas9-NLS | L | 160171 |
| LL285 | sgRNA GFP | — | — |
| LL295 | attP ssODN GFP | — | — |
| LL224 | sgRNA TRAC | — | — |
| LL294 | attP ssODN TRAC | — | — |
| pLL312 | PhiC31 expression plasmid | — | — |
| | (CMV promoter) | | |
| pLL313 | PhiC31 donor plasmid | — | — |
| | (EF1a promoter::RFP) | | |
| cpp1 | Poly-arginine[10] | L | 1580 |
| cpp2 | Poly-arginine [50] | L | 9600 |
| app4 | Poly-Lysine [20] | L | 3000 |
| app5 | Poly-Lysine [20] | D | 3000 |
| app4 + app5 (1:1) | PLE/PDE Mix | L/D | 3000 |
| cpp10 | H2B-3C | L | 2385 |
| cpp12 | H2A-3C | L | 2410 |
| cpp13 | NLS-H2A-3C-NLS | L | 3690 |
| c10 + c12 + c13 | Histone Mix | L | 2828 |
| (1:1:1) | | | |
| cl11 | CD3_Targeting | L | 3094 |
| cl24 | Alexa-647 + Endosomal | L | 4890 |
| | Escape Peptide | | |
| cl25 | CD4_Targeting | L | 2943 |
| cl11 + cl25 (1:1) | Ligand Mix | L | 3019 |
| cpp1 + [c10 + c12 + | PLR10 + Histone Mix | L | 1704 |
| c13(1:1:1)] | | | |
| (10:1) | | | |
| cpp2 + [c10 + c12 + | PLR50 + Histone Mix | L | 8922 |
| c13(1:1:1)] (10:1) | | | |

Peptide Sequences:

| ID | Sequence |
|---|---|
| cpp1 | (R)10 (SEQ ID NO: 282) |
| cpp2 | (R)50 (SEQ ID NO: 283) |
| cpp10 | CEVSSKGATICKKGFKKAVVKCA-NH2 (SEQ ID NO: 284) |
| cpp12 | CSGRGKQGCKARAKAKTRSSRCA-NH2 (SEQ ID NO: 285) |
| cpp13 | KKKRKSCRGKQGCKARAKAKTRSSRCAKKKRK (SEQ ID NO: 286) |
| app4 | (E)20 (SEQ ID NO: 287) |
| app5 | (E)20 (SEQ ID NO: 287) |
| cll11 | RRRRRRRRRGGGGSGGGGSNFYLYLRA-NH2 (SEQ ID NO: 289) |
| cll24 | KKKRKKKKRKGGGGSC(Alexa647) (SEQ ID NO: 290) |
| cll25 | GGGGSSFKFLFDIIKKIAESF-NH2 (SEQ ID NO: 290) |
| cll25 | RRRRRRRRRGGGGSGGGGSFTDNAKTI-NH2 (SEQ ID NO: 291) |

Ligands (cll11, cll25) include a 9× arginine anchor. Histone fragments (cpp10, cpp12, cpp13) are modified with cysteine groups to help form cross-linked meshes. Cpp13 is also modified with a nuclear localization signal.

Overview: This experiment was conducted iteratively in 3 subparts. 2 subparts (titled 2C.1.1.1 and 2C.1.2.1) involve a 4 component payload nanoparticle transfection of HEK293-GFP stable cells. The payload is composed of: NLS-Cas9-NLS Ribonucleoprotein with sgRNA targeting the GFP locus, ssODN encoding attP with asymmetric homology arms, PhiC31 integrase expression plasmid, and attB donor plasmid encoding RFP-T2A-PuromycinR under EF1a promoter. Another subpart (titled 2C.2.1.1) involves a 4/5-component payload nanoparticle transfection of CD3/CD28 bead stimulated peripheral blood mononuclear cells (PBMCs). The payload is composed of: GFP− Cas9-NLS Ribonucleoprotein with sgRNA targeting the TRAC locus, ssODN encoding attP with asymmetric homology arms, PhiC31 integrase expression plasmid, and attB donor plasmid encoding RFP-T2A-PuromycinR under EF1a promoter.

2C.1.1.1:

Cas9 Ribonucleoprotein (RNP) was composed using a 1.2:1 sgRNA:Cas9 ratio and incubated for 30 minutes at room temperature. LL285 and LL295 were hydrated from lyophilized state to 0.1% w/v with ultra-pure water (mili-Q water). pLL312 and pLL313 were diluted to 0.05% w/v with water and a stock 1:50 pLL313:pLL312 was created as the plasmid stock. Peptides were diluted to 0.1% w/v using 0.1M Bis-Tris pH 8.5. 48 unique nanoparticles were formed at a final volume of 100 ul in a stepwise manner with varying amounts of 50-mer Poly-L-Arginine (PLR50) to vary the charge ratio (ratio of total charge of molecule A to molecule B) between PLR50 (+50 total charge per molecule) and RNP (−151 total charge per molecule) in the following order of addition:

1. PLR50

2. RNP

3. DNA mix (ssDNA+plasmid stock)

4. PLR50

5. Buffer

Layers 1 and 2 were combined and incubated for 10 min then layer 3 was added to layers 1 and 2 and incubated for an additional 10 minutes. Layer 4 was then added and incubated with the previous layers. Finally, buffer was added to each formulation to bring the total volume to 100 uL. Charge ratios of the layer 1 (PLR50:RNP) ranged from 6-35 and charge ratio of layer 4 (PLR50:RNP) ranged from 4-40.

Each unique formulation had 2500 ng ssDNA and 800 ng pDNA implying that every dose (10 ul) delivered 250 ng ssDNA and 80 ng pDNA. Each unique formulation had 10 pmol of Cas9 implying that every dose (10 ul) delivered 1 pmol of Cas9. For this experiment, 2 plates of 40K HEK293-GFP were transfected, dosed at 10 uL and 20 uL NP per well.

2C.1.2.1:

Cas9 RNP was composed using a 1.2:1 sgRNA:Cas9 ratio and incubated for 30 minutes at room temperature. LL285 and LL295 were hydrated from lyophilized state to 0.1% w/v with ultra-pure water (mili-Q water). pLL312 and pLL313 were diluted to 0.05% w/v with water and a stock 1:50 pLL313:pLL312 was created as the plasmid stock. Peptides were diluted to 0.1% w/v using 0.1M Bis-Tris pH 8.5. 20 unique nanoparticles were formed at a final volume of 100 ul with varying orders of addition using the following components and orders:

| | | | | | | | | | |
|---|---|---|---|---|---|---|---|---|---|
| PLR50 -> | PLR10 -> | PLR50 + | PLR10 + | PLR50 -> | PLR10 -> | PLR50 + | PLR10 + | PLR50 -> | PLR10 -> |
| RNP -> | RNP -> | Histones-> | Histones -> | RNP -> | RNP -> | Histones -> | Histones -> | DNA mix -> | DNA mix -> |
| DNA mix -> | DNA mix -> | RNP -> | RNP -> | DNA mix + | DNA mix + | RNP -> | RNP -> | RNP -> | RNP -> |
| PLR50 | PLR10 | DNA mix -> | DNA mix-> | PDE/PLE -> | PDE/PLE -> | DNA mix + | DNA mix + | PLR50 | PLR10 |
| | | PLR50 + | PLR10 + | PLR50 | PLR10 | PLE/PDE -> | PDE/PLE -> | | |
| | | Histones | Histones | | | PLR50 + | PLR10 + | | |
| | | | | | | Histones | Histones | | |
| PLR50 + | PLR10+ | PLR50 -> | PLR10 -> | PLR50 + | PLR10 + | Histones -> | Histones -> | Histones -> | Histones -> |
| Histones -> | Histones -> | DNA mix + | DNA mix + | Histones -> | Histones -> | RNP -> | RNP -> | RNP -> | RNP -> |
| DNA mix -> | DNA mix -> | PDE/PLE -> | PDE/PLE -> | DNA mix + | DNA mix + | DNA mix -> | DNA mix -> | DNA mix -> | DNA mix -> |
| RNP -> | RNP -> | RNP -> | RNP -> | PDE/PLE -> | PDE/PLE -> | PLR50 | PLR10 | PLE/PDE -> | PLE/PDE -> |
| PLR50 + | PLR10+ | PLR50 | PLR10 | RNP -> | RNP -> | | | PLR50 | PLR10 |
| Histones | Histones | | | PLR50 + | PLR10 + | | | | |
| | | | | Histones | Histones | | | | |

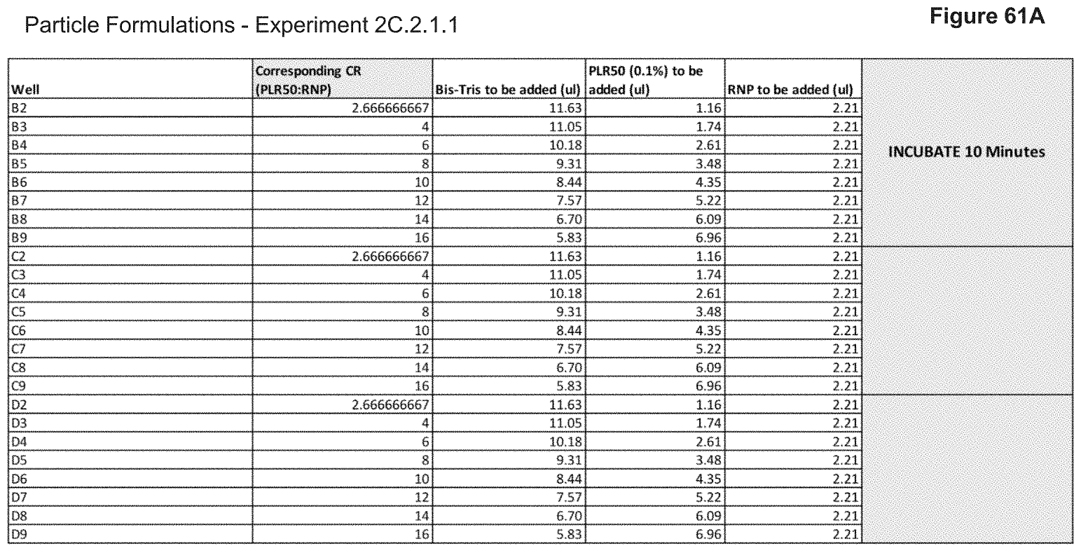

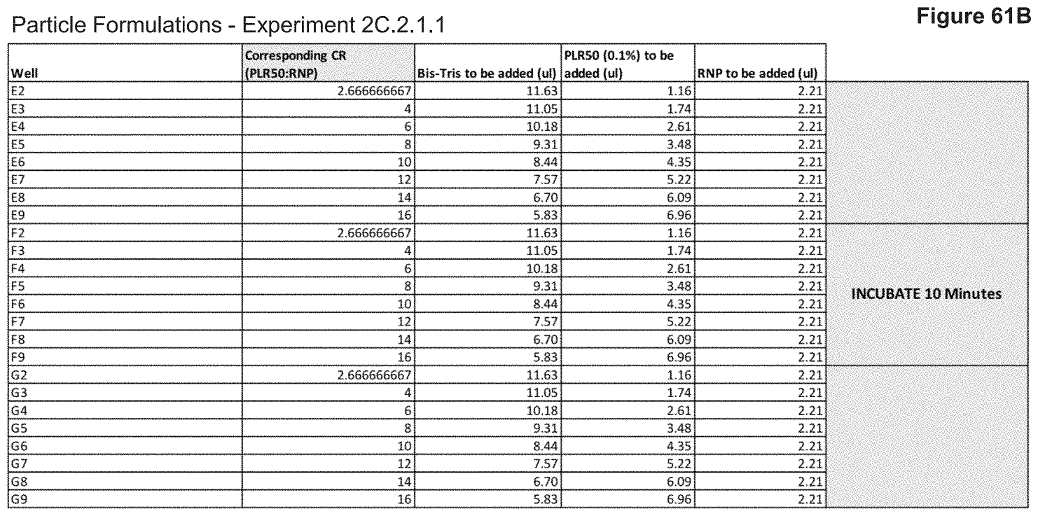

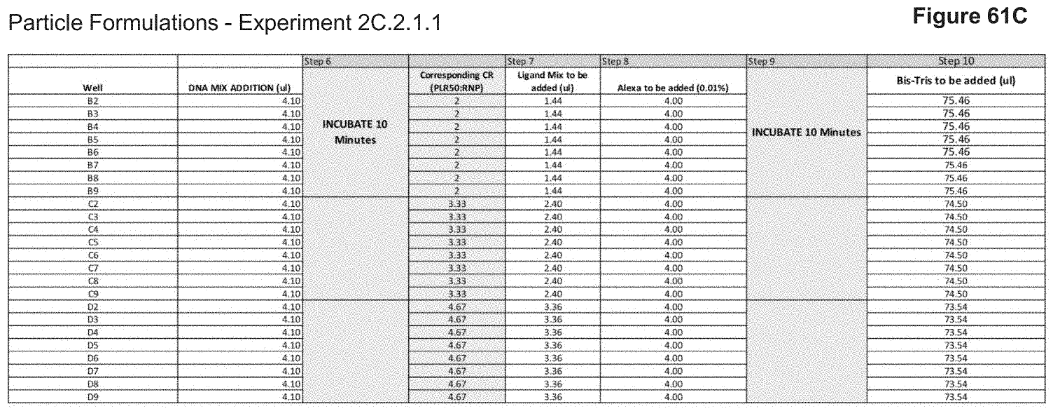

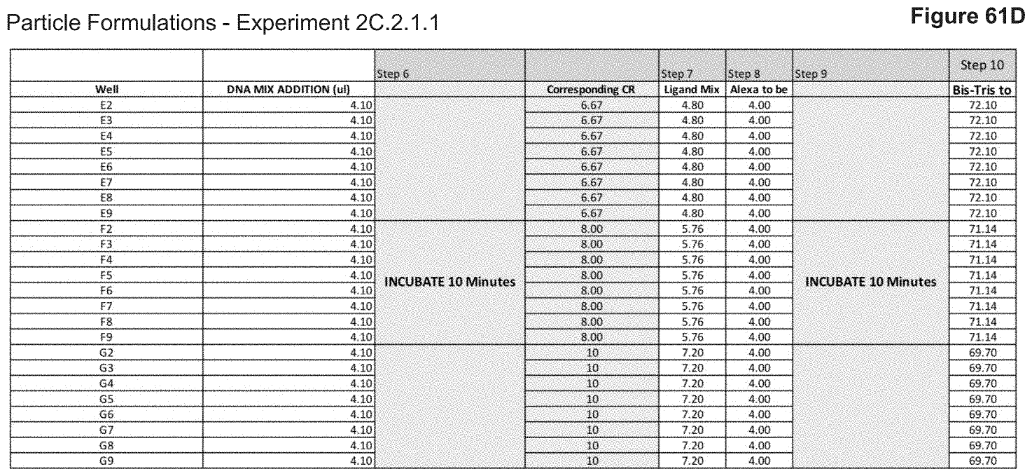

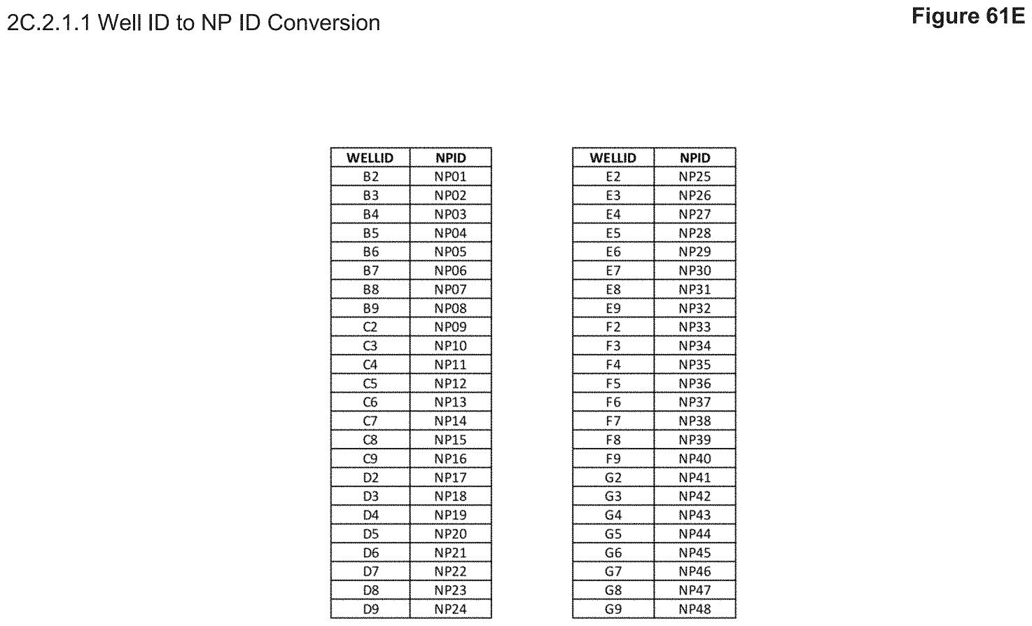

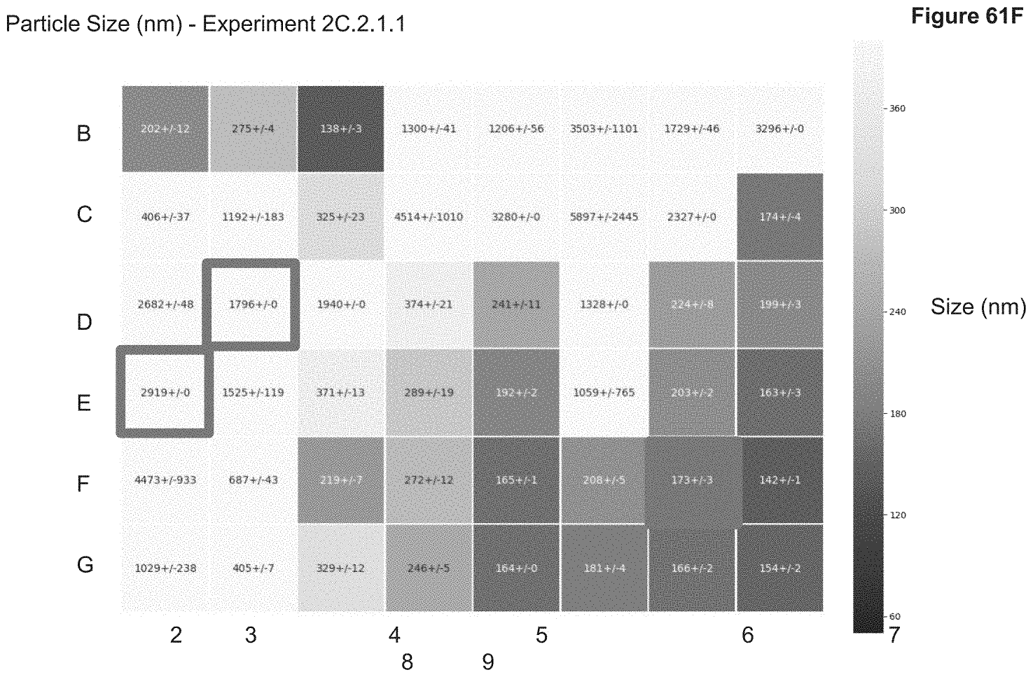

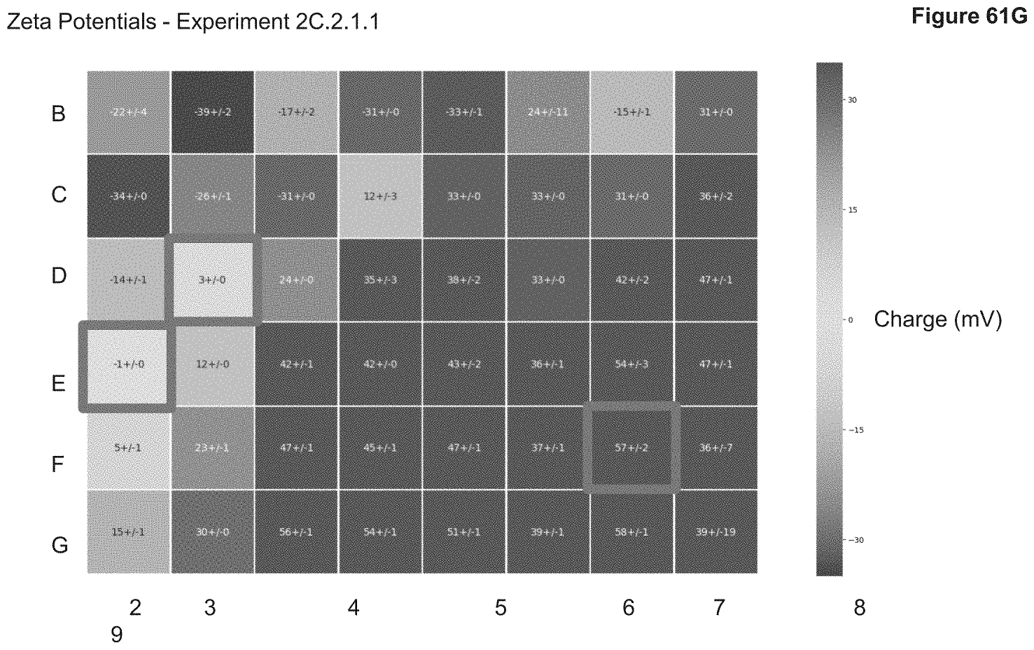

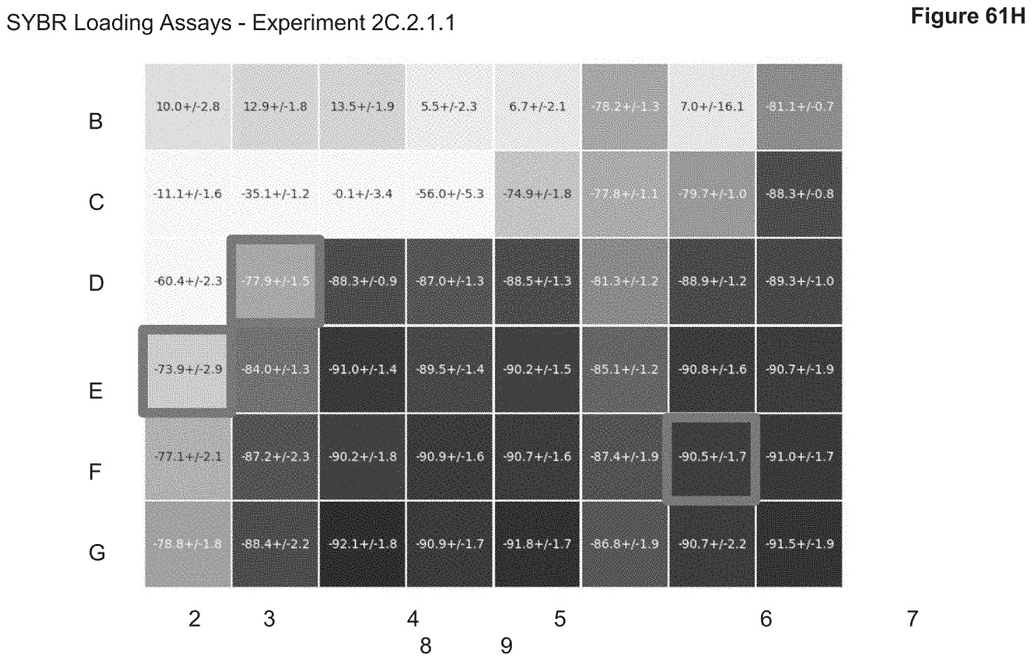

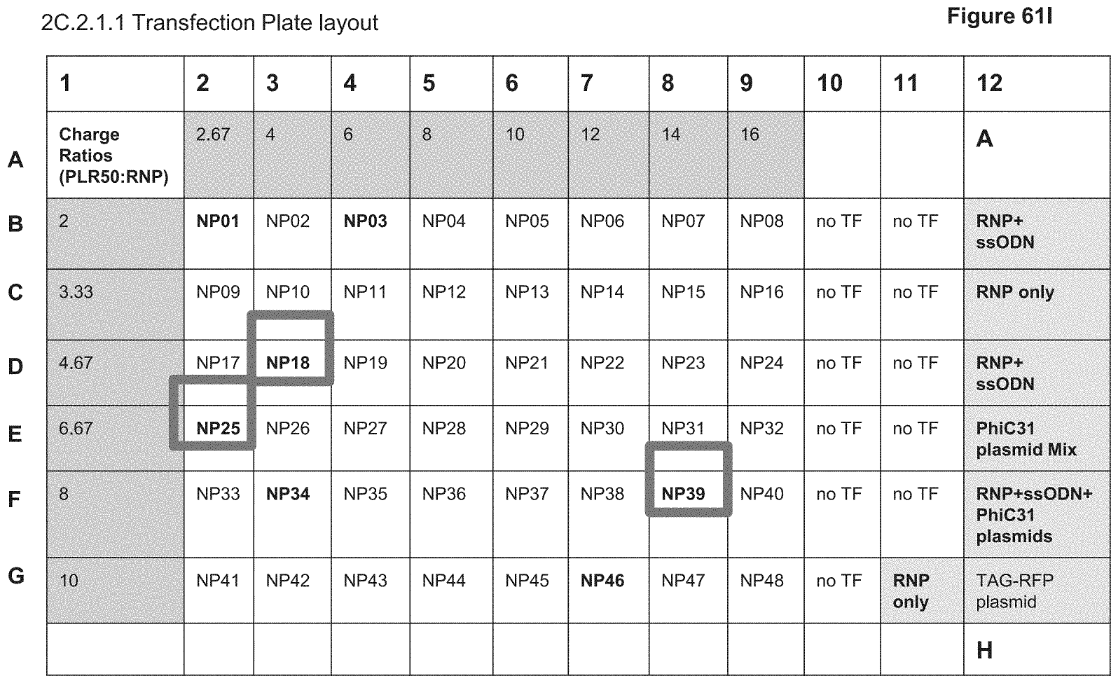

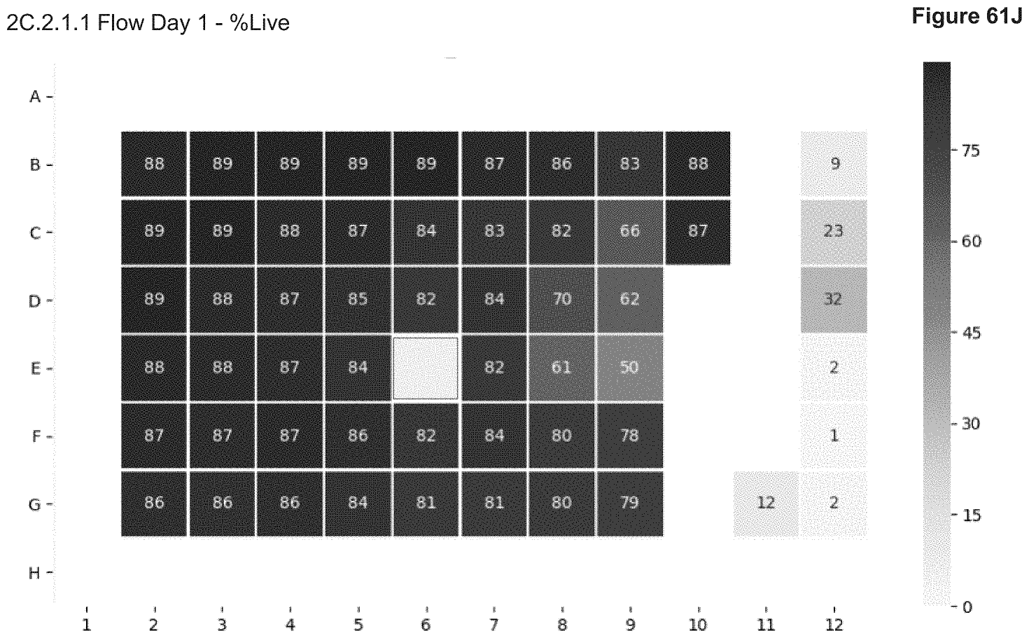

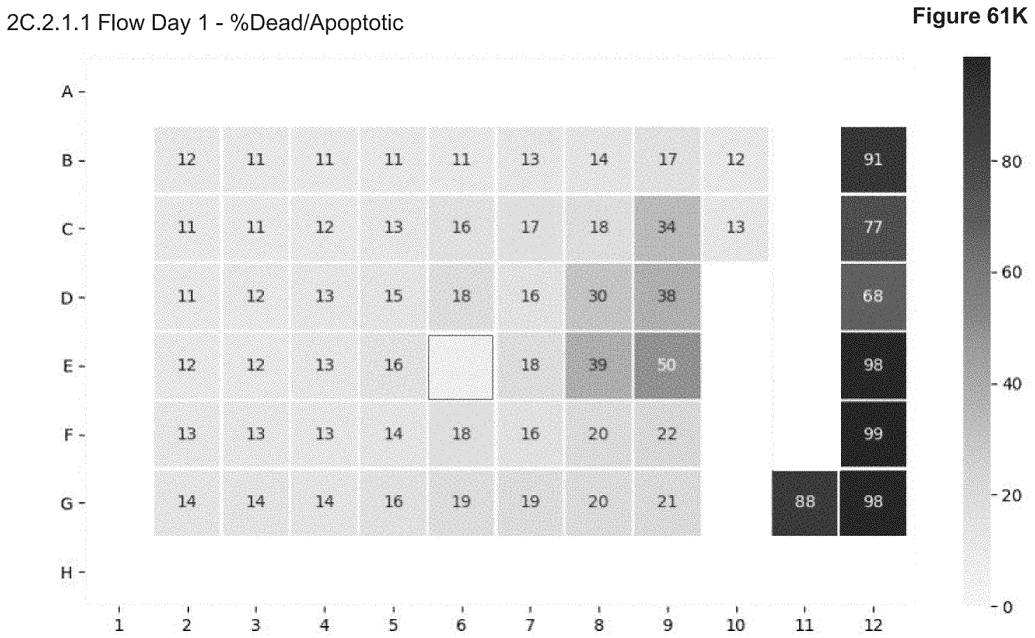

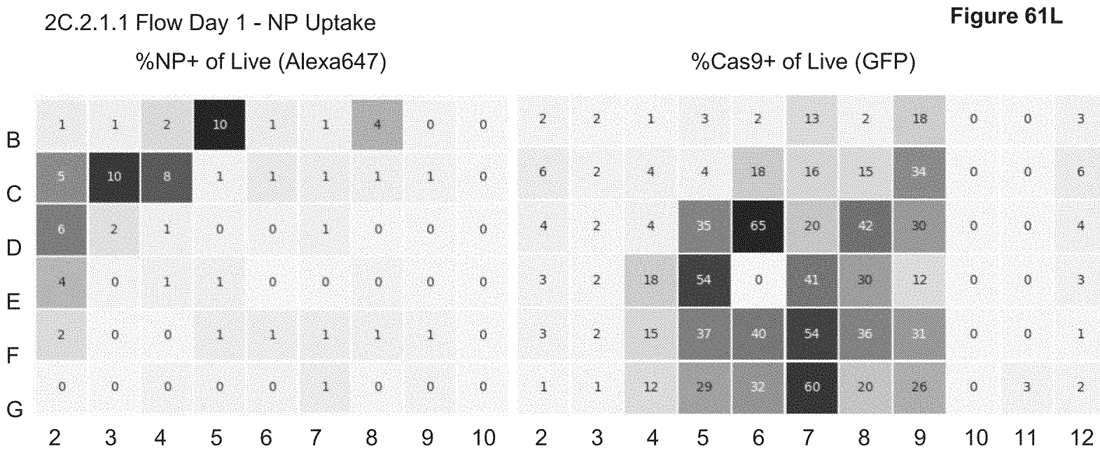

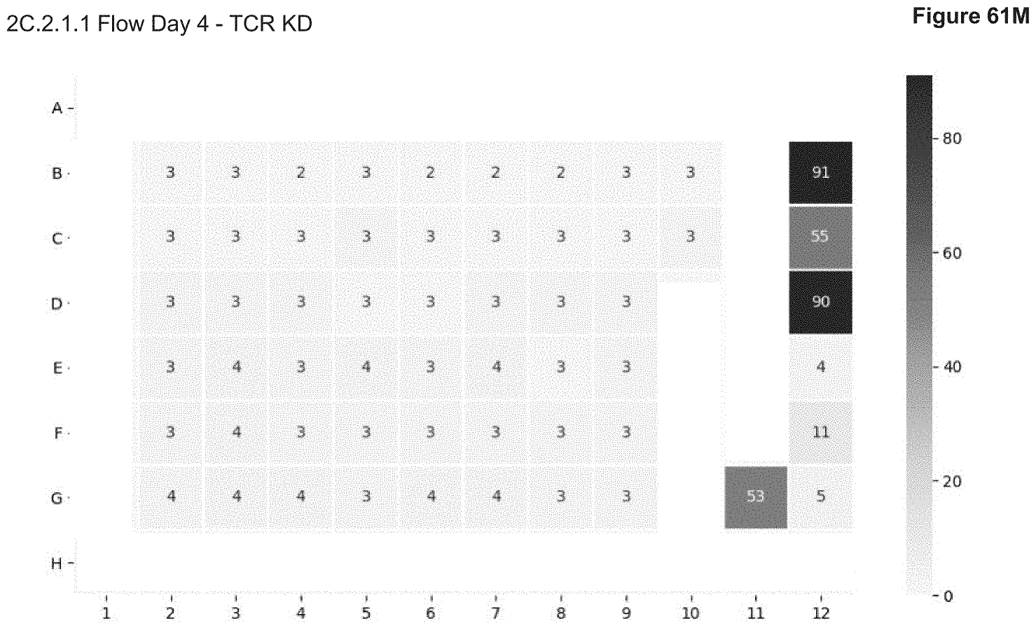

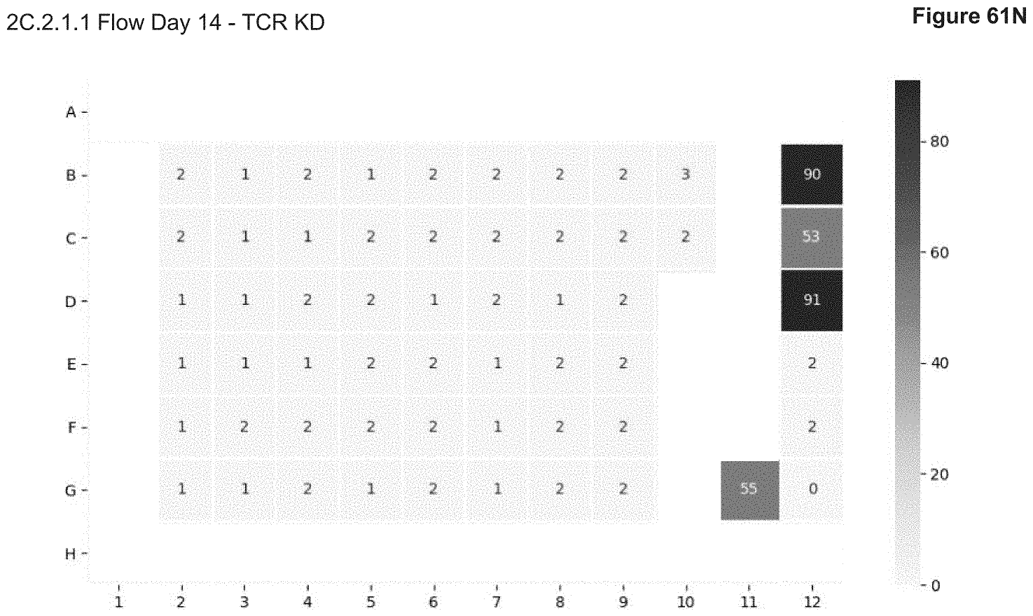

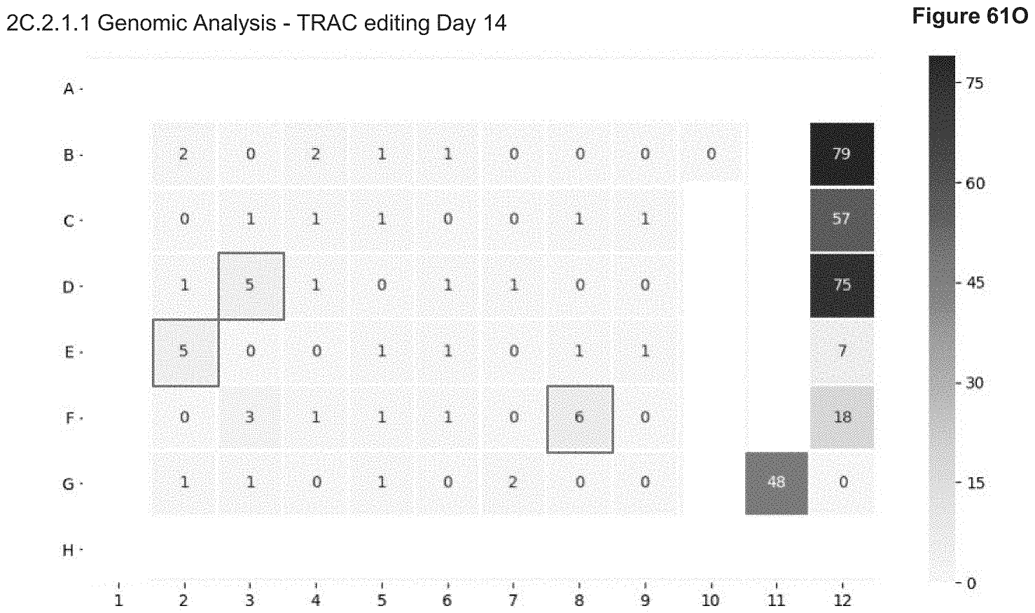

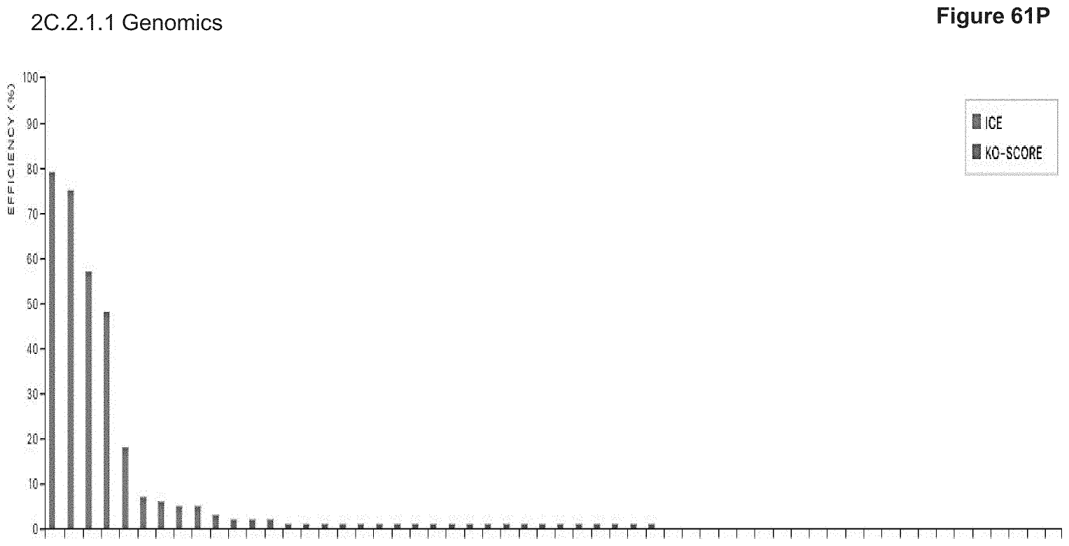

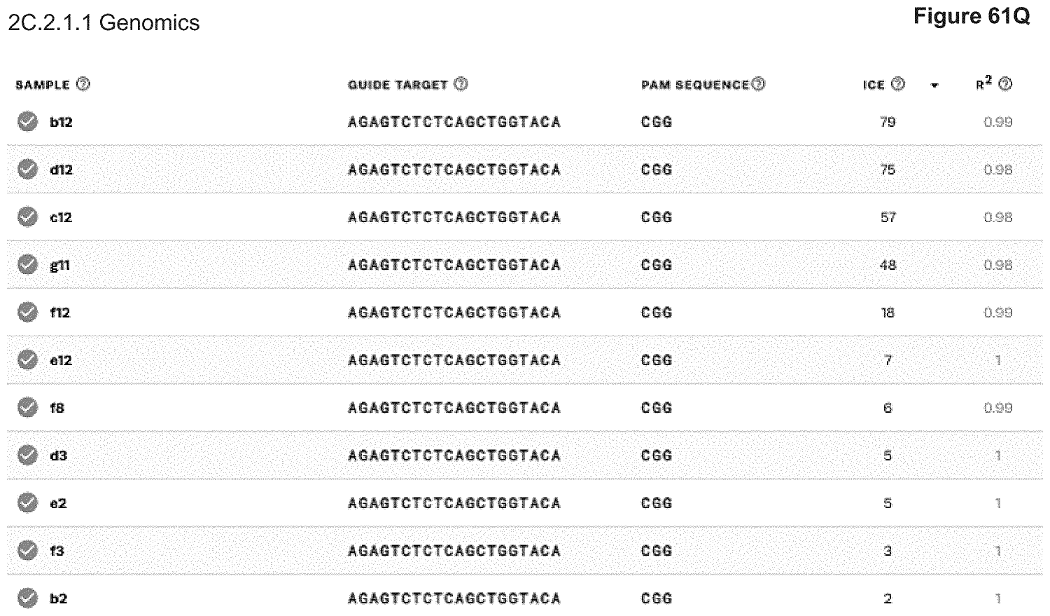

Each layer was incubated for 10 minutes prior to the addition of the next layer. Each unique formulation had 2500 ng ssDNA and 800 ng pDNA implying that every dose (10 ul) delivered 250 ng ssDNA and 80 ng pDNA. Each unique formulation had 15 pmol of Cas9 implying that every dose (10 ul) delivered 1.5 pmol of Cas9. Charge ratios between the initial and final layer of cationic peptide:RNP was constant at a ratio of 10 and 4 respectively. Detailed overview is included in FIGS. 61F and 61G:

After formation, nanoparticles were diluted to two concentrations with Opti-Mem Reduced Serum Media (10 ul NP+90 ul Media or 20 ul NP+80 ul Media). Cells that were seeded at a density of 20 k were treated with 100 ul of either of the diluted NP solutions and left overnight.

2C.2.1.1:

Cas9 RNP was composed using a 1.2:1 sgRNA:Cas9 ratio and incubated for 30 minutes at room temperature. LL224 and LL294 were hydrated from lyophilized state to 0.1% w/v with ultra-pure water (mili-Q water). pLL312 and pLL313 were diluted to 0.05% w/v with water and a stock 1:50 pLL313:pLL312 was created as the plasmid stock. Peptides were diluted to 0.1% w/v using 0.1M Bis-Tris pH 8.5. 48 unique nanoparticles were formed at a final volume of 100 ul in a stepwise manner with varying amounts of 50-mer Poly-L-Arginine (PLR50) and Ligand mix to vary the charge ratio (ratio of total charge of molecule A to molecule B) between PLR50 (+50 total charge per molecule) to RNP (−151 total charge per molecule) and Ligand Mix (+9.5 total charge per molecule) to RNP in the following order of addition:

1. PLR50

2. RNP

3. DNA mix (ssDNA+plasmid stock)

4. Ligand mix (CD3, CD4)

5. Buffer

Layers 1 and 2 were combined and incubated for 10 min then layer 3 was added to layers 1 and 2 and incubated for an additional 10 minutes. The ligand mix was then added to the previous layers and incubated for 10 minutes. Finally buffer was added to each formulation to bring the total volume to 100 uL. Charge ratios of layer one PLR50:RNP ranged from 2.6-16 and charge ratio of layer three Ligand Mix:RNP ranged from 2-10. Each unique formulation had 2500 ng ssDNA and 800 ng pDNA implying that every dose (10 ul) delivered 250 ng ssDNA and 80 ng pDNA. Each unique formulation had 15 pmol of Cas9 implying that every dose (10 ul) delivered 1.5 pmol of Cas9.

After formation, nanoparticles were diluted with Opti-Mem Reduced Serum Media (10 ul NP+90 ul Media). Cells that were seeded on 2 plates, based on the length of stimulation, were treated with 100 ul of the diluted NP solution and left overnight. Cells were then washed with PBS and fresh media was added.

### 2. SYBR Inclusion Assay

Synergy H1 Hybrid Multi-Mode Plate Reader was used to make fluorescence measurements for the SYBR Inclusion Assay. SYBR® Gold Nucleic Acid fluorescent stain (ThermoFisher Scientific) binds DNA and RNA molecules very strongly, with more than 1000-fold signal enhancement upon binding. SYBR GOLD dye was used as an indicator for condensation of the nucleic acid payloads in the nanoparticles and as an estimate for the amount of unencapsulated/free payload. In doing so, nanoparticle candidates were screened that were more promising in encapsulating their payloads (indicated by low SYBR fluorescence) compared to the others (indicated by higher fluorescence). Overnight kinetic measurements were recorded for each nanoparticle sample (N=1) to estimate for stability of the nanoparticle packaging over time. 20 uL of the finished nanoparticle product was mixed with SYBR GOLD working solution (SYBR diluted 10,00× in TE Buffer pH 7.8-8.0) to make a total volume of 100 uL for measurements. Naked/free DNA and RNA payloads were used as controls (N=3) to establish baseline fluorescence. Background subtraction was performed by measuring fluorescence of formulation buffer in SYBR working solution. All measurements were recorded at excitation 485/emission 528. The output is represented as the condensation index is calculated as [(Well of Interest Fluorescence−Free DNA Fluorescence)/Free DNA Fluorescence]*100 and is reported as average condensation index±standard deviation in a heatmap which correlates to the nanoparticle 96-well ID. The more condensed nanoparticles will have higher shielding, less fluorescence, and thus a more negative condensation index.
   3. Particle size and zeta potential determination—Wyatt Technology's Mobius was used to measure the hydrodynamic diameter and zeta potential of the nanoparticles by dynamic light scattering and electrophoretic mobility. A total of three measurements per sample were acquired using the following parameters: 2 second acquisition time, 20 acquisitions per measurement, 2V voltage amplitude, 10 Hz electric field, and 15 second PALS collection period.
   4. Cell culture—HEK293/EGFP-AAVS1 Stable cell line (SL573, GeneCopoeia, Inc., Rockville, Md.) was cultured in DMEM supplemented with 10% FBS and 0.5 ug/mL Puromycin (Gibco A1113802) and passaged with 0.25% Trypsin-EDTA (Sigma 59428C) per manufacturer's instructions. Cryopreserved Human Peripheral Blood Mononuclear cells (PBMCs) from StemCell Technologies (70025.2) were thawed in RPMI supplemented with 10% FBS, 50 I.U./mL rIL-2 (PeproTech 200-02). One day post-thaw, cells were stimulated with CD3/CD28 Dynabeads (ThermoFisher 11131D) in RPMI supplemented with 10% FBS, rIL-2 50 I.U./mL, rIL-7 (Gibco PHC0075) 5 ng/mL, rIL-15 (Gibco PHC9154) 5 ng/mL at 1×10e6 cell/mL. After 2 days of stimulation, Dynabeads were magnetically removed and the stimulated T Cells were cultured in RPMI 10% FBS 125 I.U./mL rIL-2. Transfections were performed at least one day following Dynabead removal. All cells were maintained in 100 U/mL Pen/Strep (ThermoFisher 15-140-122).
   5. Cell transfection—Nanoparticles were diluted 1:10 or 2:10 in serum-free Opti-MEM (ThermoFisher 11058021) for a dose of 10 uL or 20 uL of NP mix (in 100 uL total) per well. HEK293-GFP were plated at 20-40,000 cells/well and Stimulated T Cells at 60,000 cells/well. Cells were incubated in NP overnight, then washed in PBS and cultured as usual. Lipofection of HEK293-GFP with Lipofectamine3000 (ThermoFisher L3000008) for DNA and CRISPRMAX (ThermoFisher CMAX00008) for RNP+/−DNA was carried out per manufacturer's instructions. Nucleofection of stimulated T Cells was performed with Lonza 4D-Nucleofector and P3 Primary Cell kit. 1×10e6 cells in 20 uL cuvettes were electroporated with equivalent doses (scaled) of RNP and DNA as the nanoparticle transfections. Pulse EH-115 was used for RNP only, while pulse EO-115 was used with payloads containing DNA.
   6. Microscopy—Cells were seeded on lysine-coated plates, labeled with Hoechst 33342 (ThermoFisher H3570) and images collected daily on BioTek Cytation 5 daily to observe cell viability, nanoparticles (Alexa647), and GFP and RFP expression. Image analysis was performed on each sample through the following script in Fiji (ImageJ) to determine Pearson coefficients, overlap coefficients, and to generate Costes' maps of colocalization between channels via the following script and associated outputs. Exemplary thresholding, Costes' masks, and colocalization script outputs are shown in FIGS. 60K-60N. NP uptake is found to highly correspond to GFP− pixels in the top-performing nanoparticle groups.

Script for Calling, Enhancing Contrast and Subsequently Masking May Include:

```
function action1(input, output1, filename) {
open(input + filename);
run(“Enhance Contrast...”, “saturated=0.3 normalize”);
run(“8-bit”);
saveAs(output1 + filename);
  }
function action2(input, output, filename) {
  open(input + filename);
run(“Enhance Contrast...”, “saturated=0.3 normalize”);
run(“Remove Outliers...”, “radius=1 threshold=10000 which=Bright”);
setAutoThreshold(“MaxEntropy dark”);
run(“Convert to Mask”);
  saveAs(output + filename);
}
input = “/Users/.../2C1.2.1 Imaging/”;
output1 = “/Users/../V2.0/2C1.2.1 Enhanced Contrast (Auto)“;
output2 = “/Users/.../V2.0/2C1.2.1 Masked (Auto)”;
list = getFileList(input);
for (i = 0; i < list.length; i++)
  action1(input, output1, list[i]);
list = getFileList(output1);
for (i = 0; i < list.length; i++)
  action2(output1, output2, list[i]);
//SHOWN FOR B6
open(“/Users/.../2C1.2.1 Imaging/B6_1_GFP_001.tif);
run(“Enhance Contrast...”, “saturated=0.3 normalize”);
run(“Remove Outliers...”, “radius=1 threshold=10000 which=Bright”);
setAutoThreshold(“MaxEntropy dark”);
run(“Convert to Mask”);
saveAs(“Tiff”, “/Users/.../V2.0/B6_1_GFP_processed_001.tif”);
//SHOWN FOR B6
open(“/Users/.../2C1.2.1 Imaging/B6_1_Texas Red_001.tif”);
run(“Enhance Contrast...”, “saturated=0.3 normalize”);
run(“Remove Outliers...”, “radius=1 threshold=10000 which=Dark”);
setAutoThreshold(“MaxEntropy dark”);
run(“Convert to Mask”);
run(“Remove Outliers...”, “radius=1 threshold=100 which=Bright”);
saveAs(“Tiff', “/Users/.../V1.0/B6_1_Texas Red_processed_001.tif);
//SHOWN FOR B6
open(“/Users/.../2C1.2.1 Imaging/B6_1_DAPI_001.tif”);
run(“Enhance Contrast...”, “saturated=0.3 normalize”);
run(“Remove Outliers...”, “radius=1 threshold=10000 which=Bright”);
call(“ij.plugin.frame.ThresholdAdjustersetMode”, “Red”);
setAutoThreshold(“MaxEntropy dark”);
run(“Convert to Mask”);
saveAs(“Tiff”, “/Users/.../V2.0/B6_1_DAPI_processed_001.tif”);
//SHOWN FOR B6
open(“/Users/.../2C1.2.1 Imaging/B6_1_Cy5_001.tif”);
setThreshold(2743,65535);
run(“Convert to Mask”);
run(“Remove Outliers...”, “radius=1 threshold=100 which=Bright”);
saveAs(“Tiff
run(“Images to Stack”, “name=B6_1_Montage.tif title=B6_1 use keep”);
run(“Make Montage...”, “columns=4 rows=2 scale=0.25 label”);
saveAs(“Tiff”, “/Users/...V2.0/B6_1_montage_001.tif”);
run(“Close All”);
run(“JACoP ”);
```

Resulting Outputs:

### Image A: E5_1_GFP_8-bit-enhanced_001. tif

### Image B: E5_1_Texas Red_8-bit-enhanced_001. tif

### Pearson's Coefficient:

r=−0.086

### Overlap Coefficient:

r=0.587
r\hat{ }2=k1×k2:
k1=0.844
k2=0.409
Using thresholds (thrA=28 and thrB=196)

### Overlap Coefficient:

r=0.915
r\hat{ }2=k1×k2:
k1=1.504
k2=0.556
Manders' Coefficients (original):
M1=0.997 (fraction of A overlapping B)
M2=0.998 (fraction of B overlapping A)
Manders' Coefficients (using threshold value of 28 for imgA and 196 for imgB):
M1=0.01 (fraction of A overlapping B)
M2=0.907 (fraction of B overlapping A)
Costes' automatic threshold set to 255 for imgA & 255 for imgB

### Pearson's Coefficient:

r=0.0 (1.0 below thresholds)

### M1=0.0 & M2=0.0

7. Flow cytometry—Flow cytometry was performed on Attune N×T (ThermoFisher A24858) and data analyzed with FlowJo. Direct fluorescence was monitored for GFP, RFP, and Alexa647 (nanoparticle label). Cell viability assays included Zombie NIR Fixable Viability kit (Biolegend 423106), Annexin V Pacific Blue (ThermoFisher A35122). For PBMCs, the following antibodies were used: TCRα/β-PE-Cy7 (IP26) 1:100 (ThermoFisher 25-9986-42), CD8a-Super Bright 600 (RPA-T8) 1:33 (ThermoFisher 63-0088-42), CD4-PE (RPA-T4) 1:100 (ThermoFisher 12-0049-42), CD4-APC-eFluor780 (RPA-T4) 1:200 (ThermoFisher 47-0049-42), CD3-APC-eFluor780 (OKT3) 1:100 (ThermoFisher 47-0037-41).
8. PCR—Cells were washed with PBS and genomic DNA was harvested using Quick Extract (Lucigen, QE09050) per manufacturer's instructions. PCR was performed using primers flanking the sgRNA cutting sequence, outside of the ssODN homology arms: GFP forward—5′-atggtgagcaagggcgagg (SEQ ID NO: 324); GFP reverse—5′-cacgaactccagcaggaccatg (SEQ ID NO: 325); TRAC forward—5′-CCAGCCTAAGTTGGGGAGAC (SEQ ID NO: 292); TRAC reverse—5′-GTGACTGCGTGAGACTGACT (SEQ ID NO: 293).
Sequencing—PCR products were validated by E-gel (Thermo fisher scientific, G401001) and sent to Genewiz for Sanger sequencing using a primer upstream of the sgRNA target sequence: GFP 5′-gagctgttcaccggggtggt (SEQ ID NO: 294); TRAC 5′-CTGAGTCCCAGTCCATCACGA (SEQ ID NO: 295). Sanger sequencing chromatograms were analysed using Synthego's Inference of CRISPR Edits (ICE) and ICE knock-in program.

1. A method for inserting a donor sequence into a cell's genome, comprising:
introducing into a cell, a delivery vehicle with a payload comprising:
(a) a nuclease composition, comprising: one or more sequence specific nucleases or one or more nucleic acids that encode the one or more sequence specific nucleases, wherein the one or more sequence specific nucleases cleaves the cell's genome;
(b) a target donor composition, comprising: a first donor DNA, which comprises a nucleotide sequence that is inserted into the cell's genome, wherein insertion of said nucleotide sequence produces, in the cell's genome at the site of insertion, a target sequence for a site-specific recombinase;
(c) a recombinase composition, comprising: the site-specific recombinase, or a nucleic acid encoding the site-specific recombinase, wherein the site-specific recombinase recognizes said target sequence; and
(d) an insert donor composition, comprising: a second donor DNA, which comprises a nucleotide sequence that is inserted into the cell's genome as a result of recognition of said target sequence by the site-specific recombinase.

2. The method of claim 1, wherein insertion of the nucleotide sequence of the first donor DNA of the target donor composition produces a first target sequence for the site-specific recombinase at a first location in the cell's genome and a second target sequence for the site-specific recombinase at a second location in the cell's genome.

3. The method of claim 1, wherein the nuclease composition cleaves the cell's genome at two locations, and wherein the target donor composition comprises two of said first donor DNAs, each of which comprises a nucleotide sequence that is inserted into the cell's genome, thereby producing a first target sequence for the site-specific recombinase at a first location in the cell's genome and a second target sequence for the site-specific recombinase at a second location in the cell's genome.

4-57. (canceled)

58. A method for inserting a donor sequence into a cell's genome, comprising:
introducing into a cell, a delivery vehicle with a payload comprising:
(a) a nuclease composition, comprising: one or more sequence specific nucleases or one or more nucleic acids that encode the one or more sequence specific nucleases, wherein the one or more sequence specific nucleases cleaves the cell's genome;
(b) a target donor composition, comprising: a first donor DNA, which comprises a nucleotide sequence that is inserted into the cell's genome, wherein insertion of said nucleotide sequence produces, in the cell's genome at the site of insertion, a target sequence for a site-specific recombinase;
(c) a recombinase composition, comprising: the site-specific recombinase, or a nucleic acid encoding the site-specific recombinase, wherein the site-specific recombinase recognizes said target sequence; and
(d) an insert donor composition, comprising: a second donor DNA, which comprises a nucleotide sequence that is inserted into the cell's genome as a result of recognition of said target sequence by the site-specific recombinase, wherein the method comprises introducing a first and a second of said delivery vehicles into the cell, wherein:
(1) the nucleotide sequence of the second donor DNA of the first delivery vehicle, that is inserted into the cell's genome, encodes a T cell receptor (TCR) Alpha or Delta subunit, and the nucleotide sequence of the second donor DNA of the second delivery vehicle, that is inserted into the cell's genome, encodes a TCR Beta or Gamma subunit; or
(2) the nucleotide sequence of the second donor DNA of the first delivery vehicle, that is inserted into the cell's genome, encodes a T cell receptor (TCR) Alpha or Gamma subunit, and the nucleotide sequence of the second donor DNA of the second delivery vehicle, that is inserted into the cell's genome, encodes a TCR Beta or Delta subunit; or
(3) the nucleotide sequence of the second donor DNA of the first delivery vehicle, that is inserted into the cell's genome, encodes the K chain of an IgA, IgD, IgE, IgG, or IgM protein, and the nucleotide sequence of the second donor DNA of the second delivery vehicle, that is inserted into the cell's genome, encodes the A chain of an IgA, IgD, IgE, IgG, or IgM protein.

59. The method of claim 1, wherein the method comprises introducing a first and a second of said delivery vehicles into the cell,
wherein the nucleotide sequence of the second donor DNA of the first delivery vehicle, that is inserted into the cell's genome, encodes a T cell receptor (TCR) Alpha or Delta subunit constant region, and
wherein the nucleotide sequence of the second donor DNA of the second delivery vehicle, that is inserted into the cell's genome, encodes a TCR Beta or Gamma subunit constant region.

60. The method of claim 1, wherein the method comprises introducing a first and a second of said delivery vehicles into the cell, wherein:
(1) the nucleotide sequence of the second donor DNA of the first delivery vehicle is inserted within a nucleotide sequence that functions as a T cell receptor (TCR) Alpha or Delta subunit promoter, and the nucleotide sequence of the second donor DNA of the second delivery vehicle is inserted within a nucleotide sequence that functions as a TCR Beta or Gamma subunit promoter; or
(2) the nucleotide sequence of the second donor DNA of the first delivery vehicle is inserted within a nucleotide sequence that functions as a T cell receptor (TCR) Alpha or Gamma subunit promoter, and the nucleotide sequence of the second donor DNA of the second delivery vehicle is inserted within a nucleotide sequence that functions as a TCR Beta or Delta subunit promoter; or
(3) the nucleotide sequence of the second donor DNA of the first delivery vehicle is inserted within a nucleotide sequence that functions as a promoter for a K chain of an IgA, IgD, IgE, IgG, or IgM protein, and the nucleotide sequence of the second donor DNA of the second delivery vehicle is inserted within a nucleotide sequence that functions as a promoter for a λ chain of an IgA, IgD, IgE, IgG, or IgM protein.

61-62. (canceled)

63. A composition comprising:
(a) a nuclease composition, comprising: one or more sequence specific nucleases or one or more nucleic acids that encode the one or more sequence specific nucleases;
(b) a target donor composition, comprising: a first donor DNA that comprises a target sequence for a site-specific recombinase;
(c) a recombinase composition, comprising: the site-specific recombinase, or a nucleic acid encoding the site-specific recombinase, wherein the site-specific recombinase recognizes said target sequence; and
(d) an insert donor composition, comprising: a second donor DNA, which comprises a nucleotide sequence of interest for insertion into a target cell's genome;
wherein (a), (b), (c), and (d) are payloads as part of the same delivery vehicle.

64. The composition of claim 63, wherein the delivery vehicle is a nanoparticle.

65. The composition of claim 64, wherein the nanoparticle comprises a core comprising (a), (b), an anionic polymer composition, a cationic polymer composition, and a cationic polypeptide composition.

66. The composition of claim 64, wherein the nanoparticle comprises a targeting ligand that targets the nanoparticle to a cell surface protein.

67. The composition of claim 66, wherein the cell surface protein is CD47.

68. The composition of claim 67, wherein the targeting ligand is a SIRPα protein mimetic.

69. The composition of claim 68, wherein the nanoparticle further comprises an endocytosis-triggering ligand.

70. The composition of claim 63, wherein the payloads form one or more deoxyribonucleoprotein complexes or one or more ribo-deoxyribonucleoprotein complexes.

71. The composition of claim 63, wherein the delivery vehicle is a targeting ligand conjugated to a charged polymer polypeptide domain, wherein the targeting ligand provides for targeted binding to a cell surface protein, and wherein the charged polymer polypeptide domain is interacting electrostatically with one or more of the payloads.

72. The composition of claim 71, wherein the delivery vehicle further comprises an anionic polymer interacting with one or more of the payloads and the charged polymer polypeptide domain.

73. The composition of claim 63, wherein the delivery vehicle is a targeting ligand conjugated one or more of the payloads, wherein the targeting ligand provides for targeted binding to a cell surface protein.

74. The composition of claim 63, wherein the delivery vehicle includes a targeting ligand coated upon a water-oil-water emulsion particle, upon an oil-water emulsion micellar particle, upon a multilamellar water-oil-water emulsion particle, upon a multilayered particle, or upon a DNA origami nanobot.

75. The method of claim 66, wherein the targeting ligand is a peptide, an ScFv, a F(ab), a nucleic acid aptamer, or a peptoid.

76. The composition of claim 63, wherein the delivery vehicle is non-viral.


SEQUENCE LISTING (325 sequences)

SEQ ID NO: 1 - protein, 39 aa, Artificial sequence
  synthetic sequence
HGEGTFTSDLSKQMEEEAVRLFIEWLKNGGPSSGAPPPS

SEQ ID NO: 2 - protein, 39 aa, Artificial sequence
  synthetic sequence
HGEGTFTSDLCKQMEEEAVRLFIEWLKNGGPSSGAPPPS

SEQ ID NO: 3 - protein, 86 aa, Artificial sequence
  synthetic sequence
KRLYCKNGGFFLRIHPDGRVDGVREKSDPHIKLQLQAEERGVVSIKGVCANRYLAMKEDGRLLASKCVTDECFFFERLESNNYNTY

SEQ ID NO: 4 - protein, 22 aa, Artificial sequence
  synthetic sequence
KNGGFFLRIHPDGRVDGVREKS

SEQ ID NO: 5 - protein, 6 aa, Artificial sequence
  synthetic sequence
HFKDPK

SEQ ID NO: 6 - protein, 8 aa, Artificial sequence
  synthetic sequence
LESNNYNT

SEQ ID NO: 7 - protein, 21 aa, Artificial sequence
  synthetic sequence
MIASQFLSALTLVLLIKESGA

SEQ ID NO: 8 - protein, 38 aa, Artificial sequence
  synthetic sequence
MVFPWRCEGTYWGSRNILKLWVWTLLCCDFLIHHGTHC

SEQ ID NO: 9 - protein, 38 aa, Artificial sequence
  synthetic sequence
MIFPWKCQSTQRDLWNIFKLWGWTMLCCDFLAHHGTDC

SEQ ID NO: 10 - protein, 28 aa, Artificial sequence
  synthetic sequence
MIFPWKCQSTQRDLWNIFKLWGWTMLCC

SEQ ID NO: 11 - protein, 12 aa, Artificial sequence
  synthetic sequence
THRPPMWSPVWP

SEQ ID NO: 12 - protein, 7 aa, Artificial sequence
  synthetic sequence
RRETAWA

SEQ ID NO: 13 - protein, 86 aa, Artificial sequence; identical to SEQ ID NO: 3
  synthetic sequence

SEQ ID NO: 14 - protein, 7 aa, Artificial sequence
  synthetic sequence
  misc_feature (1)..(7): Xaa can be any naturally occurring amino acid
XXXXXXX

SEQ ID NO: 15 - protein, 9 aa, Artificial sequence
  synthetic sequence
RRRRRRRRR

SEQ ID NO: 16 - protein, 6 aa, Artificial sequence
  synthetic sequence
HHHHHH

SEQ ID NO: 17 - protein, 9 aa, Artificial sequence
  synthetic sequence
GAPGAPGAP

SEQ ID NO: 18 - protein, 10 aa, Artificial sequence
  synthetic sequence
GAPGAPGAPC

SEQ ID NO: 19 - protein, 10 aa, Artificial sequence
  synthetic sequence
CGAPGAPGAP

SEQ ID NO: 20 - protein, 5 aa, Artificial sequence
  synthetic sequence
  MISC_FEATURE (1)..(5): The amino acids from positions 1 to 5 may be repeated n times.
GSGGS

SEQ ID NO: 21 - protein, 6 aa, Artificial sequence
  synthetic sequence
  MISC_FEATURE (1)..(6): The amino acids at positions 1 to 6 may be repeated n times.
GGSGGS

SEQ ID NO: 22 - protein, 4 aa, Artificial sequence
  synthetic sequence
  MISC_FEATURE (1)..(4): The amino acids at positions 1 to 4 may be repeated n times.
GGGS

SEQ ID NO: 23 - protein, 4 aa, Artificial sequence
  synthetic sequence
GGSG

SEQ ID NO: 24 - protein, 5 aa, Artificial sequence
  synthetic sequence
GGSGG

SEQ ID NO: 25 - protein, 5 aa, Artificial sequence
  synthetic sequence
GSGSG

SEQ ID NO: 26 - protein, 5 aa, Artificial sequence
  synthetic sequence
GSGGG

SEQ ID NO: 27 - protein, 5 aa, Artificial sequence
  synthetic sequence
GGGSG

SEQ ID NO: 28 - protein, 5 aa, Artificial sequence
  synthetic sequence
GSSSG

SEQ ID NO: 29 - protein, 11 aa, Artificial sequence
  synthetic sequence
GGGGGSGGGGG

SEQ ID NO: 30 - protein, 11 aa, Artificial sequence
  synthetic sequence
GGGGGSGGGGS

SEQ ID NO: 31 - protein, 12 aa, Artificial sequence
  synthetic sequence
GGGGGSGGGGGC

SEQ ID NO: 32 - protein, 12 aa, Artificial sequence
  synthetic sequence
CGGGGGSGGGGG

SEQ ID NO: 33 - protein, 12 aa, Artificial sequence
  synthetic sequence
GGGGGSGGGGSC

SEQ ID NO: 34 - protein, 12 aa, Artificial sequence
  synthetic sequence
CGGGGGSGGGGS

SEQ ID NO: 35 - protein, 4 aa, Artificial sequence
  synthetic sequence
KALA

SEQ ID NO: 36 - protein, 4 aa, Artificial sequence
  synthetic sequence
GALA

SEQ ID NO: 37 - protein, 5 aa, Artificial sequence
  synthetic sequence
CKALA

SEQ ID NO: 38 - protein, 5 aa, Artificial sequence
  synthetic sequence
KALAC

SEQ ID NO: 39 - protein, 5 aa, Artificial sequence
  synthetic sequence
CGALA

SEQ ID NO: 40 - protein, 5 aa, Artificial sequence
  synthetic sequence
GALAC

SEQ ID NO: 41 - protein, 19 aa, Artificial sequence
  synthetic sequence
RRRRRRRRRGAPGAPGAPC

SEQ ID NO: 42 - protein, 19 aa, Artificial sequence
  synthetic sequence
CGAPGAPGAPRRRRRRRRR

SEQ ID NO: 43 - protein, 23 aa, Artificial sequence
  synthetic sequence
CKNGGFFLRIHPDGRVDGVREKS

SEQ ID NO: 44 - protein, 23 aa, Artificial sequence
  synthetic sequence
KNGGFFLRIHPDGRVDGVREKSC

SEQ ID NO: 45 - protein, 25 aa, Artificial sequence
  synthetic sequence
RRRRRRRRRGAPGAPGAPRRETAWA

SEQ ID NO: 46 - protein, 25 aa, Artificial sequence
  synthetic sequence
RRETAWAGAPGAPGAPRRRRRRRRR

SEQ ID NO: 47 - protein, 21 aa, Artificial sequence
  synthetic sequence
RRRRRRRRRGAPGAPGAPRGD

SEQ ID NO: 48 - protein, 21 aa, Artificial sequence
  synthetic sequence
RGDGAPGAPGAPRRRRRRRRR

SEQ ID NO: 49 - protein, 4 aa, Artificial sequence
  synthetic sequence
CRGD

SEQ ID NO: 50 - protein, 4 aa, Artificial sequence
  synthetic sequence
RGDC

SEQ ID NO: 51 - protein, 30 aa, Artificial sequence
  synthetic sequence
RRRRRRRRRGAPGAPGAPTHRPPMWSPVWP

SEQ ID NO: 52 - protein, 30 aa, Artificial sequence
  synthetic sequence
THRPPMWSPVWPGAPGAPGAPRRRRRRRRR

SEQ ID NO: 53 - protein, 13 aa, Artificial sequence
  synthetic sequence
CTHRPPMWSPVWP

SEQ ID NO: 54 - protein, 14 aa, Artificial sequence
  synthetic sequence
CPTHRPPMWSPVWP

SEQ ID NO: 55 - protein, 13 aa, Artificial sequence
  synthetic sequence
THRPPMWSPVWPC

SEQ ID NO: 56 - protein, 39 aa, Artificial sequence
  synthetic sequence
RRRRRRRRRGAPGAPGAPMIASQFLSALTLVLLIKESGA

SEQ ID NO: 57 - protein, 39 aa, Artificial sequence
  synthetic sequence
MIASQFLSALTLVLLIKESGAGAPGAPGAPRRRRRRRRR

SEQ ID NO: 58 - protein, 22 aa, Artificial sequence
  synthetic sequence
CMIASQFLSALTLVLLIKESGA

SEQ ID NO: 59 - protein, 22 aa, Artificial sequence
  synthetic sequence
MIASQFLSALTLVLLIKESGAC

SEQ ID NO: 60 - protein, 40 aa, Artificial sequence
  synthetic sequence
RRRRRRRRRGAPGAPGAPKNGGFFLRIHPDGRVDGVREKS

SEQ ID NO: 61 - protein, 40 aa, Artificial sequence
  synthetic sequence
KNGGFFLRIHPDGRVDGVREKSGAPGAPGAPRRRRRRRRR

SEQ ID NO: 62 - protein, 20 aa, Artificial sequence
  synthetic sequence
SGRGKQGGKARAKAKTRSSR

SEQ ID NO: 63 - protein, 39 aa, Artificial sequence
  synthetic sequence
SGRGKQGGKARAKAKTRSSRAGLQFPVGRVHRLLRKGGG

SEQ ID NO: 64 - protein, 130 aa, Artificial sequence
  synthetic sequence
MSGRGKQGGKARAKAKTRSSRAGLQFPVGRVHRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 65 - protein, 10 aa, Artificial sequence
  synthetic sequence
CKATQASQEY

SEQ ID NO: 66 - protein, 25 aa, Artificial sequence
  synthetic sequence
KKTSATVGPKAPSGGKKATQASQEY

SEQ ID NO: 67 - protein, 143 aa, Artificial sequence
  synthetic sequence
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 68 - protein, 11 aa, Artificial sequence
  synthetic sequence
  MISC_FEATURE (5)..(5): The amino acid at position 5 is crotonylated.
PEPAKSAPAPK

SEQ ID NO: 69 - protein, 11 aa, Artificial sequence
  synthetic sequence
PEPAKSAPAPK

SEQ ID NO: 70 - protein, 15 aa, Artificial sequence
  synthetic sequence
AQKKDGKKRKRSRKE

SEQ ID NO: 71 - protein, 126 aa, Artificial sequence
  synthetic sequence
MPEPAKSAPAPKKGSKKAVTKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSK

SEQ ID NO: 72 - protein, 8 aa, Artificial sequence
  synthetic sequence
ARTKQTAR

SEQ ID NO: 73 - protein, 10 aa, Artificial sequence
  synthetic sequence
  MISC_FEATURE (4)..(4): The amino acid at position 4 is attached to one methyl group.
ARTKQTARKS

SEQ ID NO: 74 - protein, 10 aa, Artificial sequence
  synthetic sequence
  MISC_FEATURE (4)..(4): The amino acid at position 4 is attached to two methyl groups.
ARTKQTARKS

SEQ ID NO: 75 - protein, 10 aa, Artificial sequence
  synthetic sequence
  MISC_FEATURE (4)..(4): The amino acid at position 4 is attached to three methyl groups.
ARTKQTARKS

SEQ ID NO: 76 - protein, 15 aa, Artificial sequence
  synthetic sequence
  MISC_FEATURE (10)..(10): The amino acid at position 10 is phosphorylated.
ARTKQTARKSTGGKA

SEQ ID NO: 77 - protein, 20 aa, Artificial sequence
  synthetic sequence
ARTKQTARKSTGGKAPRKWC

SEQ ID NO: 78 - protein, 19 aa, Artificial sequence
  synthetic sequence
  MISC_FEATURE (14)..(14): The amino acid at position 14 is attached to an acyl group.
ARTKQTARKSTGGKAPRKQ

SEQ ID NO: 79 - protein, 20 aa, Artificial sequence
  synthetic sequence
ARTKQTARKSTGGKAPRKQL

SEQ ID NO: 80 - protein, 20 aa, Artificial sequence
  synthetic sequence
  MISC_FEATURE (9)..(9): The amino acid at position 9 is attached to an acyl group.
ARTKQTARKSTGGKAPRKQL

SEQ ID NO: 81 - protein, 21 aa, Artificial sequence
  synthetic sequence
ARTKQTARKSTGGKAPRKQLA

SEQ ID NO: 82 - protein, 21 aa, Artificial sequence
  synthetic sequence
  MISC_FEATURE (9)..(9): The amino acid at position 9 is attached to an acyl group.
ARTKQTARKSTGGKAPRKQLA

SEQ ID NO: 83 - protein, 21 aa, Artificial sequence
  synthetic sequence
  MISC_FEATURE (9)..(9): The amino acid at position 9 is attached to two methyl groups.
ARTKQTARKSTGGKAPRKQLA

SEQ ID NO: 84 - protein, 21 aa, Artificial sequence
  synthetic sequence
  MISC_FEATURE (9)..(9): The amino acid at position 9 is attached to two methyl groups.
ARTKQTARKSTGGKAPRKQLA

SEQ ID NO: 85 - protein, 21 aa, Artificial sequence
  synthetic sequence
  MISC_FEATURE (9)..(9): The amino acid at position 9 is attached to two methyl groups.
ARTKQTARKSTGGKAPRKQLA

SEQ ID NO: 86 - protein, 21 aa, Artificial sequence
  synthetic sequence
  MISC_FEATURE (4)..(4): The amino acid at position 4 is attached to a methyl group.
ARTKQTARKSTGGKAPRKQLA

SEQ ID NO: 87 - protein, 21 aa, Artificial sequence
  synthetic sequence
  MISC_FEATURE (4)..(4): The amino acid at position 4 is attached to two methyl groups.
ARTKQTARKSTGGKAPRKQLA

SEQ ID NO: 88 - protein, 21 aa, Artificial sequence
  synthetic sequence
  MISC_FEATURE (4)..(4): The amino acid at position 4 is attached to three methyl groups.
ARTKQTARKSTGGKAPRKQLA

SEQ ID NO: 89 - protein, 21 aa, Artificial sequence
  synthetic sequence
  MISC_FEATURE (9)..(9): The amino acid at position 9 is attached to an acyl group.
  MISC_FEATURE (10)..(10): The amino acid at position 10 is phosphorylated.
ARTKQTARKSTGGKAPRKQLA

SEQ ID NO: 90 - protein, 21 aa, Artificial sequence
  synthetic sequence
  MISC_FEATURE (4)..(4): The amino acid at positoin 4 is attached to three methyl groups.
  MISC_FEATURE (9)..(9): The amino acid at position 9 is attached to an acyl group.
  MISC_FEATURE (10)..(10): The amino acid at position 10 is phosphorylated.
ARTKQTARKSTGGKAPRKQLA

SEQ ID NO: 91 - protein, 22 aa, Artificial sequence
  synthetic sequence
ARTKQTARKSTGGKAPRKQLAC

SEQ ID NO: 92 - protein, 24 aa, Artificial sequence
  synthetic sequence
  MISC_FEATURE (9)..(9): The amino acid at position 9 is attached to an acyl group.
ARTKQTARKSTGGKAPRKQLATKA

SEQ ID NO: 93 - protein, 24 aa, Artificial sequence
  synthetic sequence
  MISC_FEATURE (9)..(9): The amino acid at position 9 is attached to three methyl groups.
ARTKQTARKSTGGKAPRKQLATKA

SEQ ID NO: 94 - protein, 25 aa, Artificial sequence
  synthetic sequence
ARTKQTARKSTGGKAPRKQLATKAA

SEQ ID NO: 95 - protein, 25 aa, Artificial sequence
  synthetic sequence
  MISC_FEATURE (4)..(4): The amino acid at positoin 4 is attached to three methyl groups.
ARTKQTARKSTGGKAPRKQLATKAA

SEQ ID NO: 96 - protein, 15 aa, Artificial sequence
  synthetic sequence
  MISC_FEATURE (7)..(7): The amino acid at position 7 is attached to a methyl group.
TKQTARKSTGGKAPR

SEQ ID NO: 97 - protein, 15 aa, Artificial sequence
  synthetic sequence
  MISC_FEATURE (7)..(7): The amino acid at position 7 is attached to two methyl groups.
TKQTARKSTGGKAPR

SEQ ID NO: 98 - protein, 15 aa, Artificial sequence
  synthetic sequence
  MISC_FEATURE (7)..(7): The amino aid at position 7 is attached to three methyl groups.
TKQTARKSTGGKAPR

SEQ ID NO: 99 - protein, 11 aa, Artificial sequence
  synthetic sequence
  MISC_FEATURE (6)..(6): The amino acid at position 6 is attached to an acyl group.
KSTGGKAPRKQ

SEQ ID NO: 100 - protein, 19 aa, Artificial sequence
  synthetic sequence
QTARKSTGGKAPRKQLASK

SEQ ID NO: 101 - protein, 25 aa, Artificial sequence
  synthetic sequence
APRKQLATKAARKSAPATGGVKKPH

SEQ ID NO: 102 - protein, 24 aa, Artificial sequence
  synthetic sequence
ATKAARKSAPATGGVKKPHRYRPG

SEQ ID NO: 103 - protein, 9 aa, Artificial sequence
  synthetic sequence
KAARKSAPA

SEQ ID NO: 104 - protein, 12 aa, Artificial sequence
  synthetic sequence
KAARKSAPATGG

SEQ ID NO: 105 - protein, 13 aa, Artificial sequence
  synthetic sequence
KAARKSAPATGGC

SEQ ID NO: 106 - protein, 12 aa, Artificial sequence
  synthetic sequence
  MISC_FEATURE (5)..(5): The amino acid at position 5 is attached to an acyl group.
KAARKSAPATGG

SEQ ID NO: 107 - protein, 12 aa, Artificial sequence
  synthetic sequence
  MISC_FEATURE (5)..(5): The amino acid at position 5 is attached to a methyl group.
KAARKSAPATGG

SEQ ID NO: 108 - protein, 12 aa, Artificial sequence
  synthetic sequence
  MISC_FEATURE (5)..(5): The amino acid at position 5 is attached to two methyl groups.
KAARKSAPATGG

SEQ ID NO: 109 - protein, 12 aa, Artificial sequence
  synthetic sequence
  MISC_FEATURE (5)..(5): The amino acid at positoin 5 is attached to three methyl groups.
KAARKSAPATGG

SEQ ID NO: 110 - protein, 24 aa, Artificial sequence
  synthetic sequence
  MISC_FEATURE (3)..(3): The amino acid at position 3 is attached to an acyl group.
ATKAARKSAPSTGGVKKPHRYRPG

SEQ ID NO: 111 - protein, 24 aa, Artificial sequence
  synthetic sequence
  MISC_FEATURE (8)..(8): The amino acid at position 8 is phosphorylated.
ATKAARKSAPATGGVKKPHRYRPG

SEQ ID NO: 112 - protein, 35 aa, Artificial sequence
  synthetic sequence
ARTKQTARKSTGGKAPRKQLATKAARKSAPATGGV

SEQ ID NO: 113 - protein, 11 aa, Artificial sequence
  synthetic sequence
  MISC_FEATURE (6)..(6): The amino acid at position 6 is attached to a methyl group.
STGGVKKPHRY

SEQ ID NO: 114 - protein, 11 aa, Artificial sequence
  synthetic sequence
  MISC_FEATURE (6)..(6): The amino acid at positoin 6 is attached to two methyl groups.
STGGVKKPHRY

SEQ ID NO: 115 - protein, 11 aa, Artificial sequence
  synthetic sequence
  MISC_FEATURE (6)..(6): The amino acid at position 6 is attached to three methyl groups.
STGGVKKPHRY

SEQ ID NO: 116 - protein, 20 aa, Artificial sequence
  synthetic sequence
  MISC_FEATURE (13)..(13): The amino acid at position 13 is attached to an acyl group.
GTVALREIRRYQKSTELLIR

SEQ ID NO: 117 - protein, 50 aa, Artificial sequence
  synthetic sequence
ARTKQTARKSTGGKAPRKQLATKAARKSAPATGGVKKPHRYRPGTVALRE

SEQ ID NO: 118 - protein, 33 aa, Artificial sequence
  synthetic sequence
  MISC_FEATURE (22)..(22): The amino acid at position 22 is attached to a methyl group.
  misc_feature (23)..(23): Xaa can be any naturally occurring amino acid
TELLIRKLPFQRLVREIAQDFKXTDLRFQSAAI

SEQ ID NO: 119 - protein, 11 aa, Artificial sequence
  synthetic sequence
EIAQDFKTDLR

SEQ ID NO: 120 - protein, 11 aa, Artificial sequence
  synthetic sequence
  MISC_FEATURE (7)..(7): The amino acid at position 7 is attached to an acyl group.
EIAQDFKTDLR

SEQ ID NO: 121 - protein, 11 aa, Artificial sequence
  synthetic sequence
  MISC_FEATURE (7)..(7): The amino acid at position 7 is attached to three methyl groups.
EIAQDFKTDLR

SEQ ID NO: 122 - protein, 21 aa, Artificial sequence
  synthetic sequence
RLVREIAQDFKTDLRFQSSAV

SEQ ID NO: 123 - protein, 21 aa, Artificial sequence
  synthetic sequence
  MISC_FEATURE (11)..(12): A methyl group is present between the amino acids at positions 11 and 12.
RLVREIAQDFKTDLRFQSSAV

SEQ ID NO: 124 - protein, 21 aa, Artificial sequence
  synthetic sequence
  MISC_FEATURE (11)..(12): Two methyl groups are present between the amino acids at positions 11 and 12.
RLVREIAQDFKTDLRFQSSAV

SEQ ID NO: 125 - protein, 21 aa, Artificial sequence
  synthetic sequence
  MISC_FEATURE (11)..(12): Three methyl groups are positioned between the amino acids at positions 11 and 12.
RLVREIAQDFKTDLRFQSSAV

SEQ ID NO: 126 - protein, 21 aa, Artificial sequence
  synthetic sequence
KRVTIMPKDIQLARRIRGERA

SEQ ID NO: 127 - protein, 136 aa, Artificial sequence
  synthetic sequence
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARRIRGERA

SEQ ID NO: 128 - protein, 7 aa, Artificial sequence
  synthetic sequence
SGRGKGG

SEQ ID NO: 129 - protein, 12 aa, Artificial sequence
  synthetic sequence
RGKGGKGLGKGA

SEQ ID NO: 130 - protein, 21 aa, Artificial sequence
  synthetic sequence
SGRGKGGKGLGKGGAKRHRKV

SEQ ID NO: 131 - protein, 20 aa, Artificial sequence
  synthetic sequence
KGLGKGGAKRHRKVLRDNWC

SEQ ID NO: 132 - protein, 30 aa, Artificial sequence
  synthetic sequence
  MISC_FEATURE (5)..(5): The amino acid at position 5 is attached to an acyl group.
  MISC_FEATURE (8)..(8): The amino acid at position 8 is attached to an acyl group.
  MISC_FEATURE (12)..(12): The amino acid at position 12 is attached to an acyl group.
  MISC_FEATURE (16)..(16): The amino acid at position 16 is attached to an acyl group.
SGRGKGGKGLGKGGAKRHRKVLRDNGSGSK

SEQ ID NO: 133 - protein, 20 aa, Artificial sequence
  synthetic sequence
SGRGKGGKGLGKGGAKRHRK

SEQ ID NO: 134 - protein, 20 aa, Artificial sequence
  synthetic sequence
  MISC_FEATURE (5)..(5): The amino acid at position 5 is attached to an acyl group.
SGRGKGGKGLGKGGAKRHRK

SEQ ID NO: 135 - protein, 20 aa, Artificial sequence
  synthetic sequence
  MISC_FEATURE (8)..(8): The amino acid at position 8 is attached to an acyl group.
SGRGKGGKGLGKGGAKRHRK

SEQ ID NO: 136 - protein, 20 aa, Artificial sequence
  synthetic sequence
  MISC_FEATURE (12)..(12): The amino acid at position 12 is attached to an acyl group.
SGRGKGGKGLGKGGAKRHRK

SEQ ID NO: 137 - protein, 20 aa, Artificial sequence
  synthetic sequence
  MISC_FEATURE (16)..(16): The amino acid at position 16 is attached to an acyl group.
SGRGKGGKGLGKGGAKRHRK

SEQ ID NO: 138 - protein, 20 aa, Artificial sequence
  synthetic sequence
KGLGKGGAKRHRKVLRDNWC

SEQ ID NO: 139 - protein, 103 aa, Artificial sequence [sequence omitted]

SEQ ID NO: 140 - protein, 10 aa, Artificial sequence
  synthetic sequence
CKATQASQEY

SEQ ID NO: 141 - protein, 22 aa, Artificial sequence
  synthetic sequence
ARTKQTARKSTGGKAPRKQLAC

SEQ ID NO: 142 - protein, 20 aa, Artificial sequence
  synthetic sequence
ARTKQTARKSTGGKAPRKWC

SEQ ID NO: 143 - protein, 13 aa, Artificial sequence
  synthetic sequence
KAARKSAPATGGC

SEQ ID NO: 144 - protein, 20 aa, Artificial sequence
  synthetic sequence
KGLGKGGAKRHRKVLRDNWC

SEQ ID NO: 145 - protein, 136 aa, Artificial sequence; identical to SEQ ID NO: 127
  synthetic sequence

SEQ ID NO: 146 - protein, 7 aa, Artificial sequence
  synthetic sequence
  misc_feature (1)..(7): Xaa can be any naturally occurring amino acid
XXXXXXX

SEQ ID NO: 147 - protein, 7 aa, Artificial sequence
  synthetic sequence
TKPRPGP

SEQ ID NO: 148 - protein, 7 aa, Artificial sequence
  synthetic sequence
MEHFPGP

SEQ ID NO: 149 - protein, 28 aa, Artificial sequence
  synthetic sequence
PEDEIWLPEPESVDVPAKPISTSSMMMP

SEQ ID NO: 150 - protein, 7 aa, Artificial sequence
  synthetic sequence
  misc_feature (1)..(7): Xaa can be any naturally occurring amino acid
XXXXXXX

SEQ ID NO: 151 - protein, 7 aa, Artificial sequence
  synthetic sequence
PKKKRKV

SEQ ID NO: 152 - protein, 12 aa, Artificial sequence
  synthetic sequence
PKKKRKVEDPYC

SEQ ID NO: 153 - protein, 33 aa, Artificial sequence
  synthetic sequence
PKKKRKVGPKKKRKVGPKKKRKVGPKKKRKVGC

SEQ ID NO: 154 - protein, 12 aa, Artificial sequence
  synthetic sequence
CYGRKKRRQRRR

SEQ ID NO: 155 - protein, 17 aa, Artificial sequence
  synthetic sequence
CSIPPEVKFNKPFVYLI

SEQ ID NO: 156 - protein, 17 aa, Artificial sequence
  synthetic sequence
DRQIKIWFQNRRMKWKK

SEQ ID NO: 157 - protein, 12 aa, Artificial sequence
  synthetic sequence
PKKKRKVEDPYC

SEQ ID NO: 158 - protein, 9 aa, Artificial sequence
  synthetic sequence
PAAKRVKLD

SEQ ID NO: 159 - protein, 7 aa, Artificial sequence
  synthetic sequence
  misc_feature (1)..(7): Xaa can be any naturally occurring amino acid
XXXXXXX

SEQ ID NO: 160 - protein, 11 aa, Artificial sequence
  synthetic sequence
YGRKKRRQRRR

SEQ ID NO: 161 - protein, 12 aa, Artificial sequence
  synthetic sequence
RRQRRTSKLMKR

SEQ ID NO: 162 - protein, 27 aa, Artificial sequence
  synthetic sequence
GWTLNSAGYLLGKINLKALAALAKKIL

SEQ ID NO: 163 - protein, 33 aa, Artificial sequence
  synthetic sequence
KALAWEAKLAKALAKALAKHLAKALAKALKCEA

SEQ ID NO: 164 - protein, 16 aa, Artificial sequence
  synthetic sequence
RQIKIWFQNRRMKWKK

SEQ ID NO: 165 - protein, 9 aa, Artificial sequence
  synthetic sequence
RKKRRQRRR

SEQ ID NO: 166 - protein, 8 aa, Artificial sequence
  synthetic sequence
RKKRRQRR

SEQ ID NO: 167 - protein, 11 aa, Artificial sequence
  synthetic sequence
YARAAARQARA

SEQ ID NO: 168 - protein, 11 aa, Artificial sequence
  synthetic sequence
THRLPRRRRRR

SEQ ID NO: 169 - protein, 11 aa, Artificial sequence
  synthetic sequence
GGRRARRRRRR

SEQ ID NO: 170 - protein, 10 aa, Artificial sequence
  synthetic sequence
QQQQQQQQQQ

SEQ ID NO: 171 - DNA, 20 bp, Artificial sequence
  synthetic sequence
agagtctctcagctggtaca

SEQ ID NO: 172 - protein, 7 aa, Artificial sequence
  synthetic sequence
  misc_feature (1)..(7): Xaa can be any naturally occurring amino acid
XXXXXXX

SEQ ID NO: 173 - protein, 7 aa, Artificial sequence
  synthetic sequence
  misc_feature (1)..(7): Xaa can be any naturally occurring amino acid
XXXXXXX

SEQ ID NO: 174 - protein, 7 aa, Artificial sequence
  synthetic sequence
  misc_feature (1)..(7): Xaa can be any naturally occurring amino acid
XXXXXXX

SEQ ID NO: 175 - protein, 7 aa, Artificial sequence
  synthetic sequence
  misc_feature (1)..(7): Xaa can be any naturally occurring amino acid
XXXXXXX

SEQ ID NO: 176 - protein, 7 aa, Artificial sequence
  synthetic sequence
  misc_feature (1)..(7): Xaa can be any naturally occurring amino acid
XXXXXXX

SEQ ID NO: 177 - protein, 7 aa, Artificial sequence
  synthetic sequence
  misc_feature (1)..(7): Xaa can be any naturally occurring amino acid
XXXXXXX

SEQ ID NO: 178 - protein, 7 aa, Artificial sequence
  synthetic sequence
  misc_feature (1)..(7): Xaa can be any naturally occurring amino acid
XXXXXXX

SEQ ID NO: 179 - protein, 7 aa, Artificial sequence
  synthetic sequence
  misc_feature (1)..(7): Xaa can be any naturally occurring amino acid
XXXXXXX

SEQ ID NO: 180 - protein, 41 aa, Artificial sequence
  synthetic sequence
YTIWMPENPRPGTPCDIFTNSRGKRASNGGGGRRRRRRRRR

SEQ ID NO: 181 - protein, 5 aa, Artificial sequence
  synthetic sequence
RGDGW

SEQ ID NO: 182 - protein, 11 aa, Artificial sequence
  synthetic sequence
GCGYGRGDSPG

SEQ ID NO: 183 - protein, 32 aa, Artificial sequence
  synthetic sequence
YTIWMPENPRPGTPCDIFTNSRGKRASNGGGG

SEQ ID NO: 184 - protein, 164 aa, Artificial sequence
  synthetic sequence
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 185 - protein, 192 aa, Artificial sequence
  synthetic sequence
PEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 186 - protein, 138 aa, Artificial sequence
  synthetic sequence
SSGLVPRGSHMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 187 - protein, 13 aa, Artificial sequence
  synthetic sequence
DGARYCRGDCFDG

SEQ ID NO: 188 - protein, 11 aa, Artificial sequence
  synthetic sequence
GCGYGRGDSPG

SEQ ID NO: 189 - protein, 174 aa, Artificial sequence
  synthetic sequence
RRRRRRRRRMEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 190 - protein, 201 aa, Artificial sequence
  synthetic sequence
RRRRRRRRRPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 191 - protein, 201 aa, Artificial sequence
  synthetic sequence
PEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPRRRRRRRRR

SEQ ID NO: 192 - protein, 149 aa, Artificial sequence
  synthetic sequence
MGSSHHHHHHSSGLVPRGSHMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSSARSTQGTTGIREDPDVCLKAP

SEQ ID NO: 193 - protein, 147 aa, Artificial sequence
  synthetic sequence
RRRRRRRRRSSGLVPRGSHMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 194 - protein, 165 aa, Artificial sequence
  synthetic sequence
  MISC_FEATURE (1)..(1): The amino acid at position 1 may be attached to a cationic anchoring domain.
  MISC_FEATURE (165)..(165): The amino acid at position 165 may be attached to a cationic anchoring domain.
MEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 195 - protein, 192 aa, Artificial sequence; identical to SEQ ID NO: 185
  synthetic sequence
  MISC_FEATURE (1)..(1): The amino acid at position 1 may be attached to a cationic anchoring domain
  MISC_FEATURE (195)..(195): The amino acid at position 195 may be attached to a cationic anchoring domain

SEQ ID NO: 196 - protein, 138 aa, Artificial sequence; identical to SEQ ID NO: 186
  synthetic sequence
  MISC_FEATURE (1)..(1): The amino acid at position 1 may be attached to a cationic anchoring domain.

SEQ ID NO: 197 - protein, 142 aa, Artificial sequence
  synthetic sequence
  MISC_FEATURE (4)..(5): A cationic anchoring domain may be present between the amino acids at positions 4 and 5.
MGSSSSGLVPRGSHMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 198 - protein, 7 aa, Artificial sequence
  synthetic sequence
  misc_feature (1)..(7): Xaa can be any naturally occurring amino acid
XXXXXXX

SEQ ID NO: 199 - protein, 7 aa, Artificial sequence
  synthetic sequence
  misc_feature (1)..(7): Xaa can be any naturally occurring amino acid
XXXXXXX

SEQ ID NO: 200 - protein, 7 aa, Artificial sequence
  synthetic sequence
  misc_feature (1)..(7): Xaa can be any naturally occurring amino acid
XXXXXXX

SEQ ID NO: 201 - protein, 12 aa, Artificial sequence
  synthetic sequence
LPKKRKFSEISS

SEQ ID NO: 202 - protein, 10 aa, Artificial sequence
  synthetic sequence
KRKRWENDIP

SEQ ID NO: 203 - protein, 10 aa, Artificial sequence
  synthetic sequence
KRKRWENNIP

SEQ ID NO: 204 - protein, 12 aa, Artificial sequence
  synthetic sequence
TGGVMKRKRGSV

SEQ ID NO: 205 - protein, 12 aa, Artificial sequence
  synthetic sequence
PILPLKRRRGSP

SEQ ID NO: 206 - protein, 12 aa, Artificial sequence
  synthetic sequence
TYSGVKRKRNVV

SEQ ID NO: 207 - protein, 12 aa, Artificial sequence
  synthetic sequence
THIGYKRKRDSV

SEQ ID NO: 208 - protein, 12 aa, Artificial sequence
  synthetic sequence
LSGTKRKRAYFI

SEQ ID NO: 209 - protein, 12 aa, Artificial sequence
  synthetic sequence
QRRLLKRKRGSL

SEQ ID NO: 210 - protein, 12 aa, Artificial sequence
  synthetic sequence
QIGKKRKRDYLD

SEQ ID NO: 211 - protein, 12 aa, Artificial sequence
  synthetic sequence
KRGKRKRLVRPW

SEQ ID NO: 212 - protein, 12 aa, Artificial sequence
  synthetic sequence
KKGKRKRLVRPW

SEQ ID NO: 213 - protein, 12 aa, Artificial sequence
  synthetic sequence
PSRKRKRESDHI

SEQ ID NO: 214 - protein, 12 aa, Artificial sequence
  synthetic sequence
PSRKRKRDHYAV

SEQ ID NO: 215 - protein, 12 aa, Artificial sequence
  synthetic sequence
ISRKRKRDLEFV

SEQ ID NO: 216 - protein, 12 aa, Artificial sequence
  synthetic sequence
ITRKRKRDLVFT

SEQ ID NO: 217 - protein, 12 aa, Artificial sequence
  synthetic sequence
EPNPRKRKRSEL

SEQ ID NO: 218 - protein, 12 aa, Artificial sequence
  synthetic sequence
TSPSRKRKWDQV

SEQ ID NO: 219 - protein, 12 aa, Artificial sequence
  synthetic sequence
TLERKRKLAVLY

SEQ ID NO: 220 - protein, 12 aa, Artificial sequence
  synthetic sequence
RRRKRRREWEDF

SEQ ID NO: 221 - protein, 12 aa, Artificial sequence
  synthetic sequence
HRYCGKRRRRTR

SEQ ID NO: 222 - protein, 11 aa, Artificial sequence
  synthetic sequence
SVLGKRSRTWE

SEQ ID NO: 223 - protein, 12 aa, Artificial sequence
  synthetic sequence
YGRVSKRPRYQF

SEQ ID NO: 224 - protein, 11 aa, Artificial sequence
  synthetic sequence
RKRGRKRFRSV

SEQ ID NO: 225 - protein, 12 aa, Artificial sequence
  synthetic sequence
KRKYAVFLESQN

SEQ ID NO: 226 - protein, 12 aa, Artificial sequence
  synthetic sequence
KRKYSIYLGSQS

SEQ ID NO: 227 - protein, 12 aa, Artificial sequence
  synthetic sequence
KRKWMAFVMGDP

SEQ ID NO: 228 - protein, 12 aa, Artificial sequence
  synthetic sequence
KRKCAVFLEGQN

SEQ ID NO: 229 - protein, 12 aa, Artificial sequence
  synthetic sequence
IPRKRSFAELYD

SEQ ID NO: 230 - protein, 12 aa, Artificial sequence
  synthetic sequence
RLTPRKRAFSEV

SEQ ID NO: 231 - protein, 9 aa, Artificial sequence
  synthetic sequence
KRSWSMAFC

SEQ ID NO: 232 - protein, 10 aa, Artificial sequence
  synthetic sequence
KRTKAQAFTE

SEQ ID NO: 233 - protein, 12 aa, Artificial sequence
  synthetic sequence
KRPYSIAFPLGQ

SEQ ID NO: 234 - protein, 14 aa, Artificial sequence
  synthetic sequence
RRRSVLKRSWSVAF

SEQ ID NO: 235 - protein, 12 aa, Artificial sequence
  synthetic sequence
KRRYSDAFRLPV

SEQ ID NO: 236 - protein, 12 aa, Artificial sequence
  synthetic sequence
KRKYSDAFGLPV

SEQ ID NO: 237 - protein, 12 aa, Artificial sequence
  synthetic sequence
IGRKRGYSVAFG

SEQ ID NO: 238 - protein, 12 aa, Artificial sequence
  synthetic sequence
IGRKRVWAVAFY

SEQ ID NO: 239 - protein, 12 aa, Artificial sequence
  synthetic sequence
WAGRKRTWRDAF

SEQ ID NO: 240 - protein, 12 aa, Artificial sequence
  synthetic sequence
SSHRKRKFSDAF

SEQ ID NO: 241 - protein, 7 aa, Artificial sequence
  synthetic sequence
PSHRKRI

SEQ ID NO: 242 - protein, 12 aa, Artificial sequence
  synthetic sequence
TAHRKRKFSDAF

SEQ ID NO: 243 - protein, 12 aa, Artificial sequence
  synthetic sequence
RVQRKRKWSEAF

SEQ ID NO: 244 - protein, 12 aa, Artificial sequence
  synthetic sequence
RLTRKRCYDCAF

SEQ ID NO: 245 - protein, 12 aa, Artificial sequence
  synthetic sequence
LVNRKRRYWEAF

SEQ ID NO: 246 - protein, 12 aa, Artificial sequence
  synthetic sequence
LGKRYDRDWDYK

SEQ ID NO: 247 - protein, 12 aa, Artificial sequence
  synthetic sequence
RSSGILGKRKFE

SEQ ID NO: 248 - protein, 12 aa, Artificial sequence
  synthetic sequence
VHKTVLGKRKYW

SEQ ID NO: 249 - protein, 12 aa, Artificial sequence
  synthetic sequence
SILGKRKNRDPS

SEQ ID NO: 250 - protein, 12 aa, Artificial sequence
  synthetic sequence
  misc_feature (6)..(6): Xaa can be any naturally occurring amino acid
QSVLGXRKSRPF

SEQ ID NO: 251 - protein, 12 aa, Artificial sequence
  synthetic sequence
TVHLGKRRLRPW

SEQ ID NO: 252 - protein, 12 aa, Artificial sequence
  synthetic sequence
RVLGKRKTGRSP

SEQ ID NO: 253 - protein, 11 aa, Artificial sequence
  synthetic sequence
VLGKRKRDDCW

SEQ ID NO: 254 - protein, 12 aa, Artificial sequence
  synthetic sequence
HGRQVLGIKRKR

SEQ ID NO: 255 - protein, 5 aa, Artificial sequence
  synthetic sequence
SVLGI

SEQ ID NO: 256 - protein, 12 aa, Artificial sequence
  synthetic sequence
SVLGKRKRHHLD

SEQ ID NO: 257 - protein, 12 aa, Artificial sequence
  synthetic sequence
PVLGKRKRSLSS

SEQ ID NO: 258 - protein, 12 aa, Artificial sequence
  synthetic sequence
RVLGKRKREDRP

SEQ ID NO: 259 - protein, 12 aa, Artificial sequence
  synthetic sequence
ILGKRKRSHHPY

SEQ ID NO: 260 - protein, 12 aa, Artificial sequence
  synthetic sequence
PILGKRKRHLFL

SEQ ID NO: 261 - protein, 12 aa, Artificial sequence
  synthetic sequence
LLGKRKRPSIEH

SEQ ID NO: 262 - protein, 12 aa, Artificial sequence
  synthetic sequence
SMLGKRKRCIIS

SEQ ID NO: 263 - protein, 12 aa, Artificial sequence
  synthetic sequence
TLGKRKRISCVT

SEQ ID NO: 264 - protein, 12 aa, Artificial sequence
  synthetic sequence
DTRLGKRKRRPW

SEQ ID NO: 265 - protein, 7 aa, Artificial sequence
  synthetic sequence
  misc_feature (1)..(7): Xaa can be any naturally occurring amino acid
XXXXXXX

SEQ ID NO: 266 - protein, 7 aa, Artificial sequence
  synthetic sequence
  misc_feature (1)..(7): Xaa can be any naturally occurring amino acid
XXXXXXX

SEQ ID NO: 267 - protein, 7 aa, Artificial sequence
  synthetic sequence
  misc_feature (1)..(7): Xaa can be any naturally occurring amino acid
XXXXXXX

SEQ ID NO: 268 - protein, 7 aa, Artificial sequence
  synthetic sequence
  misc_feature (1)..(7): Xaa can be any naturally occurring amino acid
XXXXXXX

SEQ ID NO: 269 - protein, 7 aa, Artificial sequence
  synthetic sequence
  misc_feature (1)..(7): Xaa can be any naturally occurring amino acid
XXXXXXX

SEQ ID NO: 270 - protein, 7 aa, Artificial sequence
  synthetic sequence
  misc_feature (1)..(7): Xaa can be any naturally occurring amino acid
XXXXXXX

SEQ ID NO: 271 - protein, 428 aa, Artificial sequence
  synthetic sequence
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 272 - protein, 1203 aa, Artificial sequence
  synthetic sequence
MAACGRVRRMFRLSAALHLLLLFAAGAEKLPGQGVHSQGQGPGANFVSFVGQAGGGGPAGQQLPQLPQSSQLQQQQQQQQQQQQPQPPQPPFPAGGPPARRGGAGAGGGWKLAEEESCREDVTRVCPKHTWSNNLAVLECLQDVREPENEISSDCNHLLWNYKLNLTTDPKFESVAREVCKSTITEIKECADEPVGKGYMVSCLVDHRGNITEYQCHQYITKMTAIIFSDYRLICGFMDDCKNDINILKCGSIRLGEKDAHSQGEVVSCLEKGLVKEAEEREPKIQVSELCKKAILRVAELSSDDFHLDRHLYFACRDDRERFCENTQAGEGRVYKCLFNHKFEESMSEKCREALTTRQKLIAQDYKVSYSLAKSCKSDLKKYRCNVENLPRSREARLSYLLMCLESAVHRGRQVSSECQGEMLDYRRMLMEDFSLSPEIILSCRGEIEHHCSGLHRKGRTLHCLMKVVRGEKGNLGMNCQQALQTLIQETDPGADYRIDRALNEACESVIQTACKHIRSGDPMILSCLMEHLYTEKMVEDCEHRLLELQYFISRDWKLDPVLYRKCQGDASRLCHTHGWNETSEFMPQGAVFSCLYRHAYRTEEQGRRLSRECRAEVQRILHQRAMDVKLDPALQDKCLIDLGKWCSEKTETGQELECLQDHLDDLVVECRDIVGNLTELESEDIQIEALLMRACEPIIQNFCHDVADNQIDSGDLMECLIQNKHQKDMNEKCAIGVTHFQLVQMKDFRFSYKFKMACKEDVLKLCPNIKKKVDVVICLSTTVRNDTLQEAKEHRVSLKCRRQLRVEELEMTEDIRLEPDLYEACKSDIKNFCSAVQYGNAQIIECLKENKKQLSTRCHQKVFKLQETEMMDPELDYTLMRVCKQMIKRFCPEADSKTMLQCLKQNKNSELMDPKCKQMITKRQITQNTDYRLNPMLRKACKADIPKFCHGILTKAKDDSELEGQVISCLKLRYADQRLSSDCEDQIRIIIQESALDYRLDPQLQLHCSDEISSLCAEEAAAQEQTGQVEECLKVNLLKIKTELCKKEVLNMLKESKADIFVDPVLHTACALDIKHHCAAITPGRGRQMSCLMEALEDKRVRLQPECKKRLNDRIEMWSYAAKVAPADGFSDLAMQVMTSPSKNYILSVISGSICILFLIGLMCGRITKRVTRELKDRLQYRSETMAYKGLVWSQDVTGSPA

SEQ ID NO: 273 - protein, 742 aa, Artificial sequence
  synthetic sequence
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 274 - protein, 426 aa, Artificial sequence
  synthetic sequence
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTPPPSLAGAPWGAVQSAVPLSVAGGRVGVFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 275 - protein, 417 aa, Artificial sequence
  synthetic sequence
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 276 - protein, 410 aa, Artificial sequence
  synthetic sequence
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 277 - protein, 585 aa, Artificial sequence
  synthetic sequence
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 278 - DNA, 20 bp, Artificial sequence
  synthetic sequence
ctcgtgaccaccctgaccta

SEQ ID NO: 279 - DNA, 166 bp, Artificial sequence
  synthetic sequence
gcgatgccacctacggcaagctgaccctgaagttcatctgcaccaccggcaagctgcccgtgccctggcccaccctcgtgaccaccctgacccccaactggggtaacctttgagttctctcagttgggggctacggcgtgcagtgcttcagccgctaccccgacca

SEQ ID NO: 280 - DNA, 20 bp, Artificial sequence
  synthetic sequence
agagtctctcagctggtaca

SEQ ID NO: 281 - DNA, 166 bp, Artificial sequence
  synthetic sequence
gtcccacagatatccagaaccctgaccctgccgtgtccccaactggggtaacctttgagttctctcagttgggggaccagctgagagactctaaatccagtgacaagtctgtctgcctattcaccgattttgattctcaaacaaatgtgtcacaaagtaaggattc

SEQ ID NO: 282 - protein, 10 aa, Artificial sequence
  synthetic sequence
RRRRRRRRRR

SEQ ID NO: 283 - protein, 50 aa, Artificial sequence
  synthetic sequence
RRRRRRRRRRRRRRRRRRRRRRRRRRRRRRRRRRRRRRRRRRRRRRRRRR

SEQ ID NO: 284 - protein, 23 aa, Artificial sequence
  synthetic sequence
CEVSSKGATICKKGFKKAVVKCA

SEQ ID NO: 285 - protein, 23 aa, Artificial sequence
  synthetic sequence
CSGRGKQGCKARAKAKTRSSRCA

SEQ ID NO: 286 - protein, 32 aa, Artificial sequence
  synthetic sequence
KKKRKSCRGKQGCKARAKAKTRSSRCAKKKRK

SEQ ID NO: 287 - protein, 20 aa, Artificial sequence
  synthetic sequence
EEEEEEEEEEEEEEEEEEEE

SEQ ID NO: 288 - protein, 7 aa, Artificial sequence
  synthetic sequence
  misc_feature (1)..(7): Xaa can be any naturally occurring amino acid
XXXXXXX

SEQ ID NO: 289 - protein, 27 aa, Artificial sequence
  synthetic sequence
RRRRRRRRRGGGGSGGGGSNFYLYLRA

SEQ ID NO: 290 - protein, 37 aa, Artificial sequence
  synthetic sequence
  MISC_FEATURE (16)..(17): Alexa647 is present between the amino acids a positions 16 and 17.
KKKRKKKKRKGGGGSCGGGGSSFKFLFDIIKKIAESF

SEQ ID NO: 291 - protein, 27 aa, Artificial sequence
  synthetic sequence
RRRRRRRRRGGGGSGGGGSFTDNAKTI

SEQ ID NO: 292 - DNA, 20 bp, Artificial sequence
  synthetic sequence
ccagcctaagttggggagac

SEQ ID NO: 293 - DNA, 20 bp, Artificial sequence
  synthetic sequence
gtgactgcgtgagactgact

SEQ ID NO: 294 - DNA, 20 bp, Artificial sequence
  synthetic sequence
gagctgttcaccggggtggt

SEQ ID NO: 295 - DNA, 21 bp, Artificial sequence
  synthetic sequence
ctgagtcccagtccatcacga

SEQ ID NO: 296 - protein, 13 aa, Artificial sequence
  synthetic sequence
NPKLTRMLTFKFY

SEQ ID NO: 297 - protein, 14 aa, Artificial sequence
  synthetic sequence
TSVGKYPNTGYYGD

SEQ ID NO: 298 - protein, 8 aa, Artificial sequence
  synthetic sequence
SNRWLDVK

SEQ ID NO: 299 - protein, 14 aa, Artificial sequence
  synthetic sequence
EKFILKVRPAFKAV

SEQ ID NO: 300 - protein, 14 aa, Artificial sequence
  synthetic sequence
EKFILKVRPAFKAV

SEQ ID NO: 301 - protein, 7 aa, Artificial sequence
  synthetic sequence
  misc_feature (1)..(7): Xaa can be any naturally occurring amino acid
XXXXXXX

SEQ ID NO: 302 - protein, 24 aa, Artificial sequence
  synthetic sequence
SNYSIIDKLVNIVDDLVECVKENS

SEQ ID NO: 303 - protein, 24 aa, Artificial sequence
  synthetic sequence
  misc_feature (1)..(1): The amino acid at position 1 is attached to an acyl group.
  misc_feature (5)..(5): Xaa is aminoisobutyric acid.
  misc_feature (9)..(9): Xaa is aminoisobutyric acid.
  misc_feature (12)..(12): Xaa is aminoisobutyric acid.
  misc_feature (16)..(16): Xaa is aminoisobutyric acid.
  misc_feature (19)..(19): Xaa is aminoisobutyric acid.
SNYSXADKXANXADDXAEXAKENS

SEQ ID NO: 304 - DNA, 71 bp, Artificial sequence
  synthetic sequence
ggcccaccctcgtgaccaccctgacctacggcgtgcagtgcttcagccgctaccccgaccacatgaagcag

SEQ ID NO: 305 - DNA, 75 bp, Artificial sequence
  synthetic sequence
  misc_feature (26)..(26): n is a, c, g, or t
ggcccaccctcgtgaccaccctgacnctacggcgtgcagtgcttcagccgctaccccgaccacatgaagcagcac

SEQ ID NO: 306 - DNA, 75 bp, Artificial sequence
  synthetic sequence
ggcccaccctcgtgaccaccctgacccccaactggggtaacctttgagttctctcagttgggggctacggcgtgc

SEQ ID NO: 307 - DNA, 74 bp, Artificial sequence
  synthetic sequence
ggcccaccctcgtgaccaccctgactacggcgtgcagtgcttcagccgctaccccgaccacatgaagcagcacg

SEQ ID NO: 308 - DNA, 71 bp, Artificial sequence
  synthetic sequence
ggcccaccctcgtgaccaccctgacggcgtgcagtgcttcagccgctaccccgaccacatgaagcagcacg

SEQ ID NO: 309 - DNA, 75 bp, Artificial sequence
  synthetic sequence
  misc_feature (26)..(27): n is a, c, g, or t
ggcccaccctcgtgaccaccctgacnnctacggcgtgcagtgcttcagccgctaccccgaccacatgaagcagca

SEQ ID NO: 310 - DNA, 65 bp, Artificial sequence
  synthetic sequence
ggcccaccctcgtgaccaccctgaccagtgcttcagccgctaccccgaccacatgaagcagcacg

SEQ ID NO: 311 - DNA, 70 bp, Artificial sequence
  synthetic sequence
ggcccaccctcgtgaccaccctgacgcgtgcagtgcttcagccgctaccccgaccacatgaagcagcacg

SEQ ID NO: 312 - DNA, 55 bp, Artificial sequence
  synthetic sequence
ggcccaccctcgtgaccacccttcagccgctaccccgaccacatgaagcagcacg

SEQ ID NO: 313 - DNA, 65 bp, Artificial sequence
  synthetic sequence
ggcccaccctcgtgaccaccctgagcagtgcttcagccgctaccccgaccacatgaagcagcacg

SEQ ID NO: 314 - DNA, 52 bp, Artificial sequence
  synthetic sequence
ggcccaccctcgtgacgcttcagccgctaccccgaccacatgaagcagcacg

SEQ ID NO: 315 - DNA, 66 bp, Artificial sequence
  synthetic sequence
ggcccaccctcgtgaccaccggcgtgcagtgcttcagccgctaccccgaccacatgaagcagcacg

SEQ ID NO: 316 - DNA, 64 bp, Artificial sequence
  synthetic sequence
ggcccaccctcgtgaccaccctgacgcagtgcttcagccgcacccgaccacatgaagcagcacg

SEQ ID NO: 317 - DNA, 75 bp, Artificial sequence
  synthetic sequence
  misc_feature (26)..(40): n is a, c, g, or t
ggcccaccctcgtgaccaccctgacnnnnnnnnnnnnnnnctacggcgtgcagtgcttcagccgctaccccgacc

SEQ ID NO: 318 - DNA, 73 bp, Artificial sequence
  synthetic sequence
ggcccaccctcgtgaccaccctgacacggcgtgcagtgcttcagccgctaccccgaccacatgaagcagcacg

SEQ ID NO: 319 - DNA, 75 bp, Artificial sequence
  synthetic sequence
  misc_feature (26)..(33): n is a, c, g, or t
ggcccaccctcgtgaccaccctgacnnnnnnnnctacggcgtgcagtgcttcagccgctaccccgaccacatgaa

SEQ ID NO: 320 - protein, 7 aa, Artificial sequence
  synthetic sequence
NFYLYRA

SEQ ID NO: 321 - protein, 10 aa, Artificial sequence
  synthetic sequence
RYPLTFGWCF

SEQ ID NO: 322 - protein, 12 aa, Artificial sequence
  synthetic sequence
VVLKYEKDAFKR

SEQ ID NO: 323 - protein, 7 aa, Artificial sequence
  synthetic sequence
ENLVLNE

SEQ ID NO: 324 - DNA, 19 bp, Artificial sequence
  synthetic sequence
atggtgagcaagggcgagg

SEQ ID NO: 325 - DNA, 22 bp, Artificial sequence
  synthetic sequence
cacgaactccagcaggaccatg